(19) United States
(12) Patent Application Publication
(10) Pub. No.: US 2024/0025849 A1
(43) Pub. Date: Jan. 25, 2024

(54) INHIBITORS OF NO PRODUCTION

(71) Applicants: MAX DELBRÜCK-CENTRUM FÜR MOLEKULARE MEDIZIN IN DER HELMHOLTZ-GEMEINSCHAFT, Berlin (DE); CHARITÉ - UNIVERSITÄTSMEDIZIN BERLIN, Berlin (DE)

(72) Inventors: Helmut KETTENMANN, Berlin (DE); Susanne WOLF, Berlin (DE); Philipp JORDAN, Berlin (DE); Sabrina KLEISSLE, Berlin (DE); Martin NEUENSCHWANDER, Berlin (DE); Edgar SPECKER, Berlin (DE); Marc NAZARE, Berlin (DE)

(21) Appl. No.: 18/023,233

(22) PCT Filed: Aug. 26, 2021
(86) PCT No.: PCT/EP2021/073663

§ 371 Date: Feb. 24, 2023

(30) Foreign Application Priority Data
Aug. 26, 2020 (EP) 20192972.6

(51) Int. Cl. C07D 207/16; A61K 47/54; A61K 47/60; C07D 471/04; C07D 405/06; C07D 409/06; C07D 403/06; C07D 405/12; C07D 401/06; C07D 403/12; C07D 205/04; C07D 417/12; C07D 209/42; C07D 217/26; C07D 409/12; C07D 211/60; A61P 9/10; A61P 25/28

(52) U.S. Cl. CPC C07D 207/16; A61K 47/545; A61K 47/60; C07D 471/04; C07D 405/06; C07D 409/06; C07D 403/06; C07D 405/12; C07D 401/06; C07D 403/12; C07D 205/04; C07D 417/12; C07D 209/42; C07D 217/26; C07D 409/12; C07D 211/60; A61P 9/10; A61P 25/28

(57) ABSTRACT

The invention relates to compounds useful as inhibitors of NO production, especially inhibitors of the inducible NO synthase iNOS expressed by microglia and macrophages. The invention also relates to pharmaceutical compositions comprising these compounds and to therapeutic uses of these compounds, especially in the prophylaxis or treatment of conditions characterized by excess NO production, such as ischemic stroke and retinopathies.

The present invention relates to small molecule inhibitors of NO production and to the use of such inhibitors in a method of treatment or prevention of a disease, in particular ischemic stroke and retinal vascular disorders.

### BACKGROUND OF THE INVENTION

Microglia are the immune cells of the CNS and get activated upon pathology. Microglial activation is often linked to the release of pro-inflammation substances such as tumor necrosis factor α (TNFα), Interleukin 1β (IL1β), Interleukin 6 (IL6), and nitric oxide (NO), increased phagocytosis and increase of directed migration towards injury. In high concentrations, NO reacts non-specifically with proteins, nucleic acids and lipids, resulting in damage of the host tissue, and potentiates inflammation. NO plays a role in various pathologies including diabetes, hypertension, cancer, drug addiction, stroke, intestinal motility disorders, memory and learning disorders, retinal pathologies, septic shock, inflammatory and autoimmune diseases.

NO is produced by the nitric oxide synthase (NOS), an enzyme that catalyses the conversion of L-arginine to citrulline and NO. In human and mice, three isoforms of NOS are known: eNOS, nNOS, and iNOS. eNOS and nNOS are constitutively expressed by endothelial cells and neurons, respectively, and are regulated via the cytosolic calcium concentration. iNOS is induced by a pathologic event in microglia and macrophages, in a calcium independent manner. Upon induction, iNOS is upregulated for several days and synthesizes NO, reaching concentrations that are toxic for neurons and other brain cells (Lind et al., 2017). Since excessive NO can damage tissue and organs, NO antagonizing compounds are therapeutically relevant. However, NO has multiple functions depending on the source of production and on the target cells. The general NOS inhibitor L-NMMA raises cardiovascular safety concerns and has an unfavorable pharmacokinetic profile. Therefore a specific iNOS inhibitor would have a great advantage over general NO inhibitors. Currently three specific iNOS inhibitors are known. Aminoguanidine is a highly reactive nucleophilic reagent that reacts with many biological molecules (pyridoxal phosphate, pyruvate, glucose, malondialdehyde, and others). A clinical trial with aminoguanidine to prevent progression of diabetic nephropathy was terminated early due to safety concerns and apparent lack of efficacy. GW274150 and GW273629 are potent and highly selective inhibitors of inducible nitric oxide synthase in-vitro and in-vivo. However, GW273629 has an unfavourable pharmacokinetic profile, while GW274150 was used for Arthritis, Migraine, and Asthma in P1/2clinical trials with no significant or only unpublished results.

Thus, one aim of the present invention is to provide novel compounds targeting NO and specifically iNOS.

The inventors have discovered novel compounds that inhibit NO release from microglia cells. These compounds are useful in diseases characterized by microglial NO production and provide inter alia for: (i) selective inhibition of NO production by microglia and macrophages; (ii) selective inhibition of NO production by iNOS; (iii) reduced interference with physiological NO production by eNOS and nNOS; (iv) reduced side effects, in particular on functions of microglia and macrophages other than NO production, and on neurons, oligodendrocytes and astrocytes; (v) improved ability to pass the blood-brain-barrier.

The above-described objects are solved and the advantages are achieved by the subject-matter of the enclosed independent claims. Preferred embodiments of the invention are included in the dependent claims as well as in the following description, examples and figures.

The above overview does not necessarily describe all advantages and all problems solved by the present invention.

### SUMMARY OF THE INVENTION

In a first aspect, the present invention provides a compound for use in a method of treatment or prevention of a disease, characterized by a formula (1a) or (1b),

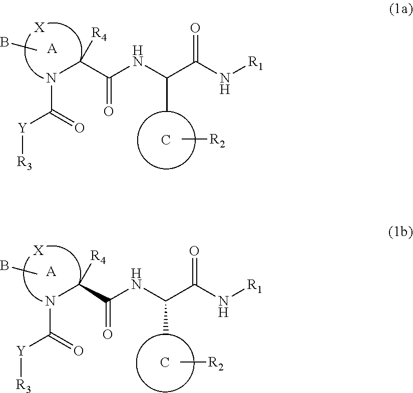

wherein
R¹ is independently selected from the group consisting of optionally substituted C₁-C₁₀-alkyl, C₁-C₁₀-heteroalkyl, C₁-C₁₀-haloalkyl, C₂-C₁₀-alkenyl, C₂-C₁₀-heteroalkenyl, C₂-C₁₀-alkynyl, C₃-C₁₀-cycloalkyl, C₃-C₁₀-heterocycloalkyl, C₄-C₁₀-cycloalkenyl, C₄-C₁₀-heterocycloalkenyl, C₅-C₁₄-aryl, C₅-C₁₄-heteroaryl, C₆-C₁₅ alicyclic system, C₆-C₁₅-aralkyl and C₆-C₁₅-heteroaralkyl;
R² is selected from the group consisting of —F, —Br, —Cl, —R⁵, —OR⁵, —COR⁵, —CO₂R⁵, —OCX₃, —N(R)R′, —N(R)—C(O)—R′, —C(O)—N(R)—C(O)—R′, —N(R)—C(O)—OR′, —C(O)N(R)R′, —N(R)—C(O)—N(R′)R″, —R═NR′, —R═NH, —CN, —NC, —ONO, —NO₂, —ONO₂, —NO, —OCN, —NCO, —SR, —SX₃, —SX₅, —S(O)R, —SO₂R, and —SO₃H, wherein
   R⁵ is selected from the group consisting of —H and optionally substituted C₁-C₁₀ alkyl, C₂-C₁₀-alkenyl, C₂-C₁₀-alkynyl, C₃-C₁₀-cycloalkyl, C₃-C₁₀-heterocycloalkyl, C₅-C₁₄-aryl, C₅-C₁₄-heteroaryl, C₆-C₁₅-aralkyl, C₆-C₁₅-heteroaralkyl, and —(OCH₂CH₂)ₙ₁—NH—R⁶, wherein
      n1 is 2-10, particularly 4-8, and R⁶ is biotinyl;
      R, R′ and R″ are independently selected from the group consisting of —H, C₁-C₃-alkyl and C₂-C₃-alkenyl;
R³ is independently selected from the group consisting of optionally substituted C₂-C₁₀-alkyl, C₂-C₁₀-heteroalkyl, C₂-C₁₀-haloalkyl, C₂-C₁₀-alkenyl, C₂-C₁₀-heteroalkenyl, C₂-C₁₀-alkynyl, C₃-C₁₀-cycloalkyl, C₃-C₁₀-heterocycloalkyl, C₄-C₁₀-cycloalkenyl, C₄-C₁₀-heterocycloalkenyl, C₅-C₁₄-aryl, C₅-C₁₄-heteroaryl, C₆-C₁₅ alicyclic system, C₆-C₁₅-aralkyl and C₆-C₁₅-heteroaralkyl;
R⁴ is selected from the group consisting of —H and optionally substituted C₁-C₁₀-alkyl, C₂-C₁₀-alkenyl;
A is a 4- to 8-membered, in particular 5- or 6-membered heterocycloalkyl or heteroaryl;
B is H or a 4- to 8-membered cycloalkyl, heterocycloalkyl, aryl or heteroaryl, wherein if B is present, A and B together form a double cycle;
C is 5- or 6-membered aryl or heteroaryl;
X is selected from the group consisting of —CH₂—, —NH—, —O—, —S—, —*CHF—, —*CHO(R^{X})—, —*CHNH(R^{X})—, —*CH—CH(R^{X})—, —*C═C(R^{X})—, —*CH—N(R^{X})—, wherein R^{X} represents hydrogen, methyl, ethyl, cyclopropyl, or —CH₂-cyclopropyl, and wherein the *C is part of heterocycloalkyl A; and
Y is selected from —CH₂—, —NH—, —O—, —*CH₂—CH(R^{Y})—, —*CH═C(R^{Y})—, —*CH₂—O—, —*CH₂—N(R^{Y})— and —*O—CH₂—, wherein R^{Y} represents hydrogen, methyl, ethyl, cyclopropyl, or —CH₂-cyclopropyl, wherein Y may optionally be absent, and wherein the *C or *O is covalently linked to the C of the carbonyl group.

In a second aspect, the present invention provides a compound characterized by a formula (1a) or (1b)

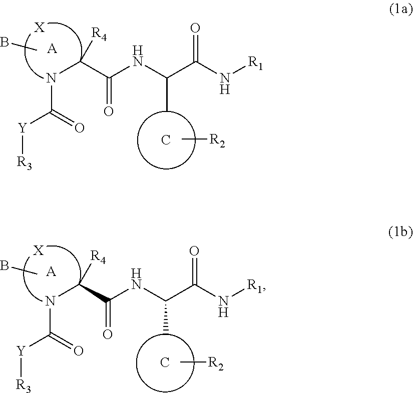

wherein
R¹ is independently selected from the group consisting of optionally substituted C₁-C₁₀-alkyl, C₁-C₁₀-heteroalkyl, C₁-C₁₀-haloalkyl, C₂-C₁₀-alkenyl, C₂-C₁₀-heteroalkenyl, C₂-C₁₀-alkynyl, C₃-C₁₀-cycloalkyl, C₃-C₁₀-heterocycloalkyl, C₄-C₁₀-cycloalkenyl, C₄-C₁₀-heterocycloalkenyl, C₅-C₁₄-aryl, C₅-C₁₄-heteroaryl, C₆-C₁₅ alicyclic system, C₆-C₁₅-aralkyl and C₆-C₁₅-heteroaralkyl;
R² is selected from the group consisting of —F, —Br, —Cl, —R⁵, —OR⁵, —COR⁵, —CO₂R⁵, —OCX₃, —N(R)R′, —N(R)—C(O)—R′, —C(O)—N(R)—C(O)—R′, —N(R)—C(O)—OR′, —C(O)N(R)R′, —N(R)—C(O)—N(R′)R″, —R═NR′, —R═NH, —CN, —NC, —ONO, —NO₂, —ONO₂, —NO, —OCN, —NCO, —SR, —SX₃, —SX₅, —S(O)R, —SO₂R, and —SO₃H, wherein
   R⁵ is selected from the group consisting of —H and optionally substituted C₁-C₁₀ alkyl, C₂-C₁₀-alkenyl, C₂-C₁₀-alkynyl, C₃-C₁₀-cycloalkyl, C₃-C₁₀-heterocycloalkyl, C₅-C₁₄-aryl, C₅-C₁₄-heteroaryl, C₆-C₁₅-aralkyl, C₆-C₁₅-heteroaralkyl, and —(OCH₂CH₂)ₙ₁—NH—R⁶, wherein
      n1 is 2-10, particularly 4-8, and R⁶ is biotinyl; R, R′ and R″ are independently selected from the group consisting of —H, C₁-C₃-alkyl and C₂-C₃-alkenyl;
R³ is independently selected from the group consisting of optionally substituted C₂-C₁₀-alkyl, C₂-C₁₀-heteroalkyl, C₂-C₁₀-haloalkyl, C₂-C₁₀-alkenyl, C₂-C₁₀-heteroalkenyl, C₂-C₁₀-alkynyl, C₃-C₁₀-cycloalkyl, C₃-C₁₀-heterocycloalkyl, C₄-C₁₀-cycloalkenyl, C₄-C₁₀-heterocycloalkenyl, C₅-C₁₄-aryl, C₅-C₁₄-heteroaryl, C₆-C₁₅ alicyclic system, C₆-C₁₅-aralkyl and C₆-C₁₅-heteroaralkyl;
R⁴ is selected from the group consisting of —H and optionally substituted C₁-C₁₀-alkyl, C₂-C₁₀-alkenyl;
A is a 4- to 8-membered, in particular 5- or 6-membered heterocycloalkyl or heteroaryl;
B is H or a 4- to 8-membered cycloalkyl, heterocycloalkyl, aryl or heteroaryl, wherein if B is present, A and B together form a double cycle;
C is a 5- or 6-membered aryl or heteroaryl;
X is selected from the group consisting of —CH₂—, —NH—, —O—, —S—, —*CHF—, —*CHO(R^{X})—, —*CHNH(R^{X})—, —*CH—CH(R^{X})—, —*C═C(R^{X})—, —*CH—N(R^{X})—, wherein R^{X} represents hydrogen, methyl, ethyl, cyclopropyl, or —CH₂-cyclopropyl, and wherein the *C is part of heterocycloalkyl A; and
Y is selected from —CH₂—, —NH—, —O—, —*CH₂—CH(R^{Y})—, —*CH═C(R^{Y})—, —*CH₂—O—, —*CH₂—N(R^{Y})— and —*O—CH₂—, wherein R^{Y} represents hydrogen, methyl, ethyl, cyclopropyl, or —CH₂-cyclopropyl, wherein Y may optionally be absent, and wherein the *C or *O is covalently linked to the C of the carbonyl group.

In a third aspect, the present invention provides a pharmaceutical composition comprising the compound according the first aspect of the invention.

### DETAILED DESCRIPTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention, which will be limited only by the appended claims. Unless defined otherwise, all technical and scientific terms used herein have the same meanings as commonly understood by one of ordinary skill in the art.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

To practice the present invention, unless otherwise indicated, conventional methods of chemistry, biochemistry, and recombinant DNA techniques are employed which are explained in the literature in the field (cf., e.g., Molecular Cloning: A Laboratory Manual, 2^{nd} Edition, J. Sambrook et al. eds., Cold Spring Harbor Laboratory Press, Cold Spring Harbor 1989).

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. As used in this specification and the appended claims, the singular forms “a”, “an”, and “the” include plural referents, unless the content clearly dictates otherwise.

In the following paragraphs, definitions of the terms: alkyl, heteroalkyl, cycloalkyl, heterocycloalkyl, aryl, aralkyl, heteroaryl, heteroaralkyl, alkenyl, cycloalkenyl, heteroalkenyl, heterocycloalkenyl, and alkynyl are provided. These terms will in each instance of its use in the remainder of the specification have the respectively defined meaning and preferred meanings. Nevertheless, in some instances of their use throughout the specification preferred meanings of these terms are indicated.

The term “alkyl” refers to a saturated straight or branched carbon chain. Preferably, the chain comprises from 1 to 10 carbon atoms, i.e. 1, 2, 3, 4, 5, 6, 7, 8, 9 or 10, e.g. methyl, ethyl propyl (n-propyl or iso-propyl), butyl (n-butyl, iso-butyl, sec-butyl, tert-butyl), pentyl, hexyl, heptyl, octyl, nonyl, decyl. Alkyl groups are optionally substituted.

The term “heteroalkyl” refers to a saturated straight or branched carbon chain. Preferably, the chain comprises from 1 to 9 carbon atoms, i.e. 1, 2, 3, 4, 5, 6, 7, 8, or 9, e.g. methyl, ethyl, propyl, iso-propyl, butyl, iso-butyl, sec-butyl, tert-butyl, pentyl, hexyl, heptyl, octyl, nonyl, which is interrupted one or more times, e.g. 1, 2, 3, 4, 5, with the same or different heteroatoms. Preferably, the heteroatoms are selected from O, S, and N, e.g. —(CH₂)ₙ—X—(CH₂)ₘCH₃, with n=0, 1, 2, 3, 4, 5, 6, 7, 8, or 9, m=0, 1, 2, 3, 4, 5, 6, 7, 8, or 9 and X═S, O or NR′ with R′═H or hydrocarbon (e.g. C₁ to C₆ alkyl). In particular, “heteroalkyl” refers to —O—CH₃, —OC₂H₅, —CH₂—O—CH₃, —CH₂—O—C₂H₅, —CH₂—O—C₃H₇, —CH₂—O—C₄H₉, —CH₂—O—C₅H₁₁, —C₂H₄—O—CH₃, —C₂H₄—O—C₂H₅, —C₂H₄—O—C₃H₇, —C₂H₄—O—C₄H₉ etc. Heteroalkyl groups are optionally substituted.

The term “haloalkyl” refers to a saturated straight or branched carbon chain in which one or more hydrogen atoms are replaced by halogen atoms, e.g. by fluorine, chlorine, bromine or iodine. Preferably, the chain comprises from 1 to 10 carbon atoms, i.e. 1, 2, 3, 4, 5, 6, 7, 8, 9 or 10. In particular, “haloalkyl” refers to —CH₂F, —CHF₂, —CF₃, —C₂H₄F, —C₂H₃F₂, —C₂H₂F₃, —C₂HF₄, —C₂F₅, —C₃H₆F, —C₃H₅F₂, —C₃H₄F₃, —C₃H₃F₄, —C₃H₂F₅, —C₃HF₆, —C₃F₇, —CH₂C₁, —CHCl₂, —CCl₃, —C₂H₄C₁, —C₂H₃Cl₂, —C₂H₂Cl₃, —C₂HCl₄, —C₂Cl₅, —C₃H₆Cl, —C₃H₅Cl₂, —C₃H₄Cl₃, —C₃H₃Cl₄, —C₃H₂Cl₅, —C₃HCl₆, and —C₃Cl₇. Haloalkyl groups are optionally substituted.

The terms “cycloalkyl” and “heterocycloalkyl”, by themselves or in combination with other terms, represent, unless otherwise stated, cyclic versions of “alkyl” and “heteroalkyl”, respectively, with preferably 3, 4, 5, 6, 7, 8, 9 or 10 atoms forming a ring, e.g. cyclopropyl, cyclobutyl, cyclopentyl, cyclohexyl, cycloheptyl, cyclooctyl etc. The terms “cycloalkyl” and “heterocycloalkyl” are also meant to include bicyclic, tricyclic and polycyclic versions thereof. If bicyclic, tricyclic or polycyclic rings are formed, it is preferred that the respective rings are connected to each other at two adjacent carbon atoms, however, alternatively the two rings are connected via the same carbon atom, i.e. they form a spiro ring system or they form “bridged” ring systems, preferably tricycle[3.3.1.1]decan. The term “heterocycloalkyl” preferably refers to a saturated ring having five members of which at least one member is an N, O or S atom and which optionally contains one additional O or one additional N; a saturated ring having six members of which at least one member is an N, O or S atom and which optionally contains one additional O or one additional N or two additional N atoms; or a saturated bicyclic ring having nine or ten members of which at least one member is an N, O or S atom and which optionally contains one, two or three additional N atoms. “Cycloalkyl” and “heterocycloalkyl” groups are optionally substituted. Additionally, for heterocycloalkyl, a heteroatom can occupy the position at which the heterocycle is attached to the remainder of the molecule. Examples of cycloalkyl include cyclopropyl, cyclobutyl, cyclopentyl, cyclohexyl, cycloheptyl, spiro[3,3]heptyl, spiro[3,4]octyl, spiro[4,3]octyl, spiro[3,5]nonyl, spiro[5,3]nonyl, spiro[3,6]decyl, spiro[6,3]decyl, spiro[4,5]decyl, spiro[5,4]decyl, bicyclo[2.2.1]heptyl, bicyclo[2.2.2]octyl, adamantyl, and the like. Examples of heterocycloalkyl include 1-(1,2,5,6-tetrahydropyridyl), 1-piperidinyl, 2-piperidinyl, 3-piperidinyl, 4-morpholinyl, 3-morpholinyl, 1,8-diazo-spiro[4,5]decyl, 1,7-diazo-spiro[4,5]decyl, 1,6-diazo-spiro[4,5]decyl, 2,8-diazo-spiro[4,5]decyl, 2,7-diazo-spiro[4,5]decyl, 2,6-diazo-spiro[4,5]decyl, 1,8-diazo-spiro[5,4]decyl, 1,7 diazo-spiro[5,4]decyl, 2,8-diazo-spiro[5,4]decyl, 2,7-diazo-spiro[5,4]decyl, 3,8-diazo-spiro[5,4]decyl, 3,7-diazo-spiro[5,4]decyl, 1,4-diazabicyclo[2.2.2]oct-2-yl, tetrahydrofuran-2-yl, tetrahydrofuran-3-yl, tetrahydrothien-2-yl, tetrahydrothien-3-yl, 1-piperazinyl, 2-piperazinyl, and the like.

The term “alicyclic system” refers to mono, bicyclic, tricyclic or polycyclic version of a cycloalkyl or heterocycloalkyl comprising at least one double and/or triple bond. However, an alicyclic system is not aromatic or heteroaromatic, i.e. does not have a system of conjugated double bonds/free electron pairs. Thus, the number of double and/or triple bonds maximally allowed in an alicyclic system is determined by the number of ring atoms, e.g. in a ring system with up to 5 ring atoms an alicyclic system comprises up to one double bond, in a ring system with 6 ring atoms the alicyclic system comprises up to two double bonds. Thus, the “cycloalkenyl” as defined below is a preferred embodiment of an alicyclic ring system. Alicyclic systems are optionally substituted if indicated.

The term “aryl” preferably refers to an aromatic monocyclic ring containing 6 carbon atoms, an aromatic bicyclic ring system containing 10 carbon atoms or an aromatic tricyclic ring system containing 14 carbon atoms. Examples are phenyl, naphthyl or anthracenyl. The aryl group is optionally substituted if indicated.

The term “aralkyl” refers to an alkyl moiety, which is substituted by aryl, wherein alkyl and aryl have the meaning as outlined above. An example is the benzyl radical. Preferably, in this context the alkyl chain comprises from 1 to 8 carbon atoms, i.e. 1, 2, 3, 4, 5, 6, 7, or 8, e.g. methyl, ethyl, propyl, iso-propyl, butyl, iso-butyl, sec-butyl, tert-butyl, pentyl, hexyl, heptyl, octyl. The aralkyl group is optionally substituted at the alkyl and/or aryl part of the group if indicated. Preferably the aryl attached to the alkyl has the meaning phenyl, naphthyl or anthracenyl.

The term “heteroaryl” preferably refers to a five or six-membered aromatic monocyclic ring wherein at least one of the carbon atoms is replaced by 1, 2, 3, or 4 (for the five membered ring) or 1, 2, 3, 4, or 5 (for the six membered ring) of the same or different heteroatoms, preferably selected from O, N and S; an aromatic bicyclic ring system with 8 to 12 members wherein 1, 2, 3, 4, 5, or 6 carbon atoms of the 8, 9, 10, 11 or 12 carbon atoms have been replaced with the same or different heteroatoms, preferably selected from O, N and S; or an aromatic tricyclic ring system with 13 to 16 members wherein 1, 2, 3, 4, 5, or 6 carbon atoms of the 13, 14, 15, or 16 carbon atoms have been replaced with the same or different heteroatoms, preferably selected from O, N and S. Examples are furanyl, thiophenyl, oxazolyl, isoxazolyl, 1,2,5-oxadiazolyl, 1,2,3-oxadiazolyl, pyrrolyl, imidazolyl, pyrazolyl, 1,2,3-triazolyl, thiazolyl, isothiazolyl, 1,2,3-thiadiazolyl, 1,2,5-thiadiazolyl, pyridinyl, pyrimidinyl, pyrazinyl, 1,2,3-triazinyl, 1,2,4-triazinyl, 1,3,5-triazinyl, 1-benzofuranyl, 2-benzofuranyl, indoyl, isoindoyl, benzothiophenyl, 2-benzothiophenyl, 1H-indazolyl, benzimidazolyl, benzoxazolyl, indoxazinyl, 2,1-benzosoxazoyl, benzothiazolyl, 1,2-benzisothiazolyl, 2,1-benzisothiazolyl, benzotriazolyl, quinolinyl, isoquinolinyl, 2,3-benzodiazinyl, quinoxalinyl, quinazolinyl, quinolinyl, 1,2,3-benzotriazinyl, or 1,2,4-benzotriazinyl.

The term “heteroaralkyl” refers to an alkyl moiety, which is substituted by heteroaryl, wherein alkyl and heteroaryl have the meaning as outlined above. An example is the 2-alkylpyridinyl, 3-alkylpyridinyl, or 2-methylpyridinyl radical. Preferably, in this context the alkyl chain comprises from 1 to 8 carbon atoms, i.e. 1, 2, 3, 4, 5, 6, 7, or 8, e.g. methyl, ethyl, propyl, iso-propyl, butyl, iso-butyl, sec-butyl, tert-butyl, pentyl, hexyl, heptyl, octyl. The heteroaralkyl group is optionally substituted at the alkyl and/or heteroaryl part of the group if indicated. Preferably the heteroaryl attached to the alkyl has the meaning oxazolyl, isoxazolyl, 1,2,5-oxadiazolyl, 1,2,3-oxadiazolyl, pyrrolyl, imidazolyl, pyrazolyl, 1,2,3-triazolyl, thiazolyl, isothiazolyl, 1,2,3-thiadiazolyl, 1,2,5-thiadiazolyl, pyridinyl, pyrimidinyl, pyrazinyl, 1,2,3-triazinyl, 1,2,4-triazinyl, 1,3,5-triazinyl, 1-benzofuranyl, 2-benzofuranyl, indoyl, isoindoyl, benzothiophenyl, 2-benzothiophenyl, 1H-indazolyl, benzimidazolyl, benzoxazolyl, indoxazinyl, 2,1-benzosoxazoyl, benzothiazolyl, 1,2-benzisothiazolyl, 2,1-benzisothiazolyl, benzotriazolyl, 2,3-benzodiazinyl, quinolinyl, isoquinolinyl, quinoxalinyl, quinazolinyl, quinolinyl, 1,2,3-benzotriazinyl, or 1,2,4-benzotriazinyl.

The terms “alkenyl” and “cycloalkenyl” refer to olefinic unsaturated carbon atoms containing chains or rings with one or more double bonds. Examples are propenyl and cyclohexenyl. Preferably, the alkenyl chain comprises from 2 to 8 carbon atoms, i.e. 2, 3, 4, 5, 6, 7, or 8, e.g. ethenyl, 1-propenyl, 2-propenyl, iso-propenyl, 1-butenyl, 2-butenyl, 3-butenyl, iso-butenyl, sec-butenyl, 1-pentenyl, 2-pentenyl, 3-pentenyl, 4-pentenyl, hexenyl, heptenyl, octenyl. Preferably the cycloalkenyl ring comprises from 3 to 8 carbon atoms, i.e. 3, 4, 5, 6, 7, or 8, e.g. 1-cyclopropenyl, 2-cyclopropenyl, 1-cyclobutenyl, 2-cyclobutenyl, 1-cyclopentenyl, 2-cyclopentenyl, 3-cyclopentenyl, 1-cyclohexenyl, 2-cyclohexenyl, 3-cyclohexenyl, cycloheptenyl, cyclooctenyl.

The terms “heteroalkenyl” and “heterocycloalkenyl” refer to unsaturated versions of “heteroalkyl” and “heterocycloalkyl”, respectively. Thus, the term “heteroalkenyl” refers to an unsaturated straight or branched carbon chain. Preferably, the chain comprises from 1 to 9 carbon atoms, i.e. 1, 2, 3, 4, 5, 6, 7, 8, 9, which is interrupted one or more times, e.g. 1, 2, 3, 4, 5, with the same or different heteroatoms. Preferably, the heteroatoms are selected from O, S, and N. In case that one or more of the interrupting heteroatoms is N, the N may be present as an —NR′— moiety, wherein R′ is hydrogen or hydrocarbon (e.g. C₁ to C₆ alkyl), or it may be present as an ═N— or —N═ group, i.e. the nitrogen atom can form a double bond to an adjacent C atom or to an adjacent, further N atom. “Heteroalkenyl” groups are optionally substituted if indicated. The term “heterocycloalkenyl” represents a cyclic version of “heteroalkenyl” with preferably 3, 4, 5, 6, 7, 8, 9 or 10 atoms forming a ring. The term “heterocycloalkenyl” is also meant to include bicyclic, tricyclic and polycyclic versions thereof. If bicyclic, tricyclic or polycyclic rings are formed, it is preferred that the respective rings are connected to each other at two adjacent atoms. These two adjacent atoms can both be carbon atoms; or one atom can be a carbon atom and the other one can be a heteroatom; or the two adjacent atoms can both be heteroatoms. However, alternatively the two rings are connected via the same carbon atom, i.e. they form a spiro ring system or they form “bridged” ring systems. The term “heterocycloalkenyl” preferably refers to an unsaturated ring having five members of which at least one member is an N, O or S atom and which optionally contains one additional O or one additional N; an unsaturated ring having six members of which at least one member is an N, O or S atom and which optionally contains one additional O or one additional N or two additional N atoms; or an unsaturated bicyclic ring having nine or ten members of which at least one member is an N, O or S atom and which optionally contains one, two or three additional N atoms. “Heterocycloalkenyl” groups are optionally substituted if indicated. Additionally, for heteroalkenyl and heterocycloalkenyl, a heteroatom can occupy the position at which the heterocycle is attached to the remainder of the molecule.

The term “aralkenyl” refers to an alkenyl moiety, which is substituted by aryl, wherein alkenyl and aryl have the meaning as outlined above.

The term “heteroaralkenyl” refers to an alkenyl moiety, which is substituted by heteroaryl, wherein alkenyl and heteroaryl have the meaning as outlined above.

The term “alkynyl” refers to unsaturated carbon atoms containing chains or rings with one or more triple bonds. Preferably, the alkynyl chain comprises from 2 to 8 carbon atoms, i.e. 2, 3, 4, 5, 6, 7, or 8, e.g. ethynyl, 1-propynyl, 2-propynyl, 1-butynyl, 2-butynyl, 3-butynyl, 1-pentynyl, 2-pentynyl, 3-pentynyl, 4-pentynyl, hexynyl, heptynyl, octynyl.

The terms “heteroalkynyl”, “cycloalkynyl”, and “heterocycloalkynyl” refer to moieties that basically correspond to “heteroalkenyl”, “cycloalkenyl”, and “heterocycloalkenyl”, respectively, as defined above but differ from “heteroalkenyl”, “cycloalkenyl”, and “heterocycloalkenyl” in that at least one double bond is replaced by a triple bond.

In one embodiment, carbon atoms or hydrogen atoms in alkyl, cycloalkyl, aryl, aralkyl, alkenyl, cycloalkenyl, alkynyl radicals may be substituted independently from each other with one or more elements selected from the group consisting of O, S, N or with groups containing one or more elements, i.e. 1, 2, 3, 4, 5, 6, or more selected from the group consisting of O, S, and N.

Embodiments include alkoxy, cycloalkoxy, aryloxy, aralkoxy, alkenyloxy, cycloalkenyloxy, alkynyloxy, alkylthio, cycloalkylthio, arylthio, aralkylthio, alkenylthio, cycloalkenylthio, alkynylthio, alkylamino, cycloalkylamino, arylamino, aralkylamino, alkenylamino, cycloalkenylamino, alkynylamino radicals.

Other embodiments include hydroxyalkyl, hydroxycycloalkyl, hydroxyaryl, hydroxyaralkyl, hydroxyalkenyl, hydroxycycloalkenyl, hydroxyalkynyl, mercaptoalkyl, mercaptocycloalkyl, mercaptoaryl, mercaptoaralkyl, mercaptoalkenyl, mercaptocycloalkenyl, mercaptoalkynyl, aminoalkyl, aminocycloalkyl, aminoaryl, aminoaralkyl, aminoalkenyl, aminocycloalkenyl, aminoalkynyl radicals.

In another embodiment, one or more hydrogen atoms, e.g. 1, 2, 3, 4, 5, 6, 7, or 8 hydrogen atoms in alkyl, heteroalkyl, cycloalkyl, heterocycloalkyl, alicyclic system, aryl, aralkyl, heteroaryl, heteroaralkyl, alkenyl, cycloalkenyl, heteroalkenyl, heterocycloalkenyl, alkynyl radicals may be substituted independently from each other with one or more halogen atoms, e.g. Cl, F, or Br. One preferred radical is the trifluoromethyl radical.

If two or more radicals can be selected independently from each other, then the term “independently” means that the radicals may be the same or may be different.

The term “optionally substituted” in each instance if not further specified refers to halogen (in particular F, Cl, Br, or I), —NO₂, —CN, —OR^{C}, —NR^{A}R^{B}, —COOR^{C}, —CONR^{A}R^{B}, —NR^{A}COR^{B}, —NR^{B}COR^{C}, —NR^{A}CONR^{A}R^{B}, —NR^{A}SO₂E, —COR^{C}; —SO₂NR^{A}R^{B}, —OOCR^{C}, —CR^{C}R^{D}OH, —R^{C}OH, and -E; R^{A} and R^{B} is each independently selected from the group consisting of hydrogen, alkyl, alkenyl, alkynyl, cycloalkyl, heterocycloalkyl, aryl, aralkyl, and heteroaryl or together form a heteroaryl, or heterocycloalkyl; R^{C} and R^{D} is each independently selected from the group consisting of hydrogen, alkyl, alkenyl, alkynyl, cycloalkyl, heterocycloalkyl, alkoxy, aryl, aralkyl, heteroaryl, and —NR^{A}R^{B}; E is selected from the group consisting of alkyl, alkenyl, alkynyl, cycloalkyl, alkoxy, alkoxyalkyl, heterocycloalkyl, an alicyclic system, aryl and heteroaryl.

Groups that are not described as substituted or optionally substituted are unsubstituted. If a group is described as optionally substituted, it is preferably unsubstituted.

“Pharmaceutically acceptable” means approved by a regulatory agency of the Federal or a state government or listed in the U.S. Pharmacopeia (United States Pharmacopeia-33/National Formulary-28 Reissue, published by the United States Pharmacopeial Convention, Inc., Rockville Md., publication date: April 2010) or other generally recognized pharmacopeia for use in animals, and more particularly in humans.

The term “pharmaceutically acceptable salt” refers to a salt of a compound of the present invention. Suitable pharmaceutically acceptable salts of the compound of the present invention include acid addition salts which may, for example, be formed by mixing a solution of a compound described herein or a derivative thereof with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compound of the invention carries an acidic moiety, suitable pharmaceutically acceptable salts thereof may include alkali metal salts (e.g., sodium or potassium salts); alkaline earth metal salts (e.g., calcium or magnesium salts); and salts formed with suitable organic ligands (e.g., ammonium, quaternary ammonium and amine cations formed using counteranions such as halide, hydroxide, carboxylate, sulfate, phosphate, nitrate, alkyl sulfonate and aryl sulfonate). Illustrative examples of pharmaceutically acceptable salts include but are not limited to: acetate, adipate, alginate, ascorbate, aspartate, benzenesulfonate, benzoate, bicarbonate, bisulfate, bitartrate, borate, bromide, butyrate, calcium edetate, camphorate, camphorsulfonate, camsylate, carbonate, chloride, citrate, clavulanate, cyclopentanepropionate, digluconate, dihydrochloride, dodecylsulfate, edetate, edisylate, estolate, esylate, ethanesulfonate, formate, fumarate, gluceptate, glucoheptonate, gluconate, glutamate, glycerophosphate, glycolylarsanilate, hemisulfate, heptanoate, hexanoate, hexylresorcinate, hydrabamine, hydrobromide, hydrochloride, hydroiodide, 2-hydroxyethanesulfonate, hydroxynaphthoate, iodide, isothionate, lactate, lactobionate, laurate, lauryl sulfate, malate, maleate, malonate, mandelate, mesylate, methanesulfonate, methylsulfate, mucate, 2-naphthalenesulfonate, napsylate, nicotinate, nitrate, N-methylglucamine ammonium salt, oleate, oxalate, pamoate (embonate), palmitate, pantothenate, pectinate, persulfate, 3-phenylpropionate, phosphate/diphosphate, picrate, pivalate, polygalacturonate, propionate, salicylate, stearate, sulfate, subacetate, succinate, tannate, tartrate, teoclate, tosylate, triethiodide, undecanoate, valerate, and the like (see, for example, Berge, S. M., et al, “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. Prodrugs of the compounds described herein are those compounds that readily undergo chemical changes under physiological conditions to provide a compound of formula (1) to (31). A prodrug is an active or inactive compound that is modified chemically through in vivo physiological action, such as hydrolysis, metabolism and the like, into a compound of this invention following administration of the prodrug to a patient. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme. The suitability and techniques involved in making and using prodrugs are well known by those skilled in the art. For a general discussion of prodrugs involving esters, see Svensson L. A. and Tunek A. (1988) Drug Metabolism Reviews 19(2): 165-194 and Bundgaard H. “Design of Prodrugs”, Elsevier Science Ltd. (1985). Examples of a masked carboxylate anion include a variety of esters, such as alkyl (for example, methyl, ethyl), cycloalkyl (for example, cyclohexyl), aralkyl (for example, benzyl, p-methoxybenzyl), and alkylcarbonyloxyalkyl (for example, pivaloyloxymethyl). Amines have been masked as arylcarbonyloxymethyl substituted derivatives which are cleaved by esterases in vivo releasing the free drug and formaldehyde (Bundgaard H. et al. (1989) J. Med. Chem. 32(12): 2503-2507). Also, drugs containing an acidic NH group, such as imidazole, imide, indole and the like, have been masked with N-acyloxymethyl groups (Bundgaard H. “Design of Prodrugs”, Elsevier Science Ltd. (1985)). Hydroxy groups have been masked as esters and ethers. EP 0 039 051 A2 discloses Mannich-base hydroxamic acid prodrugs, their preparation and use.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

As used herein, “para position” when referring to the substituent of an aryl means that the substituent occupies the position opposite to the position at which the aryl is linked to the backbone of the compound.

As used herein, a “patient” means any mammal or bird that may benefit from a treatment with the compounds described herein. Preferably, a “patient” is selected from the group consisting of laboratory animals (e.g. mouse or rat), domestic animals (including e.g. guinea pig, rabbit, chicken, turkey, pig, sheep, goat, camel, cow, horse, donkey, cat, or dog), or primates including chimpanzees and human beings. It is particularly preferred that the “patient” is a human being.

As used herein, “treat”, “treating” or “treatment” of a disease or disorder means accomplishing one or more of the following: (a) reducing the severity of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); and (e) limiting or preventing recurrence of symptoms in patients that were previously symptomatic for the disorder(s).

As used herein, “prevent”, “preventing”, “prevention”, or “prophylaxis” of a disease or disorder means preventing that a disorder occurs in a subject for a certain amount of time. For example, if a compound described herein is administered to a subject with the aim of preventing a disease or disorder, said disease or disorder is prevented from occurring at least on the day of administration and preferably also on one or more days (e.g. on 1 to 30 days; or on 2 to 28 days; or on 3 to 21 days; or on 4 to 14 days; or on 5 to 10 days) following the day of administration.

A “pharmaceutical composition” according to the invention may be present in the form of a composition, wherein the different active ingredients and diluents and/or carriers are admixed with each other, or may take the form of a combined preparation, where the active ingredients are present in partially or totally distinct form. An example for such a combination or combined preparation is a kit-of-parts.

An “effective amount” is an amount of a therapeutic agent sufficient to achieve the intended purpose. The effective amount of a given therapeutic agent will vary with factors such as the nature of the agent, the route of administration, the size and species of the animal to receive the therapeutic agent, and the purpose of the administration. The effective amount in each individual case may be determined empirically by a skilled artisan according to established methods in the art.

The term “carrier”, as used herein, refers to a diluent, adjuvant, excipient, or vehicle with which the therapeutic agent is administered. Such pharmaceutical carriers can be sterile liquids, such as saline solutions in water and oils, including those of petroleum, animal, vegetable or synthetic origin, such as peanut oil, soybean oil, mineral oil, sesame oil and the like. A saline solution is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatine, malt, rice flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents. These compositions can take the form of solutions, suspensions, emulsions, tablets, pills, capsules, powders, sustained-release formulations and the like. The composition can be formulated as a suppository, with traditional binders and carriers such as triglycerides. The compounds of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

### Embodiments of the Invention

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments, which combine the explicitly described embodiments with any number of the disclosed and/or preferred elements. Furthermore, any permutations and combinations of all described elements in this application should be considered disclosed by the description of the present application unless the context indicates otherwise.

In a first aspect the present invention relates to a compound for use in a method of treatment or prevention of a disease, characterized by a formula (1a) or (1b)

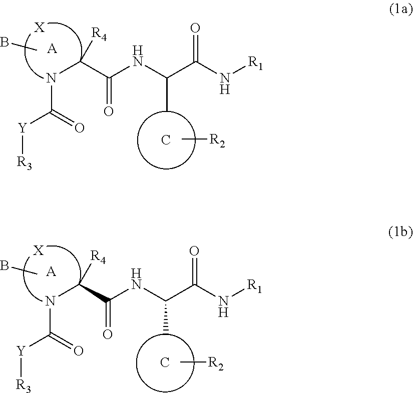

wherein
R¹ is independently selected from the group consisting of optionally substituted C₁-C₁₀-alkyl, i.e. C₁-, C₂-, C₃-, C₄-, C₅-, C₆-, C₇-, C₈-, C₉- or C₁₀-alkyl; C₁-C₁₀-heteroalkyl, i.e. C₁-, C₂-, C₃-, C₄-, C₅-, C₆-, C₇-, C₈-, C₉- or C₁₀-heteroalkyl; C₁-C₁₀-haloalkyl, i.e. C₁-, C₂-, C₃-, C₄-, C₅-, C₆-, C₇-, C₈-, C₉- or C₁₀-haloalkyl; C₂-C₁₀-alkenyl, i.e. C₂-, C₃-, C₄-, C₅-, C₆-, C₇-, C₈-, C₉- or C₁₀-alkenyl; C₂-C₁₀-heteroalkenyl, i.e. C₂-, C₃-, C₄-, C₅-, C₆-, C₇-, C₈-, C₉- or C₁₀-heteroalkenyl; C₂-C₁₀-alkynyl, i.e. C₂-, C₃-, C₄-, C₅-, C₆-, C₇-, C₈-, C₉- or C₁₀-alkynyl; C₃-C₁₀-cycloalkyl, i.e. C₃-, C₄-, C₅-, C₆-, C₇-, C₈-, C₉- or C₁₀-cycloalkyl; C₃-C₁₀-heterocycloalkyl, i.e. C₃-, C₄-, C₅-, C₆-, C₇-, C₈-, C₉- or C₁₀-heterocycloalkyl; C₄-C₁₀-cycloalkenyl, i.e. C₄-, C₅-, C₆-, C₇-, C₈-, C₉- or C₁₀-cycloalkenyl; C₄-C₁₀-heterocycloalkenyl, i.e. C₄-, C₅-, C₆-, C₇-, C₈-, C₉- or C₁₀-heterocycloalkenyl; C₅-C₁₄-aryl, i.e. C₅-, C₆-, C₇-, C₈-, C₉-, C₁₀-, C₁₁-, C₁₂-, C₁₃-, or C₁₄-aryl; C₅-C₁₄-heteroaryl, i.e. C₅-, C₆-, C₇-, C₈-, C₉-, C₁₀-, C₁-, C₁₂-, C₁₃-, or C₁₄-heteroaryl; C₆-C₁₅ alicyclic system, i.e. C₆, C₇, C₈, C₉, C₁₀, C₁₁, C₁₂, C₁₃, C₁₄, or C₁₅ alicyclic system; C₆-C₁₅-aralkyl, i.e. C₆-, C₇-, C₈-, C₉-, C₁₀-, C₁₁-C₁₂-, C₁₃-, C₁₄-, or C₁₅-aralkyl; and C₆-C₁₅-heteroaralkyl, i.e. C₆-, C₇-, C₈-, C₉-, C₁₀-, C₁₁-C₁₂-, C₁₃-, C₁₄-, or C₁₅-heteroaralkyl;
R² is selected from the group consisting of —F, —Br, —Cl, —R⁵, —OR⁵, —COR⁵, —CO₂R⁵, —OCX₃, —N(R)R′, —N(R)—C(O)—R′, —C(O)—N(R)—C(O)—R′, —N(R)—C(O)—OR′, —C(O)N(R)R′, —N(R)—C(O)—N(R′)R″, —R═NR′, —R═NH, —CN, —NC, —ONO, —NO₂, —ONO₂, —NO, —OCN, —NCO, —SR, —SX₃, —SX₅, —S(O)R, —SO₂R, and —SO₃H, wherein
   R⁵ is selected from the group consisting of —H and optionally substituted C₁-C₁₀ alkyl, i.e. C₁-, C₂-, C₃-, C₄-, C₅-, C₆-, C₇-, C₈-, C₉- or C₁₀-alkyl; C₂-C₁₀-alkenyl, i.e. C₂-, C₃-, C₄-, C₅-, C₆-, C₇-, C₈-, C₉- or C₁₀-alkenyl; C₂-C₁₀-alkynyl, i.e. C₂-, C₃-, C₄-, C₅-, C₆-, C₇-, C₈-, C₉- or C₁₀-alkynyl; C₃-C₁₀-cycloalkyl, i.e. C₃-, C₄-, C₅-, C₆-, C₇-, C₈-, C₉- or C₁₀-cycloalkyl; C₃-C₁₀-heterocycloalkyl, i.e. C₃-, C₄-, C₅-, C₆-, C₇-, C₈-, C₉- or C₁₀-heterocycloalkyl; C₅-C₁₄-aryl, i.e. C₅-, C₆-, C₇-, C₈-, C₉-, C₁₀-, C₁₁-, C₁₂-, C₁₃-, or C₁₄-aryl; C₅-C₁₄-heteroaryl, i.e. C₅-, C₆-, C₇-, C₈-, C₉-, C₁₀-, C₁₁-, C₁₂-, C₁₃-, or C₁₄-heteroaryl; C₆-C₁₅-aralkyl, i.e. C₆-, C₇-, C₈-, C₉-, C₁₀-, C₁₁-C₁₂-, C₁₃-, C₁₄-, or C₁₅-aralkyl; C₆-C₁₅-heteroaralkyl, i.e. C₆-, C₇-, C₈-, C₉-, C₁₀-, C₁₁-C₁₂-, C₁₃-, C₁₄-, or C₁₅-heteroaralkyl and —(OCH₂CH₂)ₙ₁—NH—R⁶, wherein
      n1 is 2-10, i.e. 2, 3, 4, 5, 6, 7, 8, 9, or 10, particularly 4-8, and R⁶ is a biotin moiety linked via an amide bond;
   R, R′ and R″ are independently selected from the group consisting of —H, C₁-, C₂, or C₃-alkyl and C₂-C₃-alkenyl;
R³ is independently selected from the group consisting of optionally substituted C₂-C₁₀-alkyl, i.e. C₂-, C₃-, C₄-, C₅-, C₆-, C₇-, C₈-, C₉- or C₁₀-alkyl; C₂-C₁₀-heteroalkyl, i.e. C₂-, C₃-, C₄-, C₅-, C₆-, C₇-, C₈-, C₉- or C₁₀-heteroalkyl; C₂-C₁₀-haloalkyl, i.e. C₂-, C₃-, C₄-, C₅-, C₆-, C₇-, C₈-, C₉- or C₁₀-haloalkyl; C₂-C₁₀-alkenyl, i.e. C₂-, C₃-, C₄-, C₅-, C₆-, C₇-, C₈-, C₉- or C₁₀-alkenyl; C₂-C₁₀-heteroalkenyl, i.e. C₂-, C₃-, C₄-, C₅-, C₆-, C₇-, C₈-, C₉- or C₁₀-heteroalkenyl; C₂-C₁₀-alkynyl, i.e. C₂-, C₃-, C₄-, C₅-, C₆-, C₇-, C₈-, C₉- or C₁₀-alkynyl; C₃-C₁₀-cycloalkyl, i.e. C₃-, C₄-, C₅-, C₆-, C₇-, C₈-, C₉- or C₁₀-cycloalkyl; C₃-C₁₀-heterocycloalkyl, i.e. C₃-, C₄-, C₅-, C₆-, C₇-, C₈-, C₉- or C₁₀-heterocycloalkyl; C₄-C₁₀-cycloalkenyl, i.e. C₄-, C₅-, C₆-, C₇-, C₈-, C₉- or C₁₀-cycloalkenyl; C₄-C₁₀-heterocycloalkenyl, i.e. C₄-, C₅-, C₆-, C₇-, C₈-, C₉- or C₁₀-heterocycloalkenyl; C₅-C₁₄-aryl, i.e. C₅-, C₆-, C₇-, C₈-, C₉-, C₁₀-, C₁₁-, C₁₂-, C₁₃-, or C₁₄-aryl; C₅-C₁₄-heteroaryl, i.e. C₅-, C₆-, C₇-, C₈-, C₉-, C₁₀-, C₁₁-, C₁₂-, C₁₃-, or C₁₄-heteroaryl; C₆-C₁₅ alicyclic system, i.e. C₆, C₇, C₈, C₉, C₁₀, C₁₁, C₁₂, C₁₃, C₁₄, or C₁₅ alicyclic system; C₆-C₁₅-aralkyl, i.e. C₆-, C₇-, C₈-, C₉-, C₁₀-, C₁₁-C₁₂-, C₁₃-, C₁₄-, or C₁₅-aralkyl; and C₆-C₁₅-heteroaralkyl, i.e. C₆-, C₇-, C₈-, C₉-, C₁₀-, C₁₁-C₁₂-, C₁₃-, C₁₄-, or C₁₅-heteroaralkyl;
R⁴ is selected from the group consisting of —H and optionally substituted C₁-C₁₀-alkyl, i.e. C₁-, C₂-, C₃-, C₄-, C₅-, C₆-, C₇-, C₈-, C₉- or C₁₀-alkyl; and C₂-C₁₀-alkenyl i.e. C₂-, C₃-, C₄-, C₅-, C₆-, C₇-, C₈-, C₉- or C₁₀-alkenyl;
A is a 4-, 5-, 6-, 7- or 8-membered, in particular 5- or 6-membered heterocycloalkyl or heteroaryl;
B is a 4-, 5-, 6, 7- or 8-membered cycloalkyl, heterocycloalkyl, aryl or heteroaryl, wherein B may be present or absent and wherein if B is present, A and B together form a double cycle;
C is a 5- or 6-membered aryl or heteroaryl;
X is selected from the group consisting of —CH₂—, —NH—, —O—, —S—, —*CHF—, —*CHO(R^{X})—, —*CHNH(R^{X})—, —*CH—CH(R^{X})—, —*C═C(R^{X})—, —*CH—N(R^{X})—, wherein R^{X} represents hydrogen, methyl, ethyl, cyclopropyl, or —CH₂-cyclopropyl, and wherein the *C is part of heterocycloalkyl or heteroaryl A; and
Y is selected from —*CH₂—, —*NH—, —*O—, —*CH₂—CH(R^{Y})—, —*CH═C(R^{Y})—, —*CH₂—O—, —*CH₂—N(R^{Y})— and —*O—CH₂—, wherein R^{Y} represents hydrogen, methyl, ethyl, cyclopropyl, or —CH₂-cyclopropyl, wherein Y may optionally be absent, and wherein *C, *N or *O is covalently linked to the C of the carbonyl group.

In preferred embodiments of the first aspect, the compound for use is characterized by a formula

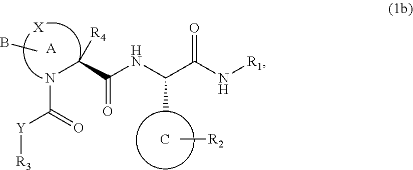

wherein
R¹, R², R³, R⁴, A, B, C, X and Y are defined as above.

Preferably, the compound for use is characterized by formula (1a) or (1b), preferably (1b), wherein R², R³, R⁴, A, B, X and Y are defined as above and R¹ is

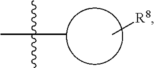

wherein

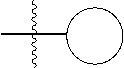

is a 5- or 6-membered aryl or heteroaryl, and R⁸ is selected from H, R⁹ and OR⁹, wherein R⁹ is selected from optionally substituted C₁-, C₂-, C₃-, or C₄-alkyl or C₃-, or C₄-cycloalkyl, wherein the aryl or heteroaryl and R⁹ may form a double cycle and wherein

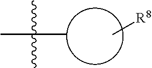

comprises at least one heteroatom.
More preferably, R¹ is selected from the group consisting of

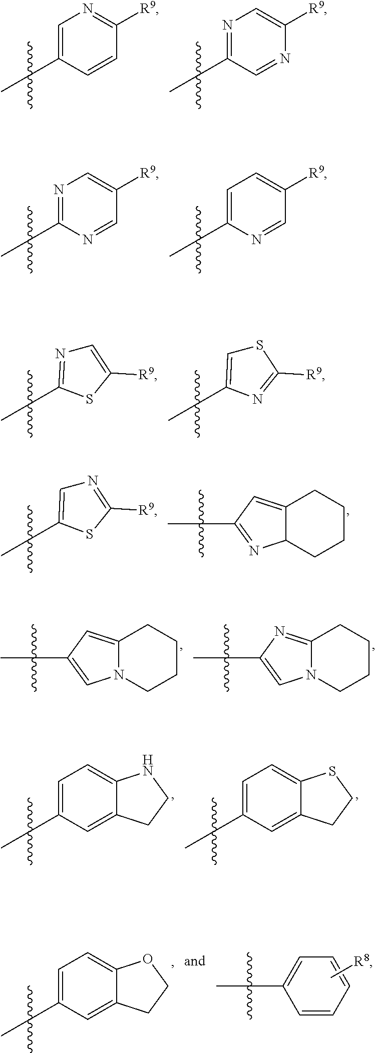

wherein R⁸ is selected from R⁹ and OR⁹, and wherein R⁹ is an unsubstituted C₁-, C₂-, C₃-, or C₄-alkyl or unsubstituted C₃-, or C₄-cycloalkyl.
Even more preferably, R¹ is selected from the group consisting of

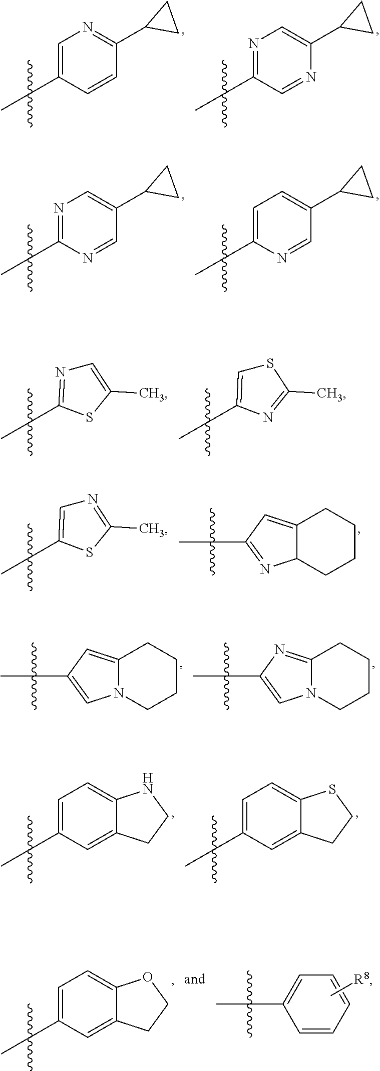

wherein R⁸ is selected from R⁹ and OR⁹, and wherein R⁹ is an unsubstituted C₁-, C₂-, C₃-, or C₄-alkyl or unsubstituted C₃-, or C₄-cycloalkyl.
Most preferably, R¹ is selected from the group consisting of

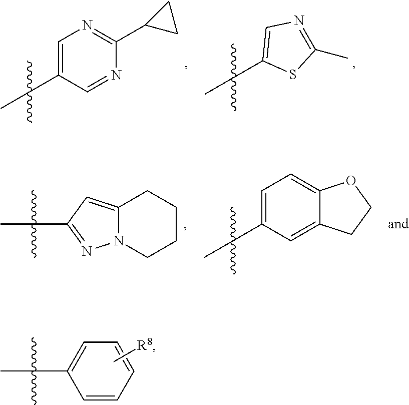

wherein R⁸ is selected from R⁹ and OR⁹, wherein R⁹ is an unsubstituted C₁-, C₂-, C₃-, or C₄-alkyl.

In more preferred embodiments of the first aspect, the compound for use is characterized by a formula (2a)

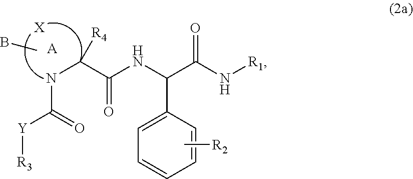

wherein
R¹, R², R³, R⁴, A, B, X and Y are defined as above.

More preferably, the compound for use is characterized by a formula (2b)

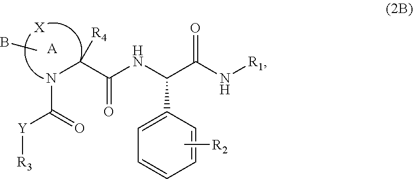

wherein
R¹, R², R³, R⁴, A, B, X and Y are defined as above.

In embodiments of the first aspect in which the compound for use is characterized by formulae (1a) or (1b), it is preferred that C is a 5- or 6-membered aryl or heteroaryl selected from the group consisting of thienyl, thiazolyl, oxazolyl, isooxazolyl, thiadiazolyl, imidazolyl, pyrazolyl, pyridinyl, pyrazinyl, pyridazinyl and pyrimidinyl.

For those skilled in the art it is well known that residues like a phenyl ring or substituted phenyl ring can be bioisosterically replaced by other residues such as 5-membered heterocycles such as thienyl or thiazolyl, oxazolyl, isooxazolyl, thiadiazolyl, imidazolyl, pyrazolyl rings, or 6-membered heterocycles like pyridinyl, pyrazinyl, pyridazinyl, pyrimidinyl rings exerting similar biological activity and function. These concepts are extensively reviewed in literature. See for example Bioisosteres in Medicinal Chemistry (Ed: N. Brown), Wiley-VCH, Weinheim, 2012; Brown, N. (2014), Bioisosteres and Scaffold Hopping in Medicinal Chemistry. Mol. Inf., 33: 458-462; Nicholas A. Meanwell (2011) Synopsis of Some Recent Tactical Application of Bioisosteres in Drug Design, Journal of Medicinal Chemistry 54: 2529-2591.

In embodiments of the first aspect in which the compound for use is characterized by any of formulae (1a), (1b), (2a) or (2b), it is preferred that A is a 4-, 5- or 6-membered heterocycloalkyl. Even more preferably, A is a 4-, 5- or 6-membered heterocycloalkyl and R⁴ is selected from the group consisting of —H and optionally substituted C₁-C₃-alkyl, i.e. C₁-, C₂-, or C₃-alkyl.

In embodiments of the first aspect in which the compound for use is characterized by any of formulae (1a), (1b), (2a) or (2b), it is preferred that B is absent or a 5- or 6-membered cycloalkyl or aryl.

In particularly preferred embodiments of the first aspect, the compound for use is characterized by any of formulae (1a), (1b), (2a) or (2b), wherein A is

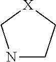

and X is defined as above. Even more preferably, A is

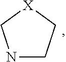

X is defined as above and R⁴ is selected from the group consisting of —H and optionally substituted C₁-C₃-alkyl, i.e. C₁-, C₂-, or C₃-alkyl.

In more preferred embodiments of the first aspect, the compound for use is characterized by a formula (3a)

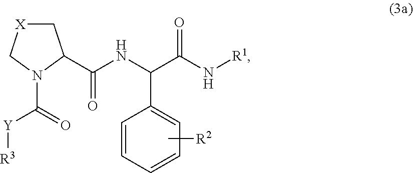

wherein
R¹, R², R³, and Y are defined as above and X is selected from the group consisting of —CH₂—, —NH—, —O—, —S—, —*CHF—, —*CHO(R^{X})—, —*CHNH(R^{X})—, —*CH—CH(R^{X})—, —*C═C(R^{X})—, —*CH—N(R^{X})—, wherein R^{X} represents hydrogen, methyl, ethyl, cyclopropyl, or —CH₂-cyclopropyl, and wherein the *C is part of the heterocycloalkyl.

More preferably, the compound for use is characterized by a formula (3b)

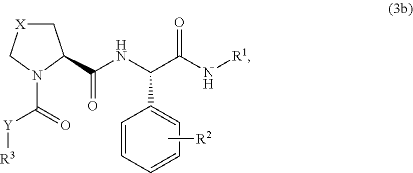

wherein
R¹, R², R³, and Y are defined as above and X is selected from the group consisting of —CH₂—, —NH—, —O—, —S—, —*CHF—, —*CHO(R^{X})—, —*CHNH(R^{X})—, —*CH—CH(R^{X})—, —*C═C(R^{X})—, —*CH—N(R^{X})—, wherein R^{X} represents hydrogen, methyl, ethyl, cyclopropyl, or —CH₂-cyclopropyl, and wherein the *C is part of the heterocycloalkyl.

In even more preferred embodiments of the first aspect, the compound for use is characterized by a formula (4a)

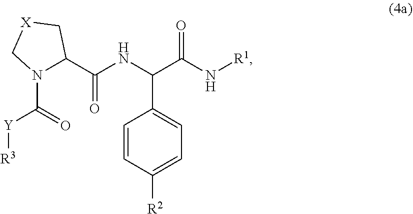

wherein
R¹, R³, and Y are defined as above, R² is O—R⁵, wherein R⁵ is defined as above, and X is selected from the group consisting of —CH₂—, —NH—, —O—, —S—, —*CHF—, —*CHO(R^{X})—, —*CHNH(R^{X})—, —*CH—CH(R^{X})—, —*C═C(R^{X})—, —*CH—N(R^{X})—, wherein R^{X} represents hydrogen, methyl, ethyl, cyclopropyl, or —CH₂-cyclopropyl, and wherein the *C is part of the heterocycloalkyl.

In most preferred embodiments of the first aspect, the compound for use is characterized by a formula (4b)

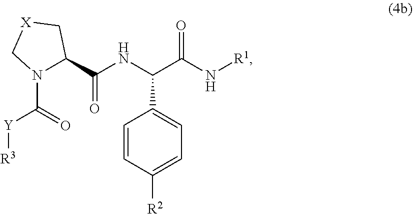

wherein
R¹, R³, and Y are defined as above, R² is O—R⁵, wherein R⁵ is defined as above, and X is selected from the group consisting of —CH₂—, —NH—, —O—, —S—, —*CHF—, —*CHO(R^{X})—, —*CHNH(R^{X})—, —*CH—CH(R^{X})—, —*C═C(R^{X})—, —*CH—N(R^{X})—, wherein R^{X} represents hydrogen, methyl, ethyl, cyclopropyl, or —CH₂-cyclopropyl, and wherein the *C is part of the heterocycloalkyl.

In preferred embodiments of the first aspect, the compound for use is characterized by any of formulae (1a), (1b), (2a), (2b), (3a), (3b), (4a) or (4b), preferably (1b), (2b), (3b) or (4b), more preferably (4b), wherein Y is absent or selected from —*CH₂—, —*O—, —*CH₂—CH₂—, —*CH₂—O—, and —*O—CH₂—, wherein the *C or *O is covalently linked to the C of the carbonyl group.

In any embodiment of the present application referring to formulae (1a), (1b), (2a), (2b), (3a), (3b), (4a) or (4b), formulae (1b), (2b), (3b) or (4b) are preferred and formula (4b) is especially preferred.

In preferred embodiments of the first aspect, the compound for use is characterized by any of formulae (1a), (1b), (2a), (2b), (3a), (3b), (4a) or (4b), wherein Y is selected from —*CH₂—, —*O—, —*CH₂—CH₂—, and —*CH₂—O—, wherein the *C or *O is covalently linked to the C of the carbonyl group.

In more preferred embodiments of the first aspect, the compound for use is characterized by any of formulae (1a), (1b), (2a), (2b), (3a), (3b), (4a) or (4b), wherein Y is selected from —*CH₂—, —*CH₂—CH₂—, and —*CH₂—O—, wherein the *C or *O is covalently linked to the C of the carbonyl group.

In preferred embodiments of the first aspect, the compound for use is characterized by any of formulae (1a), (1b), (2a), (2b), (3a), (3b), (4a) or (4b), wherein Y is selected from —*CH₂—, —*O— or absent, in particular from —*CH₂— or absent, wherein the *C or *O is covalently linked to the C of the carbonyl group.

In preferred embodiments of the first aspect, Y is absent.

In preferred embodiments of the first aspect, the compound for use is characterized by any of formulae (1a), (1b), (2a), (2b), (3a), (3b), (4a) or (4b), wherein X is selected from the group consisting of —CH₂—, —NH—, —O—, —S—, and —*CHF—, wherein the *C is part of the heterocycloalkyl.

In preferred embodiments of the first aspect, the compound for use is characterized by any of formulae (1a), (1b), (2a), (2b), (3a), (3b), (4a) or (4b), wherein X is —CH₂—.

In preferred embodiments of the first aspect, the compound for use is characterized by any of formulae (1a), (1b), (2a), (2b), (3a), (3b), (4a) or (4b), wherein R¹ is

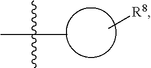

wherein

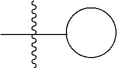

is a 5- to 12-membered, i.e. 5-, 6-, 7-, 8-, 9-, 10-, 11-, or 12-membered alicyclic system, aryl or heteroaryl, in particular a 5- or 6-membered aryl or heteroaryl, and R⁸ is selected from H, R⁹ and OR⁹, wherein R⁹ is selected from optionally substituted C₁-, C₂-, C₃-, C₄-C₅-, C₆- or C₇-alkyl and optionally substituted C₃-, C₄-C₅-, C₆- or C₇-cycloalkyl, wherein the aryl or heteroaryl and R⁹ may form a double cycle,
and wherein preferably, R¹ is not is selected from

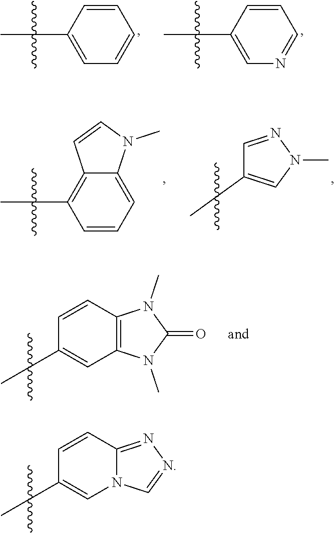

It is particularly preferred that the compound for use is not a compound characterized by any of formulae (39)-(50).

In preferred embodiments of the first aspect, the compound for use is characterized by any of formulae (1a), (1b), (2a), (2b), (3a), (3b), (4a) or (4b),

wherein R¹ is

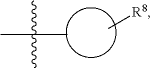

wherein

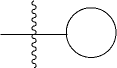

is a 5- or 6-membered aryl or heteroaryl, and R⁸ is selected from H, R⁹ and OR⁹, wherein R⁹ is selected from optionally substituted C₁-, C₂-, C₃-, or C₄-alkyl or cycloalkyl, wherein the aryl or heteroaryl and R⁹ may form a double cycle and wherein

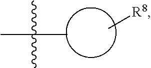

comprises at least one heteroatom.
More preferably, R¹ is selected from the group consisting of

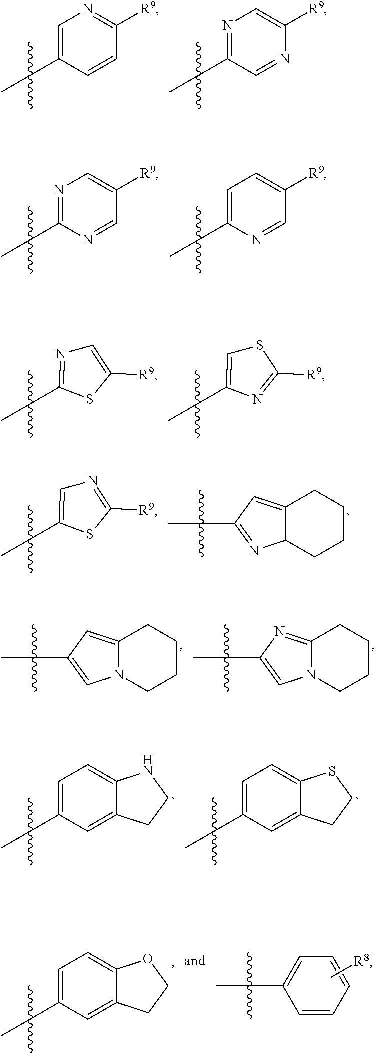

wherein R⁸ is selected from R⁹ and OR⁹, and wherein R⁹ is an unsubstituted C₁-, C₂-, C₃-, or C₄-alkyl or unsubstituted C₃-, or C₄-cycloalkyl.
Even more preferably, R¹ is selected from the group consisting of

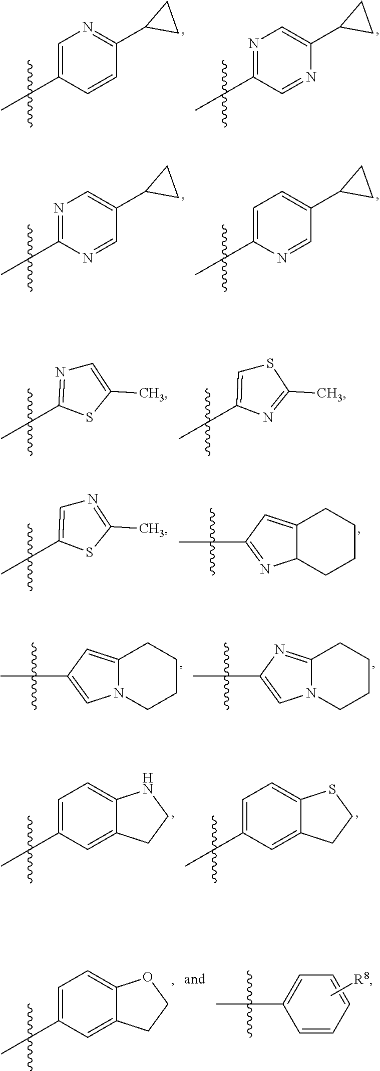

wherein R⁸ is selected from R⁹ and OR⁹, and wherein R⁹ is an unsubstituted C₁-, C₂-, C₃-, or C₄-alkyl or unsubstituted C₃-, or C₄-cycloalkyl.

In preferred embodiments of the first aspect, the compound for use is characterized by any of formulae (1a), (1b), (2a), (2b), (3a), (3b), (4a) or (4b), wherein R¹ is selected from

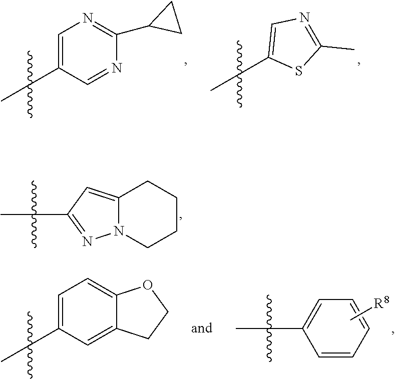

wherein R⁸ is selected from R⁹ and OR⁹, wherein R⁹ is an unsubstituted C₁-, C₂-, C₃-, or C₄-alkyl.

In preferred embodiments of the first aspect, the compound for use is characterized by any of formulae (1a), (1b), (2a), (2b), (3a), (3b), (4a) or (4b), wherein R¹ is selected from

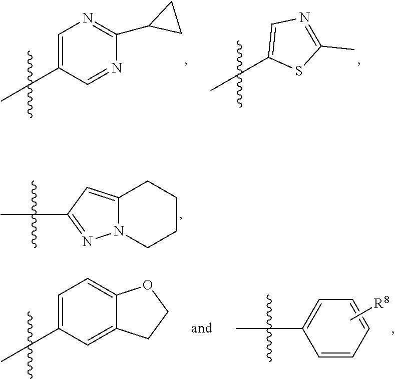

in particular wherein R⁸ is in para position, and wherein R⁸ is OR⁹, wherein R⁹ is C₁-, C₂-, C₃-, or C₄-alkyl, in particular R⁹ is —CH₃

In preferred embodiments of the first aspect, the compound for use is characterized by any of formulae (1a), (1b), (2a), (2b), (3a), (3b), (4a) or (4b), wherein R¹ is selected from

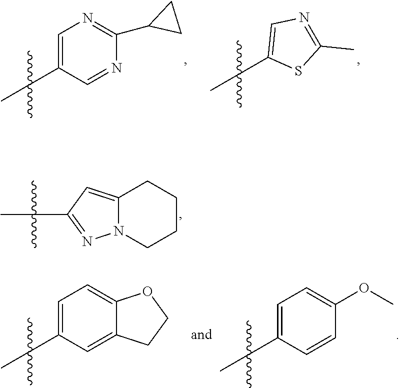

In preferred embodiments of the first aspect, the compound for use is characterized by any of formulae (1a), (1b), (2a), (2b), (3a), (3b), (4a) or (4b), wherein R¹ is

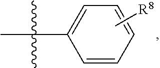

in particular wherein R⁸ is in para position, and wherein R⁸ is selected from R⁹ and OR⁹, wherein R⁹ is a C₁-, C₂-, C₃-, or C₄-alkyl.

In preferred embodiments of the first aspect, the compound for use is characterized by any of formulae (1a), (1b), (2a), (2b), (3a), (3b), (4a) or (4b), wherein R¹ is

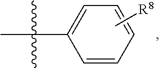

in particular wherein R⁸ is in para position, and wherein R⁸ is OR⁹, wherein R⁹ is an unsubstituted C₁-, C₂-, C₃-, or C₄-alkyl.

In preferred embodiments of the first aspect, the compound for use is characterized by any of formulae (1a), (1b), (2a), (2b), (3a), (3b), (4a) or (4b), wherein R¹ is

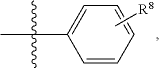

wherein R⁸ is —O—CH₃, in particular wherein R⁸ is in para position.

In preferred embodiments of the first aspect, the compound for use is characterized by any of formulae (1a), (1b), (2a), (2b), (3a) or (3b), wherein R² is in para position.

In preferred embodiments of the first aspect, the compound for use is characterized by any of formulae (1a), (1b), (2a), (2b), (3a) or (3b), wherein R² is —OR⁵ or —R⁵, wherein R⁵ is defined as above.

In more preferred embodiments of the first aspect, the compound for use is characterized by any of formulae (1a), (1b), (2a), (2b), (3a) or (3b), wherein R² is —OR⁵, wherein R⁵ is defined as above.

In more preferred embodiments of the first aspect, the compound for use is characterized by any of formulae (1a), (1b), (2a), (2b), (3a), (3b), (4a) or (4b), wherein R² is —OR⁵, wherein R⁵ is selected from the group consisting of —H and optionally substituted C₁-C₁₀ alkyl, i.e. C₁-, C₂-, C₃-, C₄-, C₅-, C₆-, C₇-, C₈-, C₉- or C₁₀-alkyl; C₂-C₁₀-alkenyl, i.e. C₂-, C₃-, C₄-, C₅-, C₆-, C₇-, C₈-, C₉- or C₁₀-alkenyl; C₂-C₁₀-alkynyl, i.e. C₂-, C₃-, C₄-, C₅-, C₆-, C₇-, C₈-, C₉- or C₁₀-alkynyl; C₃-C₁₀-cycloalkyl, i.e. C₃-, C₄-, C₅-, C₆-, C₇-, C₈-, C₉- or C₁₀-cycloalkyl; C₃-C₁₀-heterocycloalkyl, i.e. C₃-, C₄-, C₅-, C₆-, C₇-, C₈-, C₉- or C₁₀-heterocycloalkyl; C₅-C₁₄-aryl, i.e. C₅-, C₆-, C₇-, C₈-, C₉-, C₁₀-, C₁₁-, C₁₂-, C₁₃-, or C₁₄-aryl; C₅-C₁₄-heteroaryl, i.e. C₅-, C₆-, C₇-, C₈-, C₉-, C₁₀-, C₁₁-, C₁₂-, C₁₃-, or C₁₄-heteroaryl; C₆-C₁₅-aralkyl, i.e. C₆-, C₇-, C₈-, C₉-, C₁₀-, C₁₁-, C₁₂-, C₁₃-, C₁₄-, or C₁₅-aralkyl; and C₆-C₁₅-heteroaralkyl i.e. C₆-, C₇-, C₈-, C₉-, C₁₀-, C₁₁-, C₁₂-, C₁₃-, C₁₄-, or C₁₅-heteroaralkyl.

In more preferred embodiments of the first aspect, the compound for use is characterized by any of formulae (1a), (1b), (2a), (2b), (3a), (3b), (4a) or (4b), wherein R² is —OR⁵, wherein R⁵ is selected from the group consisting of —H and optionally substituted C₁-C₆ alkyl, i.e. C₁-, C₂-, C₃-, C₄-, C₅-, or C₆-alkyl; C₂-C₆-alkenyl, i.e. C₂-, C₃-, C₄-, C₅-, or C₆-alkenyl; C₂-C₆-alkynyl, i.e. C₂-, C₃-, C₄-, C₅-, or C₆-alkynyl; and C₃-C₈-cycloalkyl C₃-, C₄-, C₅-, C₆-, C₇-, or C₈-cycloalkyl.

In even more preferred embodiments of the first aspect, the compound for use is characterized by any of formulae (1a), (1b), (2a), (2b), (3a), (3b), (4a) or (4b), wherein R² is —OR⁵ and R⁵ is selected from the group consisting of —H and optionally substituted C₁-C₄-alkyl, i.e. C₁-, C₂-, C₃-, or C₄-alkyl and optionally substituted C₃-C₆-cycloalkyl, i.e. C₃-, C₄-, C₅-, or C₆-cycloalkyl.

In even more preferred embodiments of the first aspect, the compound for use is characterized by any of formulae (1a), (1b), (2a), (2b), (3a), (3b), (4a) or (4b), wherein R² is —OCH₃.

In most preferred embodiments of the first aspect, the compound for use is characterized by any of formulae (1a), (1b), (2a), (2b), (3a), (3b), (4a) or (4b), wherein R² is in para position and is —OCH₃.

In preferred embodiments of the first aspect, the compound for use is characterized by any of formulae (1a), (1b), (2a), (2b), (3a), (3b), (4a) or (4b), wherein R² is (OCH₂CH₂)ₙ₁—R⁶, wherein n1 is 2, 3, 4, 5, 6, 7, 8, or 10, particularly 4-8, more particularly 6 and R⁶ is a biotin moiety linked via an amide bond.

In preferred embodiments of the first aspect, the compound for use is characterized by any of formulae (1a), (1b), (2a), (2b), (3a), (3b), (4a) or (4b), wherein R³ is (CH₂)ₙ₂—R⁷, wherein n2 is 1-4, i.e. 1, 2, 3, or 4 and R⁷ is selected from a optionally substituted C₁-C₇-alkyl, i.e. C₁-, C₂-, C₃-, C₄-, C₅-, C₆-, or C₇-alkyl; C₄-C₇-cycloalkyl, i.e. C₄-, C₅-, C₆-, or C₇-cycloalkyl; C₅-C₁₀-aryl, i.e. C₅-, C₆-, C₇-, C₈-, C₉- or C₁₀-aryl; or C₅-C₁₀-heteroaryl, i.e. C₅-, C₆-, C₇-, C₈-, C₉- or C₁₀-heteroaryl.

In preferred embodiments of the first aspect, the compound for use is characterized by any of formulae (1a), (1b), (2a), (2b), (3a), (3b), (4a) or (4b), wherein R³ is (CH₂)ₙ₂—R⁷, wherein n2 is 1-4, i.e., 1, 2, 3 or 4 and R⁷ is selected from a optionally substituted C₁-C₇-alkyl, i.e. C₁-, C₂-, C₃-, C₄-, C₅-, C₆-, or C₇-alkyl; C₄-C₇-cycloalkyl, i.e. C₄-, C₅-, C₆-, or C₇-cycloalkyl; C₅-C₁₀-aryl, i.e. C₅-, C₆-, C₇-, C₈-, C₉- or C₁₀-aryl; or C₅-C₁₀-heteroaryl, i.e. C₅-, C₆-, C₇-, C₈-, C₉- or C₁₀-heteroaryl, and Y is absent or selected from —*CH₂—, —*O—, —*CH₂—CH₂—, —*CH₂—O—, and —*O—CH₂—, wherein the *C or *O is covalently linked to the C of the carbonyl group.

In preferred embodiments of the first aspect, the compound for use is characterized by any of formulae (1a), (1b), (2a), (2b), (3a), (3b), (4a) or (4b), wherein R³ is (CH₂)ₙ₂—R⁷, wherein n2 is 1-4, i.e. 1, 2, 3, or 4 and R⁷ is selected from a optionally substituted C₁-C₇-alkyl, i.e. C₁-, C₂-, C₃-, C₄-, C₅-, C₆-, or C₇-alkyl; C₄-C₇-cycloalkyl, i.e. C₄-, C₅-, C₆-, or C₇-cycloalkyl; C₅-C₁₀-aryl, i.e. C₅-, C₆-, C₇-, C₈-, C₉- or C₁₀-aryl; or C₅-C₁₀-heteroaryl, i.e. C₅-, C₆-, C₇-, C₈-, C₉- or C₁₀-heteroaryl, and Y is selected from —*CH₂—, —*O—, —*CH₂—CH₂—, and —*CH₂—O—, wherein the *C or *O is covalently linked to the C of the carbonyl group.

In preferred embodiments of the first aspect, the compound for use is characterized by any of formulae (1a), (1b), (2a), (2b), (3a), (3b), (4a) or (4b), wherein R³ is (CH₂)ₙ₂—R⁷, wherein n2 is 1-4, i.e. 1, 2, 3, or 4 and R⁷ is selected from c

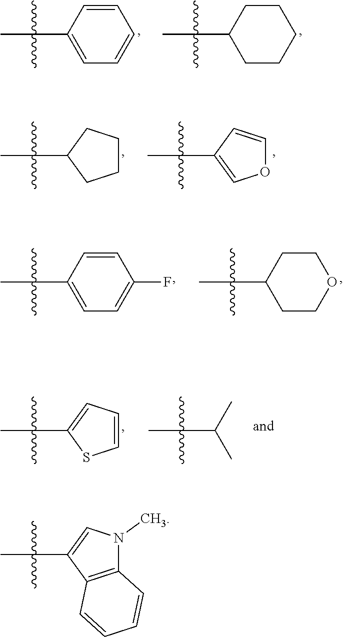

In preferred embodiments of the first aspect, the compound for use is characterized by any of formulae (1a), (1b), (2a), (2b), (3a), (3b), (4a) or (4b), wherein R³ is (CH₂)ₙ₂—R⁷, wherein n2 is 1-4, i.e. 1, 2, 3, or 4 and R⁷ is

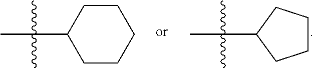 [SMILES: *C1CCCCC1 ; *C1CCCC1]

In preferred embodiments of the first aspect, the compound for use is characterized by formula (1a) or (1b), in particular (1b), wherein
A is a 4- to 8-, i.e. 4-, 5-, 6-7 or 8-membered, in particular 5- or 6-membered heterocycloalkyl or heteroaryl;
B is a 4- to 8-, i.e. 4-, 5-, 6-7 or 8-membered cycloalkyl, heterocycloalkyl, aryl or heteroaryl, wherein B may be present or absent and wherein if B is present, A and B together form a double cycle;
C is 5- or 6-membered aryl or heteroaryl;
R¹ is

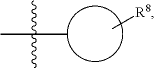

wherein

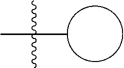

is a 5- to 12-membered, i.e. 5-, 6-, 7-, 8-, 9-, 10-, 11-, or 12-membered alicyclic system, aryl or heteroaryl, in particular a 5- or 6-membered aryl or heteroaryl, and R⁸ is selected from H, R⁹ and OR⁹, wherein R⁹ is selected from optionally substituted C₁-, C₂-, C₃-, C₄-C₅-, C₆- or C₇-alkyl and optionally substituted C₃-, C₄-C₅-, C₆- or C₇-cycloalkyl, preferably wherein the aryl or heteroaryl and R⁹ may form a double cycle,
wherein preferably

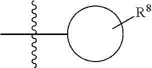

comprises at least one heteroatom;
more preferably, R¹ is selected from the group consisting of

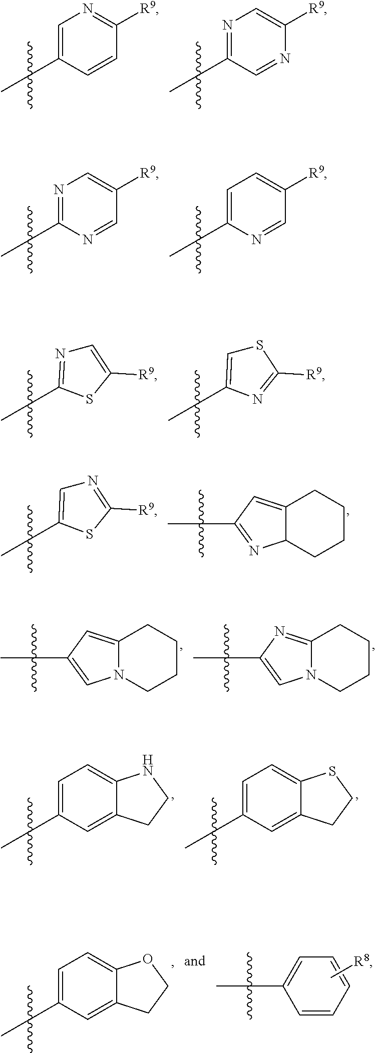

wherein R⁸ is selected from R⁹ and OR⁹, and wherein R⁹ is an unsubstituted C₁-, C₂-, C₃-, or C₄-alkyl or unsubstituted C₃-, or C₄-cycloalkyl;
even more preferably, R¹ is selected from the group consisting of

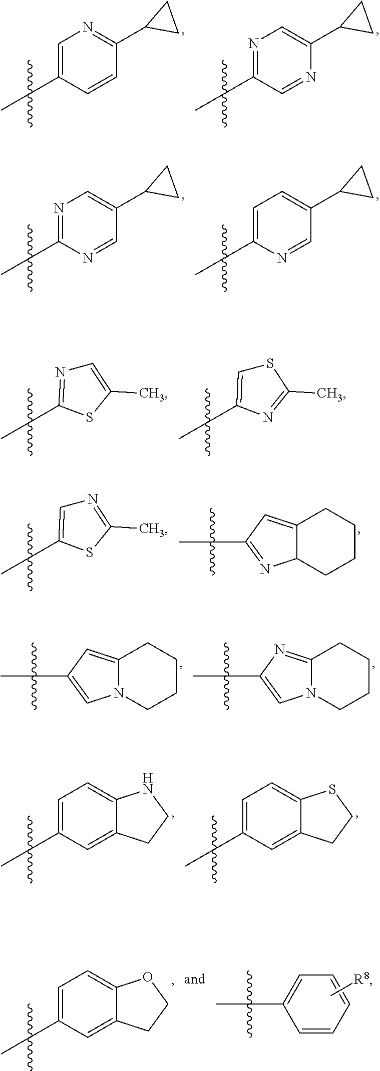

wherein R⁸ is selected from R⁹ and OR⁹, and wherein R⁹ is an unsubstituted C₁-, C₂-, C₃-, or C₄-alkyl or unsubstituted C₃-, or C₄-cycloalkyl;
R² is —OR⁵ or —R⁵, wherein R⁵ is selected from the group consisting of —H and optionally substituted, in particular unsubstituted C₁-C₁₀ alkyl, i.e. C₁-, C₂-, C₃-, C₄-, C₅-, C₆-, C₇-, C₈-, C₉- or C₁₀-alkyl; C₂-C₁₀-alkenyl, i.e. C₂-, C₃-, C₄-, C₅-, C₆-, C₇-, C₈-, C₉- or C₁₀-alkenyl; C₂-C₁₀-alkynyl, i.e. C₂-, C₃-, C₄-, C₅-, C₆-, C₇-, C₈-, C₉- or C₁₀-alkenyl; C₃-C₁₀-cycloalkyl, i.e. C₃-, C₄-, C₅-, C₆-, C₇-, C₈-, C₉- or C₁₀-cycloalkyl; C₃-C₁₀-heterocycloalkyl, i.e. C₃-, C₄-, C₅-, C₆-, C₇-, C₈-, C₉- or C₁₀-heterocycloalkyl; C₅-C₁₄-aryl, i.e. C₅-, C₆-, C₇-, C₈-, C₉-, C₁₀-, C₁₁-, C₁₂-, C₁₃-, or C₁₄-aryl; C₅-C₁₄-heteroaryl, i.e. C₅-, C₆-, C₇-, C₈-, C₉-, C₁₀-, C₁₁-, C₁₂-, C₁₃-, or C₁₄-heteroaryl; C₆-C₁₅-aralkyl, i.e. C₆-, C₇-, C₈-, C₉-, C₁₀-, C₁₁-C₁₂-, C₁₃-, C₁₄-, or C₁₅-aralkyl; C₆-C₁₅-heteroaralkyl, i.e. C₆-, C₇-, C₈-, C₉-, C₁₀-, C₁₁-C₁₂-, C₁₃-, C₁₄-, or C₁₅-heteroaralkyl; in particular R² is —OCH₂;
R³ is (CH₂)ₙ₂—R⁷, wherein n2 is 1-4, i.e. 1, 2, 3, or 4, and R⁷ is selected from a optionally substituted C₁-C₇-alkyl, C₄-C₇-cycloalkyl; C₅-C₁₀-aryl or C₅-C₁₀-heteroaryl,
Y is absent or selected from —*CH₂—, —*O—, —*CH₂—CH₂—, —*CH₂—O—, and —*O—CH₂—, in particular Y is absent or selected from —*CH₂— and —*O—, wherein the *C or *O is covalently linked to the C atom of the carbonyl group; and
X is selected from the group consisting of —*CH₂—, —NH—, —O—, —S—, and —*CHF—, in particular X is —*CH₂—, wherein the *C is part of the heterocycloalkyl.

In preferred embodiments of the first aspect, the compound for use is characterized by formula (1a) or (1b), in particular (1b), wherein
A is a 4- to 8-, i.e. 4-, 5-, 6-7 or 8-membered, in particular 5- or 6-membered heterocycloalkyl or heteroaryl;
B is a 4- to 8-, i.e. 4-, 5-, 6-7 or 8-membered cycloalkyl, heterocycloalkyl, aryl or heteroaryl, wherein B may be present or absent and wherein if B is present, A and B together form a double cycle;
C is 5- or 6-membered aryl or heteroaryl;
R¹ is selected from

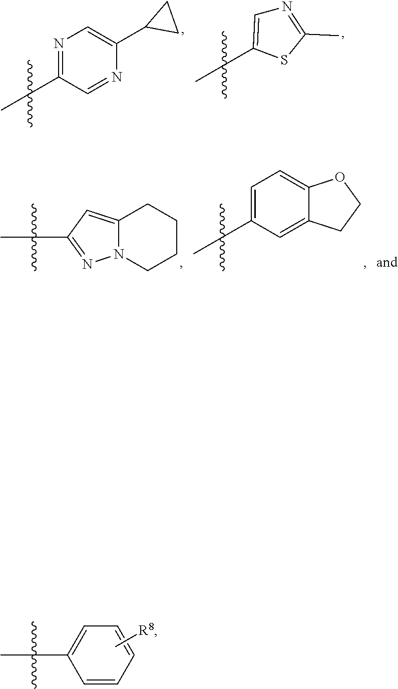

wherein R⁸ is selected from R⁹ and OR⁹, wherein R⁹ is an unsubstituted C₁-C₄-alkyl;
R² is —OR⁵ or —R⁵, wherein R⁵ is selected from the group consisting of —H and optionally substituted, in particular unsubstituted C₁-C₁₀ alkyl, C₂-C₁₀-alkenyl, C₂-C₁₀-alkynyl, C₃-C₁₀-cycloalkyl, C₃-C₁₀-heterocycloalkyl, C₅-C₁₄-aryl, C₅-C₁₄-heteroaryl, C₆-C₁₅-aralkyl, C₆-C₁₅-heteroaralkyl, in particular R² is —OCH₂;
R³ is (CH₂)ₙ₂—R⁷, wherein n2 is 1-4, i.e. 1, 2, 3, or 4, and R⁷ is selected from an optionally substituted C₁-C₇-alkyl, C₄-C₇-cycloalkyl; C₅-C₁₀-aryl or C₅-C₁₀-heteroaryl,
Y is absent or selected from —*CH₂—, —*O—, —*CH₂—CH₂—, —*CH₂—O—, and —*O—CH₂—, in particular Y is absent or selected from —*CH₂— and —*O—, wherein the *C or *O is covalently linked to the C atom of the carbonyl group; and
X is selected from the group consisting of —CH₂—, —NH—, —O—, —S—, and —*CHF—, in particular X is —*CH₂—, wherein the *C is part of the heterocycloalkyl.

In preferred embodiments of the first aspect, the compound for use is characterized by formula (1a) or (1b), in particular (1b), wherein
A is a 4- to 8-, i.e. 4-, 5-, 6-7 or 8-membered, in particular 5- or 6-membered heterocycloalkyl or heteroaryl;
B is a 4- to 8-, i.e. 4-, 5-, 6-7 or 8-membered cycloalkyl, heterocycloalkyl, aryl or heteroaryl, wherein B may be present or absent and wherein if B is present, A and B together form a double cycle;
C is 5- or 6-membered aryl or heteroaryl;
R¹ is selected from

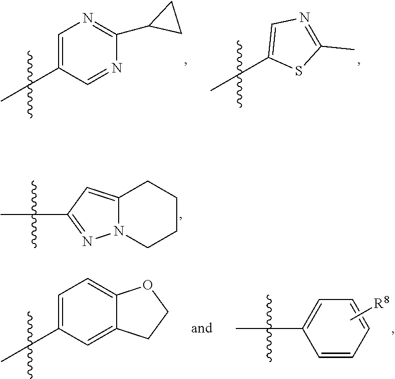

in particular wherein R⁸ is in para position, and wherein R⁸ is OR⁹, wherein R⁹ is C₁-, C₂-, C₃-, or C₄-alkyl, in particular R⁹ is —CH₃;
R² is —OR⁵ or —R⁵, wherein R⁵ is selected from the group consisting of —H and optionally substituted C₁-C₁₀ alkyl, C₂-C₁₀-alkenyl, C₂-C₁₀-alkynyl, C₃-C₁₀-cycloalkyl, C₃-C₁₀-heterocycloalkyl, C₅-C₁₄-aryl, C₅-C₁₄-heteroaryl, C₆-C₁₅-aralkyl and C₆-C₁₅-heteroaralkyl, in particular R² is —OCH₂;
R³ is (CH₂)ₙ₂—R⁷, wherein n2 is 1-4, i.e. 1, 2, 3, or 4, and R⁷ is selected from the group consisting of optionally substituted C₁-C₇-alkyl, C₄-C₇-cycloalkyl, C₅-C₁₀-aryl and C₅-C₁₀-heteroaryl,
Y is absent or selected from —*CH₂—, —*O—, —*CH₂—CH₂—, —*CH₂—O—, and —*O—CH₂—, in particular Y is absent or selected from —*CH₂— and —*O—, wherein the *C or *O is covalently linked to the C atom of the carbonyl group; and
X is selected from the group consisting of —CH₂—, —NH—, —O—, —S—, and —*CHF—, in particular X is —*CH₂—, wherein the *C is part of the heterocycloalkyl.

In preferred embodiments of the first aspect, the compound for use is characterized by formula (1a) or (1b), in particular (1b), wherein
A is a 4- to 8-, i.e. 4-, 5-, 6-7 or 8-membered, in particular 5- or 6-membered heterocycloalkyl or heteroaryl;
B is a 4- to 8-, i.e. 4-, 5-, 6-7 or 8-membered cycloalkyl, heterocycloalkyl, aryl or heteroaryl, wherein B may be present or absent and wherein if B is present, A and B together form a double cycle;
C is 5- or 6-membered aryl or heteroaryl;
R¹ is

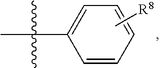

wherein R⁸ is —O—CH₃, in particular wherein R⁸ is in para position;
R² is —OR⁵ or —R⁵, wherein R⁵ is selected from the group consisting of —H and optionally substituted C₁-C₁₀ alkyl, C₂-C₁₀-alkenyl, C₂-C₁₀-alkynyl, C₃-C₁₀-cycloalkyl, C₃-C₁₀-heterocycloalkyl, C₅-C₁₄-aryl, C₅-C₁₄-heteroaryl, C₆-C₁₅-aralkyl and C₆-C₁₅-heteroaralkyl, in particular R² is —OCH₂;
R³ is (CH₂)ₙ₂—R⁷, wherein n2 is 1-4, i.e. 1, 2, 3, or 4, and R⁷ is selected from optionally substituted C₁-C₇-alkyl, C₄-C₇-cycloalkyl, C₅-C₁₀-aryl and C₅-C₁₀-heteroaryl,
Y is absent or selected from —*CH₂—, —*O—, —*CH₂—CH₂—, —*CH₂—O—, and —*O—CH₂—, in particular Y is absent or selected from —*CH₂— and —*O—, wherein the *C or *O is covalently linked to the C atom of the carbonyl group; and
X is selected from the group consisting of —CH₂—, —NH—, —O—, —S—, and —*CHF—, in particular X is —*CH₂—, wherein the *C is part of the heterocycloalkyl.

In preferred embodiments of the first aspect, the compound for use is characterized by formula (1a) or (1b), in particular (1b), wherein
A is a 4- to 8-, i.e. 4-, 5-, 6-7 or 8--membered, in particular 5- or 6-membered heterocycloalkyl or heteroaryl;
B is a 4- to 8-, i.e. 4-, 5-, 6-7 or 8-membered cycloalkyl, heterocycloalkyl, aryl or heteroaryl, wherein B may be present or absent and wherein if B is present, A and B together form a double cycle;
C is 5- or 6-membered aryl or heteroaryl;
R¹ is

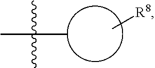

wherein

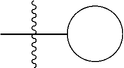

is a 5- to 12-membered, i.e. 5-, 6-, 7-, 8-, 9-, 10-, 11-, or 12-membered alicyclic system, aryl or heteroaryl, in particular a 5- or 6-membered aryl or heteroaryl, and R⁸ is selected from H, R⁹ and OR⁹, wherein R⁹ is selected from optionally substituted C₁-, C₂-, C₃-, C₄-C₅-, C₆- or C₇-alkyl and optionally substituted C₃-, C₄-C₅-, C₆- or C₇-cycloalkyl, wherein the aryl or heteroaryl and R⁹ may form a double cycle,
wherein preferably

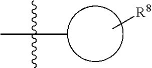

comprises at least one heteroatom;
more preferably, R¹ is selected from the group consisting of

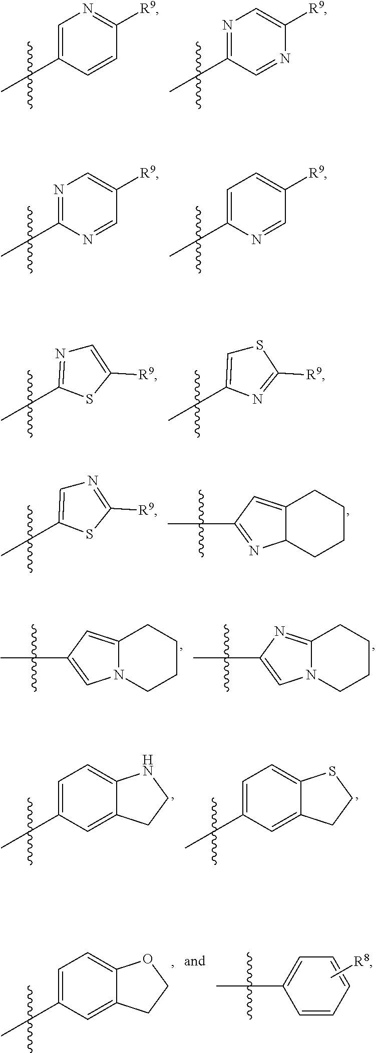

wherein R⁸ is selected from R⁹ and OR⁹, and wherein R⁹ is an unsubstituted C₁-, C₂-, C₃-, or C₄-alkyl or unsubstituted C₃-, or C₄-cycloalkyl;
even more preferably, R¹ is selected from the group consisting of

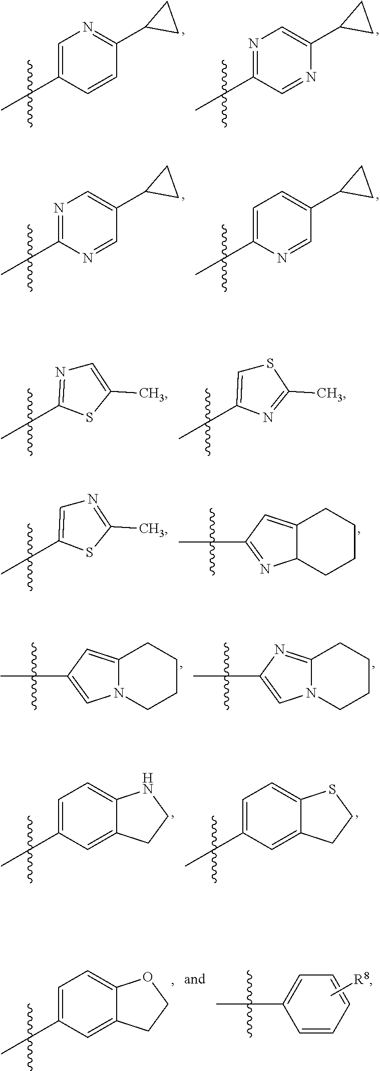

wherein R⁸ is selected from R⁹ and OR⁹, and wherein R⁹ is an unsubstituted C₁-, C₂-, C₃-, or C₄-alkyl or unsubstituted C₃-, or C₄-cycloalkyl;
R² is —OR⁵ or —R⁵, wherein R⁵ is selected from the group consisting of —H and optionally substituted C₁-C₁₀ alkyl, C₂-C₁₀-alkenyl, C₂-C₁₀-alkynyl, C₃-C₁₀-cycloalkyl, C₃-C₁₀-heterocycloalkyl, C₅-C₁₄-aryl, C₅-C₁₄-heteroaryl, C₆-C₁₅-aralkyl, and C₆-C₁₅-heteroaralkyl;
R³ is (CH₂)ₙ₂—R⁷, wherein n2 is 1-4, i.e. 1, 2, 3, or 4 and R⁷ is selected from optionally substituted C₁-C₇-alkyl, C₄-C₇-cycloalkyl, C₅-C₁₀-aryl and C₅-C₁₀-heteroaryl,
Y is absent or selected from —*CH₂—, —*O—, —*CH₂—CH₂—, —*CH₂—O—, and —*O—CH₂—, in particular Y is absent or selected from —*CH₂— and —*O—, wherein the *C or *O is covalently linked to the C atom of the carbonyl group; and
X is selected from the group consisting of —CH₂—, —NH—, —O—, —S—, and —*CHF—, in particular X is —*CH₂—, wherein the *C is part of the heterocycloalkyl.

In preferred embodiments of the first aspect, the compound for use is characterized by formula (1a) or (1b), in particular (1b), wherein
A is a 4- to 8-, i.e. 4-, 5-, 6-7 or 8-membered, in particular 5- or 6-membered heterocycloalkyl or heteroaryl;
B is a 4- to 8-, i.e. 4-, 5-, 6-7 or 8-membered cycloalkyl, heterocycloalkyl, aryl or heteroaryl, wherein B may be present or absent and wherein if B is present, A and B together form a double cycle;
C is 5- or 6-membered aryl or heteroaryl;
R¹ is selected from

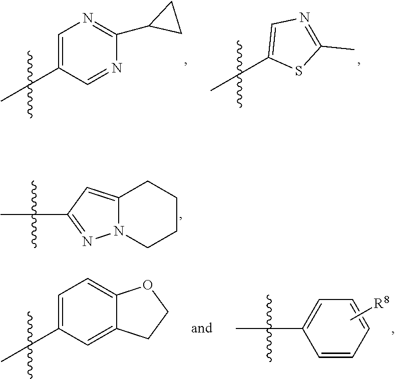

in particular wherein R⁸ is in para position, and wherein R⁸ is OR⁹, wherein R⁹ is C₁-, C₂-, C₃-, or C₄-alkyl, in particular R⁹ is —CH₃;
R² is —OR⁵, wherein R⁵ is selected from the group consisting of —H and optionally substituted C₁-C₆ alkyl, C₂-C₆-alkenyl, C₂-C₆-alkynyl and C₃-C₈-cycloalkyl, in particular from C₁-C₄-alkyl or C₃-C₆-cycloalkyl;
R³ is (CH₂)ₙ₂—R⁷, wherein n2 is 1-4, i.e. 1, 2, 3, or 4, and R⁷ is selected from the group consisting of optionally substituted C₁-C₇-alkyl, C₄-C₇-cycloalkyl, C₅-C₁₀-aryl and C₅-C₁₀-heteroaryl,
Y is absent or selected from —*CH₂—, —*O—, —*CH₂—CH₂—, —*CH₂—O—, and —*O—CH₂—, in particular Y is absent or selected from —*CH₂— and —*O—, wherein the *C or *O is covalently linked to the C atom of the carbonyl group; and
X is selected from the group consisting of —CH₂—, —NH—, —O—, —S—, and —*CHF—, in particular X is —*CH₂—, wherein the *C is part of the heterocycloalkyl.

In preferred embodiments of the first aspect, the compound for use is characterized by formula (1a) or (1b), in particular (1b), wherein
A is a 4- to 8-membered, in particular 5- or 6-membered heterocycloalkyl or heteroaryl;
B is a 4- to 8-membered cycloalkyl, heterocycloalkyl, aryl or heteroaryl, wherein B may be present or absent and wherein if B is present, A and B together form a double cycle;
C is 5- or 6-membered aryl or heteroaryl;
R¹ is selected from

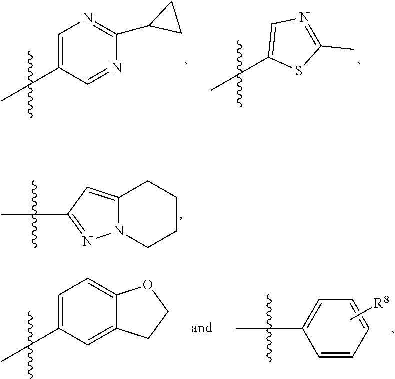

in particular wherein R⁸ is in para position, and wherein R⁸ is OR⁹, wherein R⁹ is C₁-, C₂-, C₃-, or C₄-alkyl, in particular R⁹ is —CH₃;
R² is —OCH₃;
R³ is (CH₂)ₙ₂—R⁷, wherein n2 is 1-4, i.e. 1, 2, 3 or 4, and R⁷ is selected from the group consisting of optionally substituted C₁-C₇-alkyl, C₄-C₇-cycloalkyl, C₅-C₁₀-aryl and C₅-C₁₀-heteroaryl,
Y is absent or selected from —*CH₂—, —*O—, —*CH₂—CH₂—, —*CH₂—O—, and —*O—CH₂—, in particular Y is absent or selected from —*CH₂— and —*O—, wherein the *C or *O is covalently linked to the C atom of the carbonyl group; and
X is selected from the group consisting of —CH₂—, —NH—, —O—, —S—, and —*CHF—, in particular X is —*CH₂—, wherein the *C is part of the heterocycloalkyl.

In preferred embodiments of the first aspect, the compound for use is characterized by formula (1a) or (1b), in particular (1b), wherein
A is a 4- to 8-, i.e. 4-, 5-, 6-7 or 8-membered, in particular 5- or 6-membered heterocycloalkyl or heteroaryl;
B is a 4- to 8-, i.e. 4-, 5-, 6-7 or 8-membered cycloalkyl, heterocycloalkyl, aryl or heteroaryl, wherein B may be present or absent and wherein if B is present, A and B together form a double cycle;
C is 5- or 6-membered aryl or heteroaryl;
R¹ is selected from

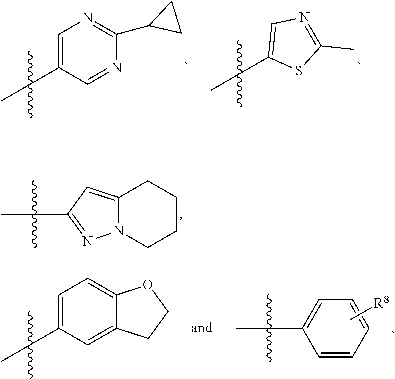

in particular wherein R⁸ is in para position, and wherein R′ is OR⁹, wherein R⁹ is C₁-, C₂-, C₃-, or C₄-alkyl, in particular R⁹ is —CH₃;
R² is —OR⁵ or —R⁵, wherein R⁵ is selected from the group consisting of —H and optionally substituted C₁-C₁₀ alkyl, C₂-C₁₀-alkenyl, C₂-C₁₀-alkynyl, C₃-C₁₀-cycloalkyl, C₃-C₁₀-heterocycloalkyl, C₅-C₁₄-aryl, C₅-C₁₄-heteroaryl, C₆-C₁₅-aralkyl and C₆-C₁₅-heteroaralkyl, in particular R² is —OCH₂;
R³ is (CH₂)ₙ₂—R⁷, wherein n2 is 1-4, i.e. 1, 2, 3 or 4, and R⁷ is selected from

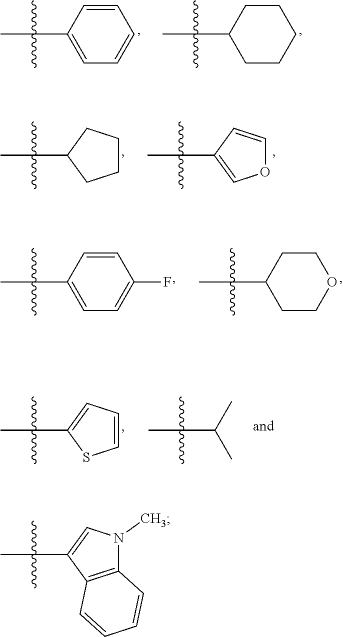

in particular R⁷ is

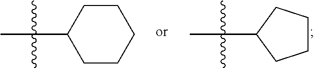 [SMILES: *C1CCCCC1 ; *C1CCCC1]

Y is absent or selected from —*CH₂—, —*O—, —*CH₂—CH₂—, —*CH₂—O—, and —*O—CH₂—, in particular Y is absent or selected from —*CH₂— and —*O—, wherein the *C or *O is covalently linked to the C atom of the carbonyl group; and
X is selected from the group consisting of —CH₂—, —NH—, —O—, —S—, and —*CHF—, in particular X is —*CH₂—, wherein the *C is part of the heterocycloalkyl.

In preferred embodiments of the first aspect, the compound for use is characterized by formula (2a) or (2b), in particular (2b), wherein
A is a 4- to 8-, i.e. 4-, 5-, 6-7- or 8-membered, in particular 5- or 6-membered heterocycloalkyl or heteroaryl;
B is a 4- to 8-, i.e. 4-, 5-, 6-7- or 8-membered cycloalkyl, heterocycloalkyl, aryl or heteroaryl, wherein B may be present or absent and wherein if B is present, A and B together form a double cycle;
R¹ is

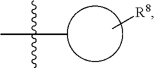

wherein

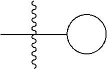

is a 5- to 12-membered, i.e. 5-, 6-, 7-, 8-, 9-, 10-, 11-, or 12-membered alicyclic system, aryl or heteroaryl, in particular a 5- or 6-membered aryl or heteroaryl, and R⁸ is selected from H, R⁹ and OR⁹, wherein R⁹ is selected from optionally substituted C₁-, C₂-, C₃-, C₄-C₅-, C₆- or C₇-alkyl and optionally substituted C₃-, C₄-C₅-, C₆- or C₇-cycloalkyl, wherein the aryl or heteroaryl and R⁹ may form a double cycle,
wherein preferably

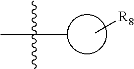

comprises at least one heteroatom;
more preferably, R¹ is selected from the group consisting of

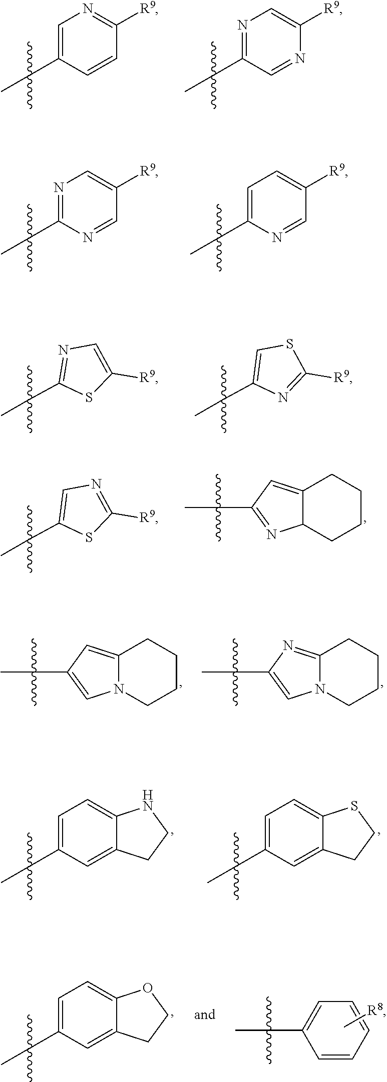

wherein R⁸ is selected from R⁹ and OR⁹, and wherein R⁹ is an unsubstituted C₁-, C₂-, C₃-, or C₄-alkyl or unsubstituted C₃-, or C₄-cycloalkyl;
even more preferably, R¹ is selected from the group consisting of

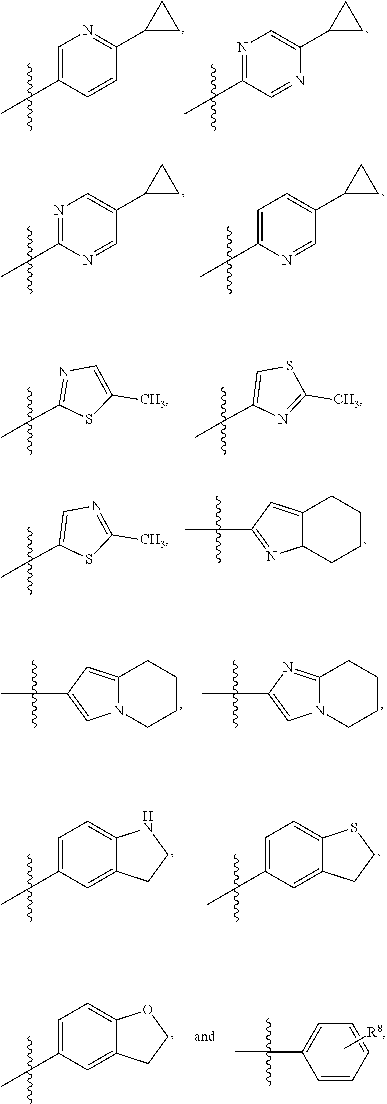

wherein R⁸ is selected from R⁹ and OR⁹, and wherein R⁹ is an unsubstituted C₁-, C₂-, C₃-, or C₄-alkyl or unsubstituted C₃-, or C₄-cycloalkyl;
R² is —OR⁵ or —R⁵, wherein R⁵ is selected from the group consisting of —H and optionally substituted C₁-C₁₀ alkyl, C₂-C₁₀-alkenyl, C₂-C₁₀-alkynyl, C₃-C₁₀-cycloalkyl, C₃-C₁₀-heterocycloalkyl, C₅-C₁₄-aryl, C₅-C₁₄-heteroaryl, C₆-C₁₅-aralkyl, and C₆-C₁₅-heteroaralkyl; in particular R² is —OCH₂;
R³ is (CH₂)ₙ₂—R⁷, wherein n2 is 1-4, i.e. 1, 2, 3, or 4, and R⁷ is selected from the group consisting of optionally substituted C₁-C₇-alkyl, C₄-C₇-cycloalkyl, C₅-C₁₀-aryl and C₅-C₁₀-heteroaryl,
Y is absent or selected from —*CH₂—, —*O—, —*CH₂—CH₂—, —*CH₂—O—, and —*O—CH₂—, in particular Y is absent or selected from —*CH₂— and —*O—, wherein the *C or *O is covalently linked to the C atom of the carbonyl group; and
X is selected from the group consisting of —*CH₂—, —NH—, —O—, —S—, and —*CHF—, in particular X is —*CH₂—, wherein the *C is part of the heterocycloalkyl.

In preferred embodiments of the first aspect, the compound for use is characterized by formula (2a) or (2b), in particular (2b), wherein
A is a 4- to 8-, i.e. 4-, 5-, 6-7 or 8-membered, in particular 5- or 6-membered heterocycloalkyl or heteroaryl;
B is a 4- to 8-, i.e. 4-, 5-, 6-7 or 8-membered cycloalkyl, heterocycloalkyl, aryl or heteroaryl, wherein B may be present or absent and wherein if B is present, A and B together form a double cycle;
R¹ is selected from

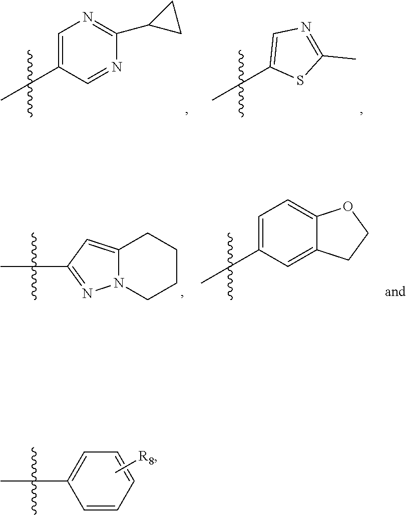

wherein R⁸ is selected from R⁹ and OR⁹, wherein R⁹ is an unsubstituted C₁-C₄-alkyl;
R² is —OR⁵ or —R⁵, wherein R⁵ is selected from the group consisting of —H and optionally substituted C₁-C₁₀ alkyl, C₂-C₁₀-alkenyl, C₂-C₁₀-alkynyl, C₃-C₁₀-cycloalkyl, C₃-C₁₀-heterocycloalkyl, C₅-C₁₄-aryl, C₅-C₁₄-heteroaryl and C₆-C₁₅-aralkyl, C₆-C₁₅-heteroaralkyl, in particular R² is —OCH₂;
R³ is (CH₂)ₙ₂—R⁷, wherein n2 is 1-4, i.e. 1, 2, 3, or 4, and R⁷ is selected from the group consisting of optionally substituted C₁-C₇-alkyl, C₄-C₇-cycloalkyl, C₅-C₁₀-aryl and C₅-C₁₀-heteroaryl,
Y is absent or selected from —*CH₂—, —*O—, —*CH₂—CH₂—, —*CH₂—O—, and —*O—CH₂—, in particular Y is absent or selected from —*CH₂— and —*O—, wherein the *C or *O is covalently linked to the C atom of the carbonyl group; and
X is selected from the group consisting of —CH₂—, —NH—, —O—, —S—, and —*CHF—, in particular X is —*CH₂—, wherein the *C is part of the heterocycloalkyl.

In preferred embodiments of the first aspect, the compound for use is characterized by formula (2a) or (2b), in particular (2b), wherein
A is a 4- to 8-, i.e. 4-, 5-, 6-7 or 8-membered, in particular 5- or 6-membered heterocycloalkyl or heteroaryl;
B is a 4- to 8-, i.e. 4-, 5-, 6-7 or 8-membered cycloalkyl, heterocycloalkyl, aryl or heteroaryl, wherein B may be present or absent and wherein if B is present, A and B together form a double cycle;
R is selected from

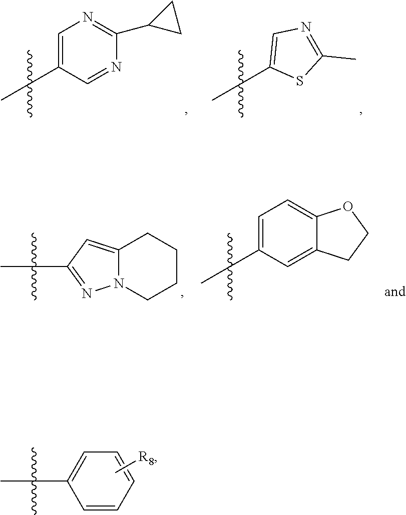

in particular wherein R⁸ is in para position, and wherein R⁸ is OR⁹, wherein R⁹ is an unsubstituted C₁-C₄-alkyl, in particular R⁹ is —CH₃;
R² is —OR⁵ or —R⁵, wherein R⁵ is selected from the group consisting of —H and optionally substituted C₁-C₁₀ alkyl, C₂-C₁₀-alkenyl, C₂-C₁₀-alkynyl, C₃-C₁₀-cycloalkyl, C₃-C₁₀-heterocycloalkyl, C₅-C₁₄-aryl, C₅-C₁₄-heteroaryl, C₆-C₁₅-aralkyl and C₆-C₁₅-heteroaralkyl, in particular R² is —OCH₂;
R³ is (CH₂)ₙ₂—R⁷, wherein n2 is 1-4, i.e. 1, 2, 3, or 4, and R⁷ is selected from the group consisting of optionally substituted C₁-C₇-alkyl, C₄-C₇-cycloalkyl, C₅-C₁₀-aryl and C₅-C₁₀-heteroaryl,
Y is absent or selected from —*CH₂—, —*O—, —*CH₂—CH₂—, —*CH₂—O—, and —*O—CH₂—, in particular Y is absent or selected from —*CH₂— and —*O—, wherein the *C or *O is covalently linked to the C atom of the carbonyl group; and
X is selected from the group consisting of —CH₂—, —NH—, —O—, —S—, and —*CHF—, in particular X is —*CH₂—, wherein the *C is part of the heterocycloalkyl.

In preferred embodiments of the first aspect, the compound for use is characterized by formula (2a) or (2b), in particular (2b), wherein
A is a 4- to 8-, i.e. 4-, 5-, 6-7 or 8-membered, in particular 5- or 6-membered heterocycloalkyl or heteroaryl;
B is a 4- to 8-, i.e. 4-, 5-, 6-7 or 8-membered cycloalkyl, heterocycloalkyl, aryl or heteroaryl, wherein B may be present or absent and wherein if B is present, A and B together form a double cycle;
R¹ is

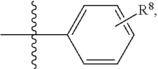

wherein R⁸ is —O—CH₃, in particular wherein R⁸ is in para position;
R² is —OR⁵ or —R⁵, wherein R⁵ is selected from the group consisting of —H and optionally substituted C₁-C₁₀ alkyl, C₂-C₁₀-alkenyl, C₂-C₁₀-alkynyl, C₃-C₁₀-cycloalkyl, C₃-C₁₀-heterocycloalkyl, C₅-C₁₄-aryl, C₅-C₁₄-heteroaryl, C₆-C₁₅-aralkyl and C₆-C₁₅-heteroaralkyl, in particular R² is —OCH₂;
R³ is (CH₂)ₙ₂—R⁷, wherein n2 is 1-4, i.e. 1, 2, 3, or 4, and R⁷ is selected from optionally substituted C₁-C₇-alkyl, C₄-C₇-cycloalkyl, C₅-C₁₀-aryl and C₅-C₁₀-heteroaryl,
Y is absent or selected from —*CH₂—, —*O—, —*CH₂—CH₂—, —*CH₂—O—, and —*O—CH₂—, in particular Y is absent or selected from —*CH₂— and —*O—, wherein the *C or *O is covalently linked to the C atom of the carbonyl group; and
X is selected from the group consisting of —CH₂—, —NH—, —O—, —S—, and —*CHF—, in particular X is —*CH₂—, wherein the *C is part of the heterocycloalkyl.

In preferred embodiments of the first aspect, the compound for use is characterized by formula (2a) or (2b), in particular (2b), wherein
A is a 4- to 8-, i.e. 4-, 5-, 6-7 or 8-membered, in particular 5- or 6-membered heterocycloalkyl or heteroaryl;
B is a 4- to 8-, i.e. 4-, 5-, 6-7 or 8-membered cycloalkyl, heterocycloalkyl, aryl or heteroaryl, wherein B may be present or absent and wherein if B is present, A and B together form a double cycle;
R¹ is

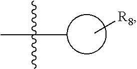

wherein

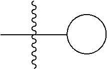

is a 5- to 12-membered, i.e. 5-, 6-, 7-, 8-, 9-, 10-, 11-, or 12-membered alicyclic system, aryl or heteroaryl, in particular a 5- or 6-membered aryl or heteroaryl, and R⁸ is selected from H, R⁹ and OR⁹, wherein R⁹ is selected from optionally substituted C₁-, C₂-, C₃-, C₄-C₅-, C₆- or C₇-alkyl and optionally substituted C₃-, C₄-C₅-, C₆- or C₇-cycloalkyl, wherein the aryl or heteroaryl and R⁹ may form a double cycle,
wherein preferably

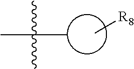

comprises at least one heteroatom;
more preferably, R¹ is selected from the group consisting of

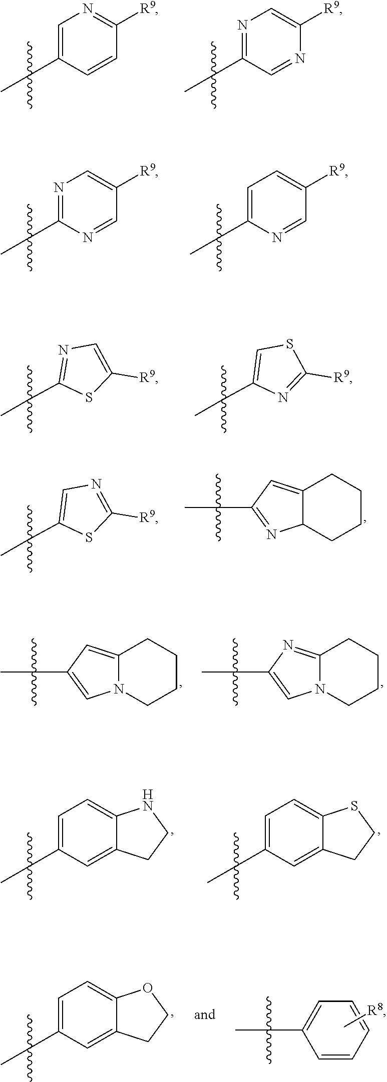

wherein R⁸ is selected from R⁹ and OR⁹, and wherein R⁹ is an unsubstituted C₁-, C₂-, C₃-, or C₄-alkyl or unsubstituted C₃-, or C₄-cycloalkyl;
even more preferably, R¹ is selected from the group consisting of

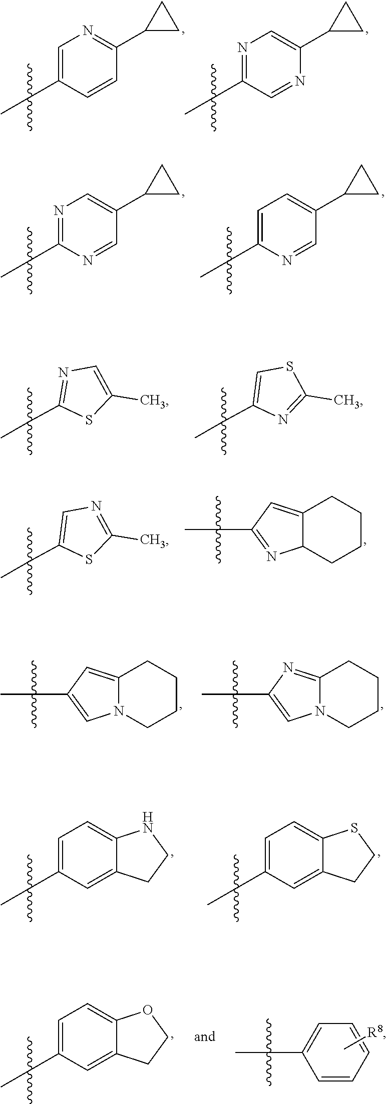

wherein R⁸ is selected from R⁹ and OR⁹, and wherein R⁹ is an unsubstituted C₁-, C₂-, C₃-, or C₄-alkyl or unsubstituted C₃-, or C₄-cycloalkyl;
R² is —OR⁵ or —R⁵, wherein R⁵ is selected from the group consisting of —H and optionally substituted C₁-C₁₀ alkyl, C₂-C₁₀-alkenyl, C₂-C₁₀-alkynyl, C₃-C₁₀-cycloalkyl, C₃-C₁₀-heterocycloalkyl, C₅-C₁₄-aryl, C₅-C₁₄-heteroaryl, C₆-C₁₅-aralkyl, C₆-C₁₅-heteroaralkyl, in particular R² is —OCH₂;
R³ is (CH₂)ₙ₂—R⁷, wherein n2 is 1-4, i.e. 1, 2, 3, or 4, and R⁷ is selected from the group consisting of optionally substituted C₁-C₇-alkyl, C₄-C₇-cycloalkyl, C₅-C₁₀-aryl and C₅-C₁₀-heteroaryl,
Y is absent or selected from —*CH₂—, —*O—, —*CH₂—CH₂—, —*CH₂—O—, and —*O—CH₂—, in particular Y is absent or selected from —*CH₂— and —*O—, wherein the *C or *O is covalently linked to the C atom of the carbonyl group; and
X is selected from the group consisting of —CH₂—, —NH—, —O—, —S—, and —*CHF—, in particular X is —*CH₂—, wherein the *C is part of the heterocycloalkyl.

In preferred embodiments of the first aspect, the compound for use is characterized by formula (2a) or (2b), in particular (2b), wherein
A is a 4- to 8-, i.e. 4-, 5-, 6-7 or 8-membered, in particular 5- or 6-membered heterocycloalkyl or heteroaryl;
B is a 4- to 8-, i.e. 4-, 5-, 6-7 or 8-membered cycloalkyl, heterocycloalkyl, aryl or heteroaryl, wherein B may be present or absent and wherein if B is present, A and B together form a double cycle;
R¹ is selected from

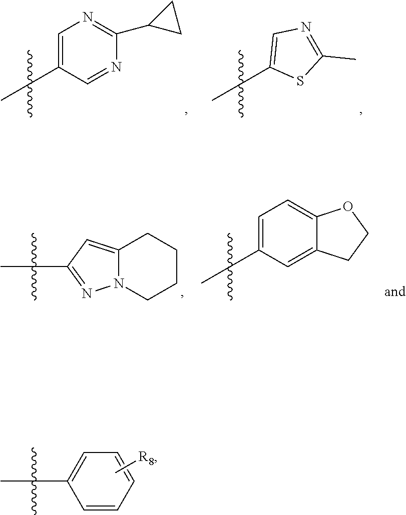

in particular wherein R⁸ is in para position, and wherein R⁸ is OR⁹, wherein R⁹ is C₁-, C₂-, C₃-, or C₄-alkyl, in particular R⁹ is —CH₃;
R² is —OR⁵, wherein R⁵ is selected from the group consisting of —H and optionally substituted C₁-C₆ alkyl, C₂-C₆-alkenyl, C₂-C₆-alkynyl and C₃-C₈-cycloalkyl, in particular from an C₁-C₄-alkyl or C₃-C₆-cycloalkyl;
R³ is (CH₂)ₙ₂—R⁷, wherein n2 is 1-4, i.e. 1, 2, 3 or 4, and R⁷ is selected from the group consisting of optionally substituted C₁-C₇-alkyl, C₄-C₇-cycloalkyl, C₅-C₁₀-aryl and C₅-C₁₀-heteroaryl,
Y is absent or selected from —*CH₂—, —*O—, —*CH₂—CH₂—, —*CH₂—O—, and —*O—CH₂—, in particular Y is absent or selected from —*CH₂— and —*O—, wherein the *C or *O is covalently linked to the C atom of the carbonyl group; and
X is selected from the group consisting of —CH₂—, —NH—, —O—, —S—, and —*CHF—, in particular X is —*CH₂—, wherein the *C is part of the heterocycloalkyl.

In preferred embodiments of the first aspect, the compound for use is characterized by formula (2a) or (2b), in particular (2b), wherein
A is a 4- to 8-, i.e. 4-, 5-, 6-7 or 8-membered, in particular 5- or 6-membered heterocycloalkyl or heteroaryl;
B is a 4- to 8-, i.e. 4-, 5-, 6-7 or 8-membered cycloalkyl, heterocycloalkyl, aryl or heteroaryl, wherein B may be present or absent and wherein if B is present, A and B together form a double cycle;
R is selected from

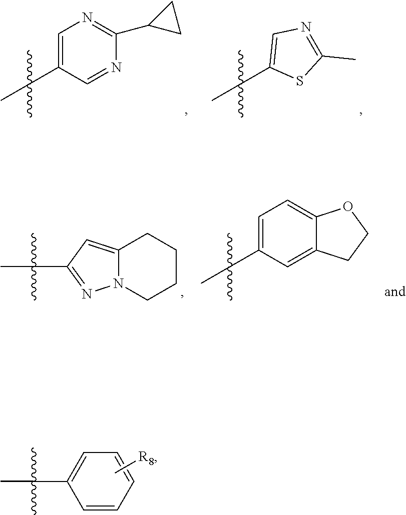

in particular wherein R⁸ is in para position, and wherein R⁸ is OR⁹, wherein R⁹ is C₁-, C₂-, C₃-, or C₄-alkyl, in particular R⁹ is —CH₃;
R² is —OCH₃—;
R³ is (CH₂)ₙ₂—R⁷, wherein n2 is 1-4, i.e. 1, 2, 3, or 4, and R⁷ is selected from the group consisting of optionally substituted C₁-C₇-alkyl, C₄-C₇-cycloalkyl, C₅-C₁₀-aryl and C₅-C₁₀-heteroaryl,
Y is absent or selected from —*CH₂—, —*O—, —*CH₂—CH₂—, —*CH₂—O—, and —*O—CH₂—, in particular Y is absent or selected from —*CH₂— and —*O—, wherein the *C or *O is covalently linked to the C atom of the carbonyl group; and
X is selected from the group consisting of —CH₂—, —NH—, —O—, —S—, and —*CHF—, in particular X is —*CH₂—, wherein the *C is part of the heterocycloalkyl.

In preferred embodiments of the first aspect, the compound for use is characterized by formula (2a) or (2b), in particular (2b), wherein
A is a 4- to 8-, i.e. 4-, 5-, 6-7 or 8-membered, in particular 5- or 6-membered heterocycloalkyl or heteroaryl;
B is a 4- to 8-, i.e. 4-, 5-, 6-7 or 8-membered cycloalkyl, heterocycloalkyl, aryl or heteroaryl, wherein B may be present or absent and wherein if B is present, A and B together form a double cycle;
R¹ is selected from

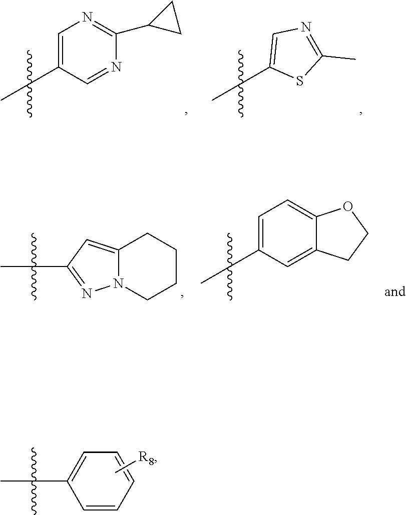

in particular wherein R⁸ is in para position, and wherein R⁸ is OR⁹, wherein R⁹ is C₁-, C₂-, C₃-, or C₄-alkyl, in particular R⁹ is —CH₃;
R² is —OR⁵ or —R⁵, wherein R⁵ is selected from the group consisting of —H and optionally substituted C₁-C₁₀ alkyl, C₂-C₁₀-alkenyl, C₂-C₁₀-alkynyl, C₃-C₁₀-cycloalkyl, C₃-C₁₀-heterocycloalkyl, C₅-C₁₄-aryl, C₅-C₁₄-heteroaryl, C₆-C₁₅-aralkyl, C₆-C₁₅-heteroaralkyl, in particular R² is —OCH₂;
R³ is (CH₂)ₙ₂—R⁷, wherein n2 is 1-4, i.e. 1, 2, 3 or 4, and R⁷ is selected from

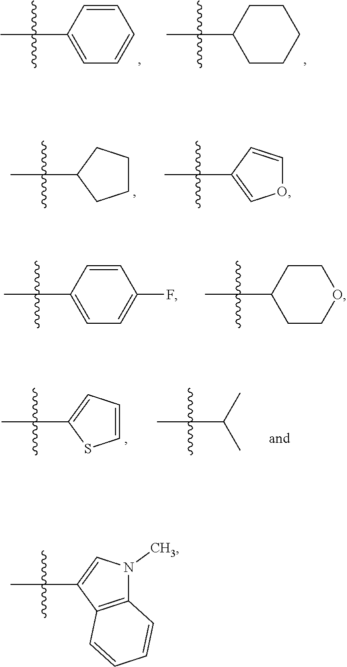

in particular R⁷ is or

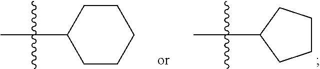 [SMILES: *C1CCCCC1 ; *C1CCCC1]

Y is absent or selected from —*CH₂—, —*O—, —*CH₂—CH₂—, —*CH₂—O—, and —*O—CH₂—, in particular Y is absent or selected from —*CH₂— and —*O—, wherein the *C or *O is covalently linked to the C atom of the carbonyl group; and
X is selected from the group consisting of —CH₂—, —NH—, —O—, —S—, and —*CHF—, in particular X is —*CH₂—, wherein the *C is part of the heterocycloalkyl.

In preferred embodiments of the first aspect, the compound for use is characterized by formula (3a) or (3b), in particular (3b), wherein
R¹ is

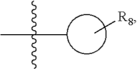

wherein

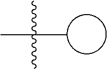

is a 5- to 12-membered, i.e. 5-, 6-, 7-, 8-, 9-, 10-, 11-, or 12-membered alicyclic system, aryl or heteroaryl, in particular a 5- or 6-membered aryl or heteroaryl, and R⁸ is selected from H, R⁹ and OR⁹, wherein R⁹ is selected from optionally substituted C₁-, C₂-, C₃-, C₄-C₅-, C₆- or C₇-alkyl and optionally substituted C₃-, C₄-C₅-, C₆- or C₇-cycloalkyl, wherein the aryl or heteroaryl and R⁹ may form a double cycle,
wherein preferably

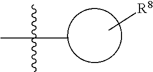

comprises at least one heteroatom;
more preferably, R¹ is selected from the group consisting of

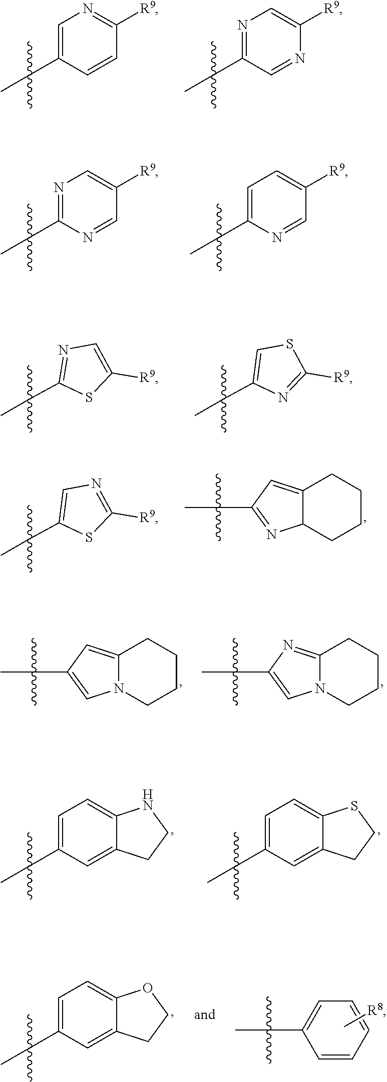

wherein R⁸ is selected from R⁹ and OR⁹, and wherein R⁹ is an unsubstituted C₁-, C₂-, C₃-, or C₄-alkyl or unsubstituted C₃-, or C₄-cycloalkyl;
even more preferably, R¹ is selected from the group consisting of

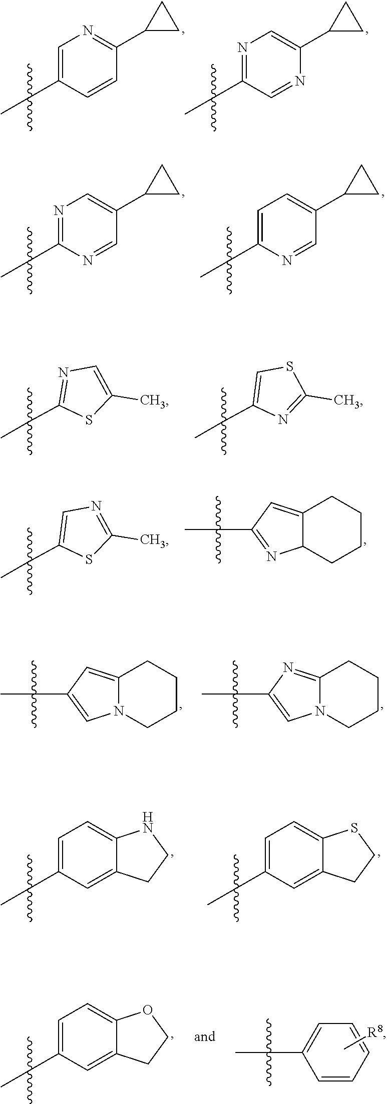

wherein R⁸ is selected from R⁹ and OR⁹, and wherein R⁹ is an unsubstituted C₁-, C₂-, C₃-, or C₄-alkyl or unsubstituted C₃-, or C₄-cycloalkyl;
R² is —OR⁵ or —R⁵, wherein R⁵ is selected from the group consisting of —H and optionally substituted C₁-C₁₀ alkyl, C₂-C₁₀-alkenyl, C₂-C₁₀-alkynyl, C₃-C₁₀-cycloalkyl, C₃-C₁₀-heterocycloalkyl, C₅-C₁₄-aryl, C₅-C₁₄-heteroaryl, C₆-C₁₅-aralkyl, C₆-C₁₅-heteroaralkyl, in particular R² is —OCH₂;
R³ is (CH₂)ₙ₂—R⁷, wherein n2 is 1-4, i.e. 1, 2, 3, or 4, and R⁷ is selected from the group consisting of optionally substituted C₁-C₇-alkyl, C₄-C₇-cycloalkyl; C₅-C₁₀-aryl or C₅-C₁₀-heteroaryl,
Y is absent or selected from —*CH₂—, —*O—, —*CH₂—CH₂—, —*CH₂—O—, and —*O—CH₂—, in particular Y is absent or selected from —*CH₂— and —*O—, wherein the *C or *O is covalently linked to the C atom of the carbonyl group; and
X is selected from the group consisting of —*CH₂—, —NH—, —O—, —S—, and —*CHF—, in particular X is —*CH₂—, wherein the *C is part of the heterocycloalkyl.

In preferred embodiments of the first aspect, the compound for use is characterized by formula (3a) or (3b), in particular (3b), wherein
R¹ is selected from

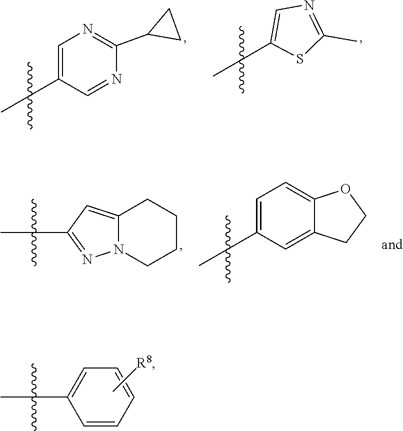

wherein R⁸ is selected from R⁹ and OR⁹, wherein R⁹ is an unsubstituted C₁-C₄-alkyl;
R² is —OR⁵ or —R⁵, wherein R⁵ is selected from the group consisting of —H and optionally substituted C₁-C₁₀ alkyl, C₂-C₁₀-alkenyl, C₂-C₁₀-alkynyl, C₃-C₁₀-cycloalkyl, C₃-C₁₀-heterocycloalkyl, C₅-C₁₄-aryl, C₅-C₁₄-heteroaryl, C₆-C₁₅-aralkyl and C₆-C₁₅-heteroaralkyl, in particular R² is —OCH₂;
R³ is (CH₂)ₙ₂—R⁷, wherein n2 is 1-4, i.e. 1, 2, 3, or 4, and R⁷ is selected from the group consisting of optionally substituted C₁-C₇-alkyl, C₄-C₇-cycloalkyl, C₅-C₁₀-aryl and C₅-C₁₀-heteroaryl,
Y is absent or selected from —*CH₂—, —*O—, —*CH₂—CH₂—, —*CH₂—O—, and —*O—CH₂—, in particular Y is absent or selected from —*CH₂— and —*O—, wherein the *C or *O is covalently linked to the C atom of the carbonyl group; and
X is selected from the group consisting of —CH₂—, —NH—, —O—, —S—, and —*CHF—, in particular X is —*CH₂—, wherein the *C is part of the heterocycloalkyl.

In preferred embodiments of the first aspect, the compound for use is characterized by formula (3a) or (3b), in particular (3b), wherein
R¹ is selected from

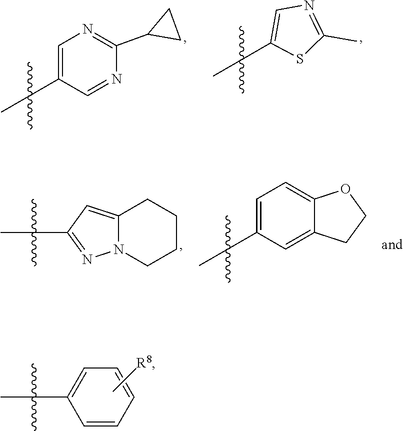

in particular wherein R⁸ is in para position, and wherein R⁸ is OR⁹, wherein R⁹ is an unsubstituted C₁-C₄-alkyl, in particular R⁹ is —CH₃;
R² is —OR⁵ or —R⁵, wherein R⁵ is selected from the group consisting of —H and optionally substituted C₁-C₁₀ alkyl, C₂-C₁₀-alkenyl, C₂-C₁₀-alkynyl, C₃-C₁₀-cycloalkyl, C₃-C₁₀-heterocycloalkyl, C₅-C₁₄-aryl, C₅-C₁₄-heteroaryl, C₆-C₁₅-aralkyl and C₆-C₁₅-heteroaralkyl, in particular R² is —OCH₂;
R³ is (CH₂)ₙ₂—R⁷, wherein n2 is 1-4, i.e. 1, 2, 3 or 4, and R⁷ is selected from the group consisting of optionally substituted C₁-C₇-alkyl, C₄-C₇-cycloalkyl; C₅-C₁₀-aryl or C₅-C₁₀-heteroaryl,
Y is absent or selected from —*CH₂—, —*O—, —*CH₂—CH₂—, —*CH₂—O—, and —*O—CH₂—, in particular Y is absent or selected from —*CH₂— and —*O—, wherein the *C or *O is covalently linked to the C atom of the carbonyl group; and
X is selected from the group consisting of —CH₂—, —NH—, —O—, —S—, and —*CHF—, in particular X is —*CH₂—, wherein the *C is part of the heterocycloalkyl.

In preferred embodiments of the first aspect, the compound for use is characterized by formula (3a) or (3b), in particular (3b), wherein
R¹ is

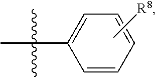

wherein R⁸ is —O—CH₃, in particular wherein R⁸ is in para position;
R² is —OR⁵ or —R⁵, wherein R⁵ is selected from the group consisting of —H and optionally substituted C₁-C₁₀ alkyl, C₂-C₁₀-alkenyl, C₂-C₁₀-alkynyl, C₃-C₁₀-cycloalkyl, C₃-C₁₀-heterocycloalkyl, C₅-C₁₄-aryl, C₅-C₁₄-heteroaryl, C₆-C₁₅-aralkyl, C₆-C₁₅-heteroaralkyl, in particular R² is —OCH₂;
R³ is (CH₂)ₙ₂—R⁷, wherein n2 is 1-4, i.e. 1, 2, 3, or 4, and R⁷ is selected from the group consiszing of optionally substituted C₁-C₇-alkyl, C₄-C₇-cycloalkyl, C₅-C₁₀-aryl and C₅-C₁₀-heteroaryl,
Y is absent or selected from —*CH₂—, —*O—, —*CH₂—CH₂—, —*CH₂—O—, and —*O—CH₂—, in particular Y is absent or selected from —*CH₂— and —*O—, wherein the *C or *O is covalently linked to the C atom of the carbonyl group; and
X is selected from the group consisting of —CH₂—, —NH—, —O—, —S—, and —*CHF—, in particular X is —*CH₂—, wherein the *C is part of the heterocycloalkyl.

In preferred embodiments of the first aspect, the compound for use is characterized by formula (3a) or (3b), in particular (3b), wherein
R¹ is

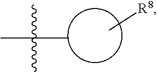

wherein

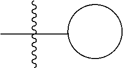

is a 5- to 12-membered, i.e. 5-, 6-, 7-, 8-, 9-, 10-, 11-, or 12-membered alicyclic system, aryl or heteroaryl, in particular a 5- or 6-membered aryl or heteroaryl, and R⁸ is selected from H, R⁹ and OR⁹, wherein R⁹ is selected from optionally substituted C₁-, C₂-, C₃-, C₄-C₅-, C₆- or C₇-alkyl and optionally substituted C₃-, C₄-C₅-, C₆- or C₇-cycloalkyl, wherein the aryl or heteroaryl and R⁹ may form a double cycle,
wherein preferably

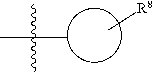

comprises at least one heteroatom;
more preferably, R¹ is selected from the group consisting of

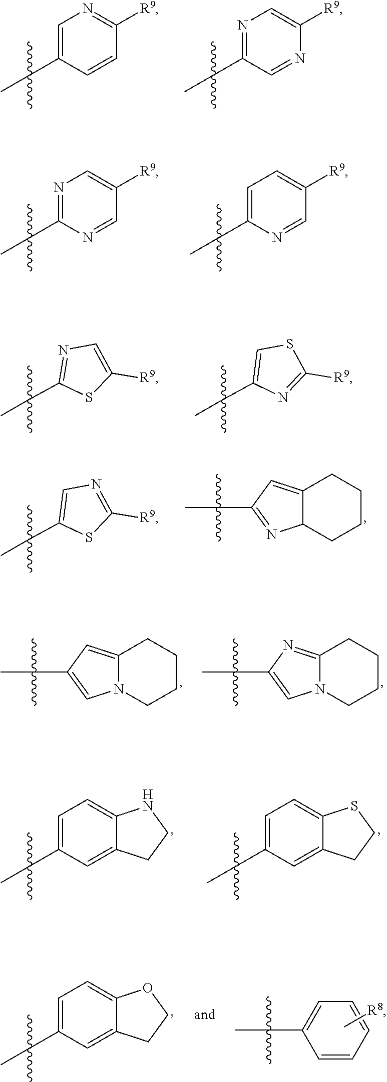

wherein R⁸ is selected from R⁹ and OR⁹, and wherein R⁹ is an unsubstituted C₁-, C₂-, C₃-, or C₄-alkyl or unsubstituted C₃-, or C₄-cycloalkyl;
even more preferably, R¹ is selected from the group consisting of

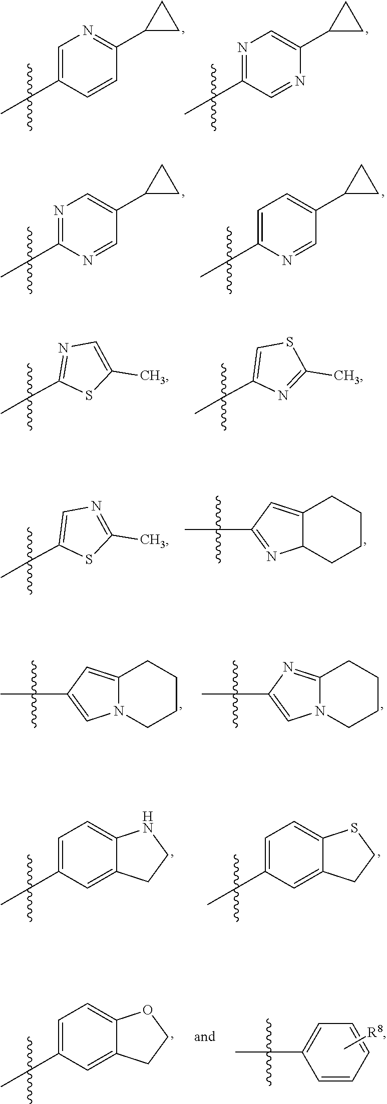

wherein R⁸ is selected from R⁹ and OR⁹, and wherein R⁹ is an unsubstituted C₁-, C₂-, C₃-, or C₄-alkyl or unsubstituted C₃-, or C₄-cycloalkyl;
R² is —OR⁵ or —R⁵, wherein R⁵ is selected from the group consisting of —H and optionally substituted C₁-C₁₀ alkyl, C₂-C₁₀-alkenyl, C₂-C₁₀-alkynyl, C₃-C₁₀-cycloalkyl, C₃-C₁₀-heterocycloalkyl, C₅-C₁₄-aryl, C₅-C₁₄-heteroaryl, C₆-C₁₅-aralkyl, C₆-C₁₅-heteroaralkyl;
R³ is (CH₂)ₙ₂—R⁷, wherein n2 is 1-4, i.e. 1, 2, 3, or 4, and R⁷ is selected from the group consisting of optionally substituted C₁-C₇-alkyl, C₄-C₇-cycloalkyl, C₅-C₁₀-aryl and C₅-C₁₀-heteroaryl,
Y is absent or selected from —*CH₂—, —*O—, —*CH₂—CH₂—, —*CH₂—O—, and —*O—CH₂—, in particular Y is absent or selected from —*CH₂— and —*O—, wherein the *C or *O is covalently linked to the C atom of the carbonyl group; and
X is selected from the group consisting of —CH₂—, —NH—, —O—, —S—, and —*CHF—, in particular X is —*CH₂—, wherein the *C is part of the heterocycloalkyl.

In preferred embodiments of the first aspect, the compound for use is characterized by formula (3a) or (3b), in particular (3b), wherein
R¹ is selected from

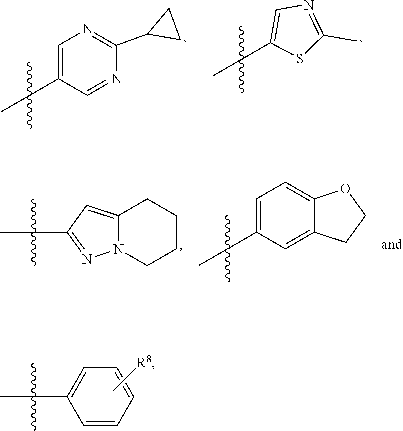

in particular wherein R⁸ is in para position, and wherein R⁸ is OR⁹, wherein R⁹ is C₁-, C₂-, C₃-, or C₄-alkyl, in particular R⁹ is —CH₃;
R² is —OR⁵, wherein R⁵ is selected from the group consisting of —H and optionally substituted C₁-C₆ alkyl, C₂-C₆-alkenyl, C₂-C₆-alkynyl and C₃-C₈-cycloalkyl, in particular from C₁-C₄-alkyl or C₃-C₆-cycloalkyl;
R³ is (CH₂)ₙ₂—R⁷, wherein n2 is 1-4, i.e. 1, 2, 3 or 4, and R⁷ is selected from the group consisting of optionally substituted C₁-C₇-alkyl, C₄-C₇-cycloalkyl, C₅-C₁₀-aryl and C₅-C₁₀-heteroaryl,
Y is absent or selected from —*CH₂—, —*O—, —*CH₂—CH₂—, —*CH₂—O—, and —*O—CH₂—, in particular Y is absent or selected from —*CH₂— and —*O—, wherein the *C or *O is covalently linked to the C atom of the carbonyl group; and
X is selected from the group consisting of —CH₂—, —NH—, —O—, —S—, and —*CHF—, in particular X is —*CH₂—, wherein the *C is part of the heterocycloalkyl.

In preferred embodiments of the first aspect, the compound for use is characterized by formula (3a) or (3b), in particular (3b), wherein
R¹ is selected from

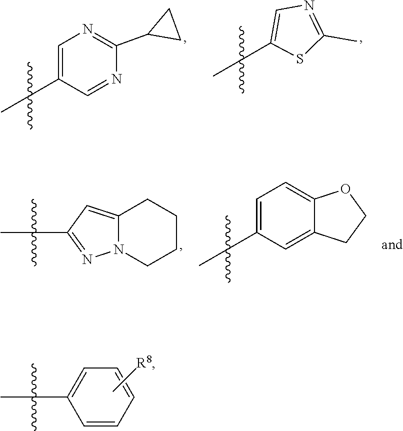

in particular wherein R⁸ is in para position, and wherein R⁸ is OR⁹, wherein R⁹ is C₁-, C₂-, C₃-, or C₄-alkyl, in particular R⁹ is —CH₃;
R² is —OCH₃—;
R³ is (CH₂)ₙ₂—R⁷, wherein n2 is 1-4, i.e. 1, 2, 3, or 4, and R⁷ is selected from the group consisting of substituted C₁-C₇-alkyl, C₄-C₇-cycloalkyl, C₅-C₁₀-aryl and C₅-C₁₀-heteroaryl,
Y is absent or selected from —*CH₂—, —*O—, —*CH₂—CH₂—, —*CH₂—O—, and —*O—CH₂—, in particular Y is absent or selected from —*CH₂— and —*O—, wherein the *C or *O is covalently linked to the C atom of the carbonyl group; and
X is selected from the group consisting of —CH₂—, —NH—, —O—, —S—, and —*CHF—, in particular X is —*CH₂—. wherein the *C is part of the heterocycloalkyl.

In preferred embodiments of the first aspect, the compound for use is characterized by formula (3a) or (3b), in particular (3b), wherein
R¹ is selected from

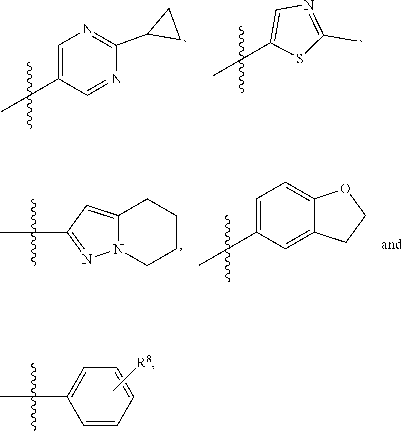

particular wherein R⁸ is in para position, and wherein R⁸ is OR⁹, wherein R⁹ is C₁-, C₂-, C₃-, or C₄-alkyl, in particular R⁹ is —CH₃;
R² is —OR⁵ or —R⁵, wherein R⁵ is selected from the group consisting of —H and optionally substituted C₁-C₁₀ alkyl, C₂-C₁₀-alkenyl, C₂-C₁₀-alkynyl, C₃-C₁₀-cycloalkyl, C₃-C₁₀-heterocycloalkyl, C₅-C₁₄-aryl, C₅-C₁₄-heteroaryl, C₆-C₁₅-aralkyl and C₆-C₁₅-heteroaralkyl, in particular R² is —OCH₂;
R³ is (CH₂)ₙ₂—R⁷, wherein n2 is 0-4, i.e. 0, 1, 2, 3, or 4, and R⁷ is selected from

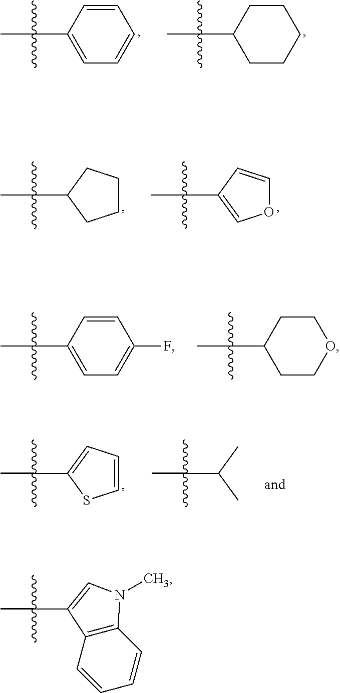

in particular R⁷ is

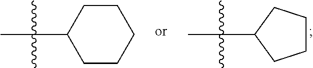 [SMILES: *C1CCCCC1 ; *C1CCCC1]

Y is absent or selected from —*CH₂—, —*O—, —*CH₂—CH₂—, —*CH₂—O—, and —*O—CH₂—, in particular Y is absent or selected from —*CH₂— and —*O—, wherein the *C or *O is covalently linked to the C atom of the carbonyl group; and
X is selected from the group consisting of —CH₂—, —NH—, —O—, —S—, and —*CHF—, in particular X is —*CH₂—, wherein the *C is part of the heterocycloalkyl.

In preferred embodiments of the first aspect, the compound for use is characterized by formula (4a) or (4b), in particular (4b), wherein
R¹ is

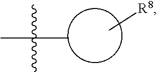

wherein

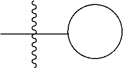

is a 5- to 12-membered, i.e. 5-, 6-, 7-, 8-, 9-, 10-, 11-, or 12-membered alicyclic system, aryl or heteroaryl, in particular a 5- or 6-membered aryl or heteroaryl, and R⁸ is selected from H, R⁹ and OR⁹, wherein R⁹ is selected from optionally substituted C₁-, C₂-, C₃-, C₄-C₅-, C₆- or C₇-alkyl and optionally substituted C₃-, C₄-C₅-, C₆- or C₇-cycloalkyl, wherein the aryl or heteroaryl and R⁹ may form a double cycle,
wherein preferably

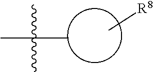

comprises at least one heteroatom;
more preferably, R is selected from the group consisting of

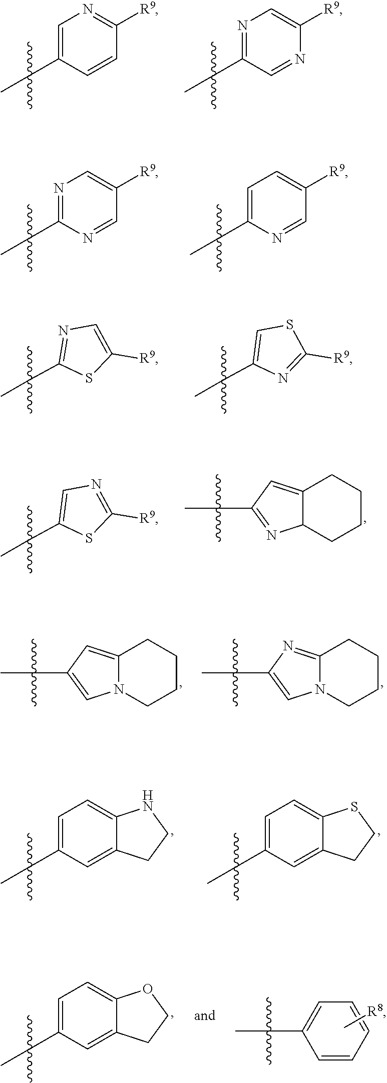

wherein R⁸ is selected from R⁹ and OR⁹, and wherein R⁹ is an unsubstituted C₁-, C₂-, C₃-, or C₄-alkyl or unsubstituted C₃-, or C₄-cycloalkyl;
even more preferably, R′ is selected from the group consisting of

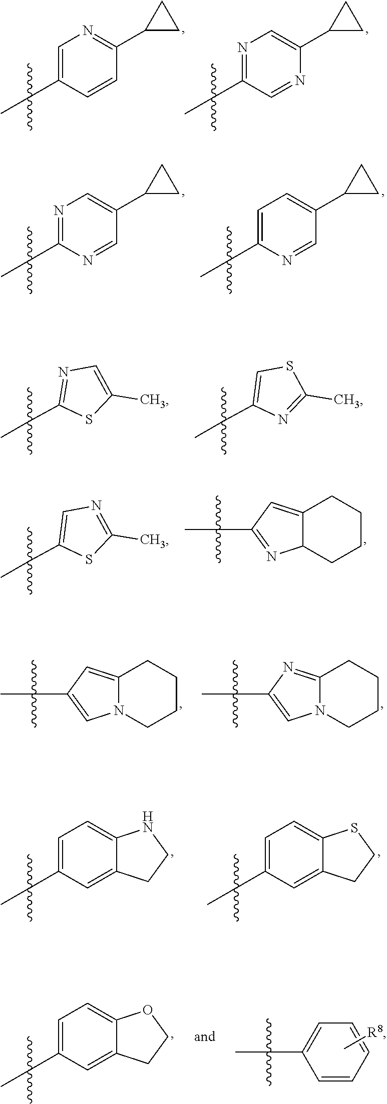

wherein R⁸ is selected from R⁹ and OR⁹, and wherein R⁹ is an unsubstituted C₁-, C₂-, C₃-, or C₄-alkyl or unsubstituted C₃-, or C₄-cycloalkyl;
R² is —OR⁵, wherein R⁵ is selected from the group consisting of —H and optionally substituted C₁-C₁₀ alkyl, C₂-C₁₀-alkenyl, C₂-C₁₀-alkynyl, C₃-C₁₀-cycloalkyl, C₃-C₁₀-heterocycloalkyl, C₅-C₁₄-aryl, C₅-C₁₄-heteroaryl, C₆-C₁₅-aralkyl, C₆-C₁₅-heteroaralkyl, in particular R² is —OCH₂;
R³ is (CH₂)ₙ₂—R⁷, wherein n2 is 1-4, i.e. 1, 2, 3, or 4, and R⁷ is selected from the group consisting of optionally substituted C₁-C₇-alkyl, C₄-C₇-cycloalkyl; C₅-C₁₀-aryl or C₅-C₁₀-heteroaryl,
Y is absent or selected from —*CH₂—, —*O—, —*CH₂—CH₂—, —*CH₂—O—, and —*O—CH₂—, in particular Y is absent or selected from —*CH₂— and —*O—, wherein the *C or *O is covalently linked to the C atom of the carbonyl group; and
X is selected from the group consisting of —*CH₂—, —NH—, —O—, —S—, and —*CHF—, in particular X is —*CH₂—, wherein the *C is part of the heterocycloalkyl.

In preferred embodiments of the first aspect, the compound for use is characterized by formula (4a) or (4b), in particular (4b), wherein
R¹ is selected from

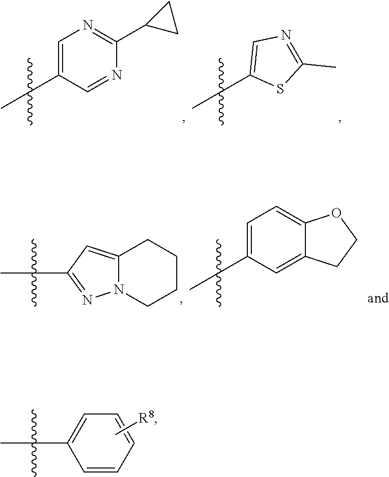

wherein R⁸ is selected from R⁹ and OR⁹, wherein R⁹ is an unsubstituted C₁-C₄-alkyl;
R² is —OR⁵, wherein R⁵ is selected from the group consisting of —H and optionally substituted C₁-C₁₀ alkyl, C₂-C₁₀-alkenyl, C₂-C₁₀-alkynyl, C₃-C₁₀-cycloalkyl, C₃-C₁₀-heterocycloalkyl, C₅-C₁₄-aryl, C₅-C₁₄-heteroaryl, C₆-C₁₅-aralkyl and C₆-C₁₅-heteroaralkyl, in particular R² is —OCH₂;
R³ is (CH₂)ₙ₂—R⁷, wherein n2 is 1-4, i.e. 1, 2, 3, or 4, and R⁷ is selected from the group consisting of substituted C₁-C₇-alkyl, C₄-C₇-cycloalkyl; C₅-C₁₀-aryl or C₅-C₁₀-heteroaryl,
Y is absent or selected from —*CH₂—, —*O—, —*CH₂—CH₂—, —*CH₂—O—, and —*O—CH₂—, in particular Y is absent or selected from —*CH₂— and —*O—, wherein the *C or *O is covalently linked to the C atom of the carbonyl group; and
X is selected from the group consisting of —CH₂—, —NH—, —O—, —S—, and —*CHF—, in particular X is —*CH₂—, wherein the *C is part of the heterocycloalkyl.

In preferred embodiments of the first aspect, the compound for use is characterized by formula (4a) or (4b), in particular (4b), wherein
R¹ is selected from

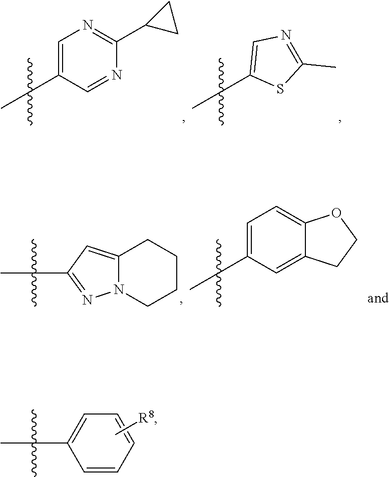

in particular wherein R⁸ is in para position, and wherein R⁸ is OR⁹, wherein R⁹ is an unsubstituted C₁-C₄-alkyl, in particular R⁹ is —CH₃;
R² is —OR⁵, wherein R⁵ is selected from the group consisting of —H and optionally substituted C₁-C₁₀ alkyl, C₂-C₁₀-alkenyl, C₂-C₁₀-alkynyl, C₃-C₁₀-cycloalkyl, C₃-C₁₀-heterocycloalkyl, C₅-C₁₄-aryl, C₅-C₁₄-heteroaryl, C₆-C₁₅-aralkyl and C₆-C₁₅-heteroaralkyl, in particular R² is —OCH₂;
R³ is (CH₂)ₙ₂—R⁷, wherein n2 is 1-4, i.e., 1, 2, 3, or 4, and R⁷ is selected from the group consisting of substituted C₁-C₇-alkyl, C₄-C₇-cycloalkyl; C₅-C₁₀-aryl or C₅-C₁₀-heteroaryl,
Y is absent or selected from —*CH₂—, —*O—, —*CH₂—CH₂—, —*CH₂—O—, and —*O—CH₂—, in particular Y is absent or selected from —*CH₂— and —*O—, wherein the *C or *O is covalently linked to the C atom of the carbonyl group; and
X is selected from the group consisting of —CH₂—, —NH—, —O—, —S—, and —*CHF—, in particular X is —*CH₂—, wherein the *C is part of the heterocycloalkyl.

In preferred embodiments of the first aspect, the compound for use is characterized by formula (4a) or (4b), in particular (4b), wherein
R¹ is

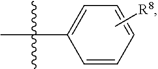

wherein R⁸ is —O—CH₃, in particular wherein R⁸ is in para position;
R² is —OR⁵, wherein R⁵ is selected from the group consisting of —H and optionally substituted C₁-C₁₀ alkyl, C₂-C₁₀-alkenyl, C₂-C₁₀-alkynyl, C₃-C₁₀-cycloalkyl, C₃-C₁₀-heterocycloalkyl, C₅-C₁₄-aryl, C₅-C₁₄-heteroaryl, C₆-C₁₅-aralkyl and C₆-C₁₅-heteroaralkyl, in particular R² is —OCH₂;
R³ is (CH₂)ₙ₂—R⁷, wherein n2 is 1-4, i.e. 1, 2, 3, or 4, and R⁷ is selected from the group consisting of optionally substituted C₁-C₇-alkyl, C₄-C₇-cycloalkyl; C₅-C₁₀-aryl or C₅-C₁₀-heteroaryl,
Y is absent or selected from —*CH₂—, —*O—, —*CH₂—CH₂—, —*CH₂—O—, and —*O—CH₂—, in particular Y is absent or selected from —*CH₂— and —*O—, wherein the *C or *O is covalently linked to the C atom of the carbonyl group; and
X is selected from the group consisting of —CH₂—, —NH—, —O—, —S—, and —*CHF—, in particular X is —*CH₂—, wherein the *C is part of the heterocycloalkyl.

In preferred embodiments of the first aspect, the compound for use is characterized by formula (4a) or (4b), in particular (4b), wherein
R¹ is

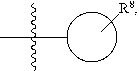

wherein

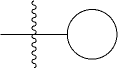

is a 5- to 12-membered, i.e. 5-, 6-, 7-, 8-, 9-, 10-, 11-, or 12-membered alicyclic system, aryl or heteroaryl, in particular a 5- or 6-membered aryl or heteroaryl, and R⁸ is selected from H, R⁹ and OR⁹, wherein R⁹ is selected from optionally substituted C₁-, C₂-, C₃-, C₄-C₅-, C₆- or C₇-alkyl and optionally substituted C₃-, C₄-C₅-, C₆- or C₇-cycloalkyl, wherein the aryl or heteroaryl and R⁹ may form a double cycle,
wherein preferably

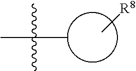

comprises at least one heteroatom;
more preferably, R¹ is selected from the group consisting of

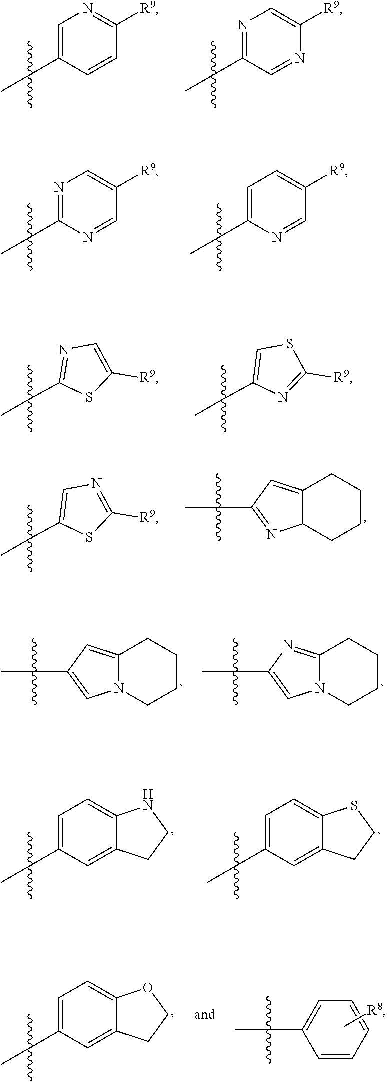

wherein R⁸ is selected from R⁹ and OR⁹, and wherein R⁹ is an unsubstituted C₁-, C₂-, C₃-, or C₄-alkyl or unsubstituted C₃-, or C₄-cycloalkyl;
even more preferably, R¹ is selected from the group consisting of

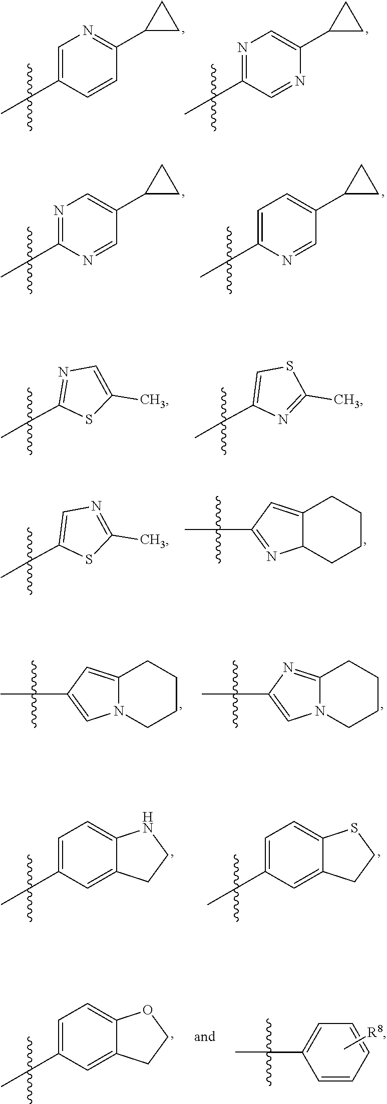

wherein R⁸ is selected from R⁹ and OR⁹, and wherein R⁹ is an unsubstituted C₁-, C₂-, C₃-, or C₄-alkyl or unsubstituted C₃-, or C₄-cycloalkyl;
R² is —OR⁵, wherein R⁵ is selected from the group consisting of —H and optionally substituted C₁-C₆ alkyl, C₂-C₆-alkenyl, C₂-C₆-alkynyl and C₃-C₈-cycloalkyl;
R³ is (CH₂)ₙ₂—R⁷, wherein n2 is 1-4, i.e. 1, 2, 3, or 4, and R⁷ is selected from the group consisting of optionally substituted C₁-C₇-alkyl, C₄-C₇-cycloalkyl; C₅-C₁₀-aryl or C₅-C₁₀-heteroaryl,
Y is absent or selected from —*CH₂—, —*O—, —*CH₂—CH₂—, —*CH₂—O—, and —*O—CH₂—, in particular Y is absent or selected from —*CH₂— and —*O—, wherein the *C or *O is covalently linked to the C atom of the carbonyl group; and
X is selected from the group consisting of —CH₂—, —NH—, —O—, —S—, and —*CHF—, in particular X is —*CH₂—, wherein the *C is part of the heterocycloalkyl.

In preferred embodiments of the first aspect, the compound for use is characterized by formula (4a) or (4b), in particular (4b), wherein
R¹ is selected from

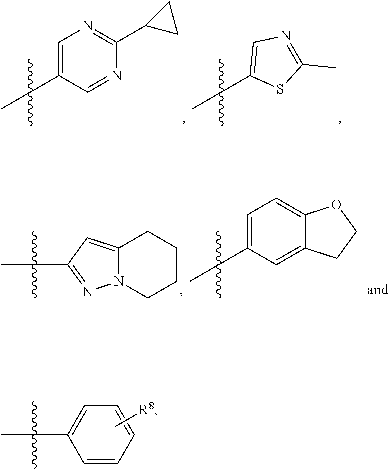

in particular wherein R⁸ is in para position, and wherein R⁸ is OR⁹, wherein R⁹ is C₁-, C₂-, C₃-, or C₄-alkyl, in particular R⁹ is —CH₃;
R² is —OR⁵, wherein R⁵ is selected from optionally substituted C₁-C₄-alkyl and C₃-C₆-cycloalkyl, in particular R⁵ is —CH₃;
R³ is (CH₂)ₙ₂—R⁷, wherein n2 is 1-4, i.e. 1, 2, 3, or 4, and R⁷ is selected from the group consisting of optionally substituted C₁-C₇-alkyl, C₄-C₇-cycloalkyl, C₅-C₁₀-aryl and C₅-C₁₀-heteroaryl,
Y is absent or selected from —*CH₂—, —*O—, —*CH₂—CH₂—, —*CH₂—O—, and —*O—CH₂—, in particular Y is absent or selected from —*CH₂— and —*O—, wherein the *C or *O is covalently linked to the C atom of the carbonyl group; and
X is selected from the group consisting of —CH₂—, —NH—, —O—, —S—, and —*CHF—, in particular X is —*CH₂—, wherein the *C is part of the heterocycloalkyl.

In preferred embodiments of the first aspect, the compound for use is characterized by formula (4a) or (4b), in particular (4b), wherein
R¹ is selected from

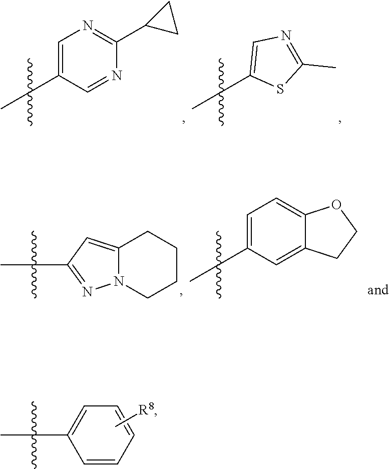

in particular wherein R⁸ is in para position, and wherein R⁸ is OR⁹, wherein R⁹ is C₁-, C₂-, C₃-, or C₄-alkyl, in particular R⁹ is —CH₃;
R² is —OCH₂;
R³ is (CH₂)ₙ₂—R⁷, wherein n2 is 1-4, i.e. 1, 2, 3, or 4, and R⁷ is selected from the group consisting of optionally substituted C₁-C₇-alkyl, C₄-C₇-cycloalkyl, C₅-C₁₀-aryl and C₅-C₁₀-heteroaryl,
Y is absent or selected from —*CH₂—, —*O—, —*CH₂—CH₂—, —*CH₂—O—, and —*O—CH₂—, in particular Y is absent or selected from —*CH₂— and —*O—, wherein the *C or *O is covalently linked to the C atom of the carbonyl group; and
X is selected from the group consisting of —CH₂—, —NH—, —O—, —S—, and —*CHF—, in particular X is —*CH₂—, wherein the *C is part of the heterocycloalkyl.

In preferred embodiments of the first aspect, the compound for use is characterized by formula (4a) or (4b), in particular (4b), wherein
R¹ is selected from

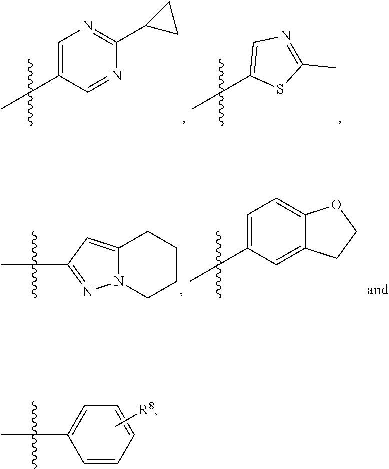

in particular wherein R⁸ is in para position, and wherein R⁸ is OR⁹, wherein R⁹ is C₁-, C₂-, C₃-, or C₄-alkyl, in particular R⁹ is —CH₃;
R² is —OR⁵, wherein R⁵ is selected from the group consisting of —H and optionally substituted C₁-C₁₀ alkyl, C₂-C₁₀-alkenyl, C₂-C₁₀-alkynyl, C₃-C₁₀-cycloalkyl, C₃-C₁₀-heterocycloalkyl, C₅-C₁₄-aryl, C₅-C₁₄-heteroaryl, C₆-C₁₅-aralkyl and C₆-C₁₅-heteroaralkyl, in particular R² is —OCH₂;
R³ is (CH₂)ₙ₂—R⁷, wherein n2 is 1-4 i.e. 1, 2, 3, or 4, and R⁷ is selected from

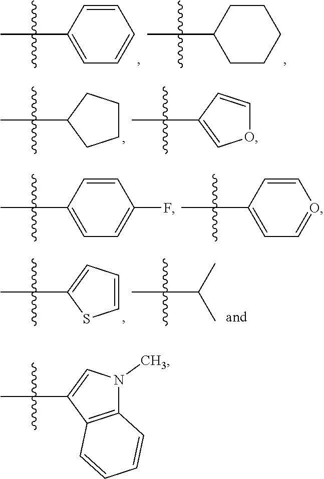

in particular R⁷ is

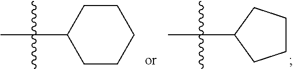 [SMILES: *C1CCCCC1 ; *C1CCCC1]

Y is absent or selected from —*CH₂—, —*O—, —*CH₂—CH₂—, —*CH₂—O—, and —*O—CH₂—, in particular Y is absent or selected from —*CH₂— and —*O—, wherein the *C or *O is covalently linked to the C atom of the carbonyl group; and
X is selected from the group consisting of —CH₂—, —NH—, —O—, —S—, and —*CHF—, in particular X is —*CH₂—, wherein the *C is part of the heterocycloalkyl.

In particularly preferred embodiments of the first aspect, the compound is characterized by formula (4a) or (4b), in particular (4b), wherein R¹ is

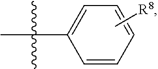

in particular wherein R⁸ is in para position, and wherein R⁸ is OR⁹, in particular wherein R⁹ is —CH₃, and R² is —OR⁵, wherein R⁵ is selected from the group consisting of —H and optionally substituted C₁-C₁₀ alkyl, in particular R² is —OCH₂, Y is absent and R³ is (CH₂)ₙ₂—R⁷, wherein n2 is 1-4, i.e. 1, 2, 3, or 4, in particular n2 is 2 or 3 and R⁷ is selected from a optionally substituted C₁-C₇-alkyl, C₄-C₇-cycloalkyl; C₅-C₁₀-aryl or C₅-C₁₀-heteroaryl, in particular R⁷ is an optionally substituted C₄-C₇-cycloalkyl.

In even more preferred embodiments of the first aspect, the compound is characterized by formula (4a) or (4b), in particular (4b), wherein R¹ is

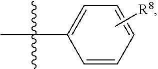

in particular wherein R⁸ is in para position, and wherein R⁸ is —OCH₃, R² is —OCH₂, Y is absent and R³ is (CH₂)ₙ₂—R⁷, wherein n2 is is 2 or 3 and R⁷ is an optionally substituted C₄-C₇-cycloalkyl.

In preferred embodiments of the first aspect, the compound for use is characterized by any of formulae (5)-(34) or (52):

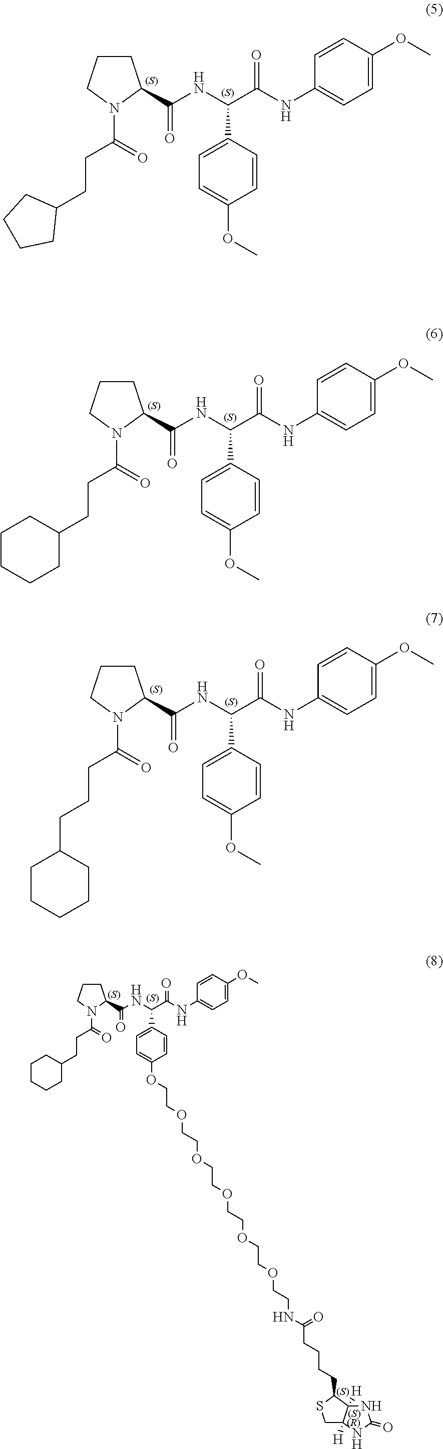

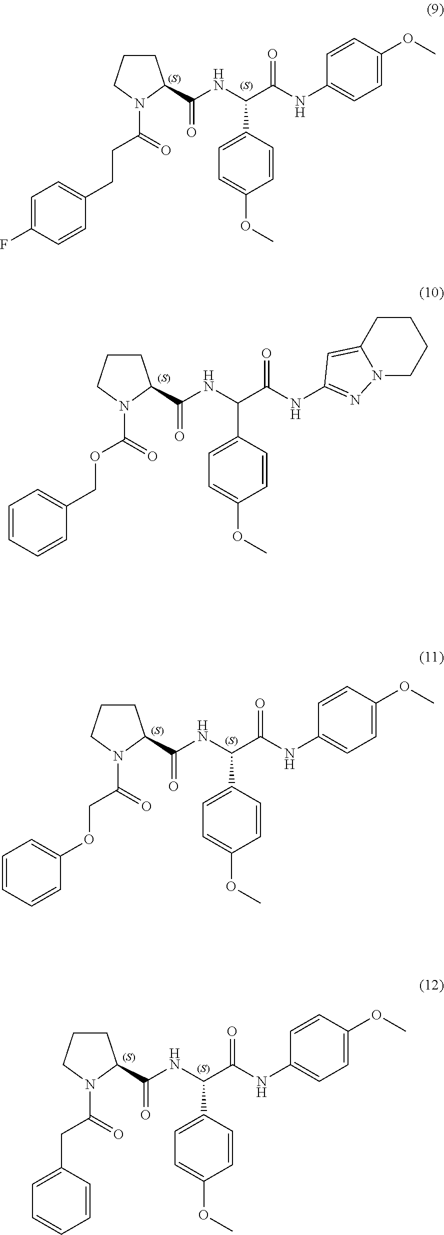

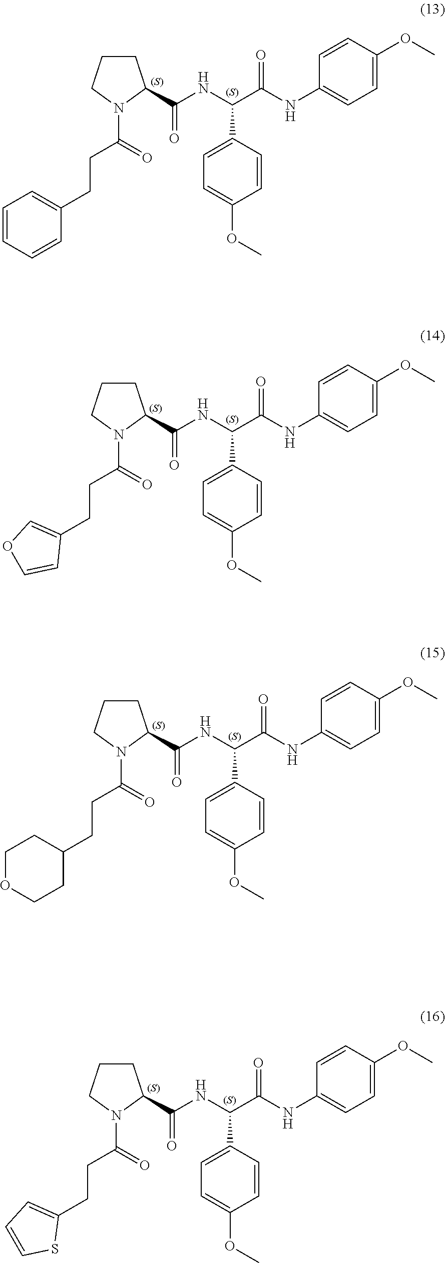

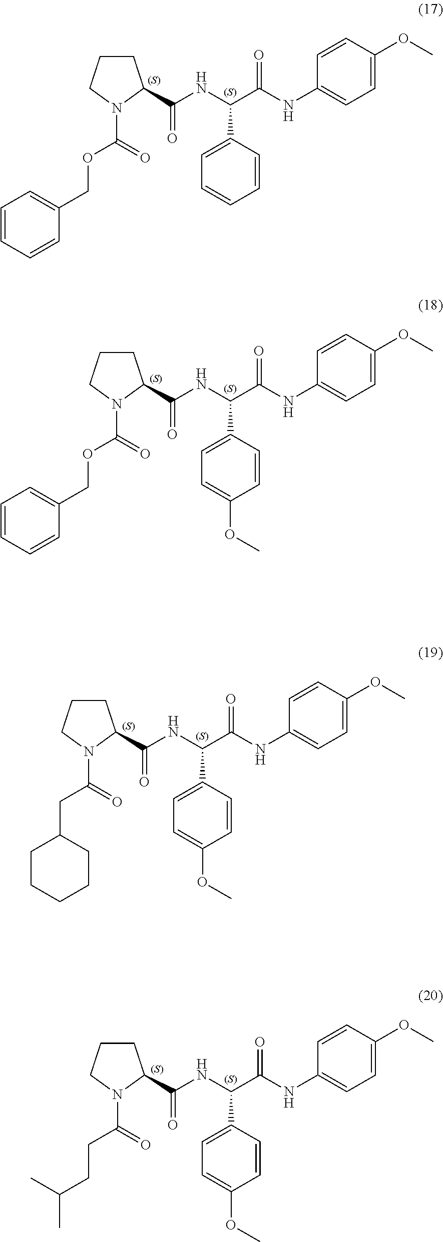

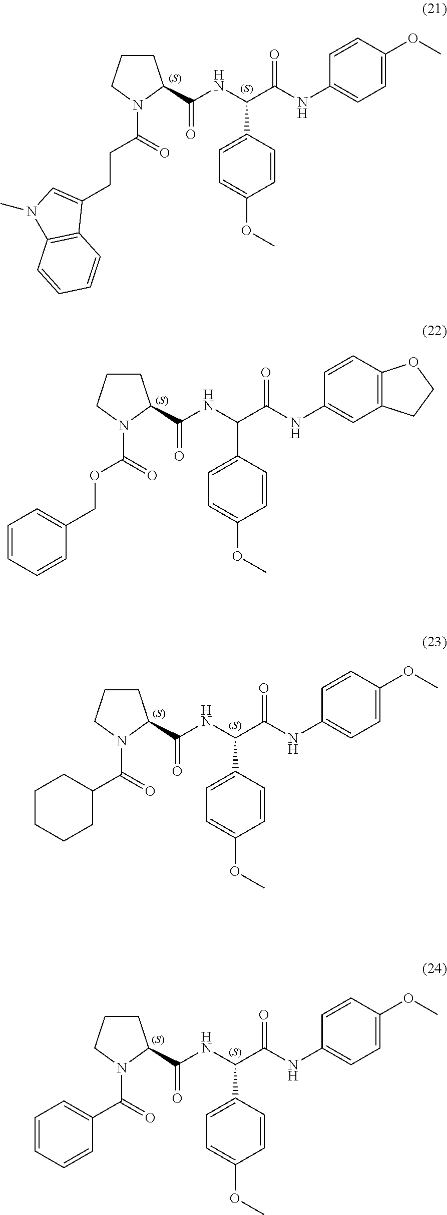

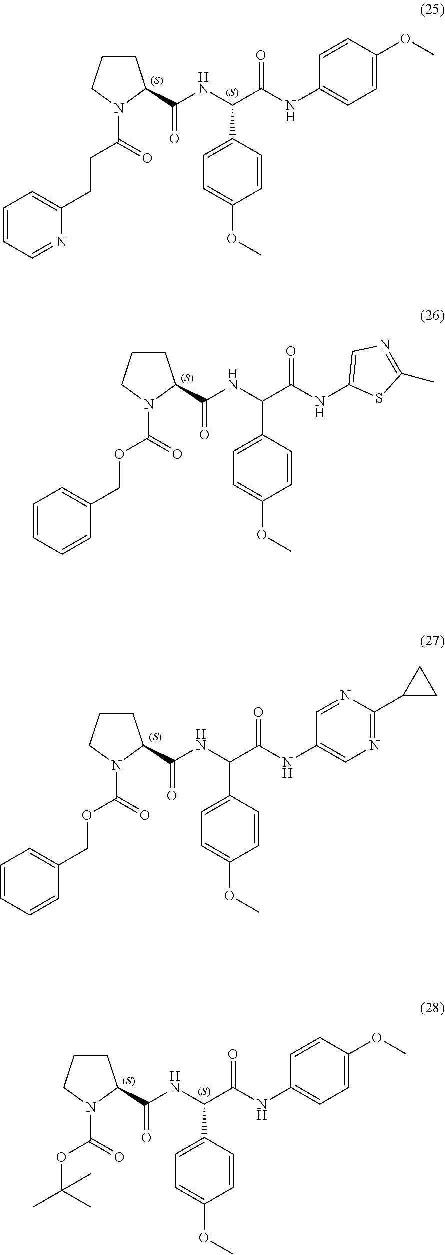

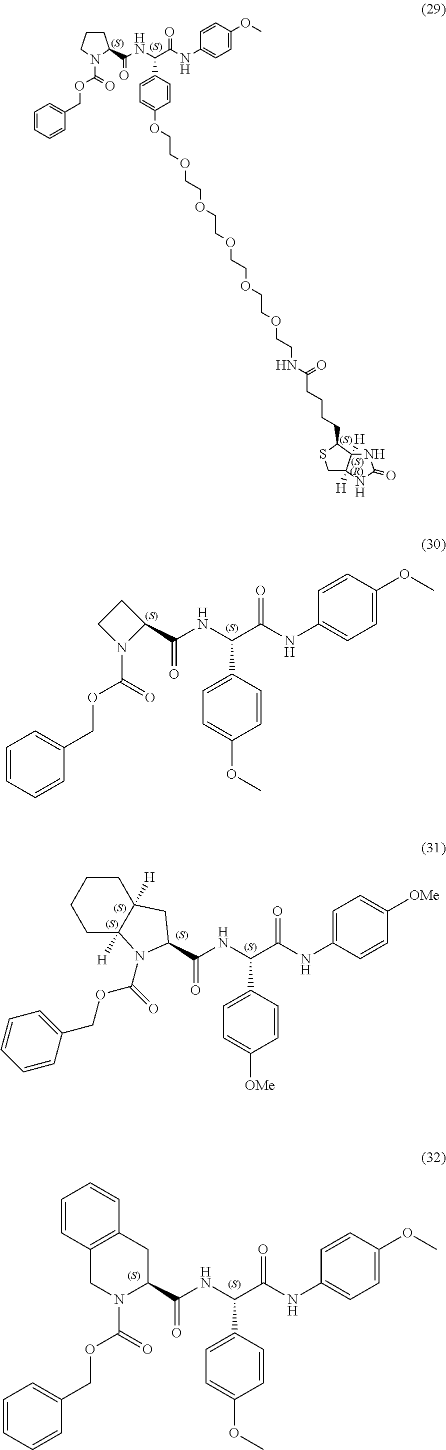

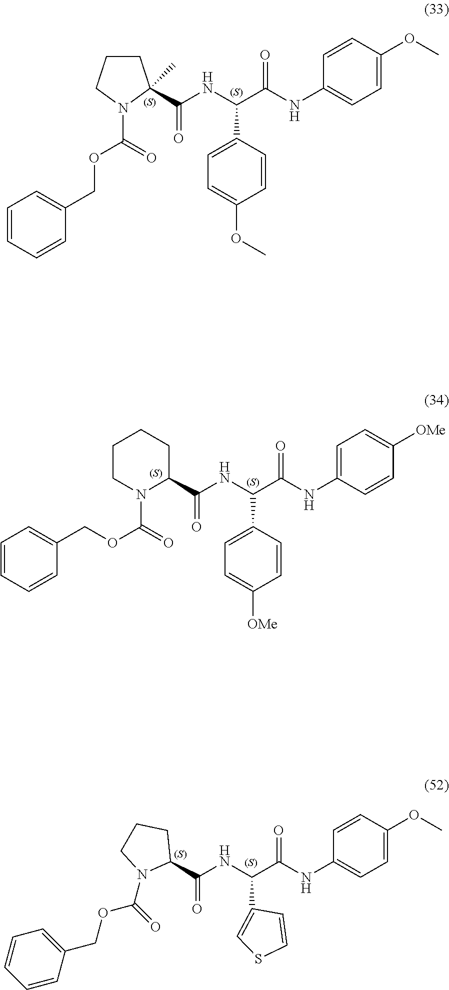

In preferred embodiments of the first aspect, the compound for use is characterized by any of formulae (5)-(34), (52) or (53), preferably (5)-(34) or (53).

In preferred embodiments of the first aspect, the compound for use is characterized by any of formulae (5)-(29).

In preferred embodiments of the first aspect, the compound for use is characterized by any of formulae (5)-(7), (9)-(28), (30)-(34) or (52), preferably (5)-(7) or (9)-(28).

In preferred embodiments of the first aspect, the compound for use is characterized by any of formulae (5)-(9), (11)-(15), (17)-(25), (28)-(34) or (52), preferably (5)-(7), (9), (11)-(15), (17)-(25), (28), (30)-(34) or (52), more preferably (5)-(7), (9), (11)-(15), (17)-(25) or (28).

In preferred embodiments of the first aspect, the compound for use is characterized by any of formulae (5)-(12), preferably (5)-(7) or (9)-(12).

In some embodiments of the first aspect, the compound for use is compound C1, which is characterized by formula (51)

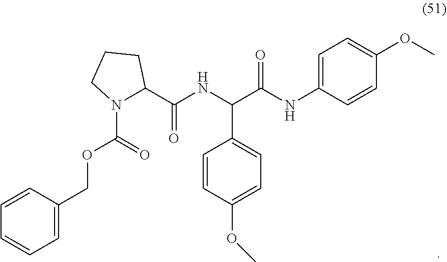 [SMILES: COc1ccc(NC(=O)C(NC(=O)C2CCCN2C(=O)OCc2ccccc2)c2ccc(OC)cc2)cc1]

More preferably, the compound for use is compound C1a, which is a stereoisomer of compound C1, characterized by formula (18).

In preferred embodiments of the first aspect, the disease is selected from the group consisting of
ischemic stroke,
heart failure with preserved ejection fraction (HFpEF), diabetic nephropathy, arthritis, asthma,
migraine, multiple sclerosis, meningitis,
optic nerve degeneration, posterior retinal degeneration, glaucoma, age-related macular degeneration, cataracts, uveitis,
retinal vascular disorder, retinal ischemic damage and reperfusion injury, thrombocytic occlusion of the retinal vein, and
retinopathy, in particular hypertensive retinopathy, ischaemic retinopathy, diabetic retinopathy, and proliferative ischaemic/diabetic retinopathy.

In preferred embodiments of the first aspect, the disease is selected from a disease characterized by inflammation in the brain. In preferred embodiments of the first aspect, the disease is selected from a disease characterized by inflammation in the eye, in particular the retina.

The inventors have shown that unexpectedly, the compounds according to the first aspect of the invention are capable of inhibiting the production of NO in microglia and macrophages. Microglia and macrophages produce NO via a specific NO synthase (NOS) called immunological NOS (iNOS or NOS2). iNOS is induced in pathological conditions by endotoxins, inflammation, and certain cytokines. NO is a free radical, which in high concentrations is toxic for all cells. The compounds according to the invention are suitable in the treatment of conditions that are characterized by a pathological NO production, i.e. an excessive or disproportionate NO production, by microglia and macrophages.

Microglia, as part of the CNS immune system, survey the CNS to maintain brain homeostasis and react to pathologic events with a complex activation process including the release of pro-inflammatory cytokines, reactive oxygen species and NO. Excessive NO production is toxic for neurons. By inhibiting the production of NO by microglia, the compounds according to the invention are capable of protecting neurons from damage. This is particularly important in conditions that are characterized by inflammation in the brain, such as stroke, multiple sclerosis and meningitis.

The inventors have shown that the compounds of the invention are capable of reducing secondary neuronal damage in stroke. As demonstrated in the examples, neuronal damage following stroke can be significantly reduced by administration of the inventive compounds, thereby ameliorating neurological deficits.

The effect of the compounds of the invention is specific to the inhibition of NO production. The compounds of the invention do not interfere with other microglial functions. In particular, the compounds of the invention do not inhibit the cytokine production or the phagocytic properties of microglia. Furthermore, the compounds of the invention do not interfere with other cell types within the CNS, such as neurons, astrocytes or oligodendrocytes.

Controlling excessive or disproportionate NO production by iNOS is also important in the treatment and prevention of several pathological conditions of the eye. NO is among the most important regulators of ocular perfusion (Schmetterer and Polak, Prog Retin Eye Res, 2001. 20(6)). If iNOS is induced, e.g. following inflammation, the enzyme produces large amounts of NO which in turn induces pathophysiological actions, such as optic nerve degeneration and posterior retinal degeneration lesion, which lead to glaucoma, age-related macular degeneration, cataracts uveitis and/or retinopathy (Pigott et al., Br J Pharmacol, 2013. 168(5)). Proliferative diabetic retinopathy (PDR) is a complication of diabetic retinopathy that can cause blindness. Concentrations of NO are elevated in the vitreous of patients with proliferative diabetic retinopathy. The NOS byproduct 1-hydroxy-arginine was increased in aqueous humor of diabetic patients with and without diabetic retinopathy, indicating that NOS activity is already increased in early stages of insulin resistance (Opatrilova et al., Acta Ophthalmol, 2018. 96(3)). Thus, selectively inhibiting the production of NO by iNOS in the eye is suitable for the treatment or prevention of pathological conditions of the eye caused or exacerbated by excessive or disproportionate production of NO by iNOS, such as optic nerve degeneration, posterior retinal degeneration, glaucoma, age-related macular degeneration, cataracts, uveitis, retinal vascular disorder, retinal ischemic damage and reperfusion injury, thrombocytic occlusion of the retinal vein, retinopathy, hypertensive retinopathy, ischaemic retinopathy, diabetic retinopathy, and proliferative ischaemic/diabetic retinopathy.

The inventors have shown that the compounds according to the first aspect of the invention are capable of crossing the blood-brain-barrier. This is beneficial if the compound is used in the treatment of conditions characterized by pathological NO production within the brain. The compounds of the invention are capable of achieving concentrations within the brain that are sufficient to exert a therapeutic effect.

The compounds of the invention are also capable of crossing the blood-retina-barrier. This is beneficial if the compound is used in the treatment of conditions characterized by pathological NO production within the retina.

The skilled person is aware of the characteristics that a compound must fulfil in order to be able to cross the blood-brain-barrier or the blood-retina-barrier. The capability of crossing the blood-brain/retina-barrier is increased for compounds having a low molecular weight and a small polar surface area (Fischer et al., J Membr Biol 165, 1998; Kaliszan and Markuszewski, International Journal of Pharmaceutics 145, 1996; Hitchcock and Pennington, J. Med. Chem. 2006).

In preferred embodiments of the first aspect, the compound for use has a molecular weight of 800 g/mol or less, particularly 700 g/mol or less, more particularly 600 g/mol or less, even more particularly 550 g/mol or less, even more particularly 500 g/mol or less, even more particularly 450 g/mol or less.

In preferred embodiments of the first aspect, the compound for use has a polar surface area (PSA) of 90 Å or less, particularly 80 Å or less.

The compounds according to the invention not only inhibit the NO production of microglia, but also the NO production of macrophages. Thus, the compounds of the invention are not limited to the treatment and prevention of conditions of the brain. Other conditions for which the compounds of the invention are suitable include heart failure with preserved ejection fraction (HFpEF), diabetic nephropathy, arthritis and/or asthma. For the treatment or prevention of these conditions it is not required that the compounds are able to cross the blood-brain-barrier.

In preferred embodiments of the first aspect, the compound for use inhibits NO production by microglia with an IC₅₀ of 10 μM or less, particularly 1 μM or less, 200 nM or less, 100 nM or less, 50 nM or less, even more particularly 25 nM or less.

In preferred embodiments of the first aspect, the compound for use inhibits NO production by macrophages with an IC₅₀ of 10 μM or less, particularly 1 μM or less, 200 nM or less, 100 nM or less, 50 nM or less, even more particularly 25 nM or less.

In the context of the present invention, the IC₅₀ is the concentration that is capable of reducing the NO release to 50%.

NO production may be assessed e.g. by an assay called “Griess assay”, which is described in the examples section of this application.

In preferred embodiments of the first aspect, the compound for use is an inhibitor of iNOS (NOS2). iNOS is a NO synthetase that is specific for microglia and macrophages.

The compounds according to the invention have a unique chemical structure that distinguishes them from other NOS inhibitors. Most nonselective NOS inhibitors, like L-NMMA, or L-NAME, mimic the NOS substrate L-arginine antagonizing the binding to the highly conserved catalytic site of NOS (Fischmann et al., 1999) and thus potently inhibit all NOS isoforms (Heemskerk et al., 2009). Inhibitors more selective for iNOS, like 1400 W or GW273629 and GW274150, show some more variance in their chemical structure. In certain embodiments, the compounds according to the invention are based on a unique protein like structure based on 4 amino acid like subgroups, resembling phenylalanine, proline, and 2 times tyrosine, which are connected via peptide bonds. Compared to the inhibitors named before, the compounds of the invention possess a unique structure, indicating a new interaction with iNOS.

In preferred embodiments of the first aspect, the compound is characterized by a specific stereoisomensm, as defined in formulae (1b), (2b), (3b), and (4b), in which the carbon atoms at position 2 and 4 are in (S) configuration (wherein the positions are defined as indicated in formula (1a) below):

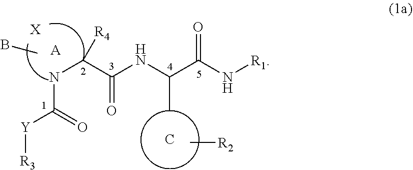

Surprisingly, the inventors found that the inhibitory effect on NO production is achieved by such enantiomers in which the carbon atoms at position 2 and 4 are in (S) configuration (FIG. 9).

The compound for use according to the first aspect of the invention may be in the form of a salt, in particular a pharmaceutically acceptable salt.

The compounds for use according to the first aspect are also claimed per se. In a second aspect, the present invention relates to a compound characterized by a formula (1a) or (1b)

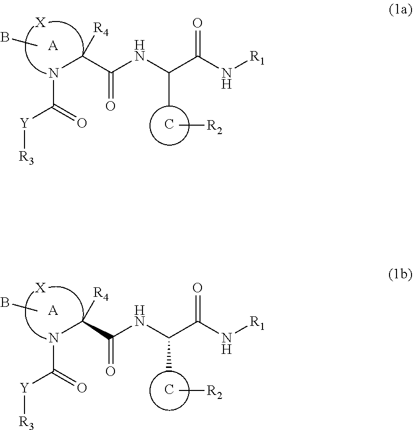

wherein
   R¹ is independently selected from the group consisting of optionally substituted C₁-C₁₀-alkyl, i.e. C₁-, C₂-, C₃-, C₄-, C₅-, C₆-, C₇-, C₈-, C₉- or C₁₀-alkyl; C₁-C₁₀-heteroalkyl, i.e. C₁-, C₂-, C₃-, C₄-, C₅-, C₆-, C₇-, C₈-, C₉- or C₁₀-heteroalkyl; C₁-C₁₀-haloalkyl, i.e. C₁-, C₂-, C₃-, C₄-, C₅-, C₆-, C₇-, C₈-, C₉- or C₁₀-haloalkyl; C₂-C₁₀-alkenyl, i.e. C₂-, C₃-, C₄-, C₅-, C₆-, C₇-, C₈-, C₉- or C₁₀-alkenyl; C₂-C₁₀-heteroalkenyl, i.e. C₂-, C₃-, C₄-, C₅-, C₆-, C₇-, C₈-, C₉- or C₁₀-heteroalkenyl; C₂-C₁₀-alkynyl, i.e. C₂-, C₃-, C₄-, C₅-, C₆-, C₇-, C₈-, C₉- or C₁₀-alkynyl; C₃-C₁₀-cycloalkyl, i.e. C₃-, C₄-, C₅-, C₆-, C₇-, C₈-, C₉- or C₁₀-cycloalkyl; C₃-C₁₀-heterocycloalkyl, i.e. C₃-, C₄-, C₅-, C₆-, C₇-, C₈-, C₉- or C₁₀-heterocycloalkyl; C₄-C₁₀-cycloalkenyl, i.e. C₄-, C₅-, C₆-, C₇-, C₈-, C₉- or C₁₀-cycloalkenyl; C₄-C₁₀-heterocycloalkenyl, i.e. C₄-, C₅-, C₆-, C₇-, C₈-, C₉- or C₁₀-heterocycloalkenyl; C₅-C₁₄-aryl, i.e. C₅-, C₆-, C₇-, C₈-, C₉-, C₁₀-, C₁₁-, C₁₂-, C₁₃-, or C₁₄-aryl; C₅-C₁₄-heteroaryl, i.e. C₅-, C₆-, C₇-, C₈-, C₉-, C₁₀-, C₁₁-, C₁₂-, C₁₃-, or C₁₄-heteroaryl; C₆-C₁₅ alicyclic system, i.e. C₆, C₇, C₈, C₉, C₁₀, C₁₁, C₁₂, C₁₃, C₁₄, or C₁₅ alicyclic system; C₆-C₁₅-aralkyl, i.e. C₆-, C₇-, C₈-, C₉-, C₁₀-, C₁₁-C₁₂-, C₁₃-, C₁₄-, or C₁₅-aralkyl; and C₆-C₁₅-heteroaralkyl, i.e. C₆-, C₇-, C₈-, C₉-, C₁₀-, C₁₁-C₁₂-, C₁₃-, C₁₄-, or C₁₅-heteroaralkyl;
R² is selected from the group consisting of —F, —Br, —Cl, —R⁵, —OR⁵, —COR⁵, —CO₂R⁵, —OCX₃, —N(R)R′, —N(R)—C(O)—R′, —C(O)—N(R)—C(O)—R′, —N(R)—C(O)—OR′, —C(O)N(R)R′, —N(R)—C(O)—N(R′)R″, —R═NR′, —R═NH, —CN, —NC, —ONO, —NO₂, —ONO₂, —NO, —OCN, —NCO, —SR, —SX₃, —SX₅, —S(O)R, —SO₂R, and —SO₃H, wherein
   R⁵ is selected from the group consisting of —H and optionally substituted C₁-C₁₀ alkyl, C₂-C₁₀-alkenyl, C₂-C₁₀-alkynyl, C₃-C₁₀-cycloalkyl, C₃-C₁₀-heterocycloalkyl, C₅-C₁₄-aryl, C₅-C₁₄-heteroaryl, C₆-C₁₅-aralkyl, C₆-C₁₅-heteroaralkyl, and —(OCH₂CH₂)ₙ₁—NH—R⁶, wherein
      n1 is 2-10, particularly 4-8, and R⁶ is a biotin moiety linked via an amide bond; R, R′ and R″ are independently selected from the group consisting of —H, C₁-C₃-alkyl and C₂-C₃-alkenyl;
R⁴ is selected from the group consisting of —H and optionally substituted C₁-C₁₀-alkyl, C₂-C₁₀-alkenyl;
A is a 4- to 8-, i.e. 4-, 5-, 6-, 7-, or 8-membered, in particular 5- or 6-membered heterocycloalkyl or heteroaryl;
B is a 4- to 8-, i.e. 4-, 5-, 6-, 7-, or 8-membered cycloalkyl, heterocycloalkyl, aryl or heteroaryl, wherein B may be present or absent and wherein if B is present, A and B together form a double cycle;
C is a 5- or 6-membered aryl or heteroaryl;
X is selected from the group consisting of —CH₂—, —NH—, —O—, —S—, —*CHF—, —*CHO(R^{X})—, —*CHNH(R^{X})—, —*CH—CH(R^{X})—, —*C═C(R^{X})—, —*CH—N(R^{X})—, wherein R^{X} represents hydrogen, methyl, ethyl, cyclopropyl, or —CH₂-cyclopropyl, and wherein the *C is part of heterocycloalkyl or heteroaryl A; and
Y is selected from —*CH₂—, —*NH—, —*O—, —*CH₂—CH(R^{Y})—, —*CH═C(R^{Y})—, —*CH₂—O—, —*CH₂—N(R^{Y})— and —*O—CH₂—, wherein R^{Y} represents hydrogen, methyl, ethyl, cyclopropyl, or —CH₂-cyclopropyl, wherein Y may optionally be absent, and wherein the *C, *N or *O is covalently linked to the C atom of the carbonyl group.

In preferred embodiments of the second aspect, the compound is characterized by formula (1a) or (1b), wherein R¹, R², R³, R⁴, A, B, C, X and Y are defined as above for the second aspect of the invention with the proviso that if Y—R³ is

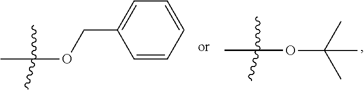

### R¹ is not

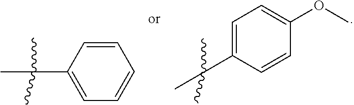

In preferred embodiments of the second aspect, the compound is characterized by formula (1b), wherein R¹, R², R³, R⁴, A, B, C, X and Y are defined as above for the second aspect of the invention, optionally with the proviso that if Y—R³ is

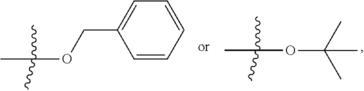

### R¹ is not

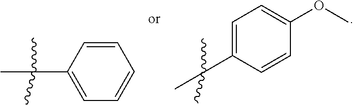

Preferably, the compound according to the second aspect of the invention can be a compound as described in any of the embodiments of the compounds for use of the first aspect of the invention, with the proviso that either the compound is characterized by any one of formulae (1b), (2b), (3b) or (4b), i.e. the C atoms at position 2 and 4 are in (S) configuration, wherein R¹, R², R³, R⁴, A, B, C, X and Y are defined as in the respective embodiment of the first aspect of the invention, or the compound is characterized by any one of formulae (1a), (2a), (3a) or (4a), but with the proviso that if Y—R³ is

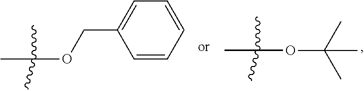

### R¹ is not

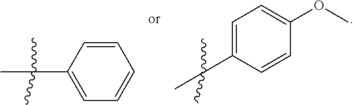

Preferably, the compound of the second aspect is characterized by formula (1b), wherein R², R³, R⁴, A B, X and Y are defined as above and 14,

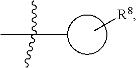

wherein

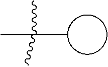

is a 5- or 6-membered aryl or heteroaryl, and R⁸ is selected from H, R⁹ and OR⁹, wherein R⁹ is selected from optionally substituted C₁-, C₂-, C₃-, or C₄-alkyl or cycloalkyl, wherein the aryl or heteroaryl and R⁹ may form a double cycle and wherein

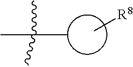

comprises at least one heteroatom.
More preferably, R¹ is selected from the group consisting of

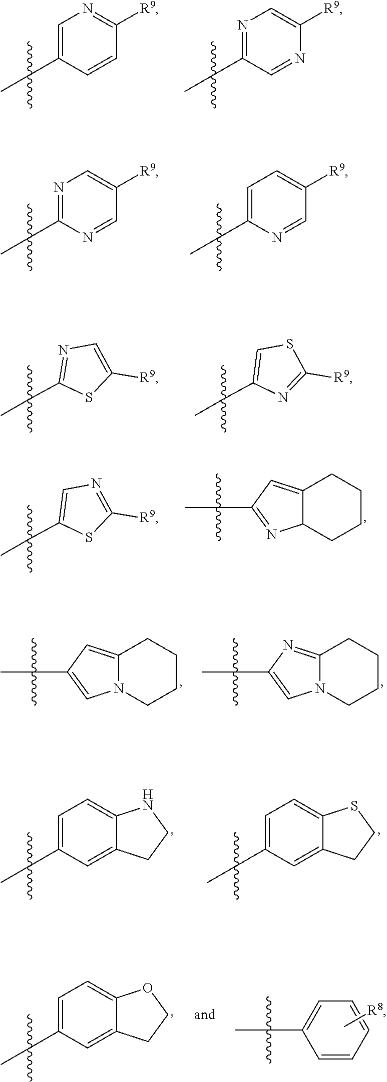

wherein R⁸ is selected from R⁹ and OR⁹, and wherein R⁹ is an unsubstituted C₁-, C₂-, C₃-, or C₄-alkyl or unsubstituted C₃-, or C₄-cycloalkyl.
Even more preferably, R¹ is selected from the group consisting of

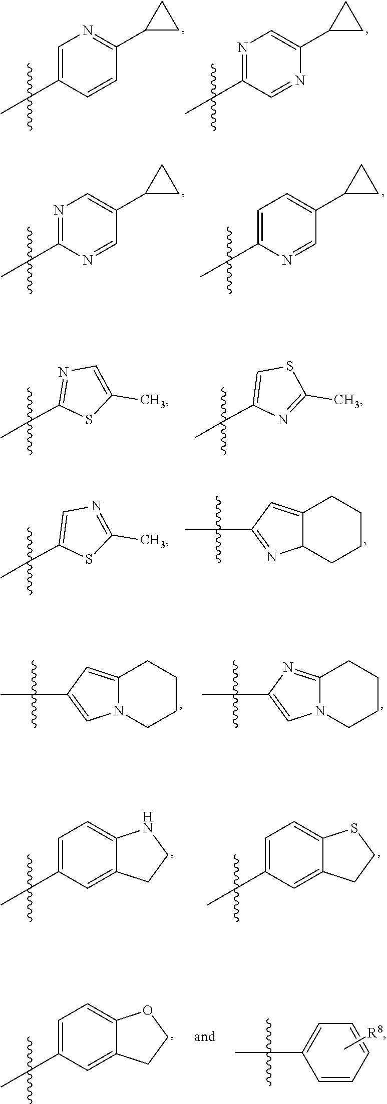

wherein R⁸ is selected from R⁹ and OR⁹, and wherein R⁹ is an unsubstituted C₁-, C₂-, C₃-, or C₄-alkyl or unsubstituted C₃-, or C₄-cycloalkyl.
Most preferably, R¹ is selected from the group consisting of

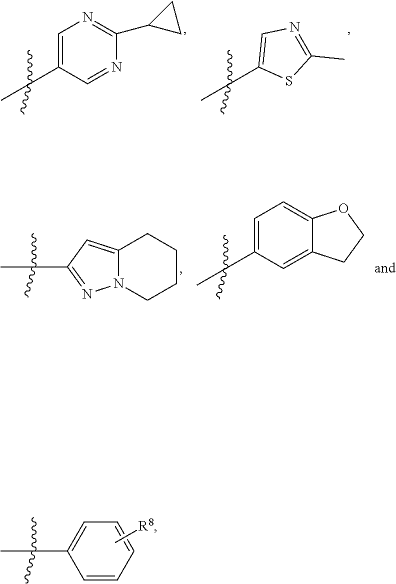

wherein R⁸ is selected from R⁹ and OR⁹, wherein R⁹ is an unsubstituted C₁-, C₂-, C₃-, or C₄-alkyl.

The compound according to the second aspect of the invention can be a compound as described in any of the embodiments of the compounds for use of the first aspect of the invention, wherein the compound is characterized by any one of formulae (1b), (2b), (3b) or (4b), wherein R¹, R², R³, R⁴, A, B, C, X and Y are defined as in the respective embodiment of the first aspect of the invention, with the proviso that if Y—R³ is

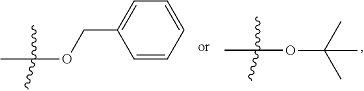

### R¹ is not

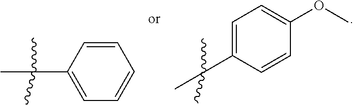

In preferred embodiments of the second aspect, Y—R³ is not

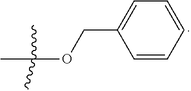 [SMILES: *OCc1ccccc1]

In preferred embodiments of the second aspect, Y—R³ is not

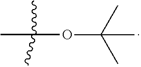 [SMILES: *OC(C)(C)C]

In more preferred embodiments of the second aspect, Y—R³ is not

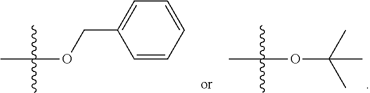

In preferred embodiments of the second aspect, Y is absent.

In preferred embodiments of the second aspect, the compound corresponds to a compound as described in any of the embodiments of the compounds for use of the first aspect of the invention, wherein Y is absent or selected from *CH₂—, *NH, *CH₂—CH(R^{Y}), *CH═C(R^{Y}), *CH₂—O and *CH₂—N(R^{Y}), wherein R^{Y} is defined as above for the second aspect and the *C or *N marked with an asterisk is covalently linked to the C atom of the carbonyl group.

Preferably, the compound according to the second aspect corresponds to a compound as described in any of the embodiments of the compounds for use of the first aspect of the invention, wherein Y is —*CH₂—(CH₂)₃— or —*CH₂—(CH₂)ₙ₄—O—, wherein n3 is 0, 1 or 2 and n4 is 0 or 1, and *C is covalently linked to the C atom of the carbonyl group.

It is also preferred that R³ is selected from optionally substituted C₅- or C₆-cycloalkyl, C₅- or C₆-heterocycloalkyl, C₅- or C₆-aryl and C₅- or C₆-heteroaryl.

In preferred embodiments of the second aspect, the compound corresponds to a compound as described in any of the embodiments of the compounds for use of the first aspect of the invention, wherein
Y is —*CH₂—(CH₂)ₙ₃— or —*CH₂—(CH₂)ₙ₄—O—,
   wherein n3 is 0, 1 or 2 and n4 is 0 or 1, and
   *C is covalently linked to the C atom of the carbonyl group, and
R³ is selected from optionally substituted C₅- or C₆-cycloalkyl, C₅- or C₆-heterocycloalkyl, C₅- or C₆-aryl and C₅- or C₆-heteroaryl.

In preferred embodiments of the second aspect, the compound corresponds to a compound as described in any of the embodiments of the compounds for use of the first aspect of the invention, wherein
Y is —*CH₂—(CH₂)ₙ₃— or —*CH₂—(CH₂)ₙ₄—O—,
   wherein n3 is 0, 1 or 2 and n4 is 0 or 1, and
   *C is covalently linked to the C atom of the carbonyl group, and
R³ is selected from unsubstituted C₅- or C₆-cycloalkyl, C₅- or C₆-heterocycloalky, C₅- or C₆-aryl, C₅- or C₆-heteroaryl and

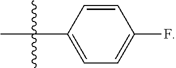 [SMILES: *c1ccc(F)cc1]

In preferred embodiments of the second aspect, the compound is characterized by any one of formulae (1a), (1b), (2a), (2b), (3a), (3b), (4a) or (4b), preferably (1b), (2b), (3b) or (4b), more preferably (3b) or (4b), even more preferably (4b), wherein
if applicable, A is a 4- to 8-, i.e. 4-, 5-, 6-7 or 8-membered, in particular 5- or 6-membered heterocycloalkyl or heteroaryl;
if applicable, B is a 4- to 8-, i.e. 4-, 5-, 6-7 or 8-membered cycloalkyl, heterocycloalkyl, aryl or heteroaryl, wherein B may be present or absent and wherein if B is present, A and B together form a double cycle;
if applicable, C is 5- or 6-membered aryl or heteroaryl;
R¹ is selected from

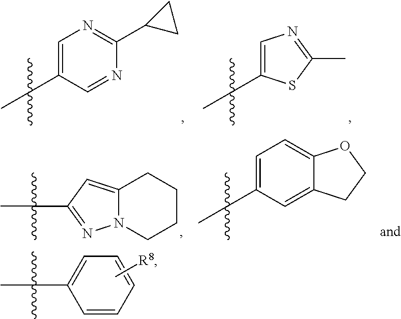

wherein R⁸ is selected from R⁹ and OR⁹, wherein R⁹ is an unsubstituted C₁-C₄-alkyl;
R² is —OR⁵, wherein R⁵ is selected from the group consisting of —H and optionally substituted C₁-C₆ alkyl, C₂-C₆-alkenyl, C₂-C₆-alkynyl and C₃-C₈-cycloalkyl, in particular from C₁-C₄-alkyl or C₃-C₆-cycloalkyl;
R³ is (CH₂)ₙ₂—R⁷, wherein n2 is 1-4, i.e. 1, 2, 3 or 4, and R⁷ is selected from the group consisting of optionally substituted C₁-C₇-alkyl, C₄-C₇-cycloalkyl, C₅-C₁₀-aryl and C₅-C₁₀-heteroaryl,
Y is absent or selected from —*CH₂—, —*O—, —*CH₂—CH₂—, —*CH₂—O—, and —*O—CH₂—, in particular Y is absent or selected from —*CH₂— and —*O—, wherein the *C or *O is covalently linked to the C atom of the carbonyl group; and
X is selected from the group consisting of —CH₂—, —NH—, —O—, —S—, and —*CHF—, in particular X is —*CH₂—, wherein the *C is part of the heterocycloalkyl.

In more preferred embodiments of the second aspect, the compound is characterized by any one of formulae (1a), (1b), (2a), (2b), (3a), (3b), (4a) or (4b), preferably (1b), (2b), (3b) or (4b), more preferably (3b) or (4b), even more preferably (4b), wherein
if applicable, A is a 4- to 8-, i.e. 4-, 5-, 6-7 or 8-membered, in particular 5- or 6-membered heterocycloalkyl or heteroaryl;
if applicable, B is a 4- to 8-, i.e. 4-, 5-, 6-7 or 8-membered cycloalkyl, heterocycloalkyl, aryl or heteroaryl, wherein B may be present or absent and wherein if B is present, A and B together form a double cycle;
if applicable, C is 5- or 6-membered aryl or heteroaryl;
R¹ is selected from

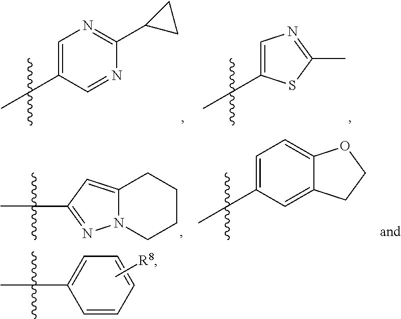

wherein R⁸ is selected from R⁹ and OR⁹, wherein R⁹ is an unsubstituted C₁-C₄-alkyl;
R² is —OR⁵, wherein R⁵ is selected from the group consisting of —H and optionally substituted C₁-C₆ alkyl, C₂-C₆-alkenyl, C₂-C₆-alkynyl and C₃-C₈-cycloalkyl, in particular from C₁-C₄-alkyl or C₃-C₆-cycloalkyl;
R³ is (CH₂)ₙ₂—R⁷, wherein n2 is 1-4, i.e. 1, 2, 3 or 4, and R⁷ is selected from

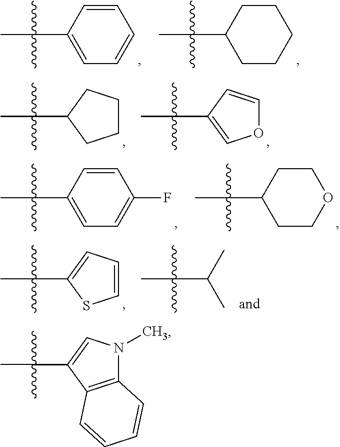

in particular R⁷ is

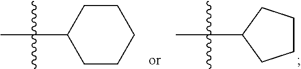 [SMILES: *C1CCCCC1 ; *C1CCCC1]

Y is absent or selected from —*CH₂—, —*O—, —*CH₂—CH₂—, —*CH₂—O—, and —*O—CH₂—, in particular Y is absent or selected from —*CH₂— and —*O—, wherein the *C or *O is covalently linked to the C atom of the carbonyl group; and
X is selected from the group consisting of —CH₂—, —NH—, —O—, —S—, and —*CHF—, in particular X is —*CH₂—, wherein the *C is part of the heterocycloalkyl.

In even more preferred embodiments of the second aspect, the compound is characterized by any one of formulae (1a), (1b), (2a), (2b), (3a), (3b), (4a) or (4b), preferably (1b), (2b), (3b) or (4b), more preferably (3b) or (4b), even more preferably (4b), wherein
if applicable, A is a 4- to 8-, i.e. 4-, 5-, 6-7 or 8-membered, in particular 5- or 6-membered heterocycloalkyl or heteroaryl;
if applicable, B is a 4- to 8-, i.e. 4-, 5-, 6-7 or 8-membered cycloalkyl, heterocycloalkyl, aryl or heteroaryl, wherein B may be present or absent and wherein if B is present, A and B together form a double cycle;
if applicable, C is 5- or 6-membered aryl or heteroaryl;
R¹ is

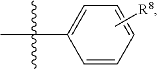

wherein R⁸ is —O—CH₃, in particular wherein R⁸ is in para position;
R² is —OR⁵, wherein R⁵ is selected from the group consisting of —H and optionally substituted C₁-C₆ alkyl, C₂-C₆-alkenyl, C₂-C₆-alkynyl and C₃-C₈-cycloalkyl, in particular from C₁-C₄-alkyl or C₃-C₆-cycloalkyl;
R³ is (CH₂)ₙ₂—R⁷, wherein n2 is 1-4, i.e. 1, 2, 3 or 4, and R⁷ is selected from

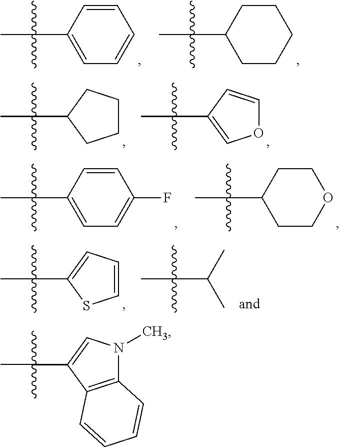

in particular R⁷ is

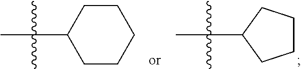 [SMILES: *C1CCCCC1 ; *C1CCCC1]

Y is absent or selected from —*CH₂—, —*O—, —*CH₂—CH₂—, —*CH₂—O—, and —*O—CH₂—, in particular Y is absent or selected from —*CH₂— and —*O—, wherein the *C or *O is covalently linked to the C atom of the carbonyl group; and
X is selected from the group consisting of —CH₂—, —NH—, —O—, —S—, and —*CHF—, in particular X is —*CH₂—, wherein the *C is part of the heterocycloalkyl.

In most preferred embodiments of the second aspect, the compound is characterized by any one of formulae (1a), (1b), (2a), (2b), (3a), (3b), (4a) or (4b), preferably (1b), (2b), (3b) or (4b), more preferably (3b) or (4b), even more preferably (4b), wherein
if applicable, A is a 4- to 8-, i.e. 4-, 5-, 6-7 or 8-membered, in particular 5- or 6-membered heterocycloalkyl or heteroaryl;
if applicable, B is a 4- to 8-, i.e. 4-, 5-, 6-7 or 8-membered cycloalkyl, heterocycloalkyl, aryl or heteroaryl, wherein B may be present or absent and wherein if B is present, A and B together form a double cycle;
if applicable, C is 5- or 6-membered aryl or heteroaryl;
R¹ is

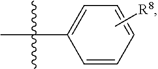

wherein R⁸ is —O—CH₃, in particular wherein R⁸ is in para position;
R² is —OCH₃;
R³ is (CH₂)₂—R⁷, wherein n2 is 1-4, i.e. 1, 2, 3 or 4, and R⁷ is selected from

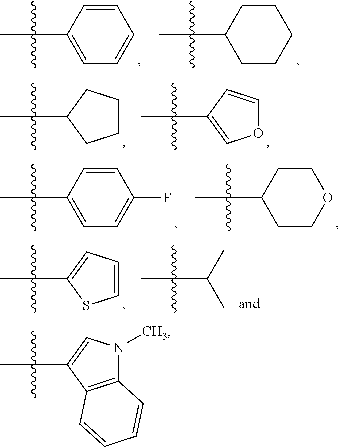

in particular R⁷ is

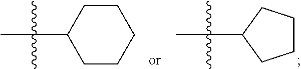 [SMILES: *C1CCCCC1 ; *C1CCCC1]

Y is absent or selected from —*CH₂—, —*O—, —*CH₂—CH₂—, —*CH₂—O—, and —*O—CH₂—, in particular Y is absent or selected from —*CH₂— and —*O—, wherein the *C or *O is covalently linked to the C atom of the carbonyl group; and
X is selected from the group consisting of —CH₂—, —NH—, —O—, —S—, and —*CHF—, in particular X is —*CH₂—, wherein the *C is part of the heterocycloalkyl.

In preferred embodiments of the second aspect, the compound is characterized by any of formulae (5)-(34), preferably (5)-(29).

In preferred embodiments of the second aspect, the compound is characterized by any of formulae (5)-(16), (19)-(27) or (29)-(34), preferably (5)-(16) and (19)-(27).

In preferred embodiments of the second aspect, the compound is characterized by any of formulae (5)-(9), (11), (12), preferably (5)-(9), (11) or (12), more preferably (5)-(8), (11) or (12).

In preferred embodiments of the second aspect, the compound is characterized by any of formulae (5)-(12), preferably (5)-(9), more preferably (5)-(8).

In a third aspect, the present invention relates to a pharmaceutical composition comprising the compound according to the first aspect of the invention.

In an additional aspect, the compounds for use according to the first aspect are claimed per se.

In another aspect, the present invention relates to a method of treating or preventing a disease comprising administration of an effective amount of the compound according the first aspect of the invention to a patient in need thereof. The diseases that can be treated or prevented are those described with regard to the first aspect of the invention.

In yet another aspect, the present invention relates to the use of the compound according the first aspect of the invention in the manufacture of a medicament.

### BRIEF DESCRIPTION OF DRAWINGS

FIG. 1: shows that C1 reduces LPS induced microglial NO release in a dose dependent manner. A) Representative illustration of the HTS setup. Initially 30 000 compounds were tested for their ability to reduce LPS induced NO release in BV2 cells resulting in 233 positive compounds, 60 of those pass the AlamarBlue assay. 30 compounds could confirm their ability to reduce the LPS induced NO release in primary cultured neonatal microglia. The followed AlamarBlue assay revealed compounds not cytotoxic for primary microglia, resulting in one compound the inventors tested further. B) C1 decreases LPS-induced production of NO in primary cultured neonatal microglia dose dependently (shown in values relative to internal positive control as stars) while the metabolic activity remained stable (shown in absolute values as dots) in the HTS setup. Two data points of the NO assay, marked with open circles, were automatically excluded using a robust fit outlier model in GraphPad prism. The calculated IC₅₀ is 224 nM. C) The 4 subgroups of the chemical structure or C1, resemble the amino acids phenylalanine, proline, and 2 times tyrosine. The compounds structure inherits 2 stereo centers, one in the amino acid proline, and the other in the amino acid like structure of tyrosine. D) C1 fulfils 4 of the 5 “Lipinski rule of five”, that include the number hydrogen bond donors (Lipinski ≤5, C1=2), and the hydrogen bond acceptors (Lipinski ≤10, C1=6), polar surface area (Lipinski 140≤A2, C1=106.2 Å2), solubility (Lipinski ≤5 A log P, C1=4.7 A log P), but not for the molecular weight (Lipinski ≤500 g/mol, C1=517.6 g/mol). E) Transferred to a laboratory scaled setup C1 in DMSO verified its ability to reduce the NO release in primary cultured neonatal microglia dose dependently in the tested log 10 concentration range from 0.002 μM up to 20 μM (bottom line in dark grey). Corresponding concentrations of DMSO (log 10 range from 0.6×10-6 v/v up to 1×10-3 v/v, top line in light grey). Microglia were treated with C1 or DMSO for 1 hour followed by an additional stimulation with 1 μg/mL LPS for 48 hours. The IC₅₀ shifted from 224 nM in the initial HTS to 252 nM. F) The metabolic activity of primary cultured neonatal microglia was measured with an AlamarBlue assay using the same protocol as noted in D. DMSO alone (shown as dots) shows no impact on the metabolic activity of the cells. 20 μM of C1 (shown as stars) reduces the metabolic activity to 84%±3.99%, however not below the unstimulated control (69%±2.47%). G) C1 and DMSO do not quench NO or have an impact on the Griess assay. NO enriched supernatant taken from LPS stimulated microglia (1 μg/mL LPS for 48 hours) was incubated with C1 in DMSO (top line in dark grey) or DMSO alone (bottom line in light grey) (concentration range is the same as in d and e) for 24 hours. Treatment with C1 or DMSO showed no dose dependent reduction on the NO concentration in the supernatant when compared to the untreated supernatant.

FIG. 2: shows that in microglia, C1 reduces NO release triggered by IFNg and Poly I C, while LPS induced cytokine release is not affected. A) Stimulation of microglia with 100 ng/mL IFNγ for 48 hours (left, in black) and 100 μg/mL PolyIC for 24 hours (right, in black) causes a significant increase in the production of NO (IFNγ: 87% and PolyIC: 53%, both p<0.0001) in comparison with unstimulated control (in white). Pre-treatment with C1 causes a dose dependent decrease of NO release in neonatal microglia upon stimulation with IFNγ (left, in dark grey) and PolyIC (right, in dark grey). Incubation with DMSO (in light grey) has no effect on the NO release. B) Pre-treating microglia for 1 hour with C1 followed by an 48 hours stimulation with 1 μg/mL LPS increases the concentration of the pro-inflammatory cytokines IL6, TNFα and IL1β comparing to the negative control (plain medium in white) Treatment with C1 (in dark grey) exceeded the LPS-induced release of IL1β in all tested concentrations compared to the LPS stimulated plain medium (in black) (0.025 μM: p=0.0037, 0.25 μM: p<0.0001, 2.5 μM: p<0.0001), when compared to DMSO (in light grey) 0.25 μM C1 (p=0.0004) and 2.5 μM C1 (p=0.0012) were significantly higher. C1 has no effect on the LPS induced TL6 and TNFα release in microglia. *** p<0.001 ** p<0.01 comparing to stimulated control, ###p<0.001 ##p<0.01 comparing to DMSO (1 way ANOVA followed by Bonferroni's post-hoc test).

FIG. 3: shows that C1 acts as a chemoattractant but does not influence microglial motility and phagocytosis, furthermore it does not influence iNOS mRNA regulation but iNOS protein regulation. A) C1 affects microglial chemotaxis (left) but not motility (right). The results were normalized to ATP induced chemotaxis or ATP induced motility respectively. A gradient of 2.5 μM C1 increased chemotaxis significantly compared to plain medium (p=0.0036) and DMSO (p=0.0012). ATP induced chemotaxis was not altered significantly in the presence of 2.5 μM C1. Microglial motility was reduced in the presence of C1 significantly compared to plain medium (p=0.0018) but not to DMSO. ATP induced motility was neither changed in the presence of C1 nor DMSO. *** p<0.001 ** p<0.01 comparing to stimulated control, ###p<0.001 ##p<0.01 comparing to DMSO (1 way ANOVA followed by Bonferroni's post-hoc test). B) The phagocytic activity both under basal conditions (left) and LPS stimulated conditions (right) was assessed using a FACS-based protocol. Microglia were pre-treated with 2.5 μM C1 in DMSO or its corresponding concentration of DMSO (125×10-5 v/v) followed by an additional stimulation with 1 μg/mL LPS for 24 hours. Under basal conditions neither C1 nor DMSO changes microglial phagocytosis significantly (plain bars). Upon LPS stimulation phagocytosis increased significantly around 60% compared to their own unstimulated control (plain medium: +55% p=0.0066, DMSO: +67% p=0.0068, CL: +45% p=0.0163, black striped bars). Within LPS stimulated conditions there were no significant differences. *** p<0.001 ** p<0.01 comparing to plain medium control (non-matching 2 way ANOVA followed by Sidak test). C) The relative expression of iNOS mRNA was evaluated under basal conditions and LPS stimulated conditions. The protocol is parallel to the one described in B, ending in the extraction and quantification of the mRNA using qRT-PCR. Under basal conditions iNOS mRNA level was barely detected. DMSO and C1 had no significant effect on the basal level of iNOS mRNA. Upon 1 μg/mL LPS stimulation iNOS mRNA level increases more than 5 000 fold independent of the treatment (induced vs basal: p<0.0001). Within the stimulated conditions treatment with C1 and DMSO did not change mRNA significantly. * p<0.001 comparing to plain medium control (non-matching 2 way ANOVA followed by Sidak test). D) C1 affects the NO release of pro-inflammatory stimulated microglia in a post-treatment setup. Microglia were stimulated for 24 hours with 1 μg/mL LPS. Afterwards the medium was changed, and microglia were treated with 2.5 μM C1 in plain medium (in dark grey, bottom line), the corresponding concentration of DMSO (125×10-5 v/v) in plain medium (in light grey, first line from top) or with plain medium only (in black, second line from top). After medium change, the NO concentration increased linear in the supernatant of untreated microglia, reaching 28 μM (±1.036 SEM) after 60 hours. DMSO treatment did not change this linear increase, reaching 29±1.119 μM. Treatment with C1 leads to an increase in NO concentration to a lower extant over time compared to DMSO and plain medium treatment. The increase in NO concentration followed a one-phase association (R2 of 0.6498) and reached a calculated plateau of 7 μM (95% confidence interval 5.999 to 8.127). After 8 hours of treatment with C1, the level of NO was significantly reduced compared to the control condition (p=0.006), and after 12 hours to the DMSO condition (p<0.0001). *** p<0.001 ** p<0.01 comparing to plain medium control, ###p<0.001 ##p<0.01 comparing to innate DMSO control (non-matching 2 way ANOVA followed by Sidak test).

FIG. 4: shows that C1 passes the blood brain barrier and improves behavioural deficits after mild brain ischemia. A) Distribution of C1 in different organs after a single intravenous injection into healthy male mice (5 mg/kg, n=3). The concentration of C1 in blood plasma (first line from top), heart (fourth line from top), liver (third line from top), kidney (second line from top), and brain (bottom line and amplified graph in the right) was monitored for 4 hours (samples taken after 30 min, 1 h, 2 h, and 4 h). C1 reached the brain and maintained a concentration above the IC₅₀ value for about 30 min (shown in the amplified graph in the right). B) Male C57Bl/6 mice were submitted to 30 minutes of middle cerebral artery occlusion (MCAO), as a model for mild ischemic injury. From the day of the MCAO surgery until day 6 after MCAO, all mice were injected i.p. daily with 5 mg/kg of C1 diluted in 125×10-5 v/v DMSO as a vehicle or with just the vehicle as control. The motor coordination (accelerated Rotarod) and extrapyramidal motor locomotion (pole test) were assessed at day 2 and 5 after MCAO, mice were trained for the both tests on day 3 and 2 before MCAO and baselines were taken on the day before MCAO. The corner test was used to test mice's laterality on day 6 after MCAO by counting the amount of turns to the left in a total of 10 trials which was then compared to each mouse individual baselines, taken 5 days before MCAO. On day 3 after MCAO, MRI scans were performed to determine the volume of the ischemic lesion. C) The treatment of mice with C1 did not cause a significant difference (p=0.3959) in the volume of the ischemic lesion, measured with MRI in between the treated mice (in dark grey) in comparison with the controls (in light grey), using an unpaired t test. D) There are no differences in between the treated group (in dark grey) and the controls (in light grey) in the Rotarod test, using a 2 way ANOVA test. E) During the pole test, the treated group was significantly (p=0.0253) faster than the control group in turning 180° upside down (1.175s versus 2.328s) on day 2 after MCAO. No differences were observed on day 5 after MCAO. A 2 way ANOVA test was used to test for statistical relevance. * p<0.05. F) Additional during the pole test, the treated group was significantly faster at descending the pole (p=0.0145; 6.592s versus 10.47s) on day 2 after MCAO. No differences were observed on day 5 after MCAO. A 2 way ANOVA test was used to test for statistical relevance. * p<0.05. G) The animals treated with C1 (in dark grey, in the right) perform fewer turns to the left (mean 7.00 in 10 turns, p=0.0213) in comparison to controls (in light grey, 8.54 in 10 turns to the left), using a 2 Way ANOVA statistical test * p<0.05.

FIG. 5: shows that C1 alone does not evoke NO release or an increase in cell activity. A) Determination of the effect of C1 on the NO release in unstimulated microglia showed no significant difference. Microglia were treated with C1 (0.025 μM, 0.25 μM, or 2.5 μM, in dark grey) or its corresponding concentration of DMSO (1.25×10-5, 12.5×10-5, or 125×10-5 v/v, in light grey) for 48 hours. Untreated microglia stimulated with 1 μg/mL LPS for 48 hours were set as positive control (in black). All values were normalised to the positive control. As shown in FIG. 1 does LPS stimulation induce a significant increase in NO release (p<0.0001). Treatment with C1 or DMSO did not show any significant difference compared to untreated microglia (in white). B) Using the same protocol as in A, the metabolic activity was assessed using AlamarBlue assay. LPS stimulation induced a significant increase in metabolic activity (in black) compared to the unstimulated microglia (in white) (p<0.0001). Treatment with C1 (in dark grey) or DMSO (in light grey) did not show any significant difference compared to untreated microglia.

FIG. 6: shows that C1 alone does not evoke cytokine release. Treatment with C1 did not show any significant changes in the release of pro-inflammatory cytokines (A: IL10, B: IL6, C: TNFα). Using the same protocol described in FIG. 2-1, the release of IL1β, IL6, and TNFα was measured using ELISA. Untreated microglia stimulated with 1 μg/mL LPS for 48 hours were set as positive control (in black). All values were normalised to the positive control respectively. As shown in FIG. 2 does LPS stimulation induce a significant increase in the release of IL1β, IL6, and TNFα (all p<0.0001). Treatment with C1 (in dark grey) or DMSO (in light grey) did not show any significant difference compared to untreated microglia in all measured cytokines. *** p<0.001 comparing to plain medium control (1 way ANOVA followed by Bonferroni's post-hoc test).

FIG. 7: shows that C1 shows similar results on macrophages as for microglia and does not interfere with cell activity, proliferation, and cell death are not affected in non-stimulated conditions. A) Adult bone marrow derived macrophages were isolated and pre-treated for 1 hour with C1 (0.025 μM, 0.25 μM, or 2.5 μM, in red) or its corresponding concentration of DMSO (1.25×10-5, 12.5×10-5, or 125×10-5 v/v, in grey), followed by 48 hours stimulation with 1 μg/mL LPS. In addition, untreated macrophages were stimulated 48 hours with 1 μg/mL LPS (in black) or kept in plain medium (negative control in white). 0.25 μM C1 reduced the NO release significantly compared to positive control (p<0.0001) and DMSO (p<0.0001) and 2.5 μM C1 reduced the NO release to similar level as the negative control (C1: 4.85%±0.65 SEM, plain medium: 6.01%±0.62 SEM, p>0.9999) significantly different to the positive control (p<0.0001) and DMSO (p<0.0001). B) The same stimulation protocol as in A was used, replacing the LPS by 100 μg/mL PolyIC for 24 hours and the NO release was measured. Similar as for the LPS stimulation, 0.25 μM showed a significant decrease compared to positive control (p<0.0001) and DMSO (p<0.0001). 2.5 μM of C1 lead to a decrease (15.01±10.32%, p=0.9751) to the level of negative control (24.30±12.91%) significantly different to the positive control (p<0.0001) and DMSO (p<0.0001). C) When the stimulus of the protocol, used in A, was exchanged by 100 ng/mL IFNγ (48 hours stimulation), C1 reduced the NO release in a dose dependent manner, reaching the same level as the negative control when using 2.5 μM C1 (control 29% versus treated 18%, p=0.1253) and DMSO reduced the IFNγ induced NO release in these cells independently of the applied dose (1.25×10-5 v/v: 85%, p<0.0043; 12.5×10-5 v/v: 83%, p<0.0016; 125×10-5 v/v: 85%, p<0.0046), similar to the effect observed in microglia when stimulated with IFNγ. D) Using the stimulation protocol as in A, B, and C respectively, the influence on macrophages metabolic activity after stimulation 1 μg/mL LPS (for 48 hours (D), 100 μg/mL PolyIC for 24 hours (E), or 100 ng/mL IFNγ for 48 hours (F) was measured using the AlamarBlue assay. After LPS stimulation (A) C1 caused a dose independent decrease in cell activity for all 3 concentrations of C1 (0.025 μM: 78.47±31.61%, p=0.0245; 0.25 μM: 69.31±36.50%, p=0.001; 2.5 μM: 66.48±31.17%, p<0.001) in comparison to the positive control (100±4.74%) and when cells were treated with 2.5 μM C1 there was a reduction of the cell activity also when compared to the DMSO control (p=0.0310). Treatment with DMSO did not show a significant decrease. Stimulation with PolyIC led to a reduction of metabolic activity after treatment with 0.025 μM C1 compared to DMSO stimulation (p=0.0053) and the positive control (p=0.0138). Higher concentrations of C1 did not show a significant difference. Stimulation with IFNγ led to significant decrease in metabolic activity after treatment with 0.25 μM (p=0.0283) and 2.5 μM (p=0.0357) compared to the positive control, but not their innate DMSO control. The pro-inflammatory cytokines IL1β, IL6, and TNFα, were measured after a 48 hours LPS (1 μg/mL) stimulation. The protocol was performed as noted in A. The LPS induced release of IL1β and IL6 was not changed in the presence of C1 or DMSO. DMSO showed a significant effect on the release of TNFα in the concentrations 1.25×10-5 v/v (p<0.0001) and 12.5×10-5 v/v (p=0.005). 2.5 μM C1 decreased the release of TNFα significantly to 91% compared the positive control (p=0.0313). *** p<0.001 ** p<0.01 * p<0.05 comparing to stimulated control, ###p<0.001 ##p<0.01 ##p<0.05 comparing to DMSO (1 way ANOVA followed by Bonferroni's post-hoc test). H) The metabolic activity of primary cultured neonatal microglia, primary cultured neonatal astrocytes and the oligodendrocyte cell line OLN-93 was evaluated under physiological condition (unstimulated). The cells were treated for 48 hours with 2.5 μM C1 (in dark grey), or its corresponding concentration of DMSO (125×10-5 v/v, in light grey) or plain medium (in white). DMSO (p=0.0439) and C1 (p=0.0402) showed a significant increase in metabolic activity on microglia compared to plain medium. Comparing DMSO and C1 showed no significant difference. DMSO and C1 showed no influence on astrocytes. C1 increased the metabolic activity in oligodendrocytes compared to DMSO (p=0.0431) but not compared to plain medium (p=0.2140). I) Using the same experimental setup as in A, the proliferation and cell death was measured using a propidium iodide based assy. C1 increased the proliferation in microglia significantly compared to plain medium (p=0.0154) and DMSO (p<0.0001), but did not show an effect on the other tested cell types. DMSO did increase the proliferation of astrocytes compared to plain medium (p=0.0060). K) DMSO did increase the percentage of dead cells in microglia significantly compared to plain medium (p=0.0096). Treatment with C1 kept the percentage of dead cells similar to plain medium (C1: 8.49%, plain medium: 8.26%) with not significant difference. However, it showed a significant reduction from the elevated DMSO level (p=0.0201). ** p<0.01 * p<0.05 comparing to stimulated control, ###p<0.001 ##p<0.01 #p<0.05 comparing to DMSO (1 way ANOVA followed by Bonferroni's post-hoc test)

FIG. 8: shows that mice survive 24 hours of compound treatment. Distribution of C1 in the blood plasma after a single intravenous injection into healthy mal mice (5 mg/kg, n=3) over the time period of 24 hours. The concentration within the blood plasma was measure 5, 15, 30 min, and 1, 2, 4, 6, 8, and 24 hours after injection. The concentration in the blood plasma declined exponential, starting at 5 min at 11.89 μM ending at 1.26 nM after 24 hours. All mice survived the 24 hours of treatment with C1.

FIG. 9 shows that compound C1a, which corresponds to the compound of formula (18), but not compound C1b, which corresponds to the compound of formula (50) shows an inhibitory effect in the NO release assay.

### EXAMPLE SECTION

### Example 1—Material and Methods

Solutions. If not otherwise stated, all cells were incubated in Dulbecco's Modified Eagle Medium (DMEM) (GIBCO, Thermo Fischer Scientific, Waltham, USA), containing 10% fetal calf serum (FCS) and Penicillin-Streptomycin-Glutamine (Thermo Fisher Scientific), in this manuscript referred to as “plain medium”.

Primary cultured neonatal microglia. For preparation of cultured neonatal microglia, P0-P3 mice were used. 10 brains were extracted, meninges and cerebellum were removed and put on ice with Hank's balanced salt solution (HBSS). The dissected brains were washed 3 times before adding 400 μL trypsin (0.1 mg/L) and DNAse (5 μg/L) in phosphate buffered saline (PBS). After 2 min of incubation with this solution, the reaction was blocked by adding plain medium. After removal of the medium, 1 mg DNAse was added and the cells were mechanically dissociated, with a Pasteur pipette followed by a glass pipette. The cells were centrifuged for 10 min at 129 g at 4° C. The supernatant was discarded. The pellet was resuspended in plain medium and plated (2.5 brains/flask) in Poly-L-Lysine coated flasks. The cells were washed after 2 days with PBS and incubated for another 7 days in plain medium. Afterwards the medium was replaced by plain medium with 33% L929 conditioned medium. After 2 days, cells were harvested by shaking them for 30 min at 150 rpm. The supernatant was centrifuged for 10 min at 129 g at 4° C. The cells were plated into a 96 well plate (100 000 cells/well, 200 μL medium per well). This procedure could be repeated 2 times with a 2 day interval.

Primary cultured neonatal astrocytes. The preparation of primary cultured neonatal astrocytes followed the preparation of the primary cultured neonatal microglia, described above. After the third shake off, the remaining astrocytes were trypsinated, washed with PBS and seeded into a 96 well plate (10 000 cells/well, 200 μL medium per well).

Primary cultured macrophages. Bone marrow macrophages were isolated from C57BL/6 adult mice (P49-56) as previously described (Marim et al., 2010). In summary, mice were killed by cervical dislocation and the femurs were removed by cutting through the tibia near the pelvic bone and below the knee. Any muscle connected to the bone was carefully removed. Ice cold sterile PBS was slowly flushed through the bone and the contents were collected in a sterile 15 mL polypropylene tube on ice. After centrifugation for 10 min at 200 g at 4° C., red blood cells were lysed by adding 3-10 mL ammonium chloride solution. After a 3-5 min recovery period, the suspension was centrifuged for 10 min at 200 g at 4° C. and the supernatant was removed. Cells were plated in a 10 cm dish with plain medium containing 10 ng/mL M-CSF and cultured for 7 days to allow differentiation. 50 000 cells were plated with 200 μL medium per well in a 96 well plate.

Cell culture of immortalized cell lines: microglia cell line BV2, and oligodendrocyte cell line OLN-93. The immortalized murine microglia cell line BV2 (Blasi et al., 1990) and the oligodendrocyte cell line OLN-93 (Richter-Landsberg and Heinrich, 1996) were incubated in T75 flasks with plain medium. The cells were split every 2 to 3 days at the dilution 1:10 before they reached confluence. To do so, the cells were washed twice with PBS, followed by up to 5 min of trypsination. The trypsination was stopped using plain medium. The cells were centrifuged at 300 g for 10 min at 4° C., the supernatant was discarded, and the cells were resuspended in new medium and seeded into a new flask.

Compound Library. A library of 16 544 compounds (ChemBioNet library) was tested at the Screening Unit core facility of the Leibniz-Forschungsinstitut für Molekulare Pharmakologie. The library consists of a diversity set that was designed on the basis of the maximum-common substructure principle. The screening libraries are arranged on 384-well microtiter plates, in which compounds are placed into columns 1-22 as 10 mM solutions dissolved in DMSO. DMSO alone is placed into columns 23 and 24, therefore permitting to screen 352 compounds per plate together with 32 controls.

High throughput screening (HTS). Using a dispenser (EL406, Biotek, Winooski, USA), BV2 cells were seeded at a cell density of 5 000 cells per well/40 μl per well into a 384-well plate (3683, Corning, New York, USA). The plates were covered with a lid and incubated for 24 hours at 37° C., 5% C02 and 95% humidity. Using a robotic liquid-handler (Freedom Evo, Tecan, Maennedorf, Switzerland), compounds were pre-diluted in cell medium to 500 μM. 500 n1 of this prediluted compound solutions were transferred in the wells of the assay plates. After 1 hour pre-incubation of the compounds with the cells, 10 μl of 5 μg/ml LPS solution was added to columns 1-23 using a dispenser. The final assay conditions were therefore 50 μl total volume containing 5 μM of test compound, 0.05% DMSO, and 1 μg/ml LPS. The positive controls were treated with LPS only in column 23 and negative controls were left in plain medium in column 24. Incubation with LPS and compounds was continued for 48 hours, followed by addition of 25 μl of 2-fold concentrated Griess reagent and absorbance reading at 540 nm in a plate reader (Safire2, Tecan, Maennedorf, Switzerland). Active compounds were identified by decreased Z-scores of the absorbance signal.

Experimental Design and Statistical Analysis for HTS. Data was normalized for each plate by using statistically robust estimators (Brideau et al., 2003).

$$Z - score = \frac{{x}_{i} - Median}{MAD * 1.48258} \qquad 1 )$$

Z-score indicates how many standard deviations an observation is above or below the mean (1). xi is the signal of a single sample, Median is the median signal on a plate without the controls, and MAD is the median absolute deviation on a plate without the controls.

$$Percent Activity = \frac{{x}_{i} - Neg}{\vert Pos - Neg \vert} \qquad 2 )$$

Percent activity is the response relative to an unperturbed state (2). Neg is the median of the negative (no LPS induction) control samples, and Pos is the median of the positive (=100% LPS-induced, non-treated) control samples on a plate.

$${Z}^{′} = 1 - \frac{3 * ( {δ}^{P} + {σ}^{N} )}{\vert {μ}^{P} - {μ}^{N} \vert} \qquad 3 )$$

Z′ is a common statistical tool to measure the effective dynamic signal range of HTS assays and serves as quality control metric. δₚ and δₙ are the standard deviations of the positive and negative controls, respectively, and μₚ and μₙ are the mean values of the positive and negative controls of a plate.

f(x)=c+(d−c)/(1+exp(b*(log(x)−log(e))) 4)

IC₅₀ determination was carried out using the four-parameter log-logistic function (4), and the Pipeline Pilot curve fit module for determining dose-response curves using ILRS algorithm. b: Hill-coefficient (steepness of the IC₅₀ curve at the inflection point), e: IC₅₀ value, c and d: left and right activity asymptotes.

Data were pre-processed (initial graphical quality control and data normalization) using in-house software, reports containing chemical structures where generated using Pipeline Pilot (Biovia).

Concentration-dependent validation and viability determination. Concentrated stock solutions of compounds identified from primary screening were rearranged onto anew 384-well plate, and 10 sequential 2-fold serial dilutions were created across multiple plates in DMSO. Starting from these diluted compound mother plates, the protocol was repeated exactly as for primary screening to span the concentration range between 5 μM and 20 nM. In order to obtain the 10 μM and 20 μM concentration points, 1000 n1 and 2000 n1 were transferred from the 500 μM pre-dilution plate, respectively. Each concentration was measured in duplicate. Normalized percent activities were plotted against the compound concentration to obtain the IC₅₀ values. To obtain also information about the impact of compounds on cell viability, a second batch of plates was prepared as described above, but 5 μl of Alamar Blue solution was added after 48 hours instead of Griess reagent and incubation was continued for additional 4 hours at 37° C. Finally, the resorufin fluorescence was detected at an excitation wavelength of 530 nm and emission of 570 nm. Since no control samples were present on the assay plate for data normalization of cell viability, the raw data values were added on the second y-scale to the IC₅₀ plots.

Griess assay. Cells were seeded according to the protocols described above, after compounds and stimuli were added, 100 μl of supernatant was transferred into a new 96 well plate and 100 μl freshly mixed Griess reagent were added. Griess reagent was composed of reagent A (100 mg Naphthylethylene in 50 ml aqua dest.) and reagent B (1 g Sulfanilamide, 6 L H3PO4 (85%) in 44 mL aqua dest.) mixed 1:1. The solution was carefully mixed and the absorbance was determined in a microplate reader at a wavelength of 550 nm.

Dissolved sodium nitrite in plain medium served as a standard. Total amount of nitrite was calculated by a linear regression of the standard curve.

AlamarBlue assay. Cells were seeded according to the protocols described above, after compounds and stimuli were added, the cells were washed once with HBSS (37° C.) and 100 μl of a 1:10 AlamarBlue (Thermo Fisher Scientific) dilution in plain medium was added. The conversion of resazurin to resorufin was measured by absorbance (absorbance wavelength of 570 nm and a reference wavelength of 600 nm) after 3 hours.

Enzyme-linked immunosorbent assay (ELISA). Cells were seeded according to the protocols described above, after compounds and stimuli were added, the supernatant was collected, centrifuged (500 g, 5 minutes) and stored at −20° C. until analysed. The following kits from Bio-Techne (Minneapolis, USA) were used to detect IL1β, IL6 and TNFα: ELISA Mouse IL-1β/IL-1F2 Duo Set, ELISA Mouse IL-6, ELISA Mouse TNFα. ELISA assays were performed according to the manufacturers protocol.

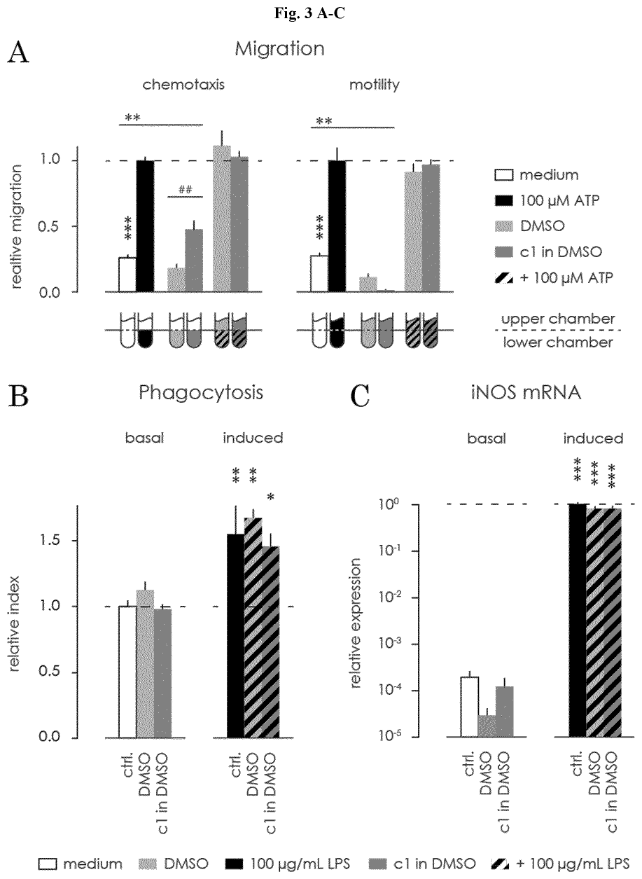

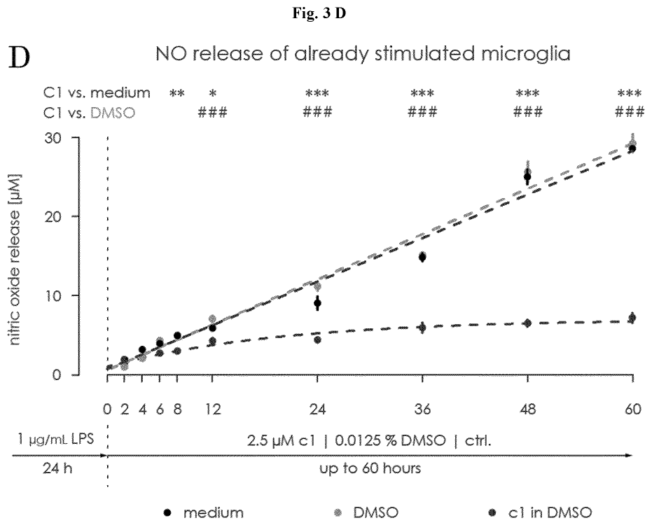

Microchemotaxis assay. The effect of the compound on directed migration and general motility was tested using a 48-well microchemotaxis Boyden chamber (Neuroprobe, Gaithersburg, USA). Upper and lower wells were separated by polycarbonate filter (8 μm pore size; Poretics). Microglial cells (2-4×10⁴ cells) in 50 μl plain medium were added to the upper compartment. In addition, 2.5 μM of compound in DMSO, 125×10-5 v/v DMSO respectively, and/or 100 μM ATP were added to the upper and/or lower chamber, as shown in FIG. 3A. Plain medium was used as a control. The chamber was incubated at 37° C. and 5% CO₂ for 6 h. Cells remaining on the upper surface of the membrane were removed by wiping, and cells in the lower compartment were fixed in methanol for 10 min and subjected to Diff-Quik stain (Medion Grifols Diagnostics AG, Dudingen, Switzerland). The rate of microglial migration was calculated by counting cells in four random fields of each well using a 20×bright-field objective. All data were normalised to ATP induced chemotaxis.

Flow cytometry-based phagocytosis assay. 10⁶ primary cultured microglial cells were seeded into 3.5 cm dishes in plain medium overnight at standard conditions. The cells were treated for 1 hour with 2.5 μM C1 in DMSO, 125×10⁻⁵ v/v DMSO respectively, or plain medium only. To stimulate microglia, LPS (1 μg/ml) was added for additional 24 hours. Fluoresbrite Carboxylate Microspheres (BrightBlue, 4.5 μm, Polyscience, Niles, USA) were coated with fetal calf serum for 30 min at room temperature, and subsequently centrifuged at 3000 g for 2 min at room temperature. The beads were resuspended in HBSS at a final concentration of 2×10⁶ beads per ml. The microglia culture was washed once with HBSS (37° C.) before 1 ml bead solution was applied. The cells were incubated with the beads for 30 min at 37° C. Afterwards, microglia were washed twice with ice cold HBSS, scratched off and pulled down at 500 g for 5 min. The cells were resuspended in a propidium iodide solution (1:200 in HBSS) to stain dead cells. The stained cells were transferred to a BD LSRFortessa Flow cytometer (BD Bioscience, Sparks, USA). The median intensity of the bright blue beads was calculated using Flowlo v10 software (Ashland, USA). The data of each experiment was normalised to the unstimulated media control.

Quantitative PCR. The same stimulation protocol as for the Flow cytometry-based phagocytosis assay was applied. Cells were seeded overnight, treated with 2.5 μM C1 in DMSO, 125×10⁻⁵ v/v DMSO respectively, or plain medium only for 1 hour, followed by an additional stimulation with 1 μg/ml LPS for 24 hours. Total RNA was isolated using the RNeasy Plus Mini Kit (Qiagen, Hilden, Germany). On-column DNase 1 (Qiagen) digestion was performed and total RNA was eluted in RNase-free water. RNA yield was measured using a Nanodrop 1000 (Thermo Fisher Scientific) spectrophotometer and quality was assessed using an Agilent 2100 Bioanalyzer (Agilent, Santa Clara, USA). Samples were stored at −80° C. until further use. First-strand cDNA synthesis was done with the SuperScript II reverse transcriptase (Thermo Fisher Scientific) using oligo-dT primers12-18 (Invitrogen) according to the manufacturer's instructions. Quantitative real-time PCR (qRT PCR) reactions were performed in a 7500 Fast Real-Time thermocycler (Thermo Fisher Scientific) using the SYBR Select Master Mix (Thermo Fisher Scientific) according to the manufacturer's instructions. cDNA input ranged between 1 and 5 ng/μl of total RNA transcribed into cDNA. The expression results were normalized to the expression of PActin of the same sample.

Propidium iodide based proliferation and cell death assay. To determine the relative proliferation and cell death, the cells were seeded into a 95 well plate, let adhere for 24 hours and afterwards treated for additional 48 hours. The supernatant was removed, and the cells were washed carefully with 37° C. HBSS once. 1/200 propidium iodide PBS solution was added and the cells were incubated for 10 minutes. The intensity of the propidium iodide signal of the dead cells was measured using a microplate reader. Afterwards, all cells were killed with a 10 min incubation of 10% DMSO and the propidium iodide signal of all cells was measured. All signals were corrected for the background noise, subtracting the blank signal. The percentage of dead cells was calculated by dividing the dead-cell-signal by the all-cell-signal. Proliferation was calculated as a relative value to the plain medium control.

Intravenous Pharmacokinetic Study in Mice. The intravenous pharmacokinetic study in mice was carried out by the company Touchstone Biosciences (Plymouth Meeting, USA) according to their standard procedures. In brief, 3 male adult mice of the CD-1 strain were fasted overnight. 5 mg/kg compound was given intravenously in one shot. After 5, 15, 30 min, and 1, 2, 4, 6, 8, and 24 hours blood samples were collected from the vein and analysed via Liquid chromatography-mass spectrometry. After 24 hours all mice were killed. The intravenous tissue distribution study in mice was carried out by the same company. In brief, 3 male adult mice of the strain CD-1 per time point (4 time points) where fasted overnight. 5 mg/kg compound was given intravenously. After 30 min, 1, 2, or 4 hours the blood, brain, heart, liver and kidney were extracted and the level of compound concentration for each organ and the blood were calculated using Liquid chromatography-mass spectrometry.

Animals and Group Allocation for middle cerebral artery occlusion (MCAO) study. C57BL/6 (13 weeks old, Charles River, Germany) male mice were handled according to governmental (LaGeSo-G0249/15) and internal (MDC/Charitd) rules and regulations, having free access to food and water. A total of 24 male mice were analysed after a 30 minutes of left-sided MCAO. Animals were randomly attributed to treatment paradigms, and experimenters were blinded at all stages of interventions. The mice were injected intraperitoneally (i.p.) every day for the 7 days, subsequently of the behavioral tests, with the compound (n=11) or with the vehicle (125×10⁵ v/v DMSO, n=13).

Induction of cerebral ischemia. Mice were anesthetized for induction with 3-4% isoflurane and maintained in 1.5% isoflurane in 70% N₂O and 30% O₂ using a vaporizer. MCAO was essentially performed as described elsewhere (Endres et al., 2000). In brief, brain ischemia was induced with a silicone rubber-coated monofilament 7-0, diameter 0.06-0.09 mm, length 20 mm; diameter with coating 0.19 0.01 mm; coating length 9-10 mm. The filament was introduced into the internal carotid artery up to the anterior cerebral artery. Thereby, the middle cerebral artery and anterior choroidal arteries were occluded. The filament was removed after 30 min to allow reperfusion.

Determination of Infarct Volume

Magnetic Resonance Imaging

MRI was performed using a 7 Tesla rodent scanner (Pharmascan 70/16, Bruker BioSpin, Bruker, Billerica, USA) with a 16 cm horizontal bore magnet and a 9 cm (inner diameter) shielded gradient with an H-resonance-frequency of 300 MHz and a maximum gradient strength of 300 mT/m. For imaging a 20 mm-1H-RF quadrature-volume resonator with an inner diameter of 20 mm was used. Data acquisition and image processing were carried out with the Bruker software Paravision 5.1. During the examinations mice were placed on a heated circulating water blanket to ensure constant body temperature of 37° C. Anaesthesia was induced with 2.5% and maintained with 2.0-1.5% isoflurane (Forene, Abbot, Wiesbaden, Germany) delivered in an O₂/N₂ mixture (0.3/0.7 L/min) via a facemask under constant ventilation monitoring (Small Animal Monitoring & Gating System, SA Instruments, New York, USA). For imaging the mouse brain a T2-weighted 2D turbo spin-echo sequence was used (imaging parameters TR/TE=4200/36 ms, rare factor 8, 4 averages, 32 axial slices with a slice thickness of 0.5 mm, field of view of 2.56×2.56 cm, matrix size 256×256).

Image Analysis

Calculation of lesion volume was carried out with the program Analyze 10.0 (AnalyzeDirect, Inc., Overland Park, USA). The hyperintense ischemic areas in axial T2-weighted images were assigned with a region of interest tool. This enables threshold based segmentation by connecting all pixels within a specified threshold range about the selected seed pixel and results in 3D object map of the whole stroke region. Further the total volume of the whole object map was automatically calculated.

Motor Deficits Assessment

Accelerated Rotarod Test

The Rotarod test was performed to access motor coordination using a treadmill with a diameter of 3 cm (TSE Systems, Chesterfield, USA). This test was performed with accelerating velocity (4-40 rpm) and maximal velocity was achieved after 300 s. The time until the animals dropped was measured. Animals were trained on day 2 and 3 before MCAO and baseline was taken on the day before MCAO. Tests were always performed four times and means were used for statistical analysis.

Pole Test

A vertical pole (80 cm high with rough surface) was used for this test, to analyse extrapyramidal motor locomotion. Mice were placed head upward on the top of the pole. The time taken to orientate the body completely downwards, making a 180° turn (t turn), and to reach the floor with all four paws (t down) were recorded. If the animal was unsuccessful for either task, it was scored the maximum time that any other animal from the same experimental group took to perform the task. Animals were trained on day 2 and 3 before MCAO and baseline was taken on the day before MCAO. Tests were always performed after the accelerated Rotarod test and repeated four times and means were used for statistical analysis.

Corner Test

Each mouse was placed on a cage containing two vertical boards attached to each other forming an angle of 300 in 2 of the corners. The side chosen to leave the corner once it made contact to the boards with its whiskers was observed within 10 trials per day. Whereas healthy animals leave the corner without side preference, mice after stroke preferentially leave the corner towards the non-impaired (i.e., left) body side (Zhang et al., 2002). Baseline side preference was accessed on day 5 before MCAO and the mice were tested again on day 6 after MCAO.

Experimental design and statistical analysis. To determine the number of mice in each group, the inventors used previous experimental data and G power analysis. Based on previous experiments, in order to detect a difference in the lesion size with 90% power, using the GPower® software (Heinrich Heine, University of Duesseldorf, Germany), the inventors determined to need 11 mice in each group. The inventors considered a 30% drop out rate due to the experimental procedure. They started with a number of 36 mice: 6 mice (4 treated and 2 controls) displayed no lesion on MRT scans and were therefore excluded from the study, 2 mice died during the MCAO procedure and 2 mice died on day 4 after MCAO (one in each group). One mouse in each group was excluded from the study since their ischemic volume was considered a significant outlier within the group, using the Grubbs' test. Thus, 24 out of 36 mice were included into the analysis (34% drop out rate). The inventors have then performed the experiments with 11 mice in the treatment group and 13 in the control group. Statistical evaluation was performed using PRISM version 5.0 software (GraphPad, La Jolla, USA). Data from experiments using animals were analysed using planned comparisons to test the following questions of primary interest 1) is the compound able to improve stroke related motor impairment and 2) does the compound have an effect on the ischemic lesion volume. Comparisons between multiple experimental groups were made using one or two-way ANOVA with Bonferroni's post-hoc test when appropriate. For comparisons between a single experimental group and a control group, the inventors used Student's t test. p<0.05 was considered to be statistically significant. Data are given as mean±SEM. Details on statistical analyses and experimental design, including which tests were performed, exact p-values (within resolution of software limits), sample sizes (n), and replicates, are provided within the legend of each figure.

NMR analysis. ¹H-NMR and ¹³C-NMR spectra were recorded either on AV 300 MHz or on AV 600 MHz from Bruker. Chemical shifts are recorded in parts per million (ppm). Spin multiplicities are described as s (singlet), d (duplet), t (triplet), q (quartet) and m (mulitplet). Coupling constant (J) are recorded in Hz. NMR data were analyzed with MestReNova software.

Mass spectrometry analysis. Mass analyses were performed with two different spectrometers using the same column. HRMS: Instrument: Agilent Technologies 6230 Accurate Mass TOF LC/MS linked to Agilent Technologies HPLC 1260 Series; Column: Thermo Accuore RP-MS; Particle Size: 2.6 μM Dimension: 30×2.1 mm; Eluent A: H₂O with 0.1% TFA Eluent B: MeCN with 0.1% TFA; Gradient: 0.00 min 95% A, 0.2 min 95% A, 1.1 min 1% A, 2.5 min Stoptime, 1.3 min Posttime; Flow rate: 0.8 ml/min; UV-detection: 220 nm, 254 nm, 300 nm. LCMS: Instrument: Agilent Technologies 6120 Quadrupole LC/MS linked to Agilent Technologies HPLC 1290 UV-detection: 215 nm, 254 nm.

### Example 2—Screening for Compounds Inhibiting Nitric Oxide Release in Microglia

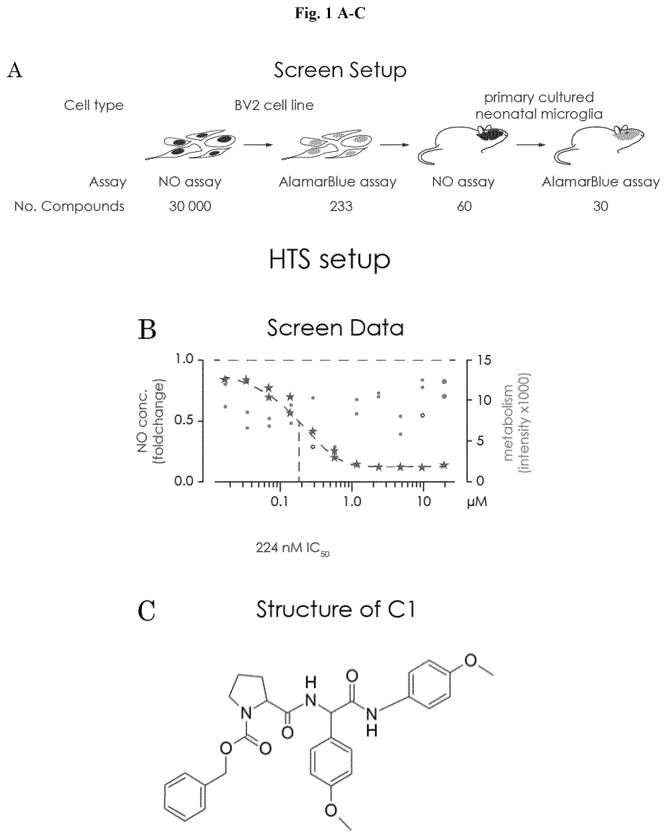

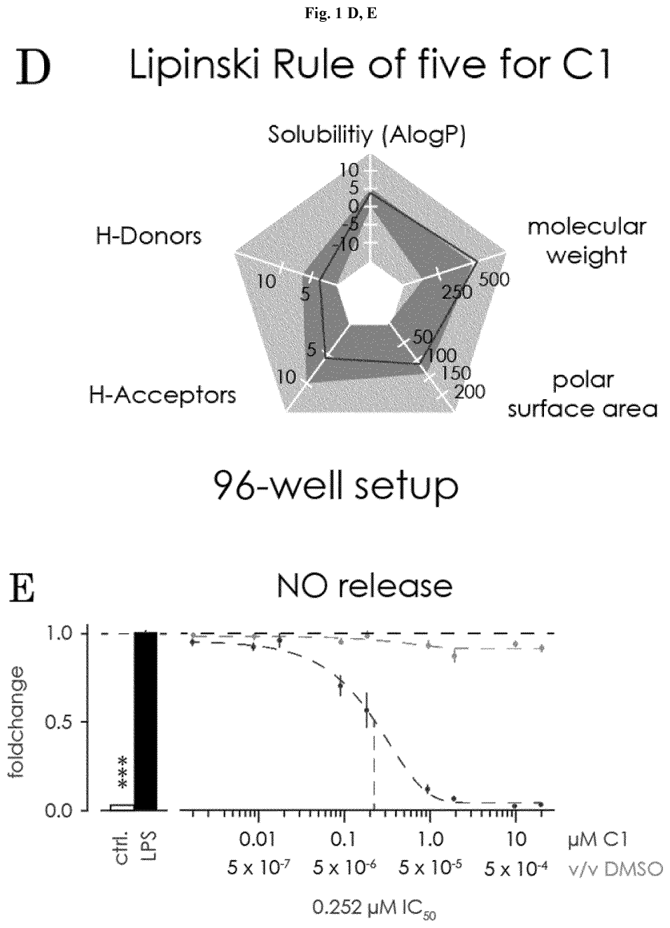

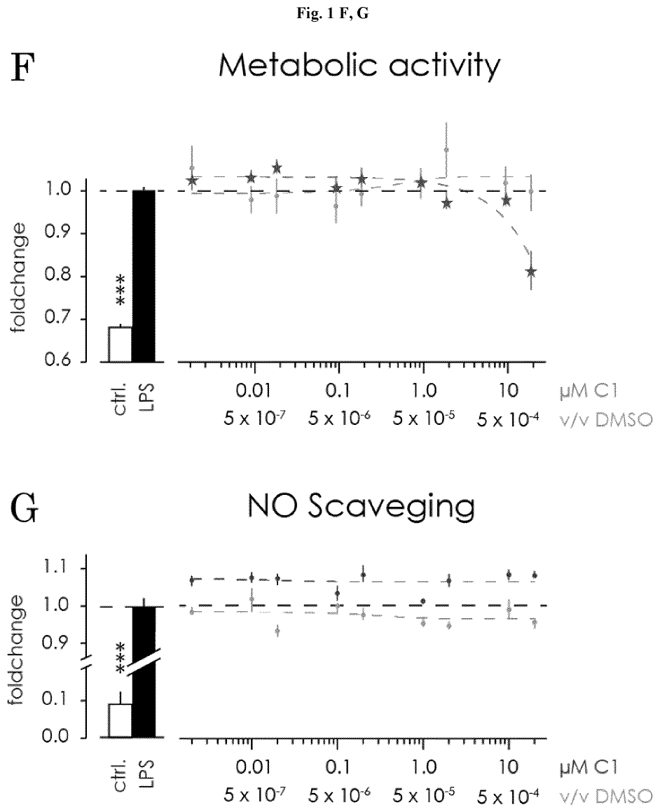

A screening from a small molecule library containing 16 544 compounds was performed in order to identify compounds that inhibit the lipopolysaccharide (LPS) induced NO release in microglia. The screen was performed with the microglial cell line BV2 which allowed a HTS approach (Das et al., 2016). The NO concentration in the supernatant was measured using a modified Griess assay (Amano and Noda, 1995). The overall experimental strategy of the HTS is outlined in FIG. 1A, the assay plates showed a signal separation suitable for HTS with a mean Z′-factor of 0.75 (based on the signals of LPS-induced versus plain medium). The compounds were preselected to cover a broad diversity of chemical structures and the “Lipinski rule of 5” was taken into account to ensure general bio availability (Lipinski et al., 2001; Lipinski, 2016). BV2 cells were incubated for 24 hours, pre-treated with the compounds of the library for 1 hour and subsequently stimulated with 1 μg/ml LPS for additional 48 hours. Primary screening was conducted at a compound concentration of 5 μM, out of 16 544 samples tested, 503 samples showed a Z-score <−5. The 352 most active samples were re-picked for concentration-dependent validation, where the compound concentration ranged from 19.5 nM up to 20 μM. The inventors identified 233 compounds that reduced the NO concentration in a dose dependent manner. Those compounds were also tested for their impact on the BV2 cell viability, using the AlamarBlue assay. 60 out of 233 compounds reduced the NO release without compromising cell viability. These non-toxic compounds were further tested on primary murine microglia derived from neonatal cultures. 30 out of 60 compounds reduced the LPS-stimulated NO concentration in primary neonatal microglia cells. Testing for cell viability using the AlamarBlue assay lead to the reduction to a number of 4 compounds, capable of reducing LPS induced NO release, while not interfering with the cell viability of primary microglia. Out of those 4 compounds, the inventors choose the one (C1) with the best IC₅₀ value, of approximately 224 nM, in reducing NO release (indicated by the red dashed line, FIG. 1B).

C1 is a peptide-like small molecule (FIG. 1C). Its structure is based on 4 subgroups of proteinogenic amino acids or proteinogenic amino acid like structures connected via peptide bonds. The 4 subgroups resemble the amino acids phenylalanine, proline, and 2 times tyrosine. C1's structure inherits 2 stereo centres. Both are located in the backbone of the peptide bonds, one in the amino acid proline, the other one in the amino acid like structure of tyrosine. The stereo centre in proline amino acid is defined in its S conformation. The stereo centre in the amino acid like structure of tyrosine occurs in both S and R conformation. In the following study, if not stated otherwise, all experiments were performed with diastereomeric mixture of the S and R conformation of the 4-Methoxyphenylgycine attached to Cbz-protected (S)-proline. Set as criteria for compounds in this screen, C1 shows a general calculated drug-ability, fulfilling all the “Lipinski rule of 5” (FIG. 1D), except the one referring to the molecular weight of the molecule. It (slightly) surpasses the set molecular weight threshold of 500 g/mol (517.57 g/mol). To verify the data obtained in the HTS setup (368 well plate, FMP screening unit), the inventors repeated the dose response curve in primary microglia (96 well plate). Microglia were treated for 1 hour with C1 and subsequently stimulated with 1 μg/ml LPS for additional 48 hours. The compound concentration ranged from 2 nM up to 20 μM. The IC₅₀ value was slightly higher in primary microglia (224 nM in the HTS versus 252 nM; 95% confidence interval from 207 nM to 323 nM, R²=0.9141; FIG. 1E, in dark grey), while there were no effects on cell viability (FIG. 1F, shown as stars). LPS stimulated microglia (FIG. 1F, in black) showed a significantly increased metabolic activity by 31% compared to the unstimulated control (FIG. 1F, in white, p<0.0001). None of the tested compound concentrations did decrease the metabolic cell activity below the negative control (FIG. 1F, in white). DMSO, which was used as the solvent for the compound, did not affect the NO release (FIG. 1E, in grey) or the cell viability of microglia (FIG. 1F, shown as dots).

In order to make sure that C1 does not quench the NO concentration, the inventors incubated the compound with NO enriched supernatant taken from microglia cells stimulated with LPS for 24 hours. Neither C1 nor DMSO altered the NO concentration in the supernatant (FIG. 1G).

The inventors further tested whether C1 alone affects the NO release and metabolic activity in microglia. They therefore treated primary microglia with plain medium (negative control, shown in white, FIG. 1-1), 1 μg/ml LPS (shown in black, FIG. 1-1), 3 different concentrations of C1 (0.025 μM, 0.25 μM, or 2.5 μM, in dark grey, FIG. 1-1) or their corresponding concentration of DMSO (1.25×10-5, 12.5×10-5, or 125×10-5 v/v, in light grey, FIG. 1-1) for 48 hours. C1 and DMSO did neither change the NO release (FIG. 1-1A) nor did it interfere with the metabolic activity (FIG. 1-1B) of microglia compared to the plain medium control.

### Example 3—C1 Reduces Nitric Oxide Release Induced by IFNγ or PolyIC

In order to test whether C1 decreases NO release induced by other stimulation agents besides LPS which is mimicking a bacterial infection, the inventors used IFNγ to mimic a Th1 cell response (Yau et al., 2016) and PolyIC to mimic an anti-viral response (Jiang and Pisetsky, 2006). Given that our aim is to determine the compound's effect on already stimulated microglia (LPS, IFNγ, and PolyIC), each individual experiment is normalized to its stimulated control.

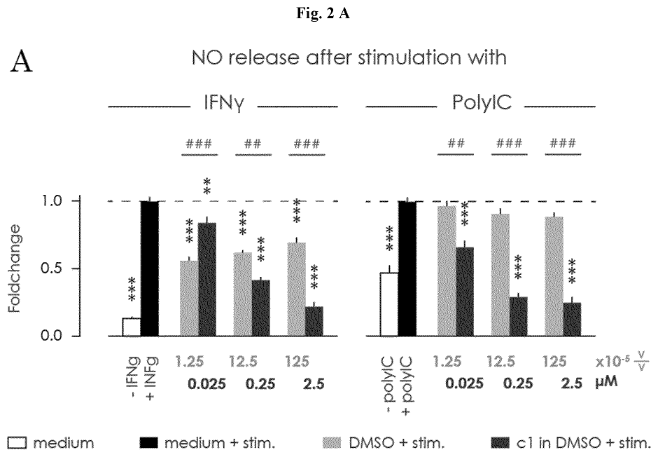

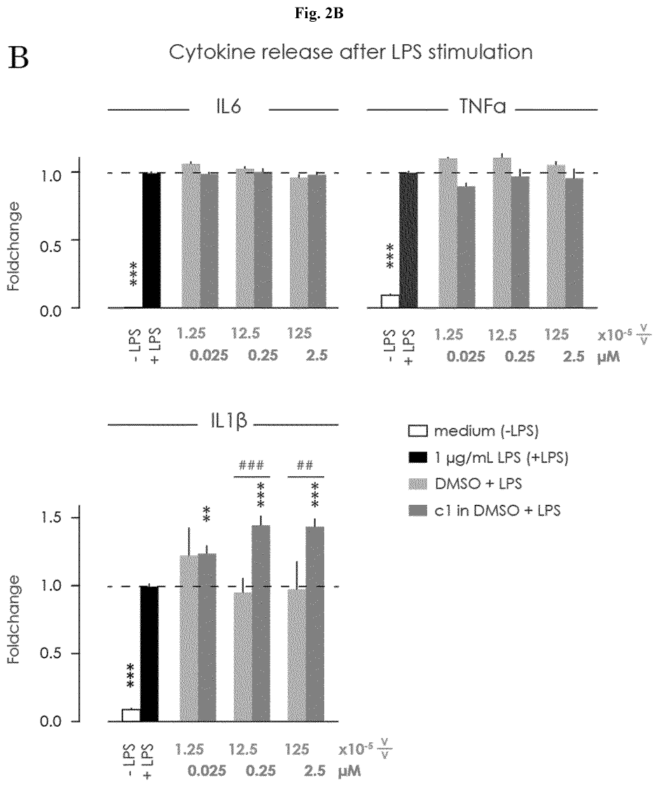

Stimulating primary microglia with IFNγ (100 ng/ml, FIG. 2A, left panel) for 48 hours evoked a significant increase in NO concentration in the supernatant compared to plain medium (−IFNγ, p<0.0001). The rise in NO production is antagonized in a dose dependent manner when the cells are treated with C1 including a pre-treatment for 1 hour. Treatment with 25 nM of C1 decreased the level of NO to 84% of its initial concentration significantly (p=0.087), 250 nM significantly to 41% (p<0.0001), and 2.5 μM to 22% (p<0.0001), a concentration indistinguishable from plain medium (p>0.9999 compared to −IFNγ). The solvent DMSO alone also decreases the IFNγ induced NO concentration by around 40% (FIG. 2A, grey bars). However, this decrease is not dose dependent and does not reach the same effect size that C1 does.

Stimulating primary microglia with PolyIC (100 μg/ml, FIG. 2A, right panel) for 24 hours increased the NO concentration in the supernatant significantly (−PolyIC, p<0.0001). The inventors used 24 hours stimulation since 48 hours stimulation decreased the cell viability (measured with the AlamarBlue assay, data not shown). Similar to the stimulation with LPS, the inventors pre-treated the cells with C1 1 hour before adding PolyIC for additional 24 hours. The concentration of NO decreased in a dose dependent manner to values below the plain medium control (−PolyIC). At a concentration of 25 nM C1 reduced the NO concentration to 66% (p<0.0001). At 250 nM C1 decreased the NO concentration to values below the plain medium control (29%, p<0.0001 compared with +PolyIC), while 2.5 μM further decreased the NO concentration to 25%, which is significantly below the plain medium control (−PolyIC, p=0.0127). DMSO showed no effect on the PolyIC induced NO levels, which were ranging from 89% to 97% of the initial concentration. Although the inventors do not investigate the effect of DMSO on microglia NO production itself, it is worthwhile to mention that the release of NO from IFNγ stimulated microglia is affected by DMSO while in LPS (FIG. 1E) and PolyIC stimulated microglia it is not. Therefore, the inventors always show the plain medium control and the DMSO control. The most important comparison is always done with the stimulated control (+LPS, +IFNγ, or +PolyIC) since the potential of C1 to inhibit NO production is investigated here.

### Example 4—C1 does not Interfere with the LPS-Induced IL6 and TNFα Release

Upon pro-inflammatory stimulation microglia produces pro-inflammatory cytokines (Kettenmann et al., 2011; Cavaillon, 2017; Wolf et al., 2017). Here, the inventors tested the effect on the release of the pro-inflammatory cytokines IL10, IL6, and TNFα. After pre-treating the microglia for 1 hour with C1 followed by additional 48 hours stimulation with 1 μg/ml LPS the supernatant was collected and the cytokine levels were measured with ELISA. In controls, levels of all cytokines increased upon stimulation as expected (for all: p<0.0001). Unlike the observed effect on the NO release, C1 did not show a reduction in the concentration of the cytokines IL6 and TNFα (FIG. 2B). Moreover, the IL1β release exceeds the stimulated control significantly (25 nM: p=0.0037; 250 nM: p<0.0001; 2.5 μM: p<0.0001; compared to +LPS, FIG. 2B). C1 alone does not alter the levels of IL1β, IL6, and TNFα (FIG. 2-1A-C) indicating that the observed increase in the IL1β concentration might be the result of a synergistic effect of the stimulation with LPS and C1. DMSO does not show any significant influence on the cytokine release, neither in stimulated (FIG. 2B, shown in grey) nor unstimulated microglia (FIG. 2-1A-C, shown in grey).

### Example 5—C1 Acts as a Chemoattractant, but Did not Affect Phagocytosis Activity

Microglial cells, as part of the innate immune system, are capable of increasing their motility, chemotaxis and phagocytic activity upon a pro-inflammatory stimulus (Kettenmann et al., 2011; Sierra et al., 2013; Wolf et al., 2017). Using the Boyden-chamber assay, it is possible to determine two types of migratory activity, chemotaxis and motility. If a gradient is present, the inventors can assess chemotactic properties, when there is no gradient, and the substance of interest is equally distributed through the chamber, the inventors can determine the effect on cell motility (principle depicted in scheme below the graph in FIG. 3A). The inventors tested both the direct effect of C1 on chemotaxis and its stimulating effect by ATP. To assess the chemoattractant potential of C1, they applied a gradient with 2.5 μM of C1 and used the corresponding 125×10-5 v/v DMSO and plain medium as controls. The number of cells migrating towards C1 was significantly higher compared to plain medium or to the DMSO (p=0.0079, FIG. 3A), indicating, that C1 has chemoattractant properties. The ATP induced chemotaxis (set to 100%) was not changed by C1 (103%) or DMSO (112%) (FIG. 3A).

Motility was assessed in the presence of 2.5 μM C1 or corresponding concentration of DMSO (125×10-5 v/v) or in plain medium on both sides of the Boyden-chamber. The solvent control, DMSO, did not significantly affect the number of migrating cells compared to the plain medium control. C1 also did not significantly affect motility compared to the DMSO control, however it did, when compared to the plain medium control (p=0.0018). The ATP induced motility was not altered in the presence of C1 or DMSO (FIG. 3A). Thus, our compound shows chemoattractant properties.

The inventors determined the phagocytic activity both under basal conditions and after LPS stimulation. Microglia were pre-incubated with 2.5 μM C1, 125×10-5 v/v DMSO respectively or with plain medium only, followed by an additional stimulation with 1 μg/ml LPS for 24 hours. Upon LPS stimulation microglial phagocytic activity increased significantly by 30% (p=0.0058, FIG. 3B) in comparison to plain medium. Treatment with C1 or DMSO did not alter the basal or stimulated phagocytic activity of microglia.

### Example 6—C1 Acts on the Posttranscriptional Level

It is known that iNOS is regulated on the transcriptional level (Aktan, 2004). Under physiological conditions iNOS's mRNA is barely detectable, while upon a pro-inflammatory stimulus the transcription of iNOS's mRNA is upregulated. Thus, the inventors further investigated whether the induction of iNOS's mRNA is affected by the treatment with C1. Microglia were pre-incubated with C1 in DMSO (2.5 μM), DMSO only (125×10-5 v/v) or plain medium for 1 hour, followed by additional 24 hour incubation with or without 1 μg/ml LPS. Since mRNA induction is rapid, the inventors did not use the 48 hours LPS stimulation time here. They quantified the amount of mRNA using qRT PCR. Stimulating the cells with LPS increased the iNOS mRNA level by 5.000 fold (p<0.0001, FIG. 3C), in comparison to plain medium. C1 or DMSO did not change the iNOS mRNA level neither under basal conditions nor after LPS stimulation.

### Example 7—C1 Also Affects NO Release after LPS Stimulation

To mimic a situation closer to a therapeutic use, the inventors investigated the effect of C1 on already stimulated microglia. After 24 hours of LPS stimulation the medium was changed and microglia were treated with 2.5 μM C1 in DMSO and compared it to 125×10-5 v/v DMSO or plain medium. The concentration of NO in the supernatant was measured at several time points up to 60 hours after medium change. The NO concentration in the supernatant of cells in plain medium or DMSO increased in a linear fashion, reaching 28 μM (+1.036 SEM) after 60 h (FIG. 3D).

When the cells were treated with C1 24 hours after LPS stimulation, there was a significant reduction in the NO concentration in the supernatant, starting at 8 hours after treatment when compared to plain medium (p=0.006), and after 12 hours when compared to the DMSO (p<0.0001). The increase of the NO concentration after C1 treatment followed a one-phase association (R² of 0.6498) and reached a calculated plateau of 7 μM (95% confidence interval 5.999 to 8.127 μM, FIG. 3D) compared to 28 μM for DMSO or plain medium. Therefore C1 is still potent to decrease the NO production by microglia even after 24 hours of LPS stimulation.

### Example 8—C1 has Similar Effects on Macrophages

Microglia and macrophages share many of their immune properties, therefore the inventors evaluated the impact of C1 on the release of NO and the pro-inflammatory cytokines IL1β, IL6, and TNFα on bone marrow derived macrophages (Amici et al., 2017; Li and Barres, 2017). Bone marrow derived macrophages were isolated from adult mice and pre-treated for 1 hour with C1 (0.025 μM, 0.25 μM, or 2.5 μM), its corresponding concentration of DMSO (1.25×10-5, 12.5×10-5, or 125×10-5 v/v), or plain medium before a stimulus was added. The NO release was measured after stimulation with either 1 μg/ml LPS for 48 hours (FIG. 2-1A), 100 μg/mL PolyIC for 24 hours (FIG. 2-1B) or 100 ng/ml IFNγ for 48 hours (FIG. 2-1C). C1 reduced the NO release in a dose dependent manner for all three stimuli. 2.5 μM C1 led to a decrease of the NO concentration to the level of plain medium (control: 6.01% versus treated: 4.85% p>0.9999), IFNγ (control 29% versus treated 18%, p=0.1253) and PolyIC (control: 24% versus treated cells: 15%, p=0.9751). DMSO had no effect on the NO release in bone marrow derived macrophages stimulated with LPS or PolyIC (FIGS. 2-1A and B). However, it reduced the IFNγ induced NO release in these cells independently of the applied dose (1.25×10-5 v/v: 85%, p<0.0043; 12.5×10-5 v/v: 83%, p<0.0016; 125×10-5 v/v: 85%, p<0.0046, FIG. 2-1C), similar to the effect observed in microglia when stimulated with IFNγ (FIG. 2A).

All tested concentration of C1 decreased the metabolic activity of macrophages stimulated with LPS significantly compared with plain medium control (0.025 μM: p=0.0245, 0.25 μM: p=0.0001, 2.5 μM: p<0.0001), but only 2.5 μM reached significance compared to its own DMSO control (125×10-5 v/v; p=0.0310) (FIG. 2-1D). The metabolic activity of PolyIC (100 μg/ml) stimulated macrophages was deceased significantly by the lowest measured compound concentration (0.025 μM) compared to plain medium (p=0.138) and to its DMSO control (1.25×10-5 v/v, p=0.0053) (FIG. 2-1E). 0.25 μM and 2.5 μM C1 decreased the metabolic of IFNγ (100 ng/ml) significantly compared to plain medium (0.25 μM: p=0.0238, 2.5 μM: p=0.0357), but not to its DMSO control (FIG. 2-1F).

The cytokine release was measured after 48 hours of stimulation with 1 μg/mL LPS using an ELISA (FIG. 2-1G). Neither the treatment with C1 nor with DMSO showed any significant changes in the release profile of IL1β and IL6 after LPS stimulation. There is, however, a dose independent decrease in the concentration of TNFα after treatment with C1 and with DMSO. 2.5 μM C1 after LPS stimulation reduced the release of TNFα by 9% (p=0.0313). 12.5×10-5 v/v DMSO reduced TNFα release by 14% (p<0.0001), 125×10-5 v/v DMSO by 10% (p=0.005). The highest concentration of C1 led to a significant reduction of the TNFα release by 5% in comparison with the stimulated control (p=0.0313). Macrophages respond similar to microglia with a slightly different metabolic profile. In order to test the effect of the compound on other cell types of the brain, the inventors next used astrocytes and oligodendrocytes.

### Example 9—Metabolism, Proliferation and Cell Death of Astrocytes and Oligodendrocytes are not Affected

The inventors evaluated the metabolic activity, proliferation and cell death of primary cultured neonatal microglia, primary cultured neonatal astrocytes and the oligodendrocyte cell line OLN-93 under physiological conditions. All cells were treated with 2.5 μM C1 in DMSO or the corresponding DMSO concentration (125×10-5 v/v) for 48 hours without any additional stimulation followed by either an AlamarBlue assay to measure the metabolic activity, or an propidium iodide based proliferation and cell death assay. The data of the AlamarBlue assay and proliferation assay were normalised to the plain medium control. The data of the cell death is given in percentage of the total amount of cells. Under physiological condition C1 and DMSO increase the metabolic activity of microglia significantly by around 15% compared to plain medium (C1: p=0.0402, DMSO: p=0.0439, FIG. 3-1 A). However, there were no significant differences between treatment with C1 and DMSO. Astrocytic metabolic activity was not affected by C1 or DMSO (FIG. 3-1 A). Compared to the plain medium control, the metabolic activity of OLN-93 cells was not affected. However, C1 showed a significant increase compared to DMSO (p=0.0431) (FIG. 3-1A). The proliferation of microglia treated with C1 increased significantly to 117% (±0.0488% SEM) compared to plain medium (100%±0.0148 SEM, p=0.0154) and DMSO (90%±0.04827 SEM, p<0.0001). DMSO, but not C1, increased the proliferation of astrocytes compared to plain medium (p=0.0060). Neither C1 nor DMSO changed the proliferation of OLN-93 cells (FIG. 3-1B). The percentage of dead microglia increased significantly in the presence of DMSO compared to plain medium (p=0.0096). Treatment with C1 in DMSO brought this value back to level of the plain medium (plain medium: 8.26%, C1: 8.49%). The difference between the elevated DMSO death rate and the C1 was significant (p=0.0201). C1 and DMSO showed no significant effect on astrocytes and OLN-93 cell death rate. The effects of the compound on the metabolism, proliferation and death of brain derived cells are minimal and thus the inventors felt safe to conduct an in vivo experiment.

### Example 10—C1 Passes the Blood Brain Barrier in an In-Vivo Mouse Model

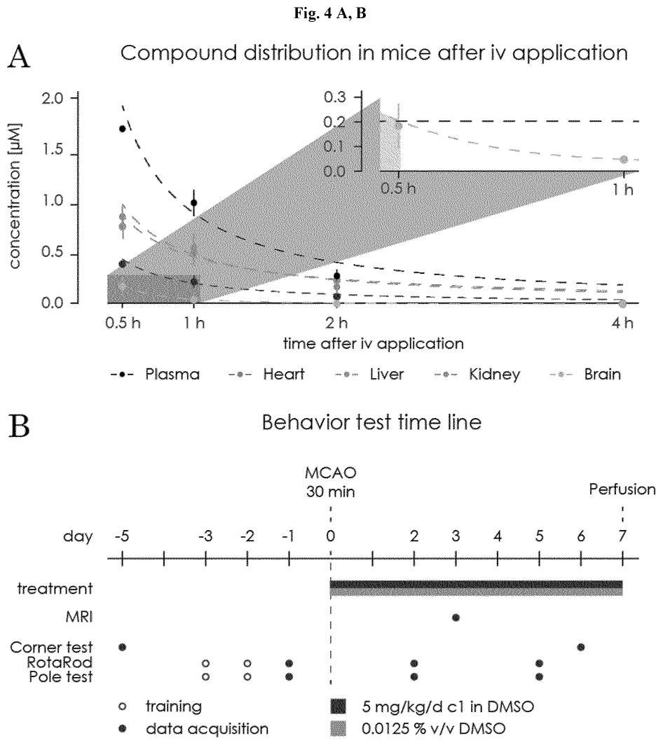

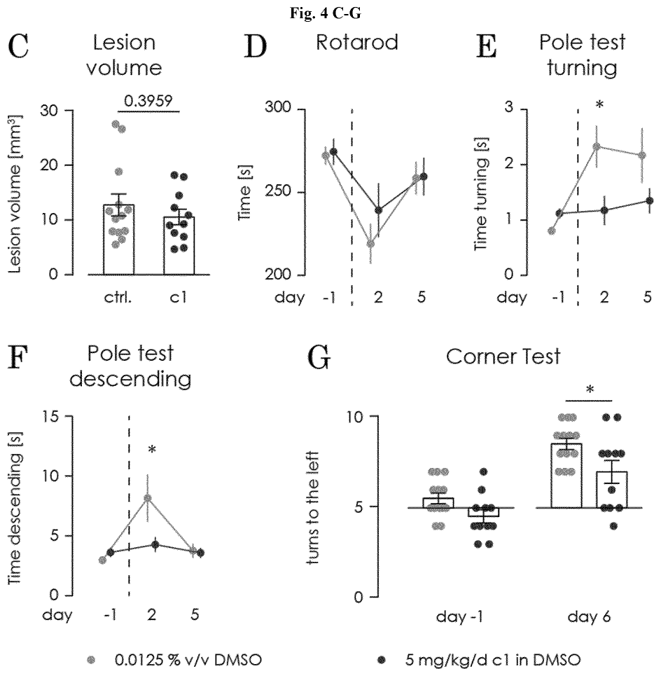

The bio availability was evaluated in-vivo in CD1 male mice performed by Touchstone Biosciences. C1's tissue distribution was monitored in the blood plasma, kidney, liver, heart and brain, over a time period of 4 hours (30 min, 1 h, 2 h, and 4 h) after an intravenous injection of 5 mg/kg into a cohort of 3 mice (FIG. 4A). After 30 minutes the concentration in brain was 1 μM/g tissue. The concentration continuously decreased and reached a concentration of 2.4 nM/g tissue, 4 hours after the injection. The level of C1 in the blood plasma was monitored for 24 hours (after 5 min, 15 min, 30 min, 1 h, 2 h, 4 h, 6 h, 8 h, and 24 h) after intravenous injection of 5 mg/kg into 3 mice (FIG. 4-1). After 5 minutes an average concentration of 12 μM was detected in the plasma and the concentration decreased within 24 hours to 1.26 nM and reached the IC₅₀ value for C1 of 250 nM between 2 and 4 hours. Within the 24 hour analysis period no health issues were reported and all mice survived.

### Example 11—C1 Improves Extrapyramidal Motor Skills and Laterality in an In-Vivo Model of Ischemic Injury

As a model for mild ischemic injury, the inventors used 30 minutes of middle cerebral artery occlusion (MCAO) in male C57Bl/6 mice (Endres et al., 2000). Mice were injected i.p. daily with 5 mg/kg of C1 diluted in 125×10-5 v/v DMSO as a vehicle in PBS or with just DMSO in PBS as control for the following 7 days after MCAO. The experimental time course of the behaviour tests, MCAO and the treatment is illustrated in FIG. 4B. On day 3 after MCAO, the inventors assessed the lesion volume using MRI. No significant difference in lesion volume was found between the groups (FIG. 4C).

As a functional read out for neurological parameters, which can also be seen in patients, the motor coordination was tested with the accelerated rotarod test and their extrapyramidal motor locomotion was assessed using the pole test. Before MCAO, the mice of both groups were trained both in the rotarod and in the pole test for 2 days and baselines were taken on the third day. On day 2 and 5 after MCAO, motor deficits were assessed and the results were compared to the baseline (FIG. 4E-G). No significant difference was found between the groups in the Rotarod test (FIG. 4E).

However, in the pole test, the treated group was significantly (p=0.0253) faster than the control group in performing the turn (1.175s versus 2.328s, FIG. 4F) and descending the pole (p=0.0145; 6.592s versus 10.47s, FIG. 4G) during the pole test on day 2 after MCAO, showing improved extrapyramidal motor locomotion.

Another important neurological parameter is laterality (the pathologic preference to turn to one side). Mice's laterality was tested with the corner test on day 6 after MCAO (see supplementary video) and the amount of turns to the left in a total of 10 trials was compared for each mouse individual baseline, taken 5 days before MCAO. The animals treated with C1 display less lateral preference (7.00 in 10 turns to the left, p=0.0213) in comparison to controls (8.538 in 10 turns to the left).

### Example 12—C1 does not Interfere with the Enzymatic Activity of iNOS, eNOS, and nNOS

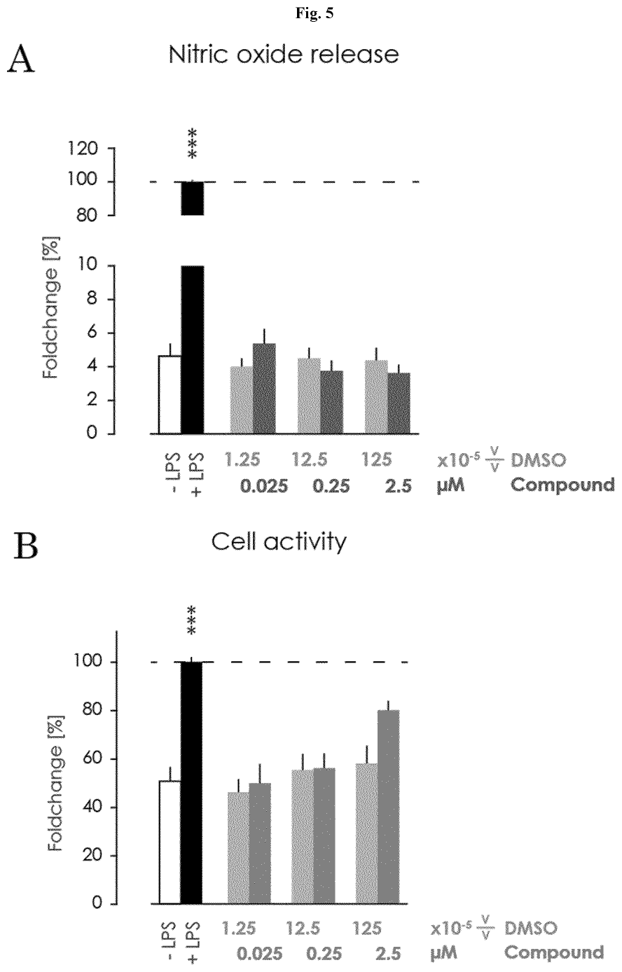

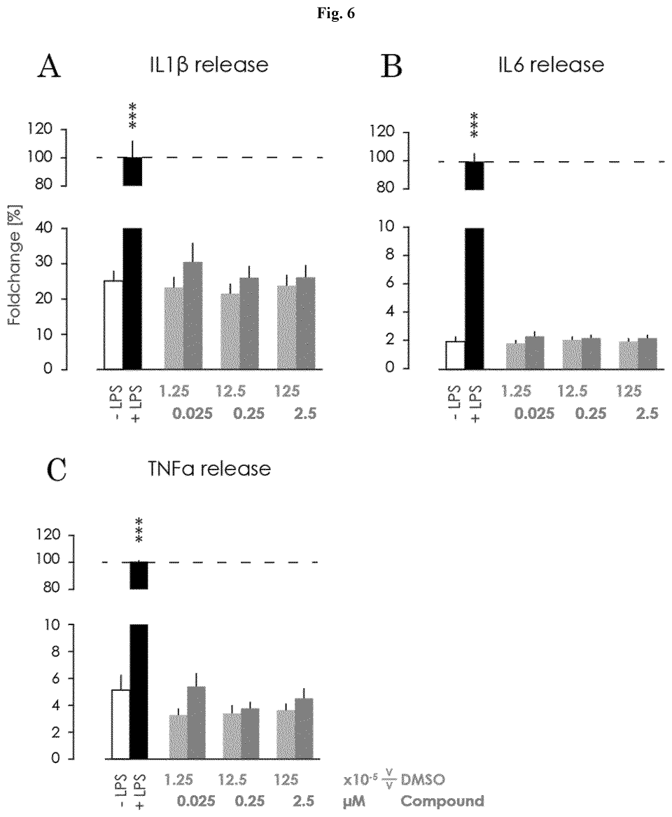

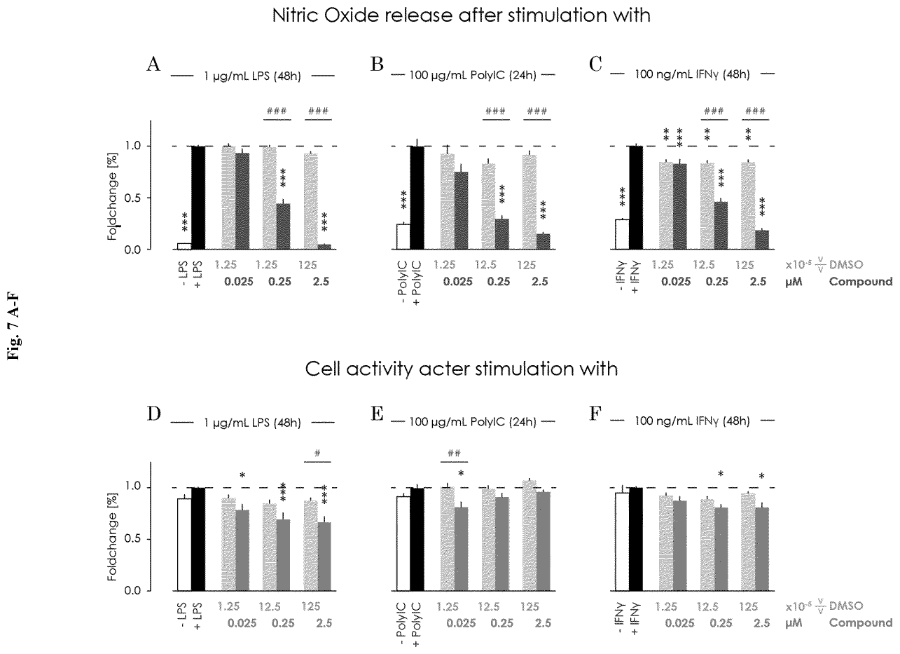

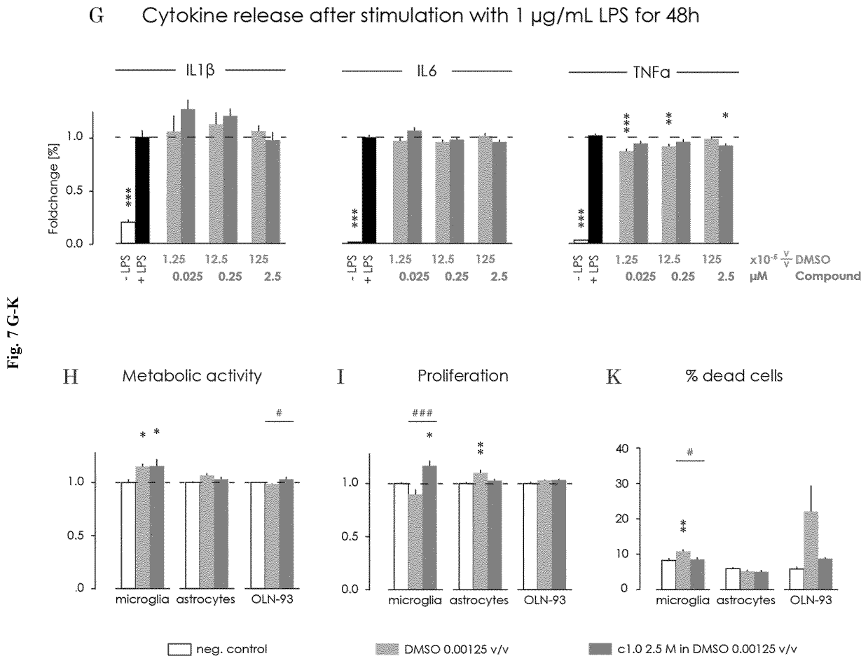

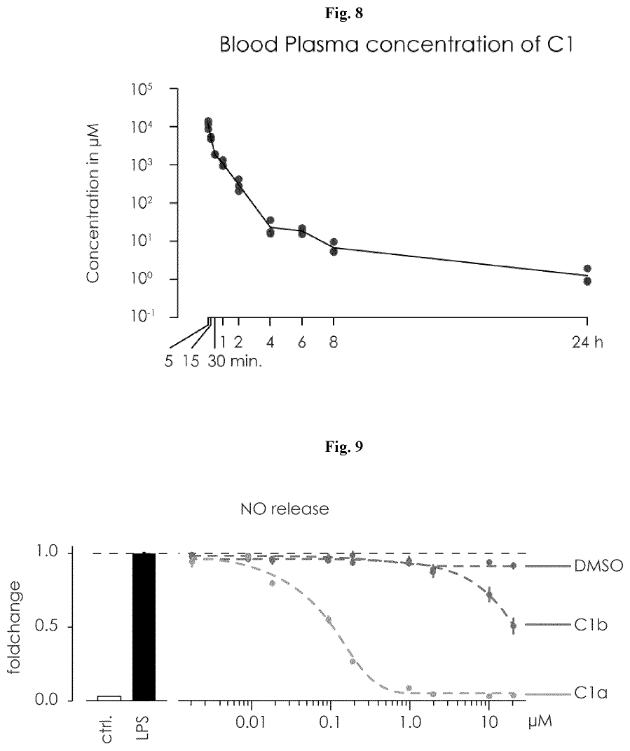

In mammalians NO is produced by the three isoforms of NOS: iNOS, eNOS, and nNOS. To evaluate the direct influence of cl on the different isoforms of NOS, an enzyme activity assay was conducted by Eurofins. C1 was mixed with the enzyme buffer, which included co-factors and substrates and by adding the enzyme the reaction was initiated. After 30 min the reaction was stopped, and NO concentration was measured. The results are normalised to positive control performed without any additional compounds and are presented as percentage in inhibition. The known inhibitors W1400 for iNOS (FIG. 5A, in black), L-NMMA for eNOS (FIG. 5B, in black), and S-Methylisothiourea for nNOS (FIG. 5C, in black) were used as reference. To test the compounds effect on iNOS a concentration range from 1 μM up to 5 μM were applied (1, 10, 100 μM, 1, 10, 100 nM, 1, and 5 μM), and for testing eNOS and nNOS 1 nM and 1 μM were applied. The known inhibitors reduce the enzyme activity as predicted (FIG. 5A-C, in black). However, C1 does not interfere with any of the three NOS isoforms within the tested concentrations (FIG. 5A, in dark grey).

### Example 13—Activity

The activity of the compounds listed in column 1 of Table 1 as inhibitors of NO production was compared in murine primary cultured neonatal microglia as described above.

TABLE 1

| No. | Synthesis No. | Mol Wt | active | IC50 μM | |
|---|---|---|---|---|---|
| 8 | KM-07-138 | 997.26 | YES | 0.008373 | +++++ |
| 5 | KM-07-120 | 507.62 | YES | 0.016740 | +++++ |
| 6 | KM-07-116 | 521.65 | YES | 0.020420 | +++++ |
| 7 | KM-07-119 | 535.67 | YES | 0.044930 | +++++ |
| 9 | ES02-03 | 533.61 | YES | 0.054810 | ++++ |
| 10 | ES02-20 | 531.62 | YES | 0.06554 | ++++ |
| 11 | ES02-02 | 517.59 | YES | 0.071680 | ++++ |
| 12 | ES02-04 | 501.59 | YES | 0.099640 | ++++ |
| 18 | KM-07-070 | 517.58 | YES | 0.1073 | +++ |
| 15 | ES02-11 | 523.63 | YES | 0.117300 | +++ |
| 16 | ES02-07 | 521.64 | YES | 0.122000 | +++ |
| 17 | ES02-21 | 487.56 | YES | 0.122 | +++ |
| 14 | ES02-08 | 505.58 | YES | 0.146800 | +++ |
| 13 | KDS-01-V73 | 515.61 | YES | 0.1513 | +++ |
| 20 | KM-07-121 | 481.58 | YES | 0.202900 | ++ |
| 19 | KM-07-118 | 507.62 | YES | 0.208500 | ++ |
| 22 | ES02-16 | 529.60 | YES | 0.2309 | ++ |
| 21 | ES02-09 | 568.68 | YES | 0.258900 | ++ |
| 23 | KM-07-117 | 493.59 | YES | 1.079000 | + |
| 24 | ES02-06 | 487.56 | YES | 1.098000 | + |
| 27 | ES02-17 | 529.60 | YES | 1.914000 | + |
| 29 | KDS-01-V83 | 993.18 | YES | 2.363 | + |
| 26 | ES02-14 | 508.60 | YES | 2.498000 | + |
| 28 | KDS-01-V74 | 483.565 | YES | 3.302 | + |
| 25 | ES02-10 | 516.60 | YES | 6.880000 | + |
| 33 | JOP-01-121 | 531.61 | YES | | +++ |
| 34 | JOP-01-125 | 531.61 | YES | | +++ |
| 30 | JOP-01-129 | 503.56 | YES | | ++ |
| 53 | KM-07-177 | 537.66 | YES | | ++ |
| 31 | JOP-01-114 | 571.67 | YES | | + |
| 32 | JOP-01-119 | 579.65 | YES | | + |
| 44 | KDS-01-V65 | 383.448 | NO | | |
| 39 | KDS-01-V72 | 501.627 | NO | | |
| 45 | KDS-01-V77 | 722.9 | NO | | |
| 46 | KM-07-115 | 464.35 | NO | | |
| 50 | KM-07-068 | 517.58 | NO | | |
| 47 | KDS-01-V80 | 425.458 | NO | | |
| 41 | ES02-18 | 540.62 | NO | | |
| 40 | ES02-13 | 491.55 | NO | | |
| 42 | ES02-19 | 571.64 | NO | | |
| 48 | ES02-15 | 528.57 | NO | | |
| 49 | ES02-12 | 488.55 | NO | | |
| 43 | ES02-22 | 487.56 | NO | | |

The activity is indicated as follows: +++++: IC₅₀ of <0.05 μM, ++++: IC₅₀ of <0.1 μM, +++: IC₅₀ of <0.2 μM, ++: IC₅₀ of <1 μM, +: IC₅₀ of <10 μM. Compounds with an IC₅₀ of ≥10 μM were considered inactive.

Compound (53) is characterized by the following formula:

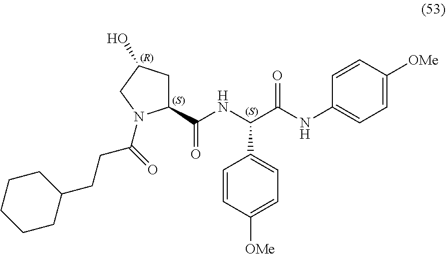

Compounds (44)-(49) are characterized by the following formulae:

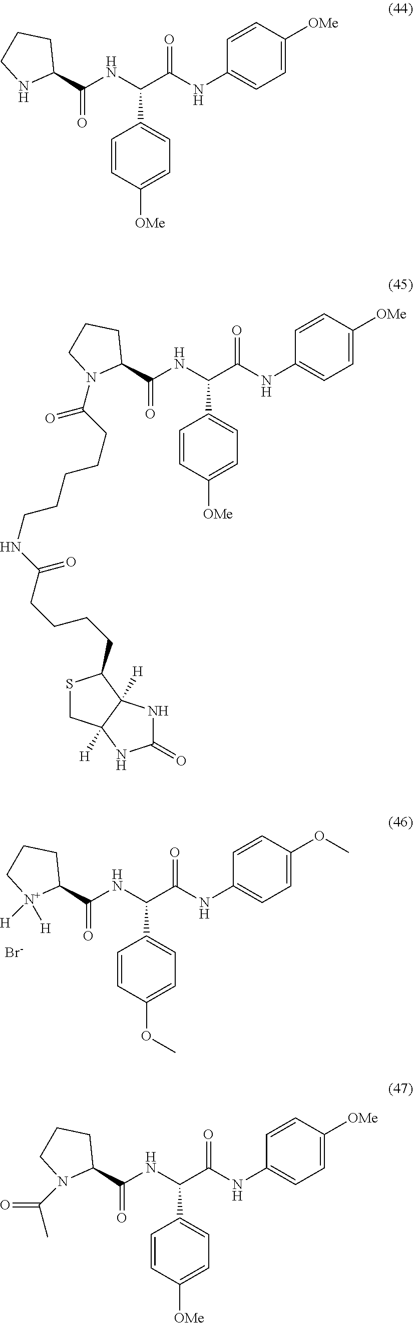 [SMILES: COc1ccc(NC(=O)[C@@H](NC(=O)[C@@H]2CCCN2)c2ccc(OC)cc2)cc1 ; COc1ccc(NC(=O)[C@@H](NC(=O)[C@@H]2CCCN2C(=O)CCCCCNC(=O)CCCC[C@@H]2SC[C@@H]3NC(=O)N[C@@H]32)c2ccc(OC)cc2)cc1 ; COc1ccc(NC(=O)[C@@H](NC(=O)[C@@H]2CCC[NH2+]2)c2ccc(OC)cc2)cc1 ; [Br-] ; COc1ccc(NC(=O)[C@@H](NC(=O)[C@@H]2CCCN2C(C)=O)c2ccc(OC)cc2)cc1]

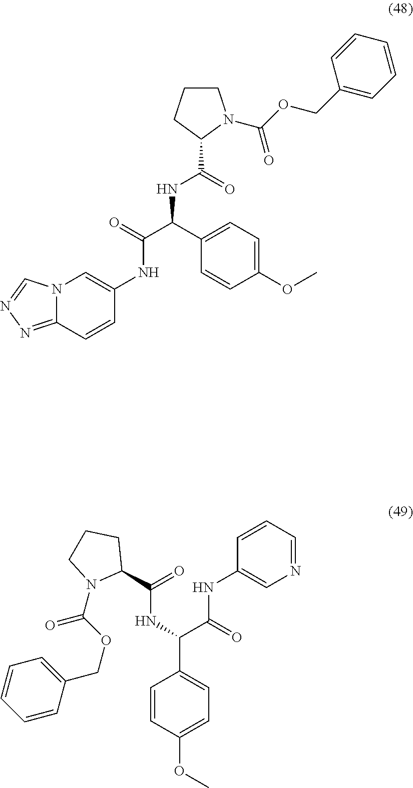 [SMILES: COc1ccc([C@H](NC(=O)[C@@H]2CCCN2C(=O)OCc2ccccc2)C(=O)Nc2ccc3nncn3c2)cc1 ; COc1ccc([C@H](NC(=O)[C@@H]2CCCN2C(=O)OCc2ccccc2)C(=O)Nc2cccnc2)cc1]

### Example 14—Activity of Stereoisomers

The effect of the two stereoisomers C1/C1a (compound 18) and C1b (compound 43) on LPS induced NO production was compared in murine primary cultured neonatal microglia as described above at concentrations of 0.002, 0.01, 0.02, 0.1, 0.2, 1, 2, 10 and 20 μM. The IC₅₀ was 0.1 M for compound C1/C1a and 40 μM for compound C1b (FIG. 9).

### Example 15—Chemical Synthesis and Analytic Data

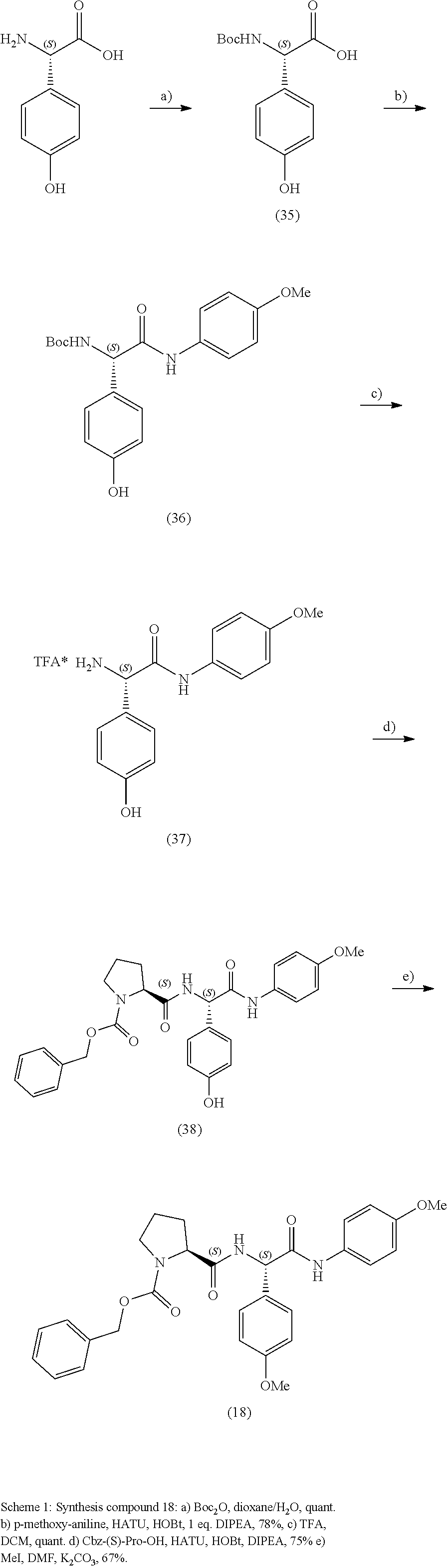

### Abbreviations

HATU, HOBt: Hydroxybenzotriazole, DIPEA: N,N-Diisopropylethylamine, DMF: N,N-Dimethylformamide, HRMS, LCMS: Liquid Chromatography Mass Spectrometry, RT: room temperature, EtOAc: Ethyl acetate, TFA: Trifluoroacetic acid, DCM: Dichloromethane, EE, MeII

According to scheme 1 the following intermediates were synthesized.

### (S)-2-((tert-butoxycarbonyl)amino)-2-(4-hydroxyphenyl)acetic acid (compound 35)

2 g (12 mmol) (S)-4-Hydroxy-phenylglycine were dissolved in dioxane/H₂O (1:1) and 3.13 g (14 mmol) of Boc₂O was added in one portion at RT. The mixture was stirred for 4 h at RT. Dioxane was removed under reduced pressure and the resulting aqueous solution was extracted EtOAc. The organic extracts were combined and dried over Na₂SO₄. After removal of the solvent 3.1 g (12 mmol) of the product was obtained as white powder in quantitative yield.

¹H NMR (300 MHz, DMSO-d₆) δ 7.40 (d, J=8.2 Hz, 1H), 7.18 (d, J=8.7 Hz, 2H), 6.71 (d, J=8.6 Hz, 2H), 4.95 (d, J=8.1 Hz, 1H), 1.38 (s, 9H). LCMS: Rₜ=1.40 min; HRMS (ESIpos): m/z [M+Na]⁺ calcd for C₁₃H₁₇NO₅ 290.0999 found 290.1000.

### tert-butyl (S)—(1-(4-hydroxyphenyl)-2-((4-methoxyphenyl)amino)-2-oxoethyl)carbamate (compound 36)

2.1 g (7.9 mmol) of compound 35 were dissolved in DMF. 986 mg (7.9 mmol) p-anisidine, 1.2 g (7.9 mmol) HOBt, 2.99 g (7.9 mmol) HATU and 1.37 ml (7.9 mmol) DIPEA were added at RT. The mixture was stirred for 4 h at RT. DMF was evaporated and the mixture was dissolved in EtOAc. The organic solution was extracted with 1N HCl solution to remove DIPEA and HOBt. The organic layer was dried over Na₂SO₄ and the crude product was purified by chromatography on silica gel eluting with a gradient of DCM/MeOH. The fractions containing the product were combined and the solvent evaporated under reduced pressure. Yield: 2.3 g (78%).

¹H NMR (300 MHz, DMSO-d₆) δ 10.00 (s, 1H), 9.43 (s, 1H), 7.48 (d, J=9.1 Hz, 2H), 7.27 (d, J=8.6 Hz, 2H), 6.87 (d, J=9.2 Hz, 2H), 6.72 (d, J=8.6 Hz, 2H), 5.19 (d, J=8.2 Hz, 1H), 3.70 (s, 3H), 1.39 (s, 9H). LCMS: Rₜ=1.67 min; HRMS (ESIpos): m/z [M+H]⁺ calcd for C₁₃H₁₇NO₅ 373.1758 found 373.1555.

### (S)-2-amino-2-(4-hydroxyphenyl)-N-(4-methoxyphenyl)acetamide (compound 37)

2.3 g (6.1 mmol) of compound 36 were dissolved in 100 ml DCM/TFA mixture (9:1) and stirred at RT for 1 h. The solvent was removed under reduced pressure and EE/hexane was added several times and iteratively removed under reduced pressure until a white foam was contained. Yield: 2.6 g (quant.)

¹H NMR (300 MHz, DMSO-d₆) δ 10.46 (s, 1H), 8.63 (s, 2H), 7.49 (d, J=9.2 Hz, 2H), 7.37 (d, J=8.8 Hz, 2H), 6.91 (d, J=9.2 Hz, 2H), 6.83 (d, J=8.7 Hz, 2H), 4.96 (s, 1H). LCMS: Rₜ=0.58 min; HRMS (ESIpos): m/z [M+H]⁺ calcd for C₁₅H₁₆N₂O₃ 273.1234 found 273.1234.

### benzyl-(S)-2-(((S)-1-(4-hydroxyphenyl)-2-((4-methoxyphenyl)amino)-2-oxoethyl)-carbamoyl)-pyrrolidine-1-carboxylate (compound 38)

2.6 g (6.7 mmol) of compound 37 were dissolved in 60 ml DMF and 1.68 g (6.7 mmol) Cbz-(S)-Pro-OH, 1.0 g (6.7 mmol) HOBt, 2.56 g (6.7 mmol) HATU and 2.3 ml (13.5 mmol) DIPEA were added. The mixture was stirred for 1 h and the solvent was removed under reduced pressure. The mixture was dissolved in EtOAc and extracted with 1N HCl. The organic layer was dried with Na₂SO₄ and removed under reduced pressure. The crude product was purified by chromatography on silica gel eluting with a gradient of DCM/MeOH. The fractions containing the product were combined and the solvent evaporated under reduced pressure. Yield: 2.55 g (75%).

¹H-NMR (300 MHz, DMSO-d₆) δ cis/trans (10.11 (s, 1H) and 10.05 (s, 1H)), cis/trans (9.48 (s, 1H) and 9.45 (s, 1H)), cis/trans (8.57 (d, J=7.4 Hz, 1H) and 8.50 (d, J=7.3 Hz, 1H)), 7.49 (d, J=8.5 Hz, 2H), 7.41-7.33 (m, 2H), 7.32-7.18 (m, 5H), 6.87 (d, J=8.5 Hz, 2H), 6.79-6.66 (m, 2H), cis/trans (5.45 (d, J=7.3 Hz, 1H) and 5.41 (d, J=7.4 Hz, 1H)), cis/trans (5.07 (s, 2H) and 5.00-4.89 (m, 2H)), cis/trans (4.47 (dd, J=8.7, 3.5 Hz, 1H) and 4.40 (dd, J=8.7, 3.1 Hz, 1H)), 3.71 (s, 3H), 3.50-3.38 (m, 2H), 2.22-2.05 (m, 1H), 1.95-1.73 (m, 3H). LCMS: Rₜ=1.71 min; HRMS (ESIpos): m/z [M+Na]⁺ calcd for C₂₈H₂₉N₃O₆ 526.1942 found 526.1949.

### (S)-benzyl-2-((S)—(4-methoxyphenylcarbamoyl)(4-methoxyphenyl)methylcarbamoyl)pyrrolidine-1-carboxylate (compound 18)

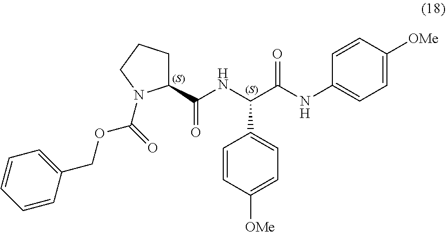

200 mg (0.4 mmol) of compound 38 were dissolved in 4 ml DMF. 110 mg K₂CO₃ (0.8 mmol) and 25 μl (0.4 mmol) Mel were added. The mixture was stirred for 16 h at RT. K₂CO₃ was filtered off, the solvent and Mel were removed under reduced pressure and the crude product was purified by chromatography on silica gel eluting with a gradient of Hex/EE. The fractions containing the product were combined and the solvent evaporated under reduced pressure. Yield: 140 mg (67%).

¹H-NMR (300 MHz, DMSO-d₆) δ cis/trans (10.11 (s, 1H) and 10.05 (s, 1H)), cis/trans (8.58 (d, J=7.3 Hz, 1H) and 7.49 (d, J=9.1 Hz, 1H)), 7.49 (d, J=8.9 Hz, 2H), 7.45-7.31 (m, 4H), 7.28-7.21 (m, 3H), 6.94 (d, J=8.9 Hz, 1H), 6.90-6.83 (m, 3H), cis/trans (5.52 (d, J=7.6 Hz, 1H) and 5.48 (d, J=7.6 Hz, 1H)), cis/trans (5.07 (s, 2H) and 5.00-4.89 (m, 2H), cis/trans (4.49 (dd, J=8.5, 3.5 Hz, 1H) and 4.41 (dd, J=8.6, 3.1 Hz, 1H)), 3.71 (s, 6H), 3. 3.49-3.36 (m, 2H), 2.27-2.04 (m, 1H), 1.94-1.73 (m, 3H). LCMS: Rₜ=1.91 min; HRMS (ESIpos): m/z [M+H]⁺ calcd for C₂₉H₃₁N₃O₆ 518.2286 found 518.2288.

### (S)-1-(3-cyclohexylpropanoyl)-N—((S)-1-(4-methoxyphenyl)-2-((4-methoxyphenyl)amino)-2-oxoethyl)pyrrolidine-2-carboxamide (compound 6)

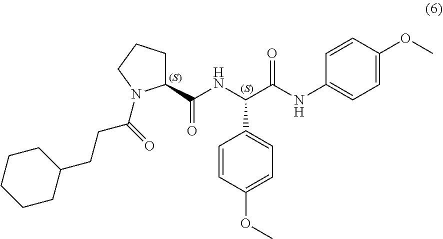

¹H NMR (300 MHz, DMSO-d₆) δ cis/trans (10.18 (s, 1H) and 10.02 (s, 1H)), cis/trans (8.88 (d, J=7.9 Hz, 1H) and 8.46 (d, J=7.5 Hz, 1H)), 7.50 (d, J=9.0 Hz, 2H), 7.43-7.35 (m, 2H), 6.92 (dd, J=8.7, 2.2 Hz, 2H), 6.87 (dd, J=9.2, 2.3 Hz, 2H), cis/trans (5.57 (d, J=7.9 Hz, 1H) and 5.43 (d, J=7.4 Hz, 1H)), cis/trans (4.58 (dd, J=8.6, 3.0 Hz, 1H) and 4.46 (dd, J=7.5, 2.7 Hz, 1H)), 3.73 (s, 3H), 3.71 (s, 3H), 3.58-3.36 (m, 2H), 2.31-2.16 (m, 2H), 2.10-1.79 (m, 4H), 1.75-1.44 (m, 6H), 1.33-0.96 (m, 6H), 0.92-0.77 (m, 1H). LCMS: Rₜ=2.022 min; HRMS (ESIpos): m/z [M+H]⁺ calcd for C₃₀H₃₉N₃O₅ 522.2962 found 522.2975.

### (S)-1-(cyclohexanecarbonyl)-N—((S)-1-(4-methoxyphenyl)-2-((4-methoxyphenyl)amino)-2-oxoethyl)pyrrolidine-2-carboxamide (compound 23)

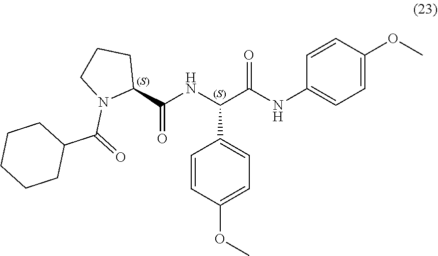

¹H NMR (300 MHz, DMSO-d₆) δ cis/trans (10.19 (s, 1H) and 10.04 (s, 1H)), cis/trans (8.91 (d, J=7.8 Hz, 1H) and 8.39 (d, J=7.3 Hz, 1H)), 7.49 (d, J=9.0 Hz, 2H), 7.40 (d, J=12.2 Hz, 2H), 6.93 (d, J=8.2 Hz, 2H), 6.87 (d, J=8.2 Hz, 2H), cis/trans (5.56 (d, J=7.7 Hz, 1H) and 5.42 (d, J=7.3 Hz, 1H)), cis/trans (4.65 (dd, J=8.7, 2.7 Hz, 1H) and 4.45 (dd, J=8.0, 2.9 Hz, 1H)), 3.78-3.67 (m, 6H), 3.61-3.50 (m, 1H), 3.35 (s, 2H), 2.50-2.36 (m, 3H), 2.29-2.11 (m, 1H), 2.08-1.75 (m, 4H), 1.74-1.55 (m, 4H), 1.54-1.40 (m, 1H), 1.38-1.02 (m, 6H), 0.84-0.66 (m, 1H). LCMS: Rₜ=1.860 min; HRMS (ESIpos): m/z [M+H]⁺ calcd for C₂₈H₃₅N₃O₅ 494.2660 found 494.2649.

### (S)-1-(2-cyclohexylacetyl)-N—((S)-1-(4-methoxyphenyl)-2-((4-methoxyphenyl)amino)-2-oxoethyl)pyrrolidine-2-carboxamide (compound 19)

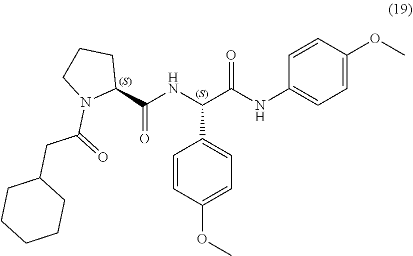

¹H NMR (300 MHz, DMSO-d₆) δ cis/trans (10.19 (s, 1H) and 10.05 (s, 1H)), cis/trans (8.87 (d, J=7.8 Hz, 1H) and 8.40 (d, J=7.3 Hz, 1H)), (7.49 (d, J=8.8 Hz, 2H), 7.44-7.36 (m, 2H), 6.93 (d, J=8.4 Hz, 2H), 6.90-6.82 (m, 2H), cis/trans (5.56 (d, J=7.9 Hz, 1H) and 5.43 (d, J=7.3 Hz, 1H)), cis/trans (4.58 (dd, J=8.4, 2.8 Hz, 1H) and 4.48 (dd, J=8.2, 3.1 Hz, 1H)), 3.79-3.65 (m, 6H), 3.57-3.36 (m, 2H), 2.20-2.10 (m, 1H), 2.06-1.79 (m, 4H), 1.78-1.43 (m, 7H), 1.29-0.97 (m, 4H), 0.96-0.84 (m, 1H). LCMS: Rₜ=1.940 min; HRMS (ESIpos): m/z [M+H]⁺ calcd for C₂₉H₃₇N₃O₅ 508.2806 found 508.2815.

### (S)-1-(2-cyclohexylacetyl)-N—((S)-1-(4-methoxyphenyl)-2-((4-methoxyphenyl)amino)-2-oxoethyl)pyrrolidine-2-carboxamide (compound 7)

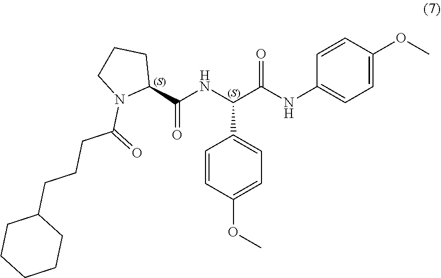

¹H NMR (300 MHz, DMSO-d₆) δ cis/trans (10.19 (s, H1) and 10.01 (s, 1H)), cis/trans (8.87 (d, J=8.0 Hz, 1H) and 8.45 (d, J=7.5 Hz, 1H)), 7.55-7.45 (m, 2H), 7.44-7.35 (m, 2H), 6.92 (dd, J=8.7, 2.1 Hz, 2H), 6.89-6.83 (m, 2H), cis/trans (5.57 (d, J=7.9 Hz, 1H) and 5.44 (d, J=7.4 Hz, 1H)), cis/trans (4.58 (dd, J=8.5, 3.0 Hz, 1H) and 4.46 (dd, J=7.9, 3.1 Hz, 1H)), 3.78-3.68 (m, 6H), 3.57-3.36 (m, 2H), 2.27-2.20 (m, 1H), 2.11-1.76 (m, 4H), 1.75-1.42 (m, 7H), 1.32-1.04 (m, 6H), 0.97-0.71 (m, 3H). LCMS: Rₜ=2.112 min; HRMS (ESIpos): m/z [M+Na]⁺ calcd for C₃₁H₄₁N₃O₅ 558.2938 found 558.2938.

### (S)-1-(3-cyclopentylpropanoyl)-N—((S)-1-(4-methoxyphenyl)-2-((4-methoxyphenyl)amino)-2-oxoethyl)pyrrolidine-2-carboxamide (compound 5)

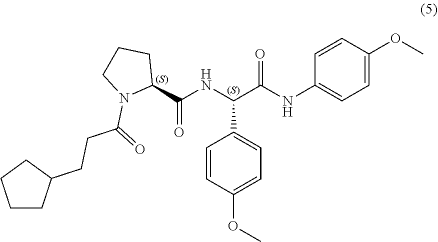

¹H NMR (300 MHz, DMSO-d₆) δ cis/trans (10.18 (s, 1H) and 10.03 (s, 1H)), cis/trans (d, J=8.0 Hz, 1H) and 8.46 (d, J=7.5 Hz, 1H)), 7.49 (d, J=9.0 Hz, 2H), 7.43-7.35 (m, 2H), 6.92 (dd, J=8.8, 2.9 Hz, 2H), 6.87 (dd, J=9.1, 1.9 Hz, 2H), cis/trans (5.57 (d, J=7.9 Hz, 1H) and 5.44 (d, J=7.4 Hz, 1H)), cis/trans (4.58 (dd, J=8.5, 3.0 Hz, 1H) and 4.47 (dd, J=8.0, 3.1 Hz, 1H)), 3.73-3.71 (m, 6H), 3.59-3.36 (m, 2H), 2.31-2.23 (m, 1H), 2.14-1.96 (m, 1H), 1.94-1.69 (m, 5H), 1.61-1.28 (m, 8H), 1.13-0.99 (m, 1H), 0.95-0.80 (m, 1H). LCMS: Rₜ=2.017 min; HRMS (ESIpos): m/z [M+H]⁺ calcd for C₂₉H₃₇N₃O₅ 508.2806 found 508.2818.

### (S)—N—((S)-1-(4-methoxyphenyl)-2-((4-methoxyphenyl)amino)-2-oxoethyl)-1-(4-methylpentanoyl)pyrrolidine-2-carboxamide (compound 20)

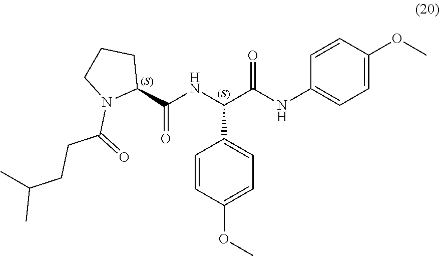

¹H NMR (300 MHz, DMSO-d₆) δ cis/trans (10.18 (s, 1H) and 10.03 (s, 1H)), cis/trans (8.87 (d, J=7.9 Hz, 1H) and 8.46 (d, J=7.5 Hz, 1H)), 7.49 (d, J=9.1 Hz, 2H), 7.39 (d, J=8.8 Hz, 2H), 6.92 (dd, J=8.7, 3.6 Hz, 2H), 6.87 (dd, J=9.1, 1.7 Hz, 2H), cis/trans (5.56 (d, J=7.8 Hz, 1H) and 5.44 (d, J=7.4 Hz, 1H)), cis/trans (4.59 (dd, J=8.5, 3.0 Hz, 1H) and 4.47 (dd, J=7.7, 3.1 Hz, 1H)), 3.73 (s, 3H), 3.71 (s, 3H), 3.58-3.35 (m, 2H), 2.31-2.20 (m, 2H), 2.14-1.93 (m, 1H), 1.92-1.70 (m, 3H), 1.56-1.21 (m, 3H), 0.87 (d, J=6.5 Hz, 4H), 0.76-0.69 (m, 2H). LCMS: Rₜ=1.925 min; HRMS (ESIpos): m/z [M+H]⁺ calcd for C₂₇H₃₅N₃O₅ 482.2649 found 482.2659.

### (S)—N—((S)-1-(4-methoxyphenyl)-2-((4-methoxyphenyl)amino)-2-oxoethyl)-1-(2-phenoxyacetyl)pyrrolidine-2-carboxamide (compound 11)

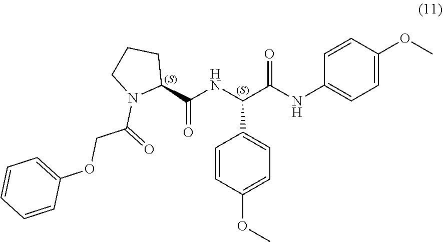

¹H NMR (500 MHz, Chloroform-d) δ 8.04 (s, 1H), 7.50 (d, J=8.6 Hz, 2H), 7.25-7.19 (m, 4H), 7.00-6.95 (m, 1H), 6.91 (d, J=8.1 Hz, 2H), 6.83 (d, J=8.5 Hz, 4H), 5.47 (d, J=7.1 Hz, 1H), 4.70 (s, 2H), 4.65 (dd, J=7.9, 4.3 Hz, 1H), 3.78 (s, 6H), 3.73-3.60 (m, 2H), 2.24-2.17 (m, 1H), 2.14-1.97 (m, 3H). LCMS: Rₜ=3.60 min; (ESIpos): m/z [M+H]⁺ calcd for C₂₉H₃₁N₃O₆ 517.2 found 518.2.

### (S)-1-(3-(4-fluorophenyl)propanoyl)-N—((S)-1-(4-methoxyphenyl)-2-((4-methoxyphenyl)amino)-2-oxoethyl)pyrrolidine-2-carboxamide(compound 9)

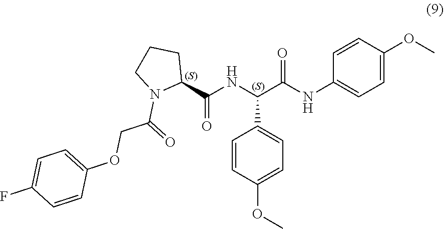

¹H NMR (500 MHz, Chloroform-d) δ 8.04 s, 1H), 7.52 (d, J=9.0 Hz, 2H), 7.42 (d, J=7.1 Hz, 1H), 7.26 (s, 2H), 7.12 (dd, J=8.4, 5.5 Hz, 2H), 6.93-6.83 (m, 6H), 5.46 (d, J=7.0 Hz, 1H), 4.61 (dd, J=7.8, 3.4 Hz, 1H), 3.80 (s, 3H), 3.79 (s, 3H), 3.46 (ddd, J=10.7, 6.9, 3.9 Hz, 1H), 3.40-3.33 (m, 1H), 2.94 (t, J=7.4 Hz, 2H), 2.62 (t, J=7.4 Hz, 2H), 2.30-2.24 (m, 1H), 2.03-1.90 (m, 3H). LCMS: Rₜ=3.83 min; (ESIpos): m/z [M+H]⁺ calcd for C₃₀H₃₂FN₃O₅ 533.2 found 534.2.

### (S)—N—((S)-1-(4-methoxyphenyl)-2-((4-methoxyphenyl)amino)-2-oxoethyl)-1-(2-phenylacetyl)pyrrolidine-2-carboxamide (compound 12)

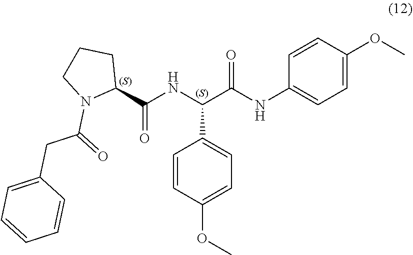

¹H NMR (500 MHz, Chloroform-d) δ 8.11 (s, 1H), 7.54 (d, J=8.7 Hz, 2H), 7.27-7.23 (m, 5H), 7.22-7.20 (m, 2H), 7.17 (d, J=8.3 Hz, 2H), 6.88-6.82 (m, 4H), 5.43 (d, J=7.3 Hz, 1H), 4.66 (dd, J=8.2, 3.5 Hz, 1H), 3.81 (s, 3H), 3.79 (s, 3H), 3.72 (s, 2H), 3.58-3.54 (m, 2H), 2.29-2.23 (m, 1H), 2.09-2.03 (m, 1H), 1.99-1.94 (m, 2H). LCMS: Rₜ=3.64 min; (ESIpos): m/z [M+H]⁺ calcd for C₂₉H₃₁N₃O₅ 533.2 found 534.2

### (S)-1-benzoyl-N—((S)-1-(4-methoxyphenyl)-2-((4-methoxyphenyl)amino)-2-oxoethyl)pyrrolidine-2-carboxamide (compound 24)

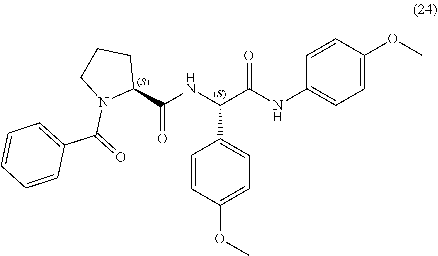

¹H NMR (500 MHz, Chloroform-d) δ 8.12 (s, 1H), 7.51 (d, J=7.5 Hz, 2H), 7.49-7.41 (m, 4H), 7.35 (d, J=8.3 Hz, 2H), 6.86 (d, J=8.3 Hz, 2H), 6.81 (d, J=8.5 Hz, 2H), 5.54 (d, J=6.9 Hz, 1H), 4.84-4.78 (m, 1H), 3.78 (s, 3H), 3.77 (s, 3H), 3.61-3.48 (m, 2H), 2.33-2.19 (m, 2H), 2.03-1.95 (m, 1H), 1.91-1.82 (m, 1H). LCMS: Rₜ=3.48 min; (ESIpos): m/z [M+H]⁺ calcd for C₂₈H₂₉N₃O₅ 487.2 found 488.2.

### (S)—N—((S)-1-(4-methoxyphenyl)-2-((4-methoxyphenyl)amino)-2-oxoethyl)-1-(3-(thiophen-2-yl)propanoyl)pyrrolidine-2-carboxamide (compound 16)

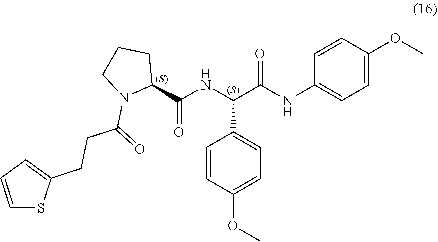

¹H NMR (500 MHz, Chloroform-d) δ 8.13 (s, 1H), 7.55 (d, J=9.0 Hz, 2H), 7.36 (d, J=7.1 Hz, 1H), 7.30 (d, J=8.7 Hz, 2H), 7.05 (d, J=5.0 Hz, 1H), 6.89 (d, J=8.7 Hz, 2H), 6.87-6.81 (m, 3H), 6.77-6.74 (m, 1H), 5.49 (d, J=7.1 Hz, 1H), 4.63 (dd, J=8.2, 3.6 Hz, 1H), 3.81 (s, 3H), 3.79 (s, 3H), 3.54-3.47 (m, 1H), 3.46-3.39 (m, 1H), 3.25-3.15 (m, 2H), 2.70 (t, J=7.1 Hz, 2H), 2.33-2.24 (m, 1H), 2.08-1.92 (m, 3H). LCMS: Rₜ=3.72 min; (ESIpos): m/z [M+H]⁺ calcd for C₂₈H₃₁N₃O₅S 521.2 found 522.2.

### (S)-1-(3-(furan-3-yl)propanoyl)-N—((S)-1-(4-methoxyphenyl)-2-((4-methoxyphenyl)amino)-2-oxoethyl)pyrrolidine-2-carboxamide (compound 14)

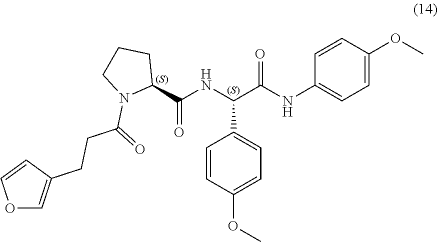

¹H NMR (500 MHz, Chloroform-d) δ 8.00 (s, 1H), 7.53 (d, J=8.8 Hz, 2H), 7.41 (d, J=7.2 Hz, 1H), 7.30 (d, J=8.3 Hz, 3H), 7.18 (s, 1H), 6.90 (d, J=8.3 Hz, 2H), 6.85 (d, J=9.0 Hz, 2H), 6.23 (s, 1H), 5.46 (d, J=7.0 Hz, 1H), 4.65-4.60 (m, 1H), 3.81 (s, 3H), 3.79 (s, 3H), 3.54-3.50 (m, 1H), 3.47-3.41 (m, 1H), 2.83-2.75 (m, 2H), 2.58 (t, J=7.3 Hz, 2H), 2.33-2.27 (m, 1H), 2.06-1.94 (m, 3H). LCMS: Rₜ=3.55 min; (ESIpos): m/z [M+H]⁺ calcd for C₂₈H₃₁N₃O₆ 505.2 found 506.2.

### (S)—N—((S)-1-(4-methoxyphenyl)-2-((4-methoxyphenyl)amino)-2-oxoethyl)-1-(3-(1-methyl-1H-indol-3-yl)propanoyl)pyrrolidine-2-carboxamide (compound 21)

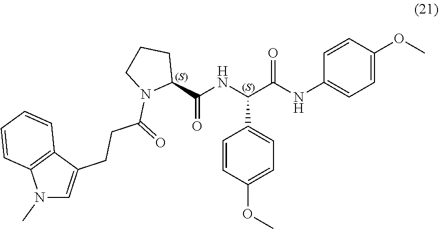

¹H NMR (500 MHz, Chloroform-d) δ 8.26 (s, 1H), 7.65 (d, J=9.0 Hz, 2H), 7.58 (d, J=7.8 Hz, 1H), 7.29 (d, J=8.2 Hz, 1H), 7.26-7.19 (m, 2H), 7.14-7.09 (m, 1H), 7.07 (d, J=8.5 Hz, 2H), 6.88 (d, J=9.0 Hz, 2H), 6.84 (s, 1H), 6.72 (d, J=8.6 Hz, 2H), 5.43 (d, J=7.2 Hz, 1H), 4.61 (dd, J=8.5, 3.8 Hz, 1H), 3.80 (s, 3H), 3.75 (s, 3H), 3.55 (s, 3H), 3.47-3.43 (m, 1H), 3.41-3.36 (m, 1H), 3.18-3.11 (m, 2H), 2.80-2.73 (m, 2H), 2.23-2.17 (m, 1H), 2.05-2.00 (m, 1H), 1.92-1.84 (m, 2H). LCMS: Rₜ=3.96 min; (ESIpos): m/z [M+H]⁺ calcd for C₃₃H₃₆N₄O₅ 568.3 found 569.2.

### (S)—N—((S)-1-(4-methoxyphenyl)-2-((4-methoxyphenyl)amino)-2-oxoethyl)-1-(3-(pyridin-2-yl)propanoyl)pyrrolidine-2-carboxamide (compound 25)

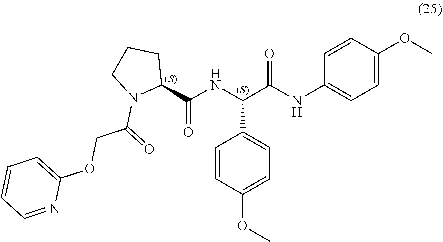

¹H NMR (500 MHz, Chloroform-d) δ 9.03 (s, 1H), 8.28 (d, J=8.1 Hz, 1H), 7.74 (d, J=9.0 Hz, 2H), 7.48-7.42 (m, 2H), 7.20 (d, J=8.7 Hz, 2H), 7.07 (d, J=7.7 Hz, 1H), 6.85 (d, J=9.0 Hz, 3H), 6.76 (d, J=8.6 Hz, 2H), 5.71 (d, J=8.0 Hz, 1H), 4.61 (dd, J=8.6, 4.5 Hz, 1H), 3.43 (ddd, J=10.1, 8.4, 6.9 Hz, 1H), 3.41-3.34 (m, 1H), 3.22 (ddd, J=10.0, 7.5, 4.5 Hz, 1H), 3.02 (ddd, J=15.0, 6.6, 3.5 Hz, 1H), 2.77 (ddd, J=14.5, 11.0, 3.6 Hz, 1H), 2.67 (ddd, J=14.5, 6.5, 3.9 Hz, 1H), 2.29-2.19 (m, 2H), 1.88 (ddd, J=22.3, 13.1, 5.9 Hz, 2H). LCMS: Rₜ=2.57 min; (ESIpos): m/z [M+H]⁺ calcd for C₂₉H₃₂N₄O₅ 516.2 found 517.2.

### (S)—N—((S)-1-(4-methoxyphenyl)-2-((4-methoxyphenyl)amino)-2-oxoethyl)-1-(3-(tetrahydro-2H-pyran-4-yl)propanoyl)pyrrolidine-2-carboxamide (compound 15)

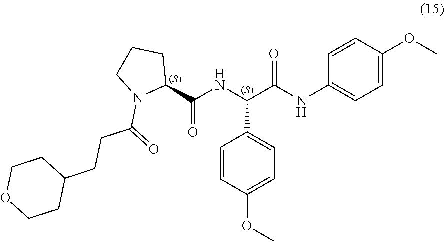

¹H NMR (500 MHz, Chloroform-d) δ 8.05 (s, 1H), 7.54 (d, J=7.0 Hz, 1H), 7.48 (d, J=8.9 Hz, 2H), 7.32 (d, J=8.4 Hz, 2H), 6.88 (d, J=8.4 Hz, 2H), 6.83 (d, J=8.5 Hz, 2H), 5.47 (d, J=6.9 Hz, 1H), 4.65-4.61 (m, 1H), 3.96-3.90 (m, 2H), 3.80 (s, 3H), 3.78 (s, 3H), 3.63-3.54 (m, 1H), 3.51-3.44 (m, 1H), 3.35-3.28 (m, 2H), 2.37-2.32 (m, 2H), 2.32-2.25 (m, 1H), 2.07-1.97 (m, 3H), 1.59-1.46 (m, 5H), 1.30-1.23 (m, 2H). LCMS: Rₜ=3.34 min; (ESIpos): m/z [M+H]⁺ calcd for C₂₉H₃₇N₃O₆ 523.3 found 524.4.

### benzyl (S)-2-(((S)-1-(4-methoxyphenyl)-2-((2-methylthiazol-5-yl)amino)-2-oxoethyl)carbamoyl)pyrrolidine-1-carboxylate (compound 26)

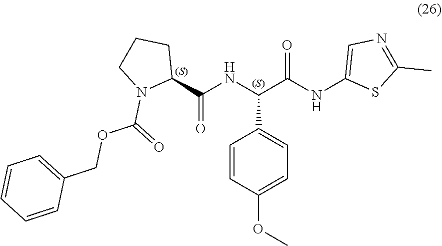

¹H NMR (500 MHz, Chloroform-d) δ 9.98 (s, 1H), 7.55 (d, J=25.3 Hz, 1H), 7.41-7.28 (m, 5H), 7.15-7.08 (m, 1H), 6.93-6.73 (m, 3H), 5.67 (d, J=6.9 Hz, OH), 5.60 (d, J=6.8 Hz, 1H), 5.29 (d, J=12.1 Hz, 1H), 5.19-5.08 (m, 1H), 4.43-4.35 (m, 1H), 4.15 (d, J=8.1 Hz, OH), 3.79 (s, 3H), 3.64-3.52 (m, 2H), 2.62 (s, 3H), 2.28-2.09 (m, 2H), 1.94-1.85 (m, 1H). LCMS: Rₜ=3.51 min; (ESIpos): m/z [M+H]⁺ calcd for C₂₆H₂₈N₄O₅S 508.2 found 509.2.

### benzyl-(S)-2-(((S)-2-((2,3-dihydrobenzofuran-5-yl)amino)-1-(4-methoxyphenyl)-2-oxoethyl)carbamoyl)pyrrolidine-1-carboxylate (compound 22)

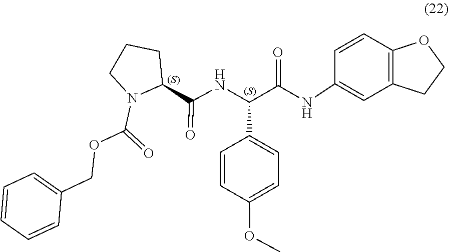

¹H NMR (400 MHz, Chloroform-d) δ 8.70-8.43 (m, 1H), 7.58-7.45 (m, 1H), 7.41-7.26 (m, 5H), 7.24-6.96 (m, 3H), 6.86-6.58 (m, 3H), 5.67-5.52 (m, 1H), 5.24-4.95 (m, 2H), 4.57-4.45 (m, 2H), 4.46-4.26 (m, 1H), 3.75 (s, 3H), 3.65-3.44 (m, 2H), 3.19-3.05 (m, 2H), 2.21-1.98 (m, 2H), 1.94-1.69 (m, 2H). LCMS: Rₜ=3.81 min; (ESIpos): m/z [M+H]⁺ calcd for C₃₀H₃₁N₃O₆ 529.2 found 530.2.

### benzyl(S)-2-(((S)-2-((2-cyclopropylpyrimidin-5-yl)amino)-1-(4-methoxyphenyl)-2-oxoethyl)carbamoyl)pyrrolidine-1-carboxylate (compound 27)

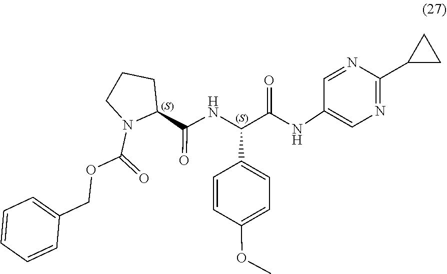

LCMS: Rₜ=3.70 min; (ESIpos): m/z [M+H]⁺ calcd for C₂₉H₃₁N₅O₅ 529.23 found 530.2.

### benzyl(S)-2-(((S)-1-(4-methoxyphenyl)-2-oxo-2-((4,5,6,7-tetrahydropyrazolo[1,5-a]pyridin-2-yl)amino)ethyl)carbamoyl)pyrrolidine-1-carboxylate (compound 10)

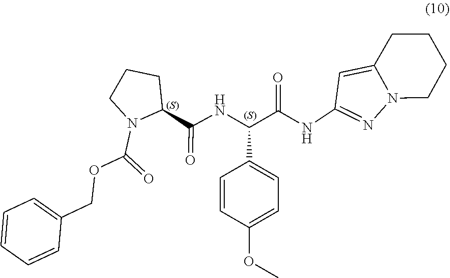

LCMS: Rₜ=3.68 min; (ESIpos): m/z [M+H]⁺ calcd for C₂₉H₃₃N₅O₅ 531.25 found 532.2.

### benzyl (S)-2-(((S)-2-((4-methoxyphenyl)amino)-2-oxo-1-phenylethyl)carbamoyl)pyrrolidine-1-carboxylate (compound 17

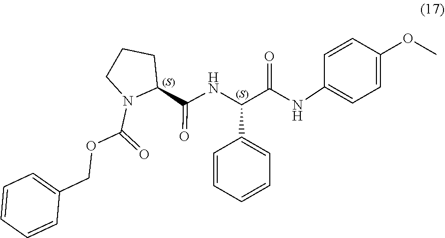

¹H NMR (400 MHz, DMSO-d₆) δ cis/trans (10.22 (s, 1H) and 10.16 (s, 1H)), cis/trans (8.73 (d, J=7.8 Hz, 1H) and 8.64 (d, J=7.3 Hz, 1H)), 7.51-7.45 (m, 3H), 7.45-7.40 (m, 1H), 7.40-7.33 (m, 3H), 7.33-7.25 (m, 3H), 7.24-7.20 (m, 2H), 6.87 (d, J=8.9 Hz, 2H), cis/trans (5.59 (d, J=7.5 Hz, 1H) and 5.56 (d, J=7.7 Hz, 1H)), 5.06 (s, 1H), 4.99-4.87 (m, 1H), cis/trans (4.49 (dd, J=9.0, 3.7 Hz, 1H) and 4.45-4.39 (m, 1H)), 3.70 (s, 3H), 3.49-3.35 (m, 2H), 2.22-2.09 (m, 1H), 1.90-1.75 (m, 3H). LCMS: Rₜ=1.43 min; (ESIpos): m/z [M+H]⁺ calcd for C₂₈H₂₉N₃O₅ 487.2 found 488.2.

### S)—N—((S)-1-(4-methoxyphenyl)-2-((4-methoxyphenyl)amino)-2-oxoethyl)-1-(3-phenylpropanoyl)pyrrolidine-2-carboxamide (compound 13)

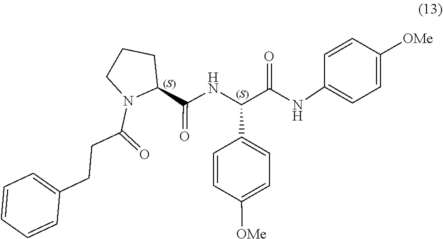

¹H NMR (300 MHz, DMSO-d₆) δ cis/trans (10.18 (s, 1H) and 10.03 (s, 1H)), cis/trans (8.85 (d, J=7.7 Hz, 1H) and 8.50 (d, J=7.6 Hz, 1H)), 7.53-7.44 (m, 2H), cis/trans (7.39 (d, J=8.7 Hz, 2H) and 7.31 (d, J=8.7 Hz, 2H), 7.28-7.13 (m, 4H), 7.13-7.06 (m, 1H), cis/trans 6.93 (d, J=8.7 Hz, 2H), 6.87 (d, J=9.0 Hz, 2H), cis/trans 6.79 (d, J=8.7 Hz, 2H), cis/trans 5.53 (d, J=7.7 Hz, 1H) and 5.44 (d, J=7.4 Hz, 1H)), cis/trans (4.60 (dd, J=8.5, 3.0 Hz, 1H) and 4.48 (dd, J=8.3, 2.9 Hz, 1H)), 3.74 (s, 3H), 3.70 (s, 3H), 3.55-3.38 (m, 2H), 2.85-2.70 (m, 2H), 2.70-2.54 (m, 2H), 2.24-2.06 (m, 1H), 1.98-1.73 (m, 3H). LCMS: Rₜ=1.844 min; HRMS (ESIpos): m/z [M+H]⁺ calcd for C₃₀H₃₃N₃O₅ 516.2493 found 516.2504.

### tert-butyl (S)-2-(((S)-1-(4-methoxyphenyl)-2-((4-methoxyphenyl)amino)-2-oxoethyl)carbamoyl)pyrrolidine-1-carboxylate compound 28)

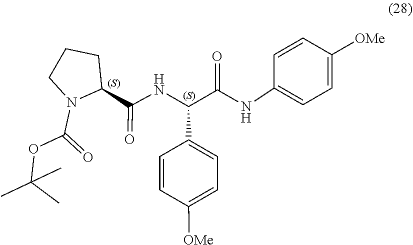

¹H NMR (300 MHz, DMSO-d₆) δ cis/trans (10.14 (s, 1H) and 10.09 (s, 1H)), cis/trans (8.47 (d, J=7.7 Hz, 1H) and 8.43 (d, J=7.1 Hz, 1H)), 7.47 (d, J=8.7 Hz, 2H), 7.40 (d, J=8.3 Hz, 2H), 6.91 (d, J=8.3 Hz, 2H), 6.86 (d, J=8.6 Hz, 2H), cis/trans (5.53 (d, J=7.5 Hz, 1H) and 5.44 (d, J=6.9 Hz, 1H)), 4.33-4.21 (m, 1H), 3.72 (s, 3H), 3.70 (s, 3H), 3.32-3.17 (m, 2H), 2.17-1.96 (m, 1H), 1.89-1.68 (m, 3H), cis/trans (1.39 (s, 9H) and 1.19 (s, 9H)). LCMS: Rₜ=1.833 min; HRMS (ESIpos): m/z [M+H]⁺ calcd for C₂₆H₃₃N₃O₅ 484.2442 found 484.2447.

### (S)-1-(3-cyclohexylpropanoyl)-N—((S)-2-((4-methoxyphenyl)amino)-2-oxo-1-(4-((19-oxo-23-((3aS,4S,6aR)-2-oxohexahydro-1H-thieno[3,4-d]imidazol-4-yl)-3,6,9,12,15-pentaoxa-18-azatricosyl)oxy)phenyl)ethyl)pyrrolidine-2-carboxamide (compound 8)

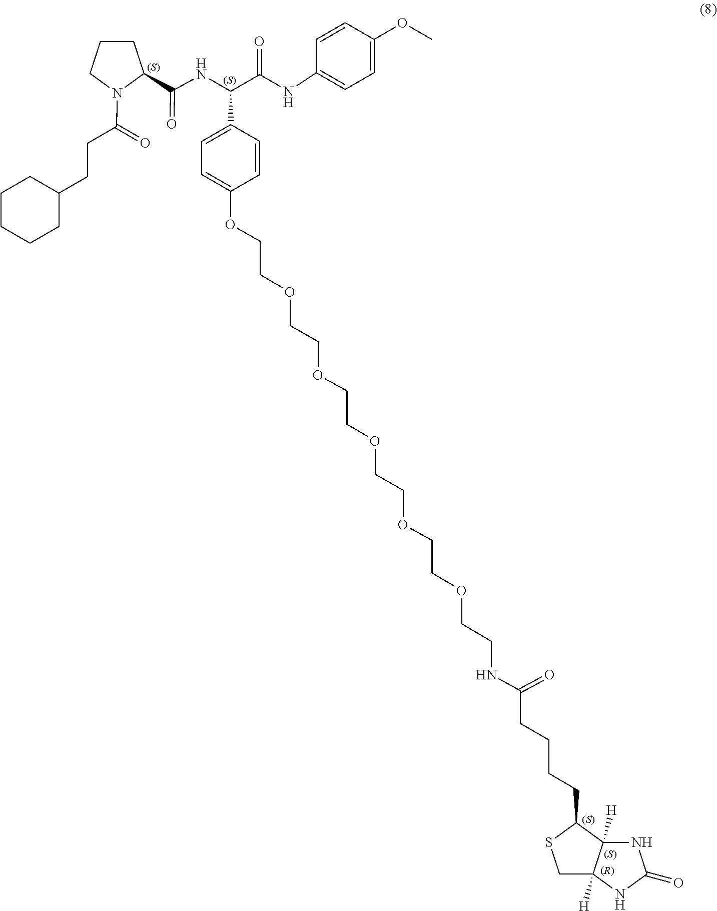

LCMS: R₁=1.149 min; (ESIpos): m/z [M+H]⁺ calcd for C₅₁H₇₆N₆O₁₂S 996.5 found 997.4.

### benzyl (S)-2-(((S)-2-((4-methoxyphenyl)amino)-2-oxo-1-(4-((19-oxo-23-((3aS,4S,6aR)-2-oxohexahydro-1H-thieno[3,4-d]imidazol-4-yl)-3,6,9,12,15-pentaoxa-18-azatricosyl)oxy)phenyl)ethyl)carbamoyl)pyrrolidine-1-carboxylate (compound 29)

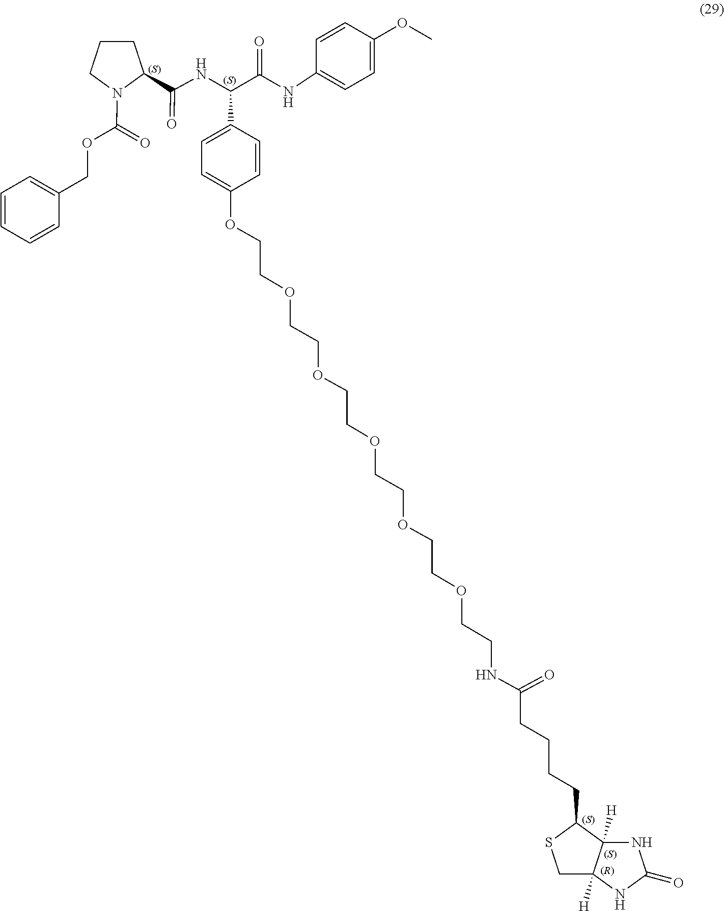

LCMS: Rₜ=1.689 min; HRMS (ESIpos): m/z [M+H]⁺ calcd for C₅₀H₆₈N₆O₁₃S 993.4638 found 993.4676. benzyl(2S,3aS,7aS)-2-(((S)-1-(4-methoxyphenyl)-2-((4-methoxyphenyl)amino)-2-oxoethyl)carbamoyl)octahydro-1H-indole-1-carboxylate (compound 31)

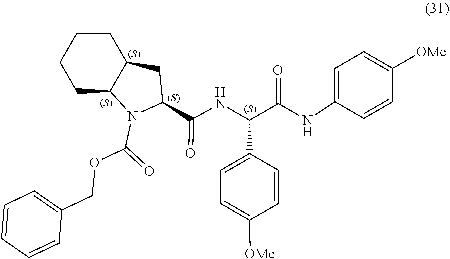

¹H NMR (300 MHz, DMSO-d₆) δ 10.16 (s, 1H), 8.61 (s, 1H), 7.54-7.47 (m, 2H), 7.43-7.17 (m, 7H), 6.98-6.80 (m, 4H), 5.51 (d, J=7.3 Hz, 1H), 5.09-4.79 (m, 2H), 4.53-4.33 (m, 1H), 3.83-3.74 (m, 1H), 3.72 (s, 6H), 2.36-2.20 (m, 1H), 2.17-2.02 (m, 1H), 1.97-1.78 (m, 2H), 1.72-1.34 (m, 5H), 1.28-1.02 (m, 2H). LCMS: Rₜ=1.27 min, (ESIpos): m/z [M+H]⁺ calcd for C₃₃H₃₇N₃O₆ 572.3 found 572.1.

### benzyl (S)-2-(((S)-1-(4-methoxyphenyl)-2-((4-methoxyphenyl)amino)-2-oxoethyl)carbamoyl)azetidine-1-carboxylate (compound 30)

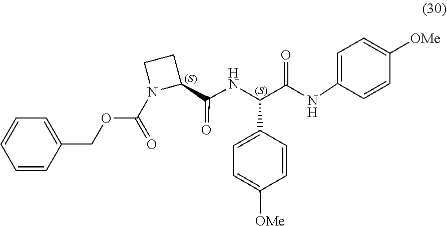

¹H NMR (600 MHz, CDCl₃) δ 8.46-7.90 (m, 2H), 7.51-7.31 (m, 9H), 6.92-6.78 (m, 4H), 5.69 (d, J=5.6 Hz, 1H), 5.26-5.11 (m, 2H), 4.91-4.76 (m, 1H), 4.06-3.99 (m, 1H), 3.93-3.86 (m, 1H), 3.81 (s, 3H), 3.79 (s, 3H), 2.56-2.36 (m, 2H). LCMS: Rₜ=1.46 min, (ESIpos): m/z [M+H]⁺ calcd for C₂₈H₂₉N₃O₆ 504.2 found 501.0.

### benzyl (S)-3-(((S)-1-(4-methoxyphenyl)-2-((4-methoxyphenyl)amino)-2-oxoethyl)carbamoyl)-3,4-dihydroisoquinoline-2(1H)-carboxylate (compound 32)

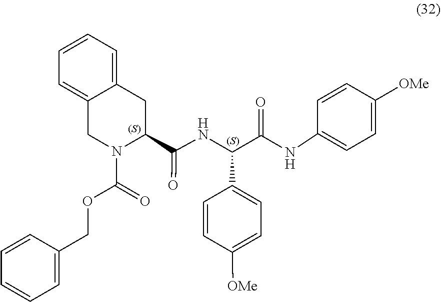

¹H NMR (300 MHz, DMSO-d₆) δ cis/trans (10.12 (s, 1H) and 10.02 (s, 1H)), cis/trans (8.51 (d, J=7.9 Hz, 1H) and 8.42 (d, J=7.7 Hz, 1H)), 7.51-7.34 (m, 4H), 7.28-7.12 (m, 9H), 6.91-6.77 (m, 4H), 5.42 (t, J=9.0 Hz, 1H), cis/trans (5.17 (s, 2H) and 5.03 (s, 2H)), 4.84 (dt, J=11.3, 5.0 Hz, 1H), 4.73-4.46 (m, 2H), 3.72 (s, 3H), 3.71 (s, 3H), 3.28-2.98 (m, 2H). LCMS: Rₜ=1.23 min, (ESIpos): m/z [M+H]⁺ calcd for C34H₃₃N₃O₆ 580.2 found 580.1.

### benzyl (S)-2-(((S)-1-(4-methoxyphenyl)-2-((4-methoxyphenyl)amino)-2-oxoethyl)carbamoyl)-2-methylpyrrolidine-1-carboxylate (compound 33)

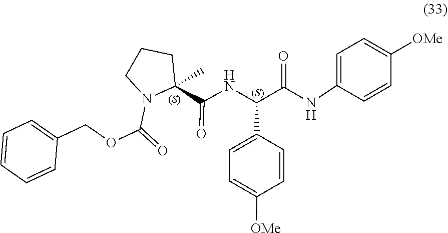

¹H NMR (300 MHz, DMSO-d₆) δ 10.22-9.68 (m, 1H), cis/trans (8.24 (d, J=7.0 Hz, 1H) and 7.91 (d, J=8.0 Hz, 1H)), 7.58-7.15 (m, 9H), 6.96-6.84 (m, 4H), cis/trans (5.56 (d, J=7.4 Hz, 1H) and 5.43 (d, J=7.0 Hz, 1H)), 5.19-4.77 (m, 2H), 3.74 (s, 3H), 3.72 (s, 3H), 3.69-3.59 (m, 1H), 3.58-3.44 (m, 1H), 2.20-2.02 (m, 1H), 1.99-1.68 (m, 3H), cis/trans (1.51 (s, 3H) and 1.48 (s, 3H)). LCMS: Rₜ=1.20 min, (ESIpos): m/z [M+H]⁺ calcd for C₃₀H₃₃N₃O₆ 532.2 found 532.1.

### benzyl (S)-2-(((S)-1-(4-methoxyphenyl)-2-((4-methoxyphenyl)amino)-2-oxoethyl)carbamoyl)piperidine-1-carboxylate (compound 34)

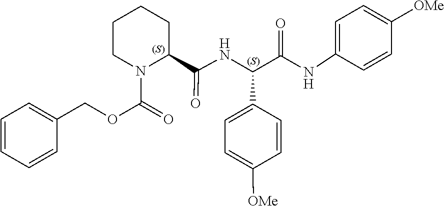

¹H NMR (300 MHz, DMSO-d₆) δ cis/trans (10.13 (s, 1H) and 10.11 (s, 1H)), cis/trans (8.54 (d, J=6.6 Hz, 1H) and 8.45 (d, J=5.5 Hz, 1H)), 7.54-7.45 (m, 2H), 7.45-7.21 (m, 7H), 6.97-6.79 (m, 4H), 5.57-5.42 (m, 1H), 5.12-4.98 (m, 2H), 4.90-4.71 (m, 1H), 3.94-3.81 (m, 1H), 3.73 (s, 3H), 3.71 (s, 3H), 3.31-3.14 (m, 1H), 2.25-2.06 (m, 1H), 1.76-1.49 (m, 3H), 1.41-1.20 (m, 2H). LCMS: Rₜ=1.20 min, (ESIpos): m/z [M+Na]⁺ calcd for C₃₀H₃₃N₃O₆ 554.2 found 554.1.

### (S)—N—((S)-1-(4-methoxyphenyl)-2-((4-methoxyphenyl)amino)-2-oxoethyl)-1-(3-phenylpropyl)pyrrolidine-2-carboxamide (compound 39)

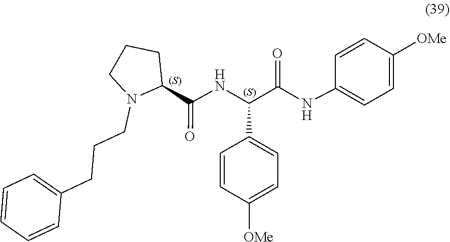

¹H NMR (300 MHz, DMSO-d₆) δ 10.28 (s, 1H), 8.45 (d, J=8.1 Hz, 1H), 7.48 (d, J=8.6 Hz, 2H), 7.35 (d, J=8.3 Hz, 2H), 7.17 (d, J=5.2 Hz, 5H), 6.93 (d, J=8.4 Hz, 2H), 6.88 (d, J=8.7 Hz, 2H), 5.52 (d, J=8.1 Hz, 1H), 3.73 (s, 3H), 3.71 (s, 3H), 3.26-3.16 (m, 1H), 2.98 (dd, J=9.7, 4.3 Hz, 1H), 2.69-2.54 (m, 4H), 2.36-2.25 (m, 1H), 2.10-1.94 (m, 1H), 1.84-1.72 (m, 3H), 1.72-1.58 (m, 2H). LCMS: Rₜ=1.536 min; HRMS (ESIpos): m/z [M+H]⁺ calcd for C₃₀H₃₅N₃O₄ 502.2700 found 502.2709.

### benzyl (S)-2-(((S)-1-(4-methoxyphenyl)-2-((1-methyl-1H-pyrazol-4-yl)amino)-2-oxoethyl)carbamoyl)pyrrolidine-1-carboxylate (compound 40)

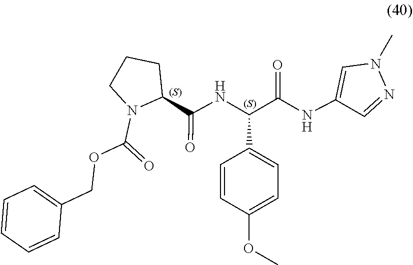

¹H NMR (400 MHz, Chloroform-d) δ 8.77 (s, 1H), 7.94 (d, J=10.7 Hz, 1H), 7.58-7.50 (m, 1H), 7.40-7.27 (m, 5H), 7.14 (d, J=8.1 Hz, 2H), 6.91-6.75 (m, 3H), 5.50 (d, J=6.2 Hz, 1H), 5.30-5.23 (m, 1H), 5.18-5.07 (m, 1H), 4.42-4.35 (m, 1H), 3.86-3.81 (m, 3H), 3.78 (s, 3H), 3.59-3.48 (m, 2H), 2.21-2.09 (m, 2H), 1.94-1.82 (m, 2H). LCMS: Rₜ=3.26 min; (ESIpos): m/z [M+H]⁺ calcd for C₂₆H₂₉N₅O₅ 491.2 found 492.2.

### benzyl (S)-2-(((S)-1-(4-methoxyphenyl)-2-((1-methyl-1H-indol-4-yl)amino)-2-oxoethyl)carbamoyl)pyrrolidine-1-carboxylate (compound 41)

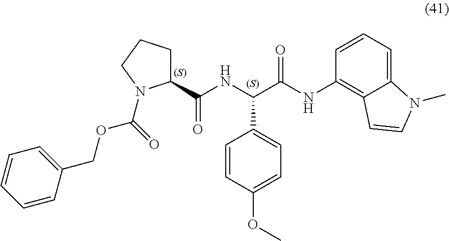

¹H NMR (400 MHz, Chloroform-d) δ 8.24 (s, 1H), 8.03-7.73 (m, 1H), 7.73-7.62 (m, 1H), 7.43-7.13 (m, 8H), 7.10 (d, J=8.2 Hz, 1H), 6.94 (dd, J=8.1, 3.3 Hz, 1H), 6.89-6.71 (m, 2H), 6.24 (dd, J=37.6, 16.7 Hz, 1H), 5.68 (dd, J=21.5, 6.8 Hz, 1H), 5.21-4.93 (m, 2H), 4.50-4.29 (m, 1H), 3.87-3.62 (m, 6H), 3.49 (d, J=38.0 Hz, 2H), 2.18 (d, J=52.5 Hz, 2H), 1.95-1.73 (m, 2H).). LCMS: Rₜ=3.81 min; (ESIpos): m/z [M+H]⁺ calcd for C₃₁H₃₂N₄O₅ 540.2 found 541.2.

### benzyl (S)-2-(((S)-2-((1,3-dimethyl-2-oxo-2,3-dihydro-1H-benzo[d]imidazol-5-yl)amino)-1-(4-methoxyphenyl)-2-oxoethyl)carbamoyl)pyrrolidine-1-carboxylate (compound 42)

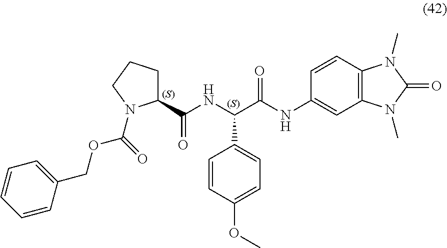

¹H NMR (400 MHz, Chloroform-d) δ 8.82 (s, 1H), 7.62-7.56 (m, 1H), 7.42-7.18 (m, 8H), 7.12-7.06 (m, 1H), 6.89-6.73 (m, 3H), 5.65-5.51 (m, 1H), 5.24-5.06 (m, 2H), 4.46-4.28 (m, 1H), 3.75 (s, 3H), 3.62-3.46 (m, 2H), 3.41-3.26 (m, 6H), 2.21-2.04 (m, 2H), 1.92-1.85 (m, 2H). LCMS: Rₜ=3.45 min; (ESIpos): m/z [M+H]⁺ calcd for C₃₁H₃₃N₅O₆ 571.2 found 572.2.

### benzyl (S)-2-(((S)-1-(4-methoxyphenyl)-2-oxo-2-(phenylamino)ethyl)carbamoyl)pyrrolidine-1-carboxylate (compound 43)

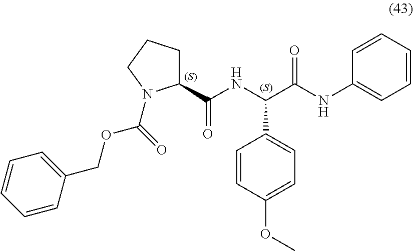

¹H NMR (500 MHz, Chloroform-d) δ 8.45 (s, 1H), 7.68-7.58 (m, 1H), 7.46-7.26 (m, 10H), 7.26-7.17 (m, 2H), 7.14-7.05 (m, 1H), 6.95-6.75 (m, 2H), 5.60-5.47 (m, 1H), 5.27-4.99 (m, 2H), 4.38-4.22 (m, 1H), 3.80 (s, 3H), 3.68-3.50 (m, 2H), 2.30-1.98 (m, 3H), 1.96-1.85 (m, 1H). LCMS: Rₜ=3.99 min; (ESIpos): m/z [M+H]⁺ calcd for C₂₈H₂₉N₃O₅ 487.2 found 488.2.

### benzyl-(S)-2-(((R)-1-(4-methoxyphenyl)-2-((4-methoxyphenyl)amino)-2-oxoethyl)carbamoyl)-pyrrolidine-1-carboxylate (compound 50)

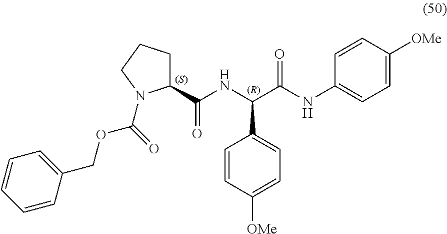

¹H NMR (300 MHz, DMSO-d₆) δ cis/trans (10.26 (s, 1H) and 10.08 (s, 1H)), cis/trans (8.78 (d, J=8.4 Hz, 1H) and 8.75 (d, J=7.9 Hz, 1H)), 7.56-7.47 (m, 2H), 7.42-7.34 (m, 4H), 7.31-7.08 (m, 3H), 6.95-6.86 (m, 4H), cis/trans (5.65 (d, J=8.3 Hz, 1H) and 5.56 (d, J=8.1 Hz, 1H)), cis/trans (5.09 (s, 2H) and 5.00 (s, 2H)), cis/trans (4.49 (dd, J=8.6, 3.5 Hz, 1H) and 4.41 (dd, J=8.6, 3.8 Hz, 1H)), 3.73 (s, 3H), 3.71 (s, 3H), 3.49-3.38 (m, 2H), 2.22-2.06 (m, 1H), 1.87-1.68 (m, 3H). LCMS: Rₜ=1.86 min; HRMS (ESIpos): m/z [M+H]⁺ calcd for C₂₉H₃₁N₃O₆ 518.2286 found 518.2293.

### benzyl (S)-2-(((S)-2-((4-methoxyphenyl)amino)-2-oxo-1-(thiophen-3-yl)ethyl)carbamoyl)pyrrolidine-1-carboxylate (compound 52)

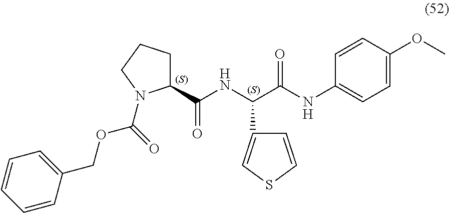

¹H NMR (300 MHz, Chloroform-d) δ cis/trans (8.89 (s, 1H) and 8.78 (s, 1H)), 7.51 (d, J=8.1 Hz, 1H), 7.47 (d, J=7.8 Hz, 1H), 7.35 (s, 4H), 7.27-7.19 (m, 3H), 7.03 (d, J=3.8 Hz, 1H), 6.82 (d, J=8.8 Hz, 2H), cis/trans (5.93 (d, J=8.7 Hz, 1H) and 5.86 (d, J=7.6 Hz, 1H)), 5.25-5.00 (m, 2H), 4.51-4.35 (m, 1H), 3.78 (s, 3H), 3.63-3.46 (m, 2H), 2.26-2.13 (m, 2H), 1.96-1.83 (m, 2H). LCMS: Rₜ=1.86 min; HRMS (ESIpos): m/z [M+H]⁺ calcd for C₂₆H₂₇N₃O₅S 494.1744 found 494.1758.

### (2S,4R)-1-(3-cyclohexylpropanoyl)-4-hydroxy-N—((S)-1-(4-methoxyphenyl)-2-((4-methoxyphenyl)amino)-2-oxoethyl)pyrrolidine-2-carboxamide (compound 53)

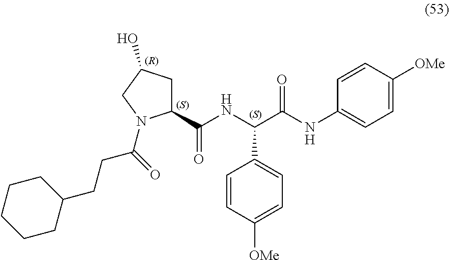

LCMS: Rₜ=1.16 min; (ESIpos): m/z [M+H]⁺ calcd for C₃₀H₃₉N₃O₆ 538.1 found 538.1.

1. A compound for use in a method of treatment or prevention of a disease, characterized by a formula (1a) or (1b) in particular (1b), 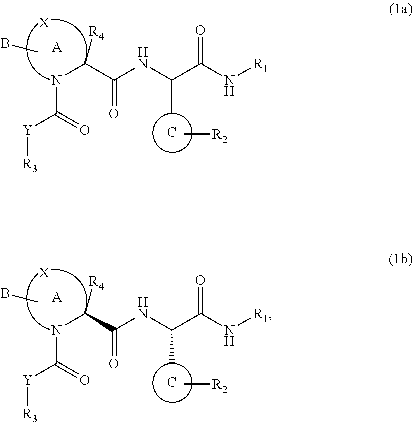
wherein
R¹ is independently selected from the group consisting of optionally substituted C₁-C₁₀-alkyl, C₁-C₁₀-heteroalkyl, C₁-C₁₀-haloalkyl, C₂-C₁₀-alkenyl, C₂-C₁₀-heteroalkenyl, C₂-C₁₀-alkynyl, C₃-C₁₀-cycloalkyl, C₃-C₁₀-heterocycloalkyl, C₄-C₁₀-cycloalkenyl, C₄-C₁₀-heterocycloalkenyl, C₅-C₁₄-aryl, C₅-C₁₄-heteroaryl, C₆-C₁₅ alicyclic system, C₆-C₁₅-aralkyl and C₆-C₁₅-heteroaralkyl;
R² is selected from the group consisting of —F, —Br, —Cl, —R⁵, —OR⁵, —COR⁵, —CO₂R⁵, —OCX₃, —N(R)R′, —N(R)—C(O)—R′, —C(O)—N(R)—C(O)—R′, —N(R)—C(O)—OR′, —C(O)N(R)R′, —N(R)—C(O)—N(R′)R″, —R═NR′, —R═NH, —CN, —NC, —ONO, —NO₂, —ONO₂, —NO, —OCN, —NCO, —SR, —SX₃, —SX₅, —S(O)R, —SO₂R, and —SO₃H, wherein
R⁵ is selected from the group consisting of —H and optionally substituted C₁-C₁₀ alkyl, C₂-C₁₀-alkenyl, C₂-C₁₀-alkynyl, C₃-C₁₀-cycloalkyl, C₃-C₁₀-heterocycloalkyl, C₅-C₁₄-aryl, C₅-C₁₄-heteroaryl, C₆-C₁₅-aralkyl, C₆-C₁₅-heteroaralkyl, and —(OCH₂CH₂)ₙ₁—NH—R⁶, wherein
n1 is 2-10, particularly 4-8, and R⁶ is a biotin moiety linked via an amide bond;
R, R′ and R″ are independently selected from the group consisting of —H, C₁-C₃-alkyl and C₂-C₃-alkenyl;
R³ is independently selected from the group consisting of optionally substituted C₂-C₁₀-alkyl, C₂-C₁₀-heteroalkyl, C₂-C₁₀-haloalkyl, C₂-C₁₀-alkenyl, C₂-C₁₀-heteroalkenyl, C₂-C₁₀-alkynyl, C₃-C₁₀-cycloalkyl, C₃-C₁₀-heterocycloalkyl, C₄-C₁₀-cycloalkenyl, C₄-C₁₀-heterocycloalkenyl, C₅-C₁₄-aryl, C₅-C₁₄-heteroaryl, C₆-C₁₅ alicyclic system, C₆-C₁₅-aralkyl and C₆-C₁₅-heteroaralkyl R⁴ is selected from the group consisting of —H and optionally substituted C₁-C₁₀-alkyl, C₂-C₁₀-alkenyl;
A is a 4- to 8-membered, in particular 5- or 6-membered heterocycloalkyl or heteroaryl;
B is a 4- to 8-membered cycloalkyl, heterocycloalkyl, aryl or heteroaryl, wherein B may be present or absent and wherein if B is present, A and B together form a double cycle;
C is a 5- or 6-membered aryl or heteroaryl;
X is selected from the group consisting of —CH₂—, —NH—, —O—, —S—, —*CHF—, —*CHO(R^{X})—, —*CHNH(R^{X})—, —*CH—CH(R^{X})—, —*C═C(R^{X})—, —*CH—N(R^{X})—, wherein R^{X} represents hydrogen, methyl, ethyl, cyclopropyl, or —CH₂-cyclopropyl, and wherein the *C is part of heterocycloalkyl or heteroaryl A; and
Y is selected from —*CH₂—, —*NH—, —*O—, —*CH₂—CH(R^{Y})—, —*CH═C(R^{Y})—, —*CH₂—O—, —**CH₂—N(R^{Y})— and —*O—CH₂—, wherein R^{Y} represents hydrogen, methyl, ethyl, cyclopropyl, or —CH₂-cyclopropyl, wherein Y may optionally be absent, and wherein the *C, *N or *O is covalently linked to the C atom of the carbonyl group.

2. The compound for use according to claim 1, characterized by a formula (3a) or (3b), in particular (3b), 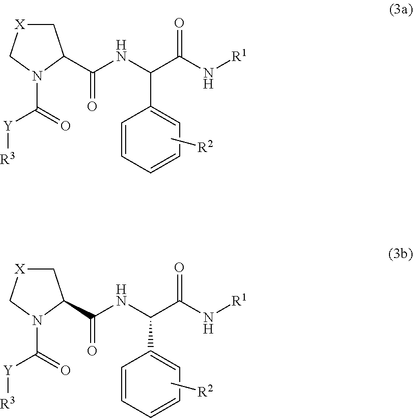
wherein R¹, R², R³, and Y are defined as in claim 1 and X is selected from the group consisting of —CH₂—, —NH—, —O—, —S—, —*CHF—, —*CHO(R^{X})—, —*CHNH(R^{X})—, —*CH—CH(R^{X})—, —*C═C(R^{X})—, —*CH—N(R^{X})—, wherein R^{X} represents hydrogen, methyl, ethyl, cyclopropyl, or —CH₂-cyclopropyl, and wherein the *C is part of the heterocycloalkyl.

3. The compound for use according to claim 1 or 2, wherein R¹ is 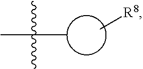
wherein 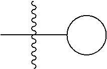
is a 5- to 12-membered, i.e. 5-, 6-, 7-, 8-, 9-, 10-, 11-, or 12-membered alicyclic system, aryl or heteroaryl, in particular a 5- or 6-membered aryl or heteroaryl, and R⁸ is selected from H, R⁹ and OR⁹, wherein R⁹ is selected from optionally substituted C₁-, C₂-, C₃-, C₄-C₅-, C₆- or C₇-alkyl and optionally substituted C₃-, C₄-C₅-, C₆- or C₇-cycloalkyl, wherein the aryl or heteroaryl and R⁹ may form a double cycle.

4. The compound for use according to any one of claims 1 to 3, wherein
R¹ is selected from 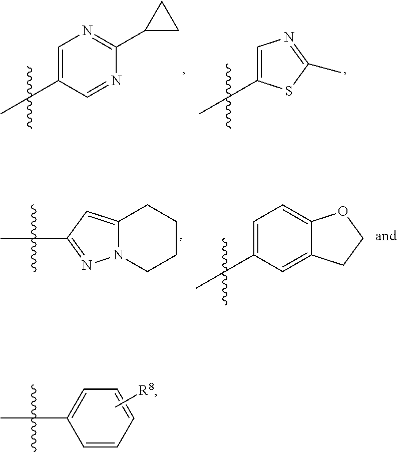
and wherein R⁸ is selected from R⁹ and OR⁹, preferably OR⁹, wherein R⁹ is an unsubstituted C₁-, C₂-, C₃-, or C₄-alkyl.

5. The compound for use according to any one of claims 1 to 4, wherein
R³ is (CH₂)ₙ₂—R⁷, wherein
n2 is 1-4, i.e. 1, 2, 3, or 4, and
R⁷ is selected from the group consisting of optionally substituted C₁-C₇-alkyl, C₄-C₇-cycloalkyl, C₅-C₁₀-aryl and C₅-C₁₀-heteroaryl, and
Y is absent or selected from —CH₂—, —O—, —*CH₂—CH₂—, —*CH₂—O—, and —*O—CH₂—, particularly from —CH₂—, —O—, —*CH₂—CH₂—, and —*CH₂—O—, more particularly from CH₂—, —*CH₂—CH₂—, and —*CH₂—O— wherein the *C or *O is covalently linked to the C atom of the carbonyl group.

6. The compound for use according to claim 5, wherein R⁷ is selected from

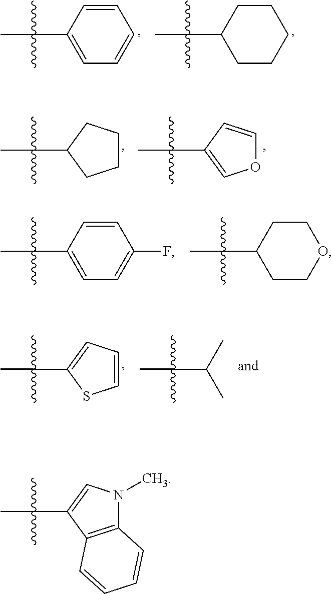

7. The compound for use according to any one of claim 1 to 6, wherein R² is —OR⁵ or —R⁵ and R⁵ is defined as in claim 1, particularly R⁵ is selected from the group consisting of —H, optionally substituted C₁-C₄-alkyl and optionally substituted C₃-C₄-cycloalkyl, more particularly R⁵ is —CH₃.

8. The compound for use according to any one of claims 1 to 7, wherein the compound is characterized by any one of formulae (5)-(34) or (52):

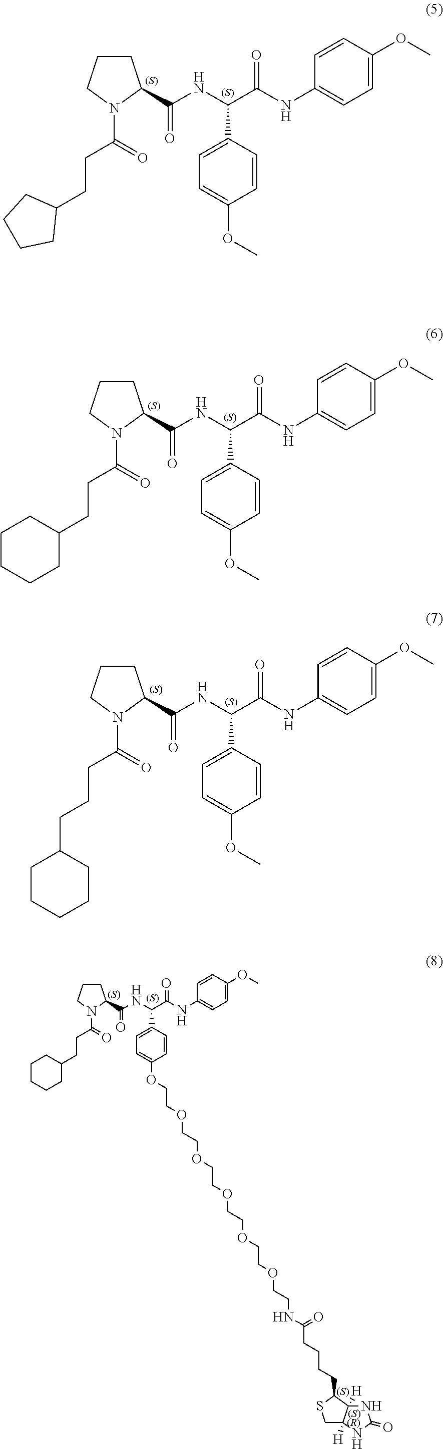

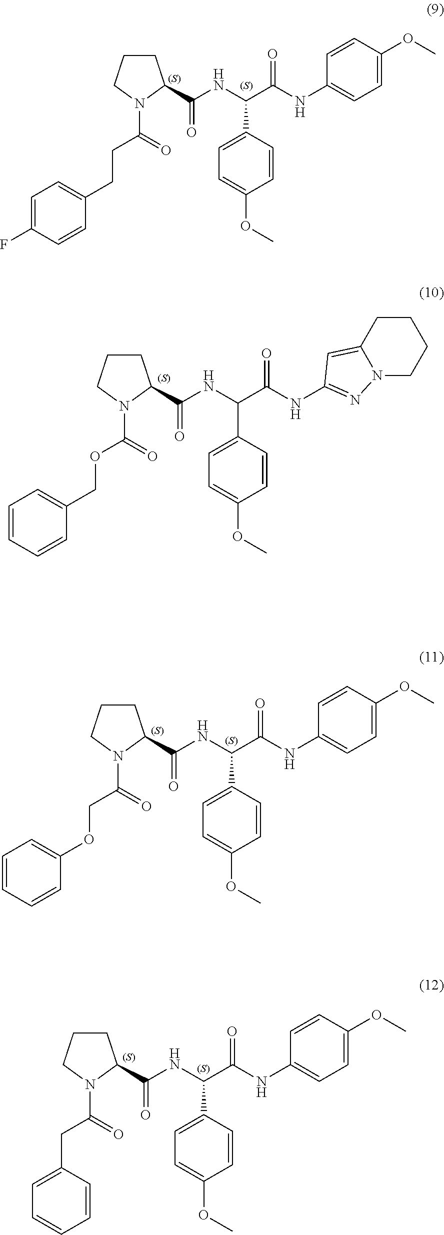

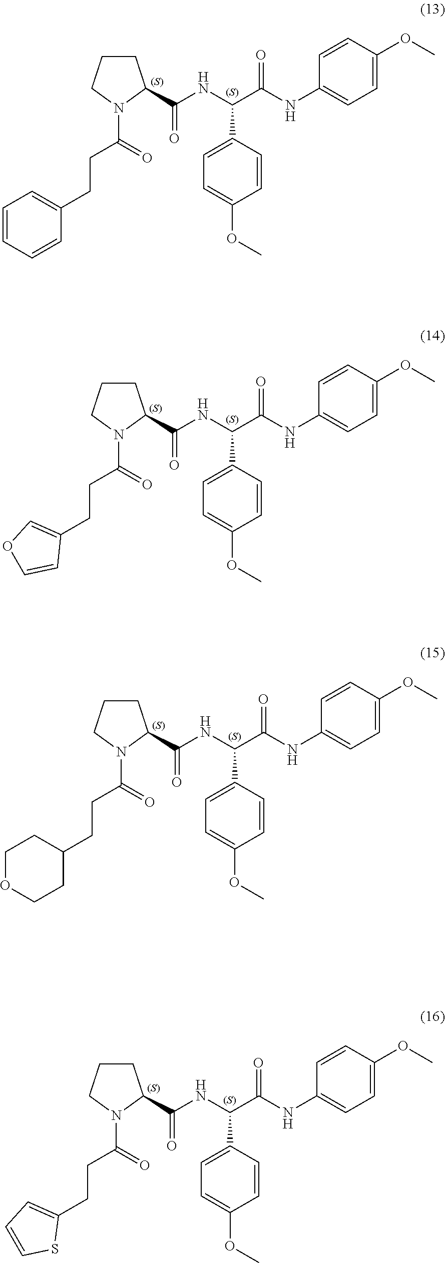

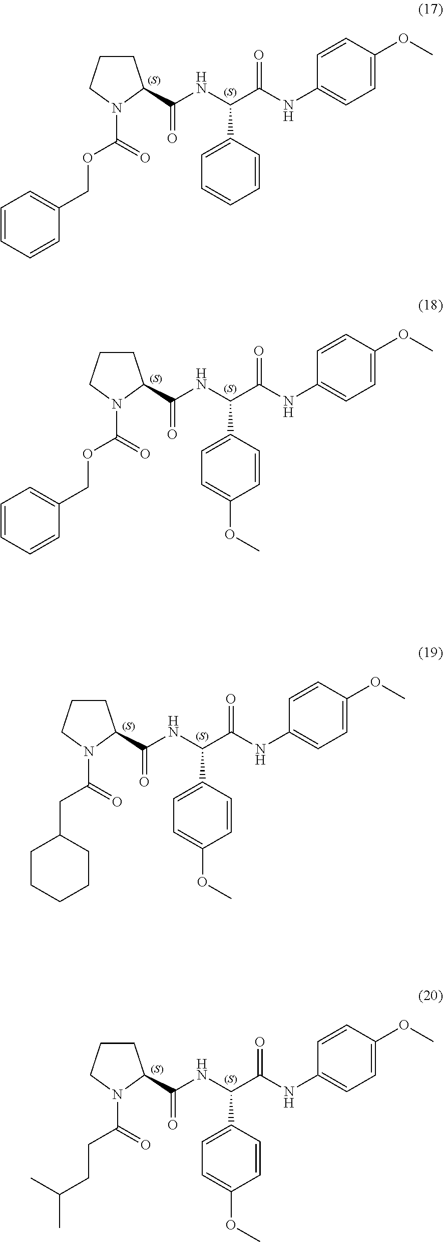

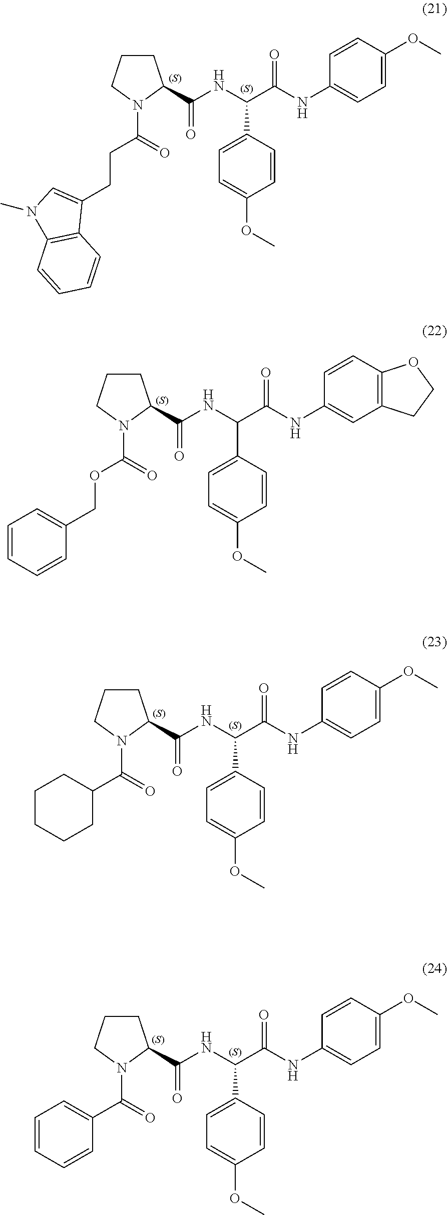

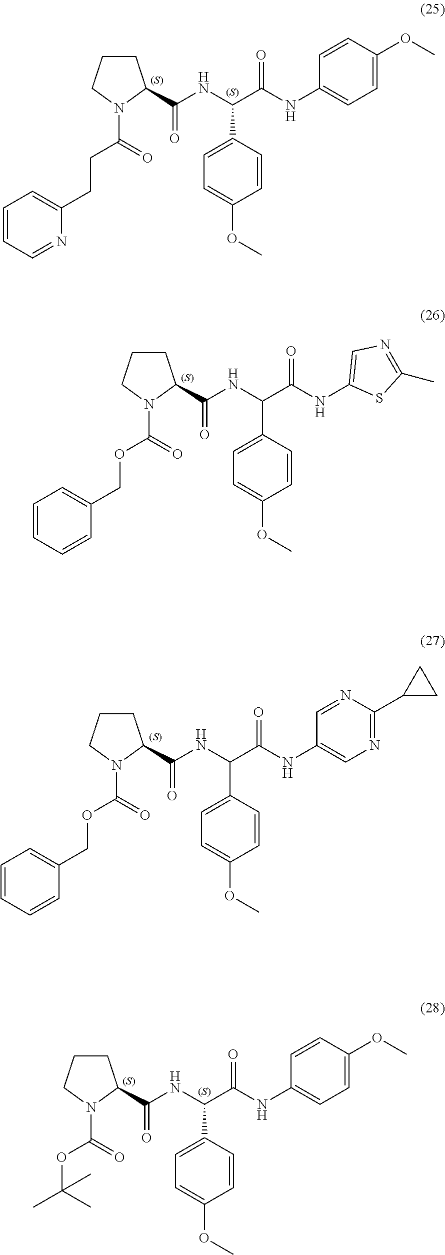

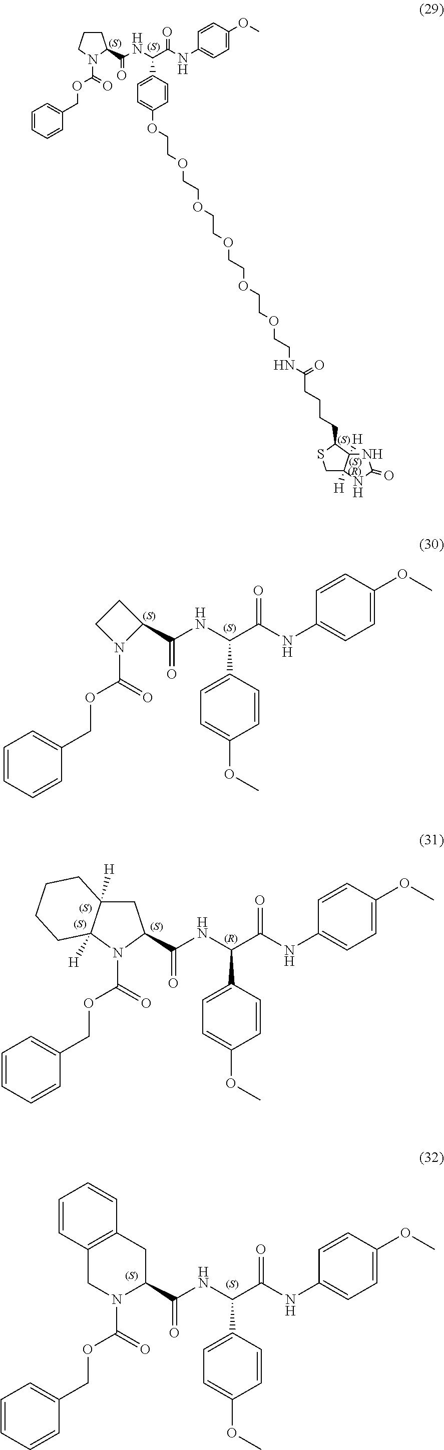

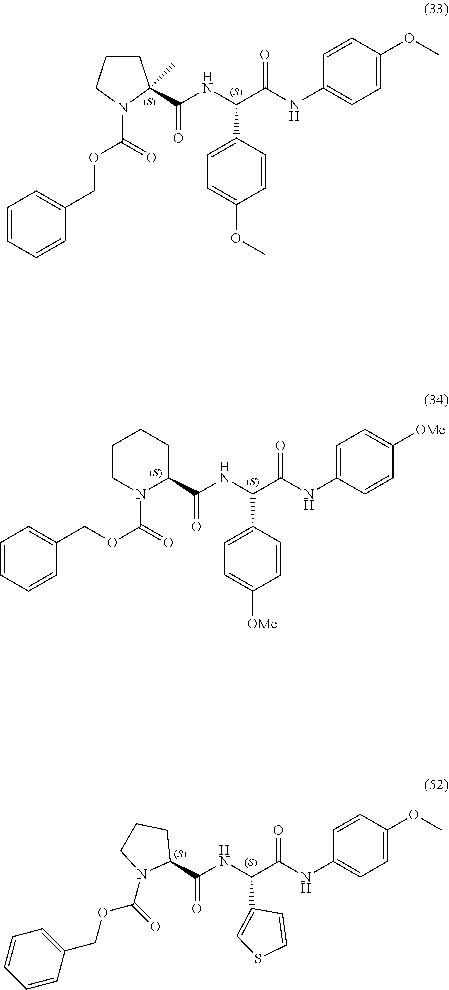

9. The compound for use according to any one of claims 1 to 8, wherein the disease is selected from the group consisting of ischemic stroke, heart failure with preserved ejection fraction (HFpEF), diabetic nephropathy, arthritis, migraine, multiple sclerosis, meningitis, asthma, optic nerve degeneration, posterior retinal degeneration, glaucoma, age-related macular degeneration, cataracts, uveitis, retinal vascular disorder, retinal ischemic damage and reperfusion injury, thrombocytic occlusion of the retinal vein, retinopathy, hypertensive retinopathy, ischaemic retinopathy, diabetic retinopathy, and proliferative ischaemic/diabetic retinopathy.

10. The compound for use according to any one of claims 1 to 9, wherein the compound is an inhibitor of NO production.

11. A compound characterized by a formula (1a) or (1b), in particular (1b), 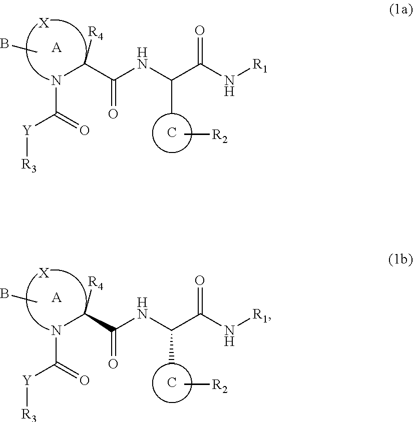
wherein
R¹ is independently selected from the group consisting of optionally substituted C₁-C₁₀-alkyl, C₁-C₁₀-heteroalkyl, C₁-C₁₀-haloalkyl, C₂-C₁₀-alkenyl, C₂-C₁₀-heteroalkenyl, C₂-C₁₀-alkynyl, C₃-C₁₀-cycloalkyl, C₃-C₁₀-heterocycloalkyl, C₄-C₁₀-cycloalkenyl, C₄-C₁₀-heterocycloalkenyl, C₅-C₁₄-aryl, C₅-C₁₄-heteroaryl, C₆-C₁₅ alicyclic system, C₆-C₁₅-aralkyl and C₆-C₁₅-heteroaralkyl;
R² is selected from the group consisting of —F, —Br, —Cl, —R⁵, —OR⁵, —COR⁵, —CO₂R⁵, —OCX₃, —N(R)R′, —N(R)—C(O)—R′, —C(O)—N(R)—C(O)—R′, —N(R)—C(O)—OR′, —C(O)N(R)R′, —N(R)—C(O)—N(R′)R″, —R═NR′, —R═NH, —CN, —NC, —ONO, —NO₂, —ONO₂, —NO, —OCN, —NCO, —SR, —SX₃, —SX₅, —S(O)R, —SO₂R, and —SO₃H, wherein
R⁵ is selected from the group consisting of —H and optionally substituted C₁-C₁₀ alkyl, C₂-C₁₀-alkenyl, C₂-C₁₀-alkynyl, C₃-C₁₀-cycloalkyl, C₃-C₁₀-heterocycloalkyl, C₅-C₁₄-aryl, C₅-C₁₄-heteroaryl, C₆-C₁₅-aralkyl, C₆-C₁₅-heteroaralkyl, and —(OCH₂CH₂)ₙ₁—NH—R⁶, wherein
n1 is 2-10, particularly 4-8, and R⁶ is a biotin moiety linked via an amide bond;
R, R′ and R″ are independently selected from the group consisting of —H, C₁-C₃-alkyl and C₂-C₃-alkenyl;
R³ is independently selected from the group consisting of optionally substituted C₂-C₁₀-alkyl, C₂-C₁₀-heteroalkyl, C₂-C₁₀-haloalkyl, C₂-C₁₀-alkenyl, C₂-C₁₀-heteroalkenyl, C₂-C₁₀-alkynyl, C₃-C₁₀-cycloalkyl, C₃-C₁₀-heterocycloalkyl, C₄-C₁₀-cycloalkenyl, C₄-C₁₀-heterocycloalkenyl, C₅-C₁₄-aryl, C₅-C₁₄-heteroaryl, C₆-C₁₅ alicyclic system, C₆-C₁₅-aralkyl and C₆-C₁₅-heteroaralkyl;
R⁴ is selected from the group consisting of —H and optionally substituted C₁-C₁₀-alkyl and C₂-C₁₀-alkenyl;
A is a 4- to 8-membered, in particular 5- or 6-membered heterocycloalkyl or heteroaryl;
B is a 4- to 8-membered cycloalkyl, heterocycloalkyl, aryl or heteroaryl, wherein B may be present or absent and wherein if B is present, A and B together form a double cycle;
C is a 5- or 6-membered aryl or heteroaryl;
X is selected from the group consisting of —CH₂—, —NH—, —O—, —S—, —*CHF—, —*CHO(R^{X})—, —*CHNH(R^{X})—, —*CH—CH(R^{X})—, —*C═C(R^{X})—, —*CH—N(R^{x})—, wherein R^{X} represents hydrogen, methyl, ethyl, cyclopropyl, or —CH₂-cyclopropyl, and wherein the *C is part of heterocycloalkyl or heteroaryl A; and
Y is selected from —*CH₂—, —*NH—, —*O—, —*CH₂—CH(R^{Y})—, —*CH═C(R^{Y})—, —*CH₂—O—, —**CH₂—N(R^{Y})— and —*O—CH₂—, wherein R^{Y} represents hydrogen, methyl, ethyl, cyclopropyl, or —CH₂-cyclopropyl, wherein Y may optionally be absent, and wherein the *C, *N or *O is covalently linked to the C atom of the carbonyl group.

12. The compound according to claim 11, wherein the compound is characterized by formula (1b).

13. The compound according to claim 11 or 12, wherein
if Y—R³ is 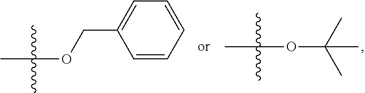
R¹ is not

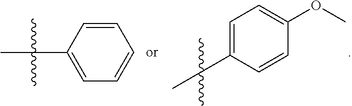

14. The compound according to any one of claims 11 to 13, wherein
Y is —*CH₂—(CH₂)ₙ₃— or —*CH₂—(CH₂)ₙ₄—O—, wherein n3 is 0, 1 or 2 and n4 is 0 or 1, and *C is covalently linked to the C atom of the carbonyl group,
R³ is selected from optionally substituted C₅-C₆ cycloalkyl, C₅-C₆ heterocycloalkyl, C₅-C₆ aryl and C₅-C₅ heteroaryl.

15. A pharmaceutical composition comprising the compound according to any one of claims 1-10.
